(19) United States
(12) Patent Application Publication
(10) Pub. No.: US 2023/0023009 A1
(43) Pub. Date: Jan. 26, 2023

(54) HETEROCYCLIC COMPOUNDS AND METHODS OF USE THEREOF

(71) Applicant: SPARCBIO LLC, San Francisco, CA (US)

(72) Inventors: Jayakanth KANKANALA, Plymouth, MN (US); Jeremy D. PETTIGREW, Burnaby (CA); Son Minh PHAM, San Francisco, CA (US); Sarvajit CHAKRAVARTY, Edmond, OK (US); Jiyun CHEN, Moraga, CA (US)

(21) Appl. No.: 17/339,925

(22) Filed: Jun. 4, 2021

Related U.S. Application Data
(60) Provisional application No. 63/173,323, filed on Apr. 9, 2021.
(60) Provisional application No. 63/035,648, filed on Jun. 5, 2020.

(51) Int. Cl. C07D 498/04; A61P 35/00; C07D 491/052; C07D 491/056; C07D 471/04; C07D 519/00

(52) U.S. Cl. CPC C07D 498/04; A61P 35/00; C07D 519/00; C07D 491/056; C07D 471/04; C07D 491/052

(57) ABSTRACT

The present disclosure relates to inhibitors of one or more isoforms of RAS, such as inhibitors of one or more of KRAS, HRAS and NRAS, or mutants thereof, such as G12D, G12V, G13D or G12C mutants thereof. Therapeutic methods of treating conditions and diseases using these inhibitors are also provided.

### CROSS-REFERENCE TO RELATED APPLICATIONS

This application claims priority to and the benefit of U.S. Provisional Patent Application Ser. No. 63/173,323, filed Apr. 9, 2021, and U.S. Provisional Patent Application Ser. No. 63/035,648, filed Jun. 5, 2020, each of which is hereby incorporated herein by reference in its entirety.

### FIELD

The present disclosure relates to inhibitors of one or more isoforms of RAS, such as inhibitors of one or more of KRAS, HRAS and NRAS, or mutants thereof, such as G12D, G12V, G13D or G12C mutants thereof. Therapeutic methods of treating conditions and diseases using these inhibitors are also provided.

### BACKGROUND

KRAS, HRAS and NRAS are members of the family of RAS genes that were the first human oncogenes to be discovered, and are frequently mutated in cancer. A critical node in growth factor signaling pathways, KRAS, for example, regulates the proliferation, survival, migration and differentiation of cells. The protein is a monomeric GTPase that cycles between an inactive GDP-bound form and an active GTP-bound form, the active form interacting with downstream effector proteins to promote proliferation and other cellular processes. Activating mutations in KRAS drive many cancers, including pancreatic cancer, lung adenocarcinoma and colorectal cancer. Activating mutations often occur in codon 12 or 13 (P-loop), with the G12D, G12V, G13D and G12C mutations being the most common. Although KRAS has been recognized as an important cancer target for decades, intensive research efforts have not yet resulted in an approved therapeutic that targets it. Thus, there remains a need to develop safe and effective inhibitors against members of the RAS family, such as inhibitors against one or more of KRAS, HRAS and NRAS.

### SUMMARY

The present disclosure provides compounds that inhibit activity of one or more members of the RAS family of proteins, such as one or more of the KRAS, HRAS and NRAS proteins, or mutants thereof, such as a G12D, G12V, G13D or G12C mutant thereof. These compounds can be useful in treating cancer, in particular those cancers that are driven by activating mutations in either KRAS, HRAS or NRAS such as the G12C mutation.

In one aspect, provided is a compound of formula (I):

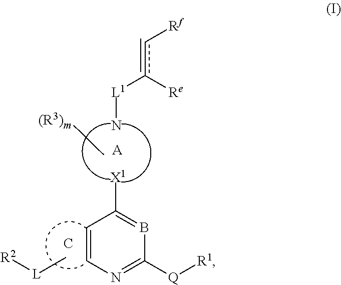

or a pharmaceutically acceptable salt, stereoisomer, mixture of stereoisomers, solvate, or tautomer thereof, wherein [symbol] , A, B, [symbol] , Q, X¹, R¹, R², R³, L¹, L, m, R^{e}, and R^{f} are as detailed herein.

In another aspect, provided is a method of treating a KRAS-, HRAS- or NRAS-mediated disease in an individual in need thereof, comprising administering an effective amount of a compound as described herein, or pharmaceutically acceptable salt thereof, to the individual. In some embodiments, provided is a method of treating a KRAS-, HRAS- or NRAS-mediated disease in an individual at risk of developing the disease, comprising administering an effective amount of a compound as described herein, or pharmaceutically acceptable salt thereof, to the individual. In some embodiments, the disease expresses a mutant RAS, such as a disease that expresses a G12D, G12V, G13D or G12C mutant. In some embodiments, the disease expresses KRAS G12C. In some embodiments, the disease expresses HRAS G12C. In some embodiments, the disease expresses NRAS G12C. In some embodiments, the disease is a cancer. In some embodiments, the cancer is lung, colorectal, or pancreatic cancer. In some embodiments, the cancer is MYH-associated polyposis, biliary tract cancer or hematologic malignancies. In some embodiments, the method further comprises administering an additional anti-cancer therapeutic agent, such as a chemotherapeutic agent.

Also provided herein are compositions, including pharmaceutical compositions, that comprise a compound as detailed herein or a pharmaceutically acceptable salt thereof. Also provided are kits that comprise a compound as detailed herein or a pharmaceutically acceptable salt thereof, and methods of using (or administering) and making such compounds and pharmaceutically acceptable salts thereof. The disclosure further provides compounds or compositions thereof for use in a method of treating a RAS-mediated disease, including a KRAS-, HRAS- or NRAS-mediated disease. Moreover, the disclosure provides uses of the compounds or compositions thereof in the manufacture of a medicament for the treatment of a KRAS-, HRAS- or NRAS-mediated disease.

### BRIEF DESCRIPTION OF THE DRAWINGS

FIG. 1 illustrates the tumor growth curves of different treatment groups of female BALB/c nude mice bearing NCI-H358 subcutaneous xenograft tumors.

FIG. 2 illustrates the tumor growth curves of different treatment groups of female BALB/c nude mice bearing MIA PaCa-2 subcutaneous xenograft tumors.

FIG. 3 illustrates the survival curves of different treatment groups of female BALB/c nude mice bearing MIA PaCa-2 subcutaneous xenograft tumors.

FIG. 4A depicts unit cell a of compound 43 (Isomer A); FIG. 4B depicts unit cell b of compound 43 (Isomer A); FIG. 4C depicts unit cell c of compound 43 (Isomer A).

FIG. 5 depicts a simulated XRPD (100K) of compound 43 (Isomer A).

### DETAILED DESCRIPTION

The following description sets forth exemplary embodiments of the present technology. It should be recognized, however, that such description is not intended as a limitation on the scope of the present disclosure but is instead provided as a description of exemplary embodiments.

### Definitions

As used in the present specification, the following words, phrases and symbols are generally intended to have the meanings as set forth below, except to the extent that the context in which they are used indicates otherwise.

The term “about” refers to a variation of ±1%, ±3%, ±5%, or ±10% of the value specified. For example, “about 50” can in some embodiments include a range of from 45 to 55. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. Reference to “about” a value or parameter herein includes (and describes) embodiments that are directed to that value or parameter per se. For example, description referring to “about X” includes description of “X”.

The singular forms “a” and “the” include plural references unless the context clearly dictates otherwise. Thus, e.g., reference to “the compound” includes a plurality of such compounds and includes reference to one or more compounds and equivalents thereof known to those skilled in the art.

“Alkyl” refers to an unbranched or branched saturated hydrocarbon chain. As used herein, alkyl has 1 to 10 carbon atoms (i.e., C₁₋₁₀ alkyl or C₁-C₁₀ alkyl), 1 to 8 carbon atoms (i.e., C₁₋₈ alkyl or C₁-C₈ alkyl), 1 to 6 carbon atoms (i.e., C₁₋₆ alkyl or C₁-C₆ alkyl), or 1 to 4 carbon atoms (i.e., C₁₋₄ alkyl or C₁-C₄ alkyl). Examples of alkyl groups include, without limitation, methyl, ethyl, propyl, isopropyl, n-butyl, sec-butyl, isobutyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e. —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

“Alkylene” refers to a divalent alkyl group as defined herein.

“Haloalkyl” refers to an unbranched or branched alkyl group as defined above, wherein one or more hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include difluoromethyl (—CHF₂) and trifluoromethyl (—CF₃).

“Hetero alkyl” refers to an alkyl group in which one or more of the carbon atoms (and any associated hydrogen atoms) are each independently replaced with the same or different heteroatomic group. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NH—, —O—, —S—, —S(O)—, —S(O)₂— and the like. As used herein, heteroalkyl includes 1 to 8 carbon atoms, or 1 to 4 carbon atoms; and 1 to 3 heteroatoms, 1 to 2 heteroatoms, or 1 heteroatom.

“Alkoxy” refers to the group “—O-alkyl”. Examples of alkoxy groups include, without limitation, methoxy, ethoxy, n-propoxy, isopropoxy, n-butoxy, tert-butoxy, sec-butoxy, n-pentoxy, n-hexoxy and 1,2-dimethylbutoxy.

“Alkenyl” refers to an alkyl group containing at least one carbon-carbon double bond and having from 2 to 20 carbon atoms (i.e., C₂₋₂₀ alkenyl or C₂-C₂₀ alkenyl), 2 to 8 carbon atoms (i.e., C₂₋₈ alkenyl or C₂-C₈ alkenyl), 2 to 6 carbon atoms (i.e., C₂₋₆ alkenyl or C₂-C₆ alkenyl) or 2 to 4 carbon atoms (i.e., C₂₋₄ alkenyl or C₂-C₄ alkenyl). Examples of alkenyl groups include, without limitation, ethenyl, propenyl, and butadienyl (e.g., 1,2-butadienyl and 1,3-butadienyl).

“Alkynyl” refers to an alkyl group containing at least one carbon-carbon triple bond and having from 2 to 20 carbon atoms (i.e., C₂₋₂₀ alkynyl or C₂-C₂₀ alkynyl), 2 to 8 carbon atoms (i.e., C₂₋₈ alkynyl or C₂-C₈ alkynyl), 2 to 6 carbon atoms (i.e., C₂₋₆ alkynyl or C₂-C₆ alkynyl) or 2 to 4 carbon atoms (i.e., C₂₋₄ alkynyl or C₂-C₄ alkynyl). The term “alkynyl” also includes those groups having one triple bond and one double bond.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl or C₆-C₂₀ aryl), 6 to 12 carbon ring atoms (i.e., C₆₋₁₂ aryl or C₆-C₁₂ aryl), or 6 to 10 carbon ring atoms (i.e., C₆₋₁₀ aryl or C₆-C₁₀ aryl). Examples of aryl groups include, without limitation, phenyl, naphthyl, fluorenyl and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl or C₃-C₂₀ cycloalkyl), 3 to 12 ring carbon atoms (i.e., C₃₋₁₂ cycloalkyl or C₃-C₁₂ cycloalkyl), 3 to 10 ring carbon atoms (i.e., C₃₋₁₀ cycloalkyl or C₃-C₁₀ cycloalkyl), 3 to 8 ring carbon atoms (i.e., C₃₋₈ cycloalkyl or C₃-C₈ cycloalkyl), or 3 to 6 ring carbon atoms (i.e., C₃₋₆ cycloalkyl or or C₃-C₆ cycloalkyl). Monocyclic groups include, without limitation, cyclopropyl, cyclobutyl, cyclopentyl, cyclohexyl, cycloheptyl and cyclooctyl. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom.

“Heteroaryl” refers to an aromatic group having a single ring, multiple rings or multiple fused rings, with one or more ring heteroatoms independently selected from nitrogen, oxygen and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl) and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 5-12 membered ring systems, 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” refers to a saturated or partially unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro and may comprise one or more (e.g., 1 to 3) oxo (═O) or N-oxide (N⁺—O⁻) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂₋₂₀ or C₂-C₂₀ heterocyclyl), 2 to 12 ring carbon atoms (i.e., C₂₋₁₂ or C₂-C₁₂ heterocyclyl), 2 to 10 ring carbon atoms (i.e., C₂₋₁₀ or C₂-C₁₀ heterocyclyl), 2 to 8 ring carbon atoms (i.e., C₂₋₈ or C₂-C₈ heterocyclyl), 3 to 12 ring carbon atoms (i.e., C₃₋₁₂ or C₃-C₁₂ heterocyclyl), 3 to 8 ring carbon atoms (i.e., C₃₋₈ or C₃-C₈ heterocyclyl), or 3 to 6 ring carbon atoms (i.e., C₃₋₆ or C₃-C₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. In certain instances, heterocyclyl includes 3-12 membered ring systems, 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur. The term “heterocyclyl” also includes “spiroheterocyclyl” when there are two positions for substitution on the same carbon atom.

“Oxo” refers to ═O.

“Halogen” or “halo” includes fluoro, chloro, bromo and iodo.

The terms “optional” or “optionally” means that the subsequently described event or circumstance may or may not occur. The term “optionally substituted” refers to any one or more hydrogen atoms on the designated atom or group may or may not be replaced by a moiety other than hydrogen.

“Substituted” as used herein means one or more (e.g., 1-5, 1-4, 1-3, 1-2, 2-5, 2-4, 2-3, 3-5, or 3-4) hydrogen atoms of the group is replaced with a substituent atom or group commonly used in pharmaceutical chemistry. Each substituent can be the same or different. Examples of suitable substituents include, but are not limited to, hydrazide, halo, —CN, —NO₂, alkyl, alkenyl, alkynyl, cycloalkyl, aryl, heterocyclyl, heteroaryl, —OR⁵⁶, —C(O)OR⁵⁶, —C(O)R⁵⁶, —O-alkyl-OR⁵⁶, alkyl-OR⁵⁶, haloalkyl, haloalkoxy, —SR⁵⁶, —S(O)R⁵⁶, —SO₂R⁵⁶, —NR⁵⁶R⁵⁷, —C(O)NR⁵⁶R⁵⁷, —NR⁵⁶C(O)R⁵⁷, including seleno- and thio-derivatives thereof, wherein each R⁵⁶ and R⁵⁷ are independently hydrogen, alkyl, alkenyl, alkynyl, heteroalkyl, cycloalkyl, cycloalkyl-alkyl-, heterocyclyl, heterocyclyl-alkyl-, aryl, aryl-alkyl-, heteroaryl, or heteroaryl-alkyl- and wherein each of the substituents can be optionally further substituted.

Provided are also are stereoisomers, mixture of stereoisomers, tautomers, hydrates, solvates, isotopically enriched analogs and pharmaceutically acceptable salts of the compounds described herein.

The compounds disclosed herein, or their pharmaceutically acceptable salts, may include an asymmetric center and may thus give rise to enantiomers, diastereomers and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)- isomers may be prepared using chiral synthons or chiral reagents or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-performance liquid chromatography (HPLC). When the compounds described herein contain olefinic double bonds or other centers of geometric asymmetry and unless specified otherwise, it is intended that the compounds include both E- and Z- geometric isomers.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are nonsuperimposable mirror images of one another and “diastereomers,” which refers to stereoisomers that have at least two asymmetric atoms, but which are not mirror-images of each other. Thus, all stereoisomers (for example, geometric isomers, optical isomers and the like) of the present compounds (including those of the salts, solvates and hydrates of the compounds), such as those which may exist due to asymmetric carbons on various substituents, including enantiomeric forms (which may exist even in the absence of asymmetric carbons), rotameric forms, atropisomers and diastereomeric forms, are contemplated.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds disclosed herein, e.g., Formula I, may be atropisomers and are considered as part of this disclosure. Stereoisomers can also be separated by use of chiral HPLC.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Any compound or structure given herein, is also intended to represent unlabeled forms as well as isotopically labeled forms of the compounds. These forms of compounds may also be referred to as an “isotopically enriched analog.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I and ¹²⁵I, respectively. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients. Such compounds may exhibit increased resistance to metabolism and are thus useful for increasing the half-life of any compound when administered to a mammal, particularly a human. Such compounds are synthesized by means well known in the art, for example by employing starting materials in which one or more hydrogens have been replaced by deuterium.

Certain compounds disclosed herein contain one or more ionizable groups (groups from which a proton can be removed (e.g., —COOH) or added (e.g., amines) or which can be quaternized (e.g., amines). All possible ionic forms of such molecules and salts thereof are intended to be included individually in the disclosure herein. With regard to salts of the compounds described herein, one of ordinary skill in the art can select from among a wide variety of available counterions those that are appropriate. In specific applications, the selection of a given anion or cation for preparation of a salt may result in increased or decreased solubility of that salt.

The terms “inhibit,” “inhibiting,” and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

“Individual” as used herein is a mammal, including humans. In some embodiments, individuals includes pig, bovine, feline, canine, primate, rodent, or human. In some embodiments, the individual is human.

As used herein, “treatment” or “treating” is an approach for obtaining beneficial or desired results including clinical results. For purposes of this disclosure, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delaying or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of this disclosure contemplate any one or more of these aspects of treatment.

As used herein, by “combination therapy” is meant a therapy that includes two or more different compounds or therapeutic agents. Thus, in one aspect, a combination therapy comprising a compound detailed herein and another compound or therapeutic agent is provided. In some variations, the combination therapy optionally includes one or more pharmaceutically acceptable carriers or excipients, non-pharmaceutically active compounds and/or inert substances. In various embodiments, treatment with a combination therapy may result in an additive or even synergistic (e.g., greater than additive) result compared to administration of a single compound of the disclosure alone. In some embodiments, a lower amount of each compound is used as part of a combination therapy compared to the amount generally used for individual therapy. Preferably, the same or greater therapeutic benefit is achieved using a combination therapy than by using any of the individual compounds alone. In some embodiments, the same or greater therapeutic benefit is achieved using a smaller amount (e.g., a lower dose or a less frequent dosing schedule) of a compound in a combination therapy than the amount generally used for individual compound or therapy. Preferably, the use of a small amount of compound results in a reduction in the number, severity, frequency and/or duration of one or more side-effects associated with the compound.

The term “effective amount” used herein refers to an amount of a compound or composition sufficient to treat a specified disorder, condition or disease such as ameliorate, palliate, lessen, and/or delay one or more of its symptoms. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay occurrence and/or recurrence. An effective amount can be administered in one or more administrations. In the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

The term “carrier,” as used herein, refers to relatively nontoxic chemical compounds or agents that facilitate the incorporation of a compound into cells or tissues.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide and the like. Further examples of pharmaceutically acceptable salts include those listed in Berge et al., Pharmaceutical Salts, J. Pharm. Sci. 1977 January; 66(1):1-19. Pharmaceutically acceptable salts can be prepared in situ in the manufacturing process, or by separately reacting a purified compound of the disclosure in its free acid or base form with a suitable organic or inorganic base or acid, respectively and isolating the salt thus formed during subsequent purification. It should be understood that a reference to a pharmaceutically acceptable salt includes the solvent addition forms or crystal forms thereof, particularly solvates or polymorphs. Solvates contain either stoichiometric or non-stoichiometric amounts of a solvent and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability and solubility. Various factors such as the recrystallization solvent, rate of crystallization and storage temperature may cause a single crystal form to dominate.

The term “excipient” as used herein means an inert or inactive substance that may be used in the production of a drug or pharmaceutical, such as a tablet containing a compound of the disclosure as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

### Compounds

In one aspect, provided herein is a compound of formula (I):

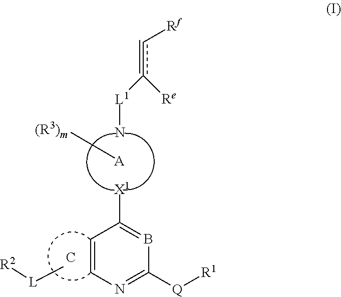

or a pharmaceutically acceptable salt, stereoisomer, mixture of stereoisomers, solvate, or tautomer thereof, wherein:
A is a 4-12 membered saturated or partially saturated monocyclic, bridged or spiro ring; B is N or CR^{a}; [symbol]
is

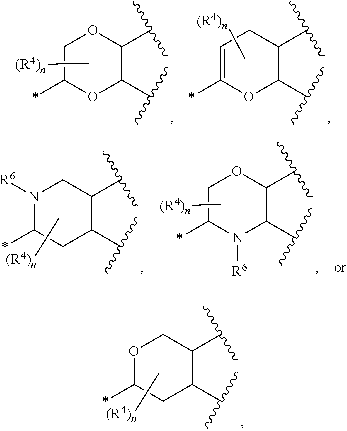

wherein * denotes the point of attachment to L;

### X¹ is C, CH, or N;

Q is —O—, —S—, —NR⁵—, C₁-C₃ alkylene, or a bond;
m is 0, 1, 2, or 3;
n is 0, 1, 2, or 3;
R¹ is hydrogen, C₁-C₆ alkyl, C₁-C₆ haloalkyl, —(C₁-C₆ alkylene)OR^{c}, —(C₁-C₆ alkylene)NR^{c}R^{d}, —NR^{c}R^{d}, 3-12 membered heterocyclyl, C₃-C₁₂ cycloalkyl, —(C₁-C₆ alkylene) 3-12 membered heterocyclyl, C₆-C₁₂ aryl, 5-12 membered heteroaryl, or —(C₁-C₆ alkylene) 5-12 membered heteroaryl, each of which is optionally substituted with one or more R^{1a};
R² is C₃-C₁₂ cycloalkyl, 3-12 membered heterocyclyl, 5-12 membered heteroaryl, or C₆-C₁₂ aryl, each of which is optionally substituted with one or more R^{2a};
L is a bond, —C(O)—, C₁-C₃ alkylene, —O—, —S—, —S(O)—, —S(O)₂—, or —NR⁵—; L¹ is —C(O)— or —S(O)₂—;
R^{2a} and R³ are each independently oxo, C₃-C₈ cycloalkyl, 3-12 membered heterocyclyl, halogen, hydroxyl, C₁-C₆ alkoxy, C₁-C₆ haloalkyl, C₁-C₆ haloalkoxy, C₁-C₆ alkyl, C₂-C₆ alkynyl, C₁-C₆ heteroalkyl, —CN, —OR^{g}, —C(O)OR^{g}, —C(O)N(R^{g}R^{h}), or —N(R^{g}R^{h}), each of which is optionally substituted with one or more substituents selected from —CN, halogen, —ORⁱ, —N(RⁱR^{j}) and 5-12 membered heteroaryl;
R^{1a} and R⁴ are each independently hydrogen, oxo, —C(O)R^{g}, hydroxyl, —(C₁-C₆ alkylene)OR^{g}, —CN, halogen, C₁-C₆ alkyl, —(C₁-C₆ alkylene) C₆-C₁₂ aryl, C₁-C₆ haloalkyl, C₁-C₆ heteroalkyl, C₃-C₈ cycloalkyl, 3-12 membered heterocyclyl, —(C₁-C₆ alkylene) 3-12 membered heterocyclyl, C₁-C₆ alkoxy, —(C₁-C₆ alkylene)C(O)N(R^{g}R^{h}), —(C₁-C₆ alkylene)N(R^{g}R^{h}), —S(O)₂R^{g}, —C(O)OR^{g}, —C(O)N(R^{g}R^{h}), or —N(R^{g}R^{h}), each of which is optionally substituted with one or more substituents selected from —ORⁱ, C₃-C₁₂ cycloalkyl, and —CN;
each R⁵ is independently hydrogen, C₁-C₃ alkyl, or C₃-C₆ cycloalkyl;
each R⁶ is independently hydrogen, C₁-C₆ haloalkyl, C₁-C₆ alkyl, C₃-C₈ cycloalkyl, C₁-C₆ haloalkoxy, C₁-C₆ alkoxy, —C(O)R^{c}, —S(O)₂R^{b}, —C(O)OR^{c}, —C(O)N(R^{c}R^{d}), C₆-C₁₂ aryl, 3-12 membered heterocyclyl, or 5-12 membered heteroaryl, each of which is optionally substituted with one or more R³;
each R^{a} is independently hydrogen, —CN, C₁-C₆ alkoxy, C₁-C₆ haloalkoxy, —C(O)R^{c}, —S(O)₂R^{b}, —C(O)OR^{c}, —C(O)N(R^{c}R^{d}), —N(R^{c}R^{d}), C₆-C₁₂ aryl, 3-12 membered heterocyclyl, 5-12 membered heteroaryl, C₁-C₆ alkoxy, halogen, —N(R^{c})C(O)N(R^{c}R^{d}), or —N(R^{c})C(O)R^{d}, each of which is optionally substituted with one or more R³;
each R^{b} is independently hydrogen, —CN, C₁-C₆ alkyl, C₁-C₆ haloalkyl, C₃-C₈ cycloalkyl, —C(O)R^{c}, —C(O)OR^{c}, —C(O)N(R^{c}R^{d}), —N(R^{c}R^{d}), C₆-C₁₂ aryl, 3-12 membered heterocyclyl, 5-12 membered heteroaryl, C₁-C₆ alkoxy, halogen, —N(R^{c})C(O)N(R^{c}R^{d}), or —N(R^{c})C(O)R^{d}, each of which is optionally substituted with one or more R³;
R^{c} and R^{d} are each independently hydrogen, C₁-C₆ alkyl, C₆-C₁₂ aryl, 3-12 membered heterocyclyl, 5-12 membered heteroaryl, C₁-C₆ haloalkyl, or C₃-C₈ cycloalkyl, each of which is optionally substituted with one or more R³,

or R^{c} and R^{d} are taken together with the atom to which they attach to form a 3-12 membered heterocyclyl or 5-12 membered heteroaryl,

[symbol]

is a double bond or a triple bond, provided that

when [symbol] is a double bond, then R^{e} and R^{f} are each independently H, halogen, —CN, —C(O)OR^{g}, C₁-C₆ haloalkyl, C₁-C₆ alkyl, C₁-C₆ heteroalkyl, —C(O)N(R^{g}R^{h}), C₆-C₁₂ aryl, 5-12 membered heteroaryl, 3-12 membered heterocyclyl, —(C₁-C₆ alkylene)OR^{g}, or —(C₁-C₆ alkylene)N(R^{g}R^{h}), or

R^{e} and R^{f} are taken together with the atoms to which they attach to form a C₃-C₁₂ cycloalkyl, 3-12 membered heterocyclyl, or 5-12 membered heteroaryl, and

when [symbol] is a triple bond, then R^{e} is absent and R^{f} is H, halogen, —CN, —C(O)OR^{g}, C₁-C₆ haloalkyl, C₁-C₆ alkyl, C₁-C₆ heteroalkyl, —C(O)N(R^{g}R^{h}), C₆-C₁₂ aryl, 5-12 membered heteroaryl, 3-12 membered heterocyclyl, —(C₁-C₆ alkylene)OR^{g}, or —(C₁-C₆ alkylene)N(R^{g}R^{h}); and R^{g}, R^{h}, Rⁱ, and R^{j} are each independently H, C₁-C₆ alkyl, C₆-C₁₂ aryl, 3-12 membered heterocyclyl, 5-12 membered heteroaryl, C₁-C₆ haloalkyl, C₃-C₈ cycloalkyl, or —NH₂, or

R^{g} and R^{h} or Rⁱ and R^{j} are taken together with the atom to which they attach to form a 3-12 membered heterocyclyl or 5-12 membered heteroaryl.

In some embodiments of formula (I), or a pharmaceutically acceptable salt, stereoisomer, mixture of stereoisomers, solvate, or tautomer thereof, moiety L-R² is connected to a carbon atom of the C ring and the carbon atom to which L-R² is connected is in the (S) stereochemical configuration. In some embodiments of formula (I), or a pharmaceutically acceptable salt, stereoisomer, mixture of stereoisomers, solvate, or tautomer thereof, moiety L-R² is connected to a carbon atom of the C ring and the carbon atom to which L-R² is connected is in the (R) stereochemical configuration.

In some embodiments of a compound of formula (I) or any related formula where applicable, or a pharmaceutically acceptable salt, stereoisomer, mixture of stereoisomers, solvate, or tautomer thereof, is provided wherein the carbon atom to which the R¹ is connected is in the (S) stereochemical configuration. In other such embodiments, a compound of formula (I) or any related formula where applicable, or a pharmaceutically acceptable salt, stereoisomer, mixture of stereoisomers, solvate, or tautomer thereof, is provided wherein the carbon atom to which the R¹ is connected is in the (R) stereochemical configuration. In some embodiments of a compound of formula (I) or any related formula where applicable, or a pharmaceutically acceptable salt, stereoisomer, mixture of stereoisomers, solvate, or tautomer thereof, is provided wherein the carbon atom to which the R² is connected is in the (S) stereochemical configuration. In other such embodiments, a compound of formula (I) or any related formula where applicable, or a pharmaceutically acceptable salt, stereoisomer, mixture of stereoisomers, solvate, or tautomer thereof, is provided wherein the carbon atom to which the R² is connected is in the (R) stereochemical configuration. In some embodiments of a compound of formula (I) or any related formula where applicable, or a pharmaceutically acceptable salt, stereoisomer, mixture of stereoisomers, solvate, or tautomer thereof, is provided wherein the carbon atom to which the R³ is connected is in the (S) stereochemical configuration. In other such embodiments, a compound of formula (I) or any related formula where applicable, or a pharmaceutically acceptable salt, stereoisomer, mixture of stereoisomers, solvate, or tautomer thereof, is provided wherein the carbon atom to which the R³ is connected is in the (R) stereochemical configuration.

It is understood that a spiro ring system has at least two rings with one common atom. It is also understood that a fused ring system has at least two rings with two adjacent common atoms.

In some embodiments of a compound of formula (I), or a pharmaceutically acceptable salt, stereoisomer, mixture of stereoisomers, solvate, or tautomer thereof, [symbol] is.

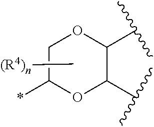

In some embodiments, [symbol] is

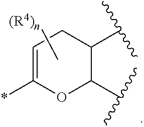

In some embodiments, [symbol] is

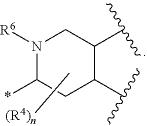

In some embodiments, [symbol] is

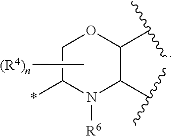

In some embodiments, [symbol] is

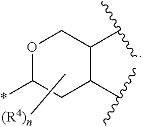

In some embodiments of a compound of formula (I), or a pharmaceutically acceptable salt, stereoisomer, mixture of stereoisomers, solvate, or tautomer thereof, the compound is of formula (II), wherein A, B, m, n, Q, L, L¹, X¹, R¹, R², R³, R⁴, R^{e} and R^{f} are as detailed herein for formula (I).

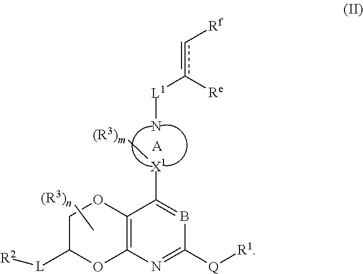

In some embodiments of a compound of formula (I), or a pharmaceutically acceptable salt, stereoisomer, mixture of stereoisomers, solvate, or tautomer thereof, the compound is of formula (III), wherein A, B, m, n, Q, L, L¹, X¹, R¹, R², R³, R⁴, R^{e} and R^{f} are as detailed herein for formula (I). In some embodiments of formula (II), or a pharmaceutically acceptable salt, stereoisomer, mixture of stereoisomers, solvate, or tautomer thereof, the carbon atom to which L-R² is connected is in the (S) stereochemical configuration. In some embodiments of formula (II), or a pharmaceutically acceptable salt, stereoisomer, mixture of stereoisomers, solvate, or tautomer thereof, the carbon atom to which L-R² is connected is in the (R) stereochemical configuration.

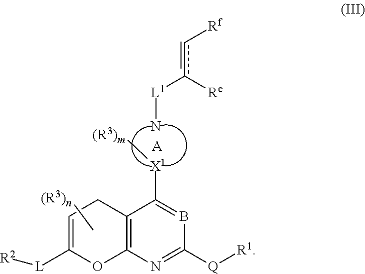

In some embodiments of a compound of formula (I), or a pharmaceutically acceptable salt, stereoisomer, mixture of stereoisomers, solvate, or tautomer thereof, the compound is of formula (IV), wherein A, B, m, n, Q, L, L¹, X¹, R¹, R², R³, R⁴, R⁶, R^{e} and R^{f} are as detailed herein for formula (I). In some embodiments of formula (IV), or a pharmaceutically acceptable salt, stereoisomer, mixture of stereoisomers, solvate, or tautomer thereof, the carbon atom to which L-R² is connected is in the (S) stereochemical configuration. In some embodiments of formula (IV), or a pharmaceutically acceptable salt, stereoisomer, mixture of stereoisomers, solvate, or tautomer thereof, the carbon atom to which L-R² is connected is in the (R) stereochemical configuration.

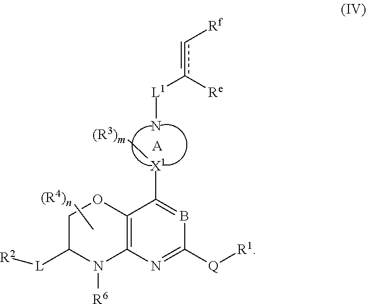

In some embodiments of a compound of formula (I), or a pharmaceutically acceptable salt, stereoisomer, mixture of stereoisomers, solvate, or tautomer thereof, the compound is of formula (V), wherein A, B, m, n, Q, L, L¹, X¹, R¹, R², R³, R⁴, R⁶, R^{e} and R^{f} are as detailed herein for formula (I). In some embodiments of formula (V), or a pharmaceutically acceptable salt, stereoisomer, mixture of stereoisomers, solvate, or tautomer thereof, the carbon atom to which L-R² is connected is in the (S) stereochemical configuration. In some embodiments of formula (V), or a pharmaceutically acceptable salt, stereoisomer, mixture of stereoisomers, solvate, or tautomer thereof, the carbon atom to which L-R² is connected is in the (R) stereochemical configuration.

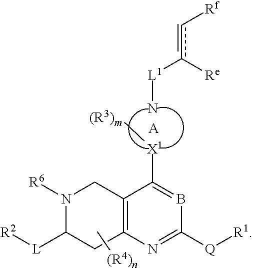

In some embodiments of a compound of formula (I), or a pharmaceutically acceptable salt, stereoisomer, mixture of stereoisomers, solvate, or tautomer thereof, the compound is of formula (VI), wherein A, B, m, n, Q, L, L¹, X¹, R¹, R², R³, R⁴, R^{e} and R^{f} are as detailed herein for formula (I). In some embodiments of formula (VI), or a pharmaceutically acceptable salt, stereoisomer, mixture of stereoisomers, solvate, or tautomer thereof, the carbon atom to which L-R² is connected is in the (S) stereochemical configuration. In some embodiments of formula (VI), or a pharmaceutically acceptable salt, stereoisomer, mixture of stereoisomers, solvate, or tautomer thereof, the carbon atom to which L-R² is connected is in the (R) stereochemical configuration.

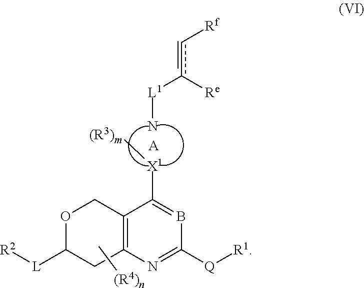

Specific values described herein are values for a compound of formula (I) or any related formula where applicable, such as any one of formulae (I-a)-(I-n), (II), (II-a)-(II-n), (III), (Ill-a)-(III-n), (IV), (IV-a)-(IV-n), (V), (V-a)-(V-n), (VI), and (VI-a)-(VI-n). It is to be understood that two or more values may combined. Thus, it is to be understood that any variable for a compound of formula (I) or any related formula may be combined with any other variable for a compound of formula (I) or any related formula the same as if each and every combination of variables were specifically and individually listed.

In some embodiments of a compound of formula (I) or any related formula where applicable, or a pharmaceutically acceptable salt, stereoisomer, mixture of stereoisomers, solvate, or tautomer thereof, B is N. In some embodiments, B is CR^{a}.

In some embodiments of a compound of formula (I), or a pharmaceutically acceptable salt, stereoisomer, mixture of stereoisomers, solvate, or tautomer thereof, the compound is of formula (I-a) or (I-b), wherein A, [symbol] , m, Q, L, L¹, X¹, R¹, R², R³, R^{a}, R^{e}, and R^{f} are as detailed herein for formula (I).

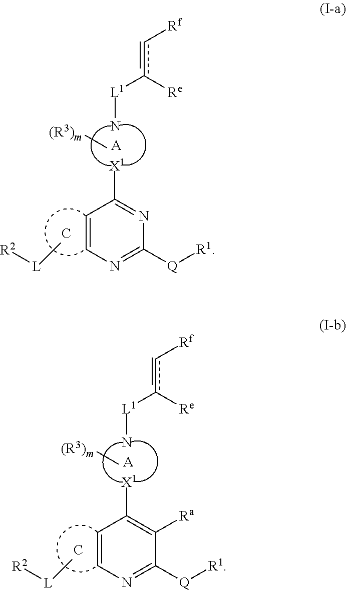

In some embodiments of a compound of formula (I) or (II), or a pharmaceutically acceptable salt, stereoisomer, mixture of stereoisomers, solvate, or tautomer thereof, the compound is of formula (II-a) or (II-b), wherein A, m, n, Q, L, L¹, X¹, R¹, R², R³, R⁴, R^{a}, R^{e}, and R^{f} are as detailed herein for formula (I).

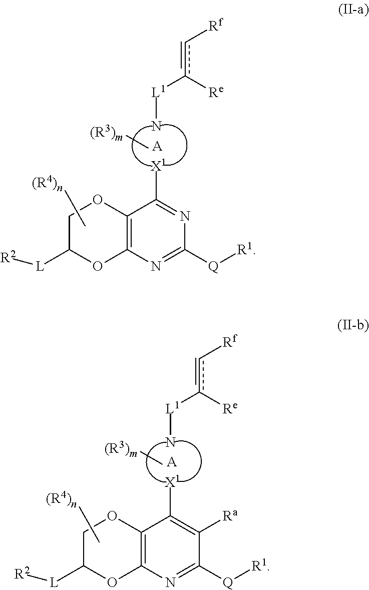

In some embodiments of a compound of formula (I) or (III), or a pharmaceutically acceptable salt, stereoisomer, mixture of stereoisomers, solvate, or tautomer thereof, the compound is of formula (III-a) or (Ill-b), wherein A, m, n, Q, L, L¹, X¹, R¹, R², R³, R⁴, R^{a}, R^{e}, and R^{f} are as detailed herein for formula (I).

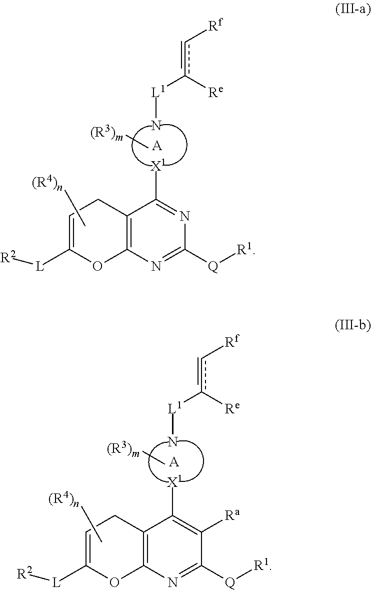

In some embodiments of a compound of formula (I) or (IV), or a pharmaceutically acceptable salt, stereoisomer, mixture of stereoisomers, solvate, or tautomer thereof, the compound is of formula (IV-a) or (IV-b), wherein A, m, n, Q, L, L¹, X¹, R¹, R², R³, R⁴, R⁶, R^{a}, R^{e}, and R^{f} are as detailed herein for formula (I).

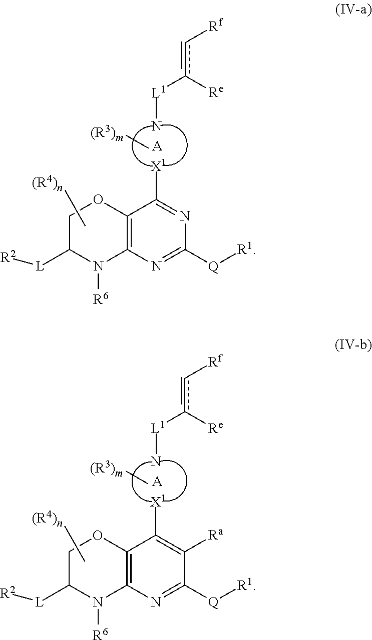

In some embodiments of a compound of formula (I) or (V), or a pharmaceutically acceptable salt, stereoisomer, mixture of stereoisomers, solvate, or tautomer thereof, the compound is of formula (V-a) or (V-b), wherein A, m, n, Q, L, L¹, X¹, R¹, R², R³, R⁴, R⁶, R^{a}, R^{e}, and R^{f} are as detailed herein for formula (I).

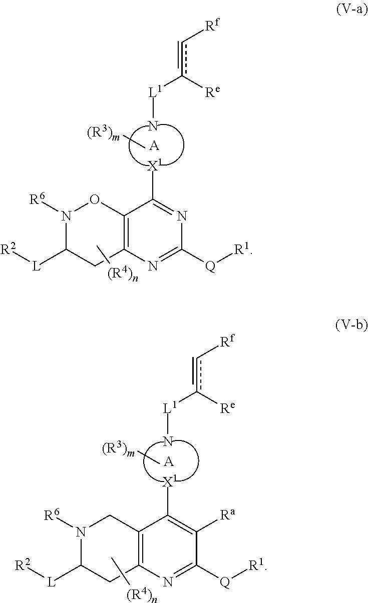

In some embodiments of a compound of formula (I) or (VI), or a pharmaceutically acceptable salt, stereoisomer, mixture of stereoisomers, solvate, or tautomer thereof, the compound is of formula (VI-a) or (VI-b), wherein A, m, n, Q, L, L¹, X¹, R¹, R², R³, R⁴, R^{a}, R^{e}, and R^{f} are as detailed herein for formula (I).

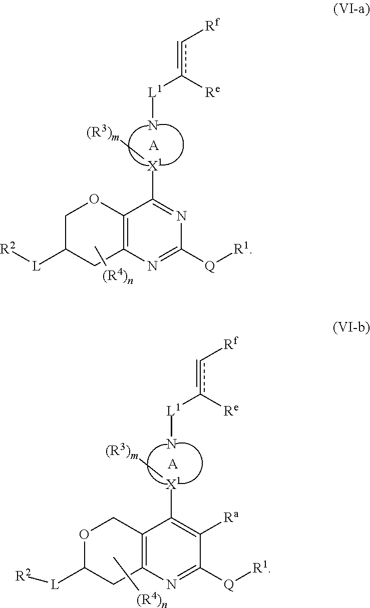

In some embodiments of a compound of formula (I) or any related formula where applicable, or a pharmaceutically acceptable salt, stereoisomer, mixture of stereoisomers, solvate, or tautomer thereof, Q is —O—. In some embodiments, Q is —S—. In some embodiments, Q is —NR⁵—. In some embodiments, Q is —NR⁵—; and R⁵ is hydrogen. In some embodiments, Q is —NR⁵—; and R⁵ is C₁-C₃ alkyl. In some embodiments, Q is —NR⁵—; and R⁵ is C₃-C₆ cycloalkyl. In some embodiments, Q is a bond. In some embodiments, Q is C₁-C₃ alkylene. In some embodiments, B is N; and Q is —O—. In some embodiments, B is N; and Q is —S—. In some embodiments, B is N; and Q is —NR⁵—. In some embodiments, B is N; Q is —NR⁵—; and R⁵ is hydrogen. In some embodiments, B is N; Q is —NR⁵—; and R⁵ is C₁-C₃ alkyl. In some embodiments, B is N; Q is —NR⁵—; and R⁵ is C₃-C₆ cycloalkyl. In some embodiments, B is N; and Q is a bond. In some embodiments, B is N; and Q is C₁-C₃ alkylene. In some embodiments, B is CR^{a}; and Q is —O—. In some embodiments, B is CR^{a}; and Q is —S—. In some embodiments, B is CR^{a}; and Q is —NR⁵—. In some embodiments, B is CR^{a}; Q is —NR⁵—; and R⁵ is hydrogen. In some embodiments, B is CR^{a}; Q is —NR⁵—; and R⁵ is C₁-C₃ alkyl. In some embodiments, B is CR^{a}; Q is —NR⁵—; and R⁵ is C₃-C₆ cycloalkyl. In some embodiments, B is CR^{a}; and Q is a bond. In some embodiments, B is CR^{a}; and Q is C₁-C₃ alkylene.

In some embodiments of a compound of formula (I) or any related formula where applicable, or a pharmaceutically acceptable salt, stereoisomer, mixture of stereoisomers, solvate, or tautomer thereof, L is a bond. In some embodiments, L is —C(O)—. In some embodiments, L is C₁-C₃ alkylene. In some embodiments, L is —O—. In some embodiments, L is —S—. In some embodiments, L is —S(O)—. In some embodiments, L is —S(O)₂—.

In some embodiments of a compound of formula (I), or a pharmaceutically acceptable salt, stereoisomer, mixture of stereoisomers, solvate, or tautomer thereof, the compound is of any one of formulae (I-c)-(I-f), wherein A, B, [symbol] , m, L, L¹, X¹, R¹, R², R³, R⁵, R^{e} and R^{f} are as detailed herein for formula (I).

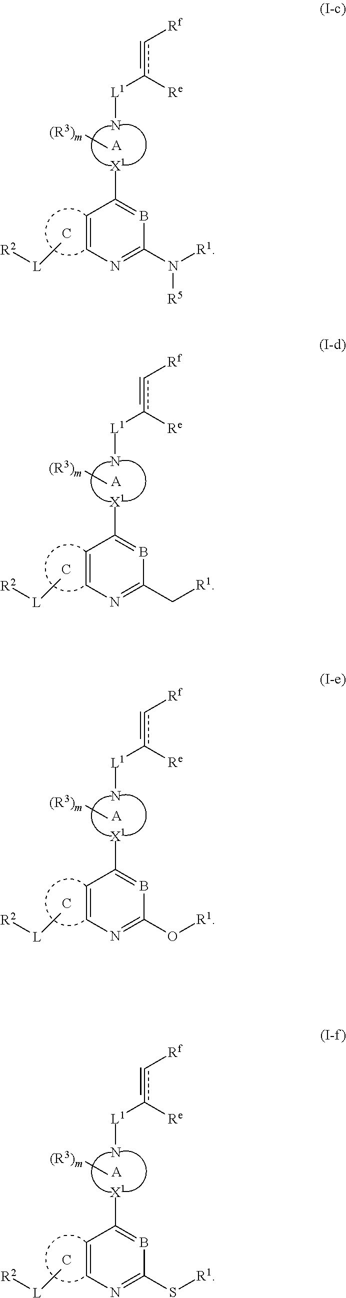

In some embodiments of a compound of formula (I) or (II), or a pharmaceutically acceptable salt, stereoisomer, mixture of stereoisomers, solvate, or tautomer thereof, the compound is of any one of formulae (II-c)-(II-f), wherein B, m, n, L¹, X¹, R¹, R², R³, R⁴, R⁵, R^{e} and R^{f} are as detailed herein for formula (I).

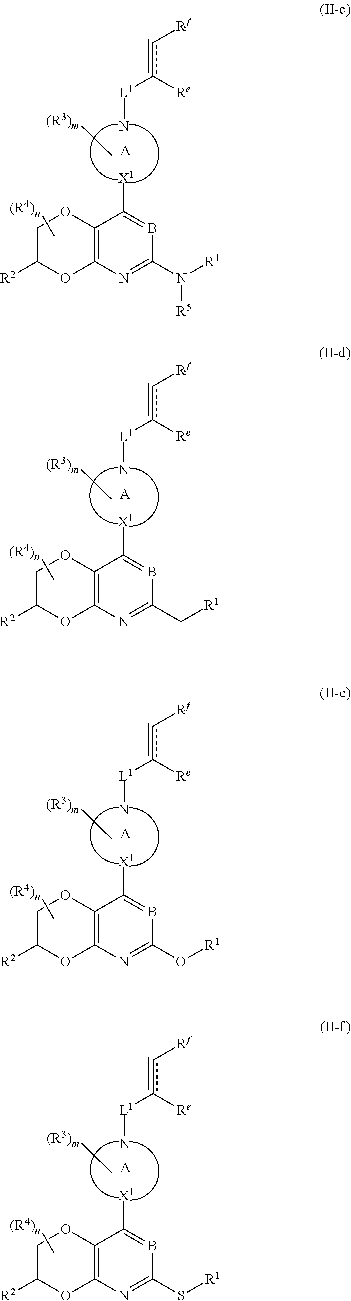

In some embodiments of a compound of formula (I) or (III), or a pharmaceutically acceptable salt, stereoisomer, mixture of stereoisomers, solvate, or tautomer thereof, the compound is of any one of formulae (III-c)-(III-f), wherein A, B, m, n, L¹, X¹, R¹, R², R³, R⁴, R⁵, R^{e} and R^{f} are as detailed herein for formula (I).

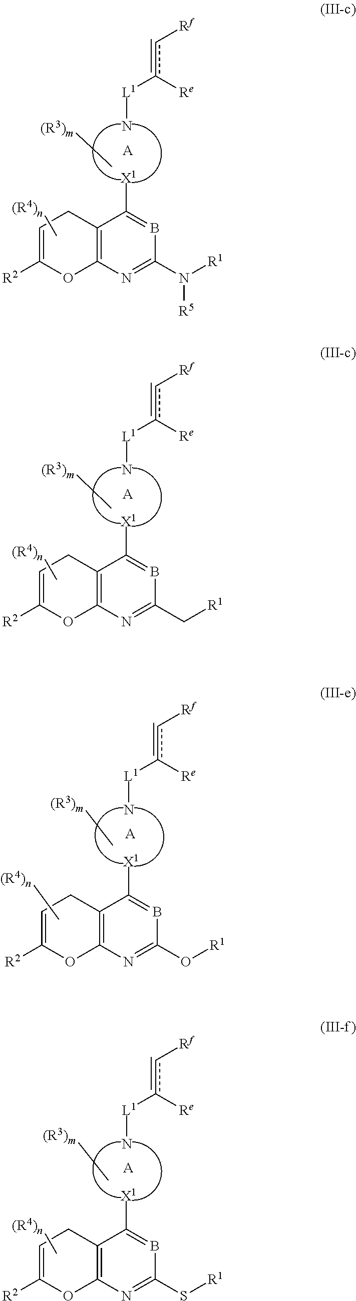

In some embodiments of a compound of formula (I) or (IV), or a pharmaceutically acceptable salt, stereoisomer, mixture of stereoisomers, solvate, or tautomer thereof, the compound is of any one of formulae (IV-c)-(IV-f), wherein A, B, m, n, L¹, X¹, R¹, R², R³, R⁴, R⁵, R⁶, R^{e} and R^{f} are as detailed herein for formula (I).

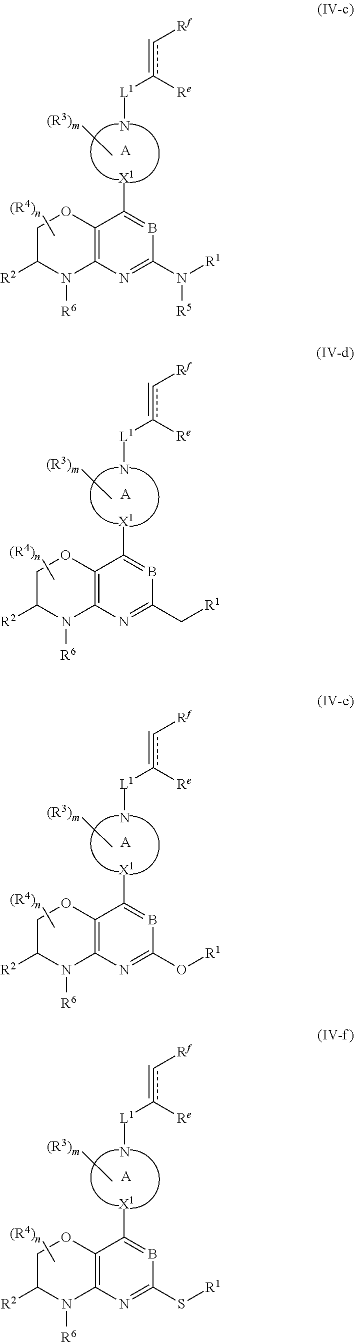

In some embodiments of a compound of formula (I) or (V), or a pharmaceutically acceptable salt, stereoisomer, mixture of stereoisomers, solvate, or tautomer thereof, the compound is of any one of formulae (V-c)-(V-f), wherein A, B, m, n, L¹, X¹, R¹, R², R³, R⁴, R⁵, R⁶, R^{e} and R^{f} are as detailed herein for formula (I).

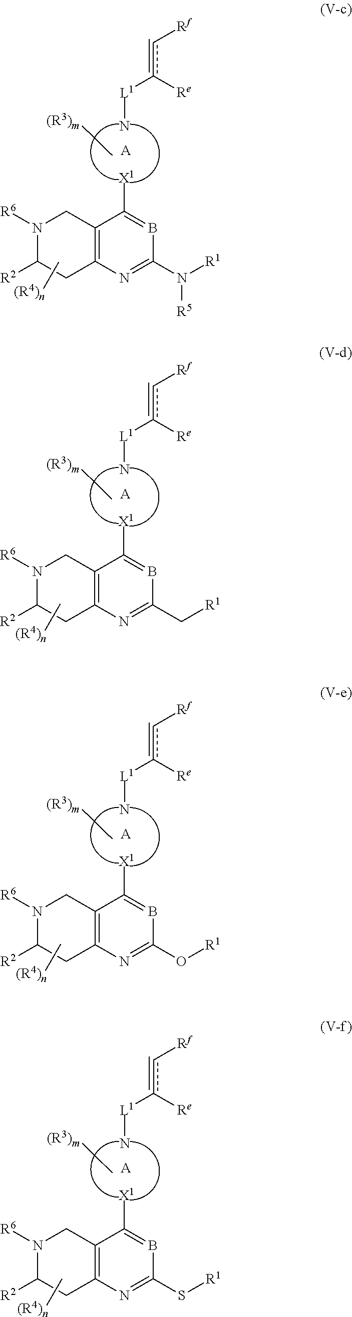

In some embodiments of a compound of formula (I) or (VI), or a pharmaceutically acceptable salt, stereoisomer, mixture of stereoisomers, solvate, or tautomer thereof, the compound is of any one of formulae (VI-c)-(VI-f), wherein A, B, m, n, L¹, X¹, R¹, R², R³, R⁴, R⁵, R^{e} and R^{f} are as detailed herein for formula (I).

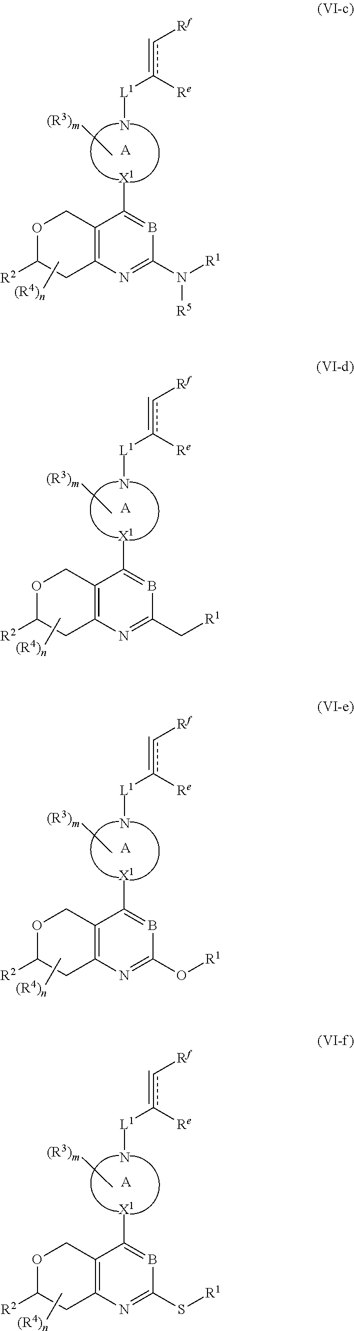

In some embodiments of a compound of formula (I) or any related formula where applicable, or a pharmaceutically acceptable salt, stereoisomer, mixture of stereoisomers, solvate, or tautomer thereof, X¹ is C. In some embodiments, X¹ is N. In some embodiments, X¹ is CH.

In some embodiments of a compound of formula (I) or any related formula where applicable, or a pharmaceutically acceptable salt, stereoisomer, mixture of stereoisomers, solvate, or tautomer thereof, A is a 4-12 membered saturated or partially saturated monocyclic ring. In some embodiments, A is 6-membered saturated or partially saturated monocyclic ring. In some embodiments, A is a 5-12 membered saturated or partially saturated bridged ring. In some embodiments, A is a 4-12 membered saturated or partially saturated fused ring. In some embodiments, A is a 4-12 membered saturated monocyclic ring. In some embodiments, A is 6-membered saturated monocyclic ring. In some embodiments, A is a 5-12 membered saturated bridged ring. In some embodiments, A is a 4-12 membered saturated fused ring.

In some embodiments of a compound of formula (I) or any related formula where applicable, or a pharmaceutically acceptable salt, stereoisomer, mixture of stereoisomers, solvate, or tautomer thereof, A and R³ together are

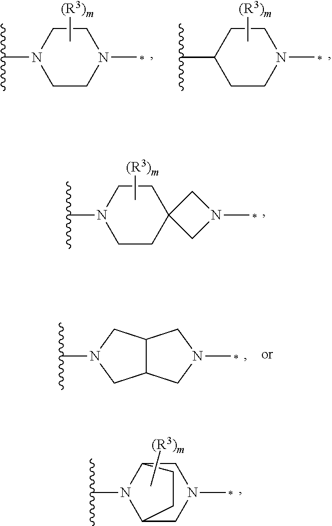

wherein * denotes point of attachment to L¹. It is understood that when A is a spiro or fused ring, any part of the ring can be substituted by R³. In some embodiments, A and R³ together are

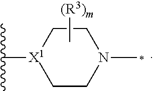

In some embodiments, A and R³ together are

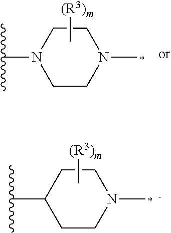

In some embodiments, A and R³ together are

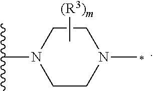

In some embodiments, A and R³ together are

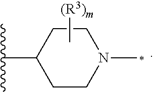

In some embodiments, A and R³ together are

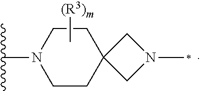

In some embodiments, A and R³ together are

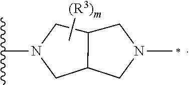

In some embodiments, A and R³ together are

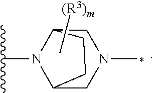

In some embodiments, m is 0. In some embodiments, m is 1. In some embodiments, m is 2. In some embodiments, m is 3. In some embodiments, m is 0, 1, or 2. In some embodiments, m is 0 or 1. In some embodiments, m is 1, 2, or 3. In some embodiments, each R³ substituent on A is independently C₁-C₆ alkyl optionally substituted with one or more substituents selected from —CN, halogen, —ORⁱ, —N(RⁱR^{j}) and 5-12 membered heteroaryl. In some embodiments, each R³ substituent on A is independently methyl or —CH₂CN.

In some embodiments of a compound of formula (I) or any related formula where applicable, or a pharmaceutically acceptable salt, stereoisomer, mixture of stereoisomers, solvate, or tautomer thereof, A and R³ together are

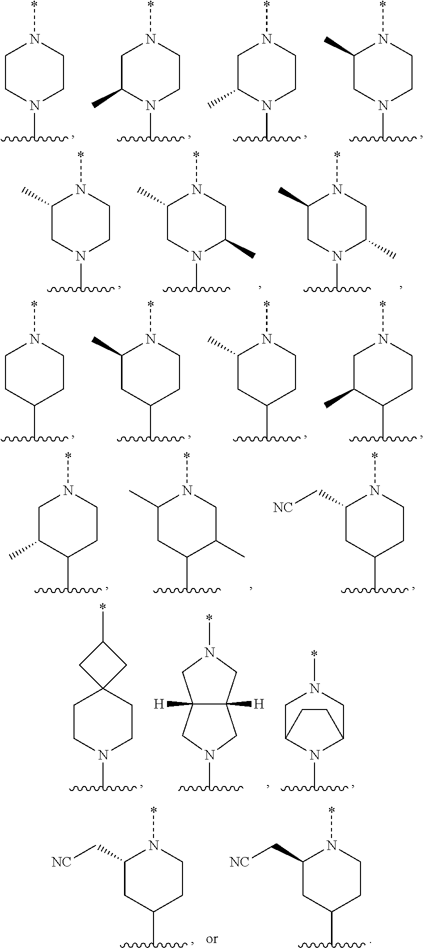

In some embodiments of a compound of formula (I) or any related formula where applicable, or a pharmaceutically acceptable salt, stereoisomer, mixture of stereoisomers, solvate, or tautomer thereof, A and R³ together are

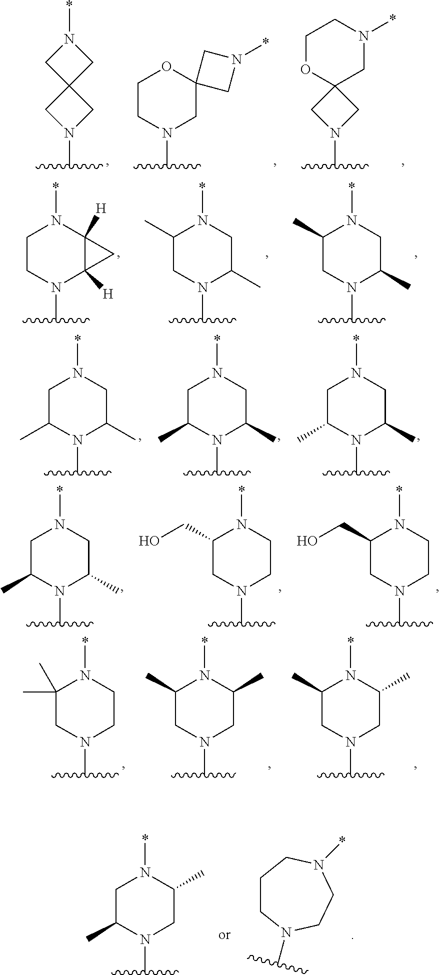

In some embodiments of a compound of formula (I) or any related formula where applicable, or a pharmaceutically acceptable salt, stereoisomer, mixture of stereoisomers, solvate, or tautomer thereof, A and R³ together are

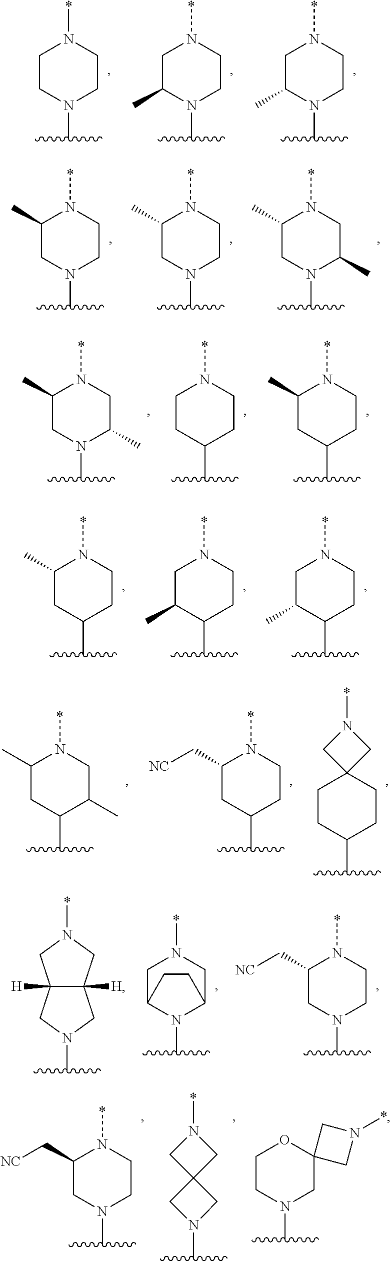

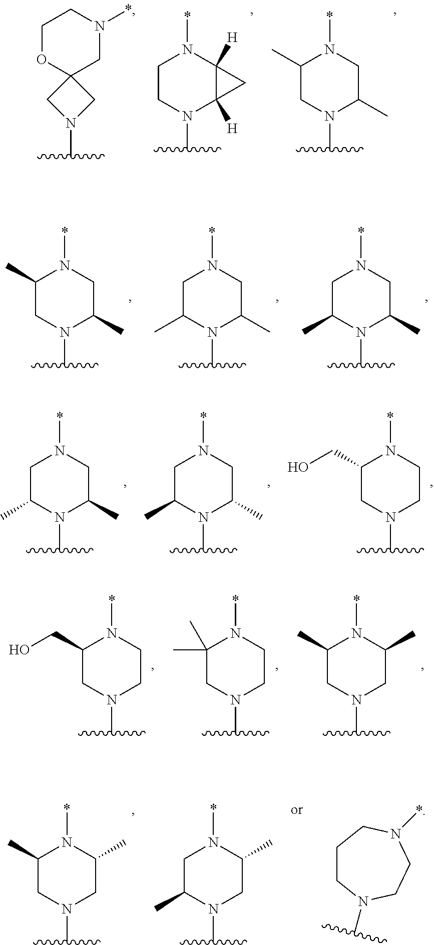

In some embodiments of a compound of formula (I) or any related formula where applicable, or a pharmaceutically acceptable salt, stereoisomer, mixture of stereoisomers, solvate, or tautomer thereof, L¹ is —C(O)—. In some embodiments, L¹ is —S(O)₂—.

In some embodiments of a compound of formula (I) or any related formula where applicable, or a pharmaceutically acceptable salt, stereoisomer, mixture of stereoisomers, solvate, or tautomer thereof, [symbol] is a double bond. In some embodiments, [symbol] is a triple bond and R^{e} is absent. In some embodiments, L¹ is —C(O)—; and [symbol] is a double bond. In some embodiments, L¹ is —C(O)—; and [symbol] is a triple bond and R^{e} is absent. In some embodiments, L¹ is —S(O)₂—; and [symbol] is a double bond. In some embodiments, L¹ is —S(O)₂—; and [symbol] is a triple bond and R^{e} is absent.

In some embodiments of a compound of formula (I), or a pharmaceutically acceptable salt, stereoisomer, mixture of stereoisomers, solvate, or tautomer thereof, the compound is of any one of formulae (I-g)-(I-n), wherein [symbol] , m, L, Q, R¹, R², R³, R^{e} and R^{f} are as detailed herein for formula (I).

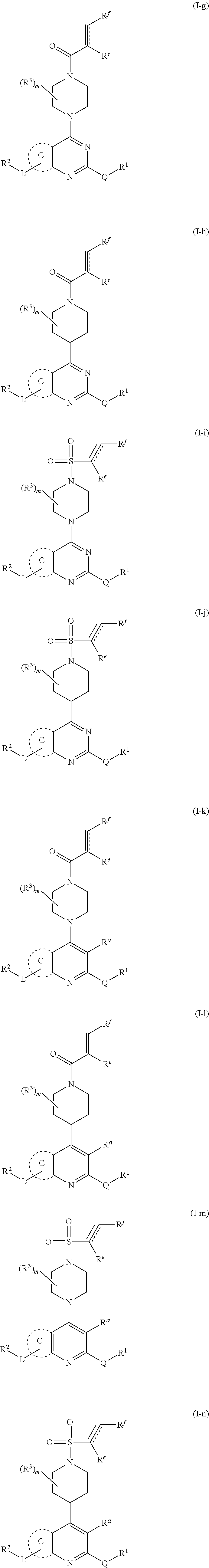

In some embodiments of a compound of formula (I) or (II), or a pharmaceutically acceptable salt, stereoisomer, mixture of stereoisomers, solvate, or tautomer thereof, the compound is of any one of formulae (II-g)-(II-n), wherein m, n, Q, R¹, R², R³, R⁴, R^{e} and R^{f} are as detailed herein for formula (I).

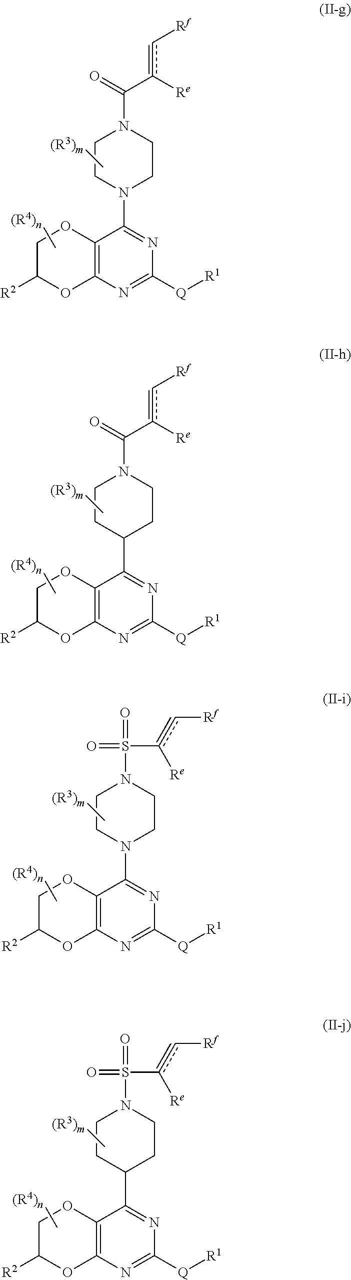

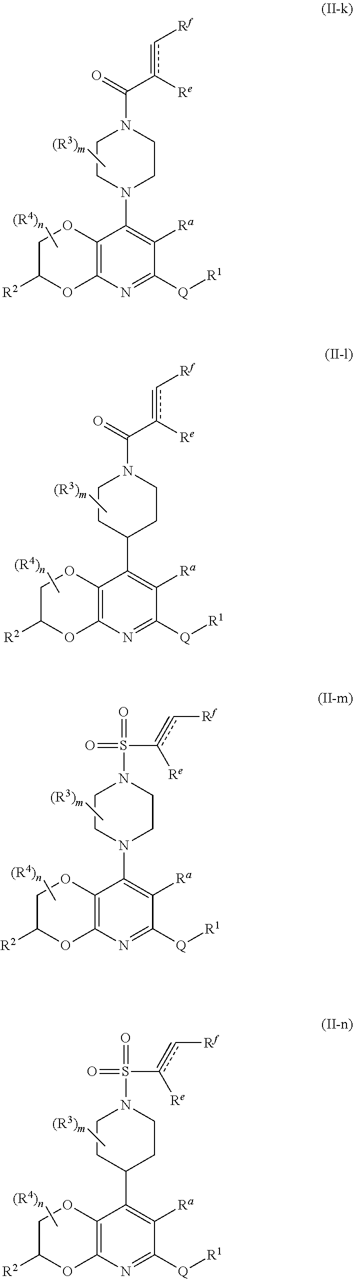

In some embodiments of a compound of formula (I) or (III), or a pharmaceutically acceptable salt, stereoisomer, mixture of stereoisomers, solvate, or tautomer thereof, the compound is of any one of formulae (III-g)-(III-n), wherein m, n, Q, R¹, R², R³, R⁴, R^{e} and R^{f} are as detailed herein for formula (I).

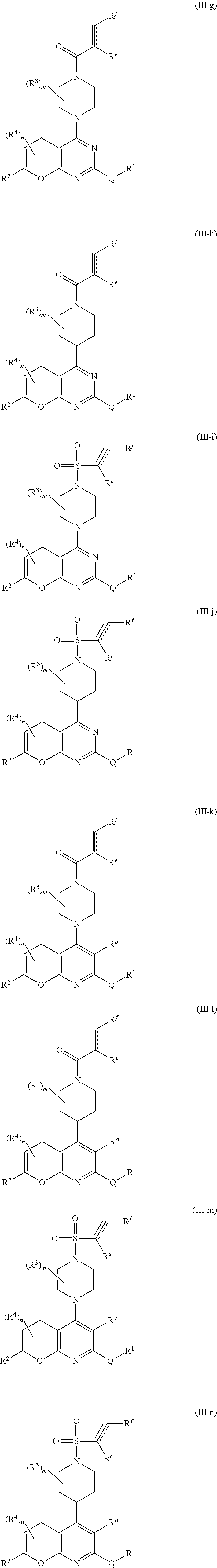

In some embodiments of a compound of formula (I) or (IV), or a pharmaceutically acceptable salt, stereoisomer, mixture of stereoisomers, solvate, or tautomer thereof, the compound is of any one of formulae (IV-g)-(IV-n), wherein m, n, Q, R¹, R², R³, R⁴, R⁶, R^{e} and R^{f} are as detailed herein for formula (I).

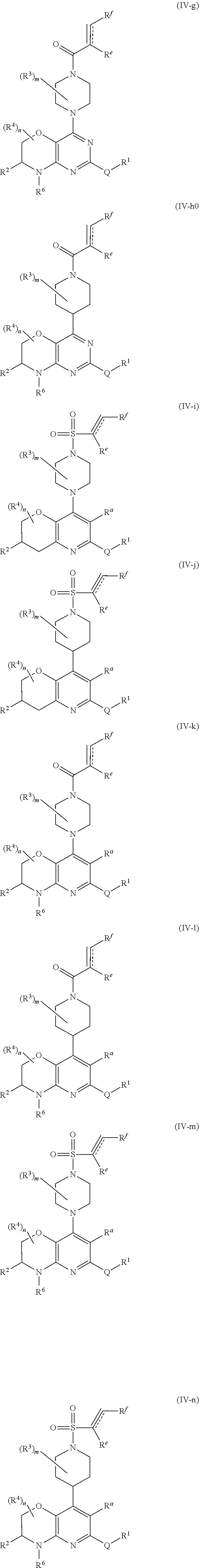

In some embodiments of a compound of formula (I) or (V), or a pharmaceutically acceptable salt, stereoisomer, mixture of stereoisomers, solvate, or tautomer thereof, the compound is of any one of formulae (V-g)-(V-n), wherein m, n, Q, R¹, R², R³, R⁴, R⁶, R^{e} and R^{f} are as detailed herein for formula (I).

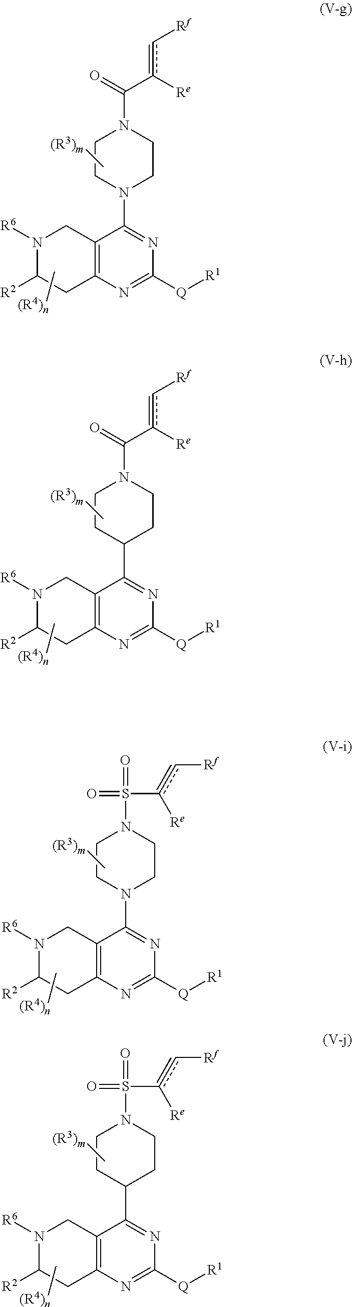

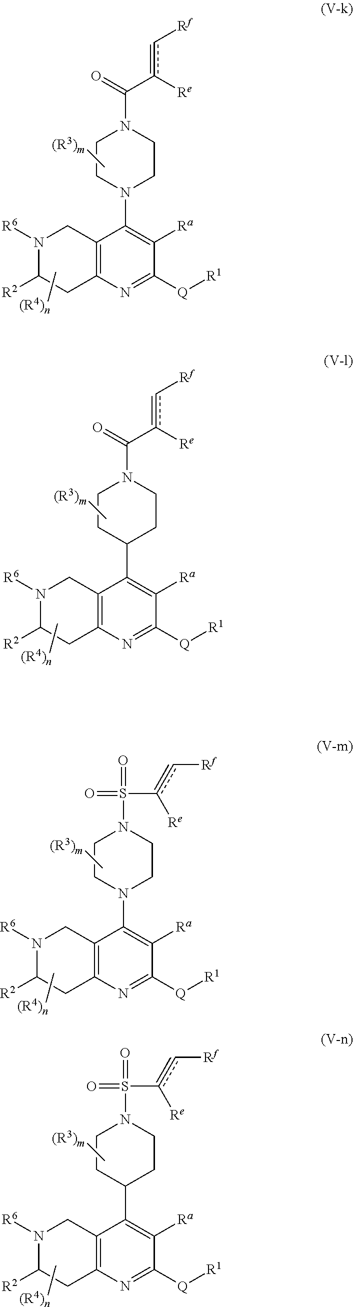

In some embodiments of a compound of formula (I) or (VI), or a pharmaceutically acceptable salt, stereoisomer, mixture of stereoisomers, solvate, or tautomer thereof, the compound is of any one of formulae (VI-g)-(VI-n), wherein m, n, Q, R¹, R², R³, R⁴, R^{e} and R^{f} are as detailed herein for formula (I).

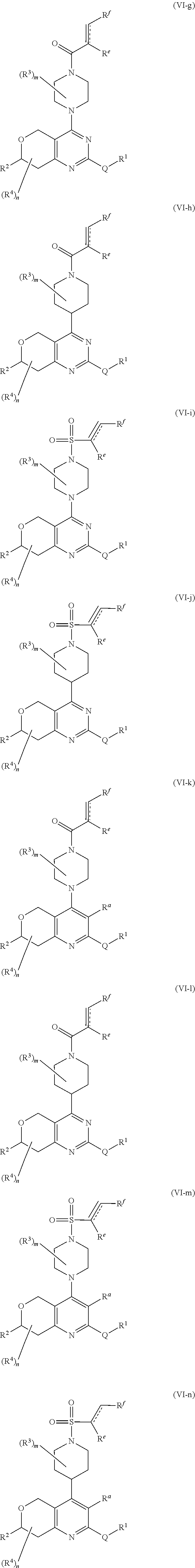

In some embodiments of a compound of formula (I) or any related formula where applicable, or a pharmaceutically acceptable salt, stereoisomer, mixture of stereoisomers, solvate, or tautomer thereof, R¹ is hydrogen. In some embodiments, R¹ is C₁-C₆ alkyl which is optionally substituted with one or more R^{1a}. In some embodiments, R¹ is C₁-C₆ haloalkyl which is optionally substituted with one or more R^{1a}. In some embodiments, R¹ is —(C₁-C₆ alkylene)OR^{c} which is optionally substituted with one or more R^{1a}. In some embodiments, R¹ is —(C₁-C₆ alkylene)NR^{c}R^{d} which is optionally substituted with one or more R^{1a}. In some embodiments, R¹ is —NR^{c}R^{d} which is optionally substituted with one or more R^{1a}. In some embodiments, R¹ is 3-12 membered heterocyclyl which is optionally substituted with one or more R^{1a}. In some embodiments, R¹ is C₃-C₁₂ cycloalkyl which is optionally substituted with one or more R^{1a}. In some embodiments, R¹ is —(C₁-C₆ alkylene) 3-12 membered heterocyclyl which is optionally substituted with one or more R^{1a}. In some embodiments, R¹ is C₆-C₁₂ aryl which is optionally substituted with one or more R^{1a}. In some embodiments, R¹ is 5-12 membered heteroaryl which is optionally substituted with one or more R^{1a}. In some embodiments, R¹ is —(C₁-C₆ alkylene) 5-12 membered heteroaryl which is optionally substituted with one or more R^{1a}. In some embodiments, R¹ is C₁-C₆ alkyl, —(C₁-C₆ alkylene)NR^{c}R^{d}, 3-12 membered heterocyclyl, or —(C₁-C₆ alkylene) 3-12 membered heterocyclyl, each of which is optionally substituted with one or more R^{1a}. In some embodiments, R¹ is ethyl, propyl,

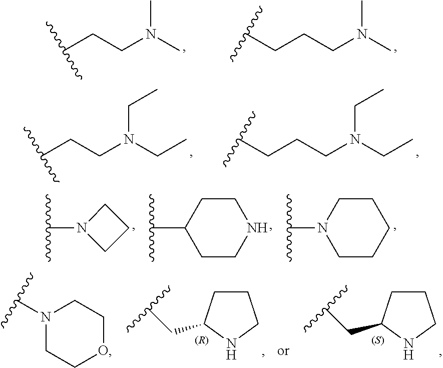

each of which is optionally substituted with one or more R^{1a}. In some embodiments, each R^{1a} is independently —N(R^{g}R^{h}). C₁-C₆ alkyl, or —C(O)N(R^{g}R^{h}). In some embodiments, R¹ is

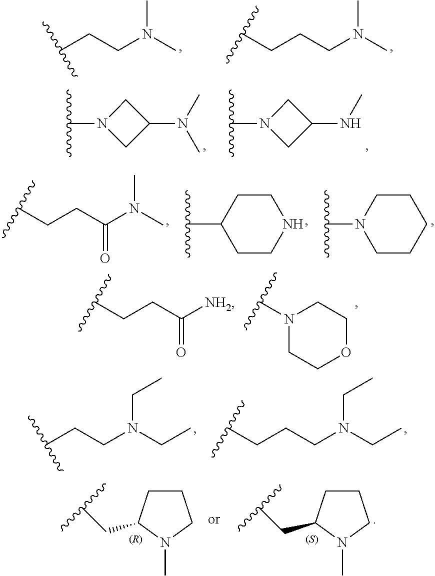

In some embodiments, R¹ is

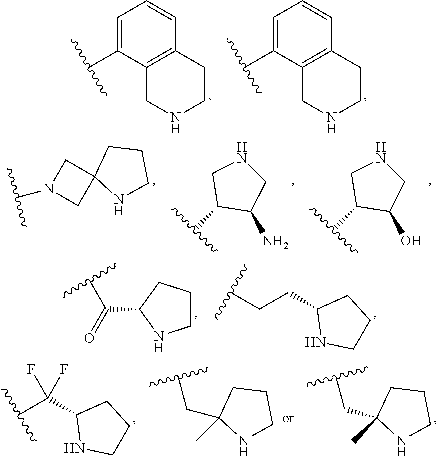

each of which is optionally substituted with one or more R^{1a}. In some embodiments, each R^{1a} is independently —N(R^{f}R^{g}), C₁-C₆ alkyl, or —C(O)N(R^{f}R^{g}). In some embodiments, R¹ is

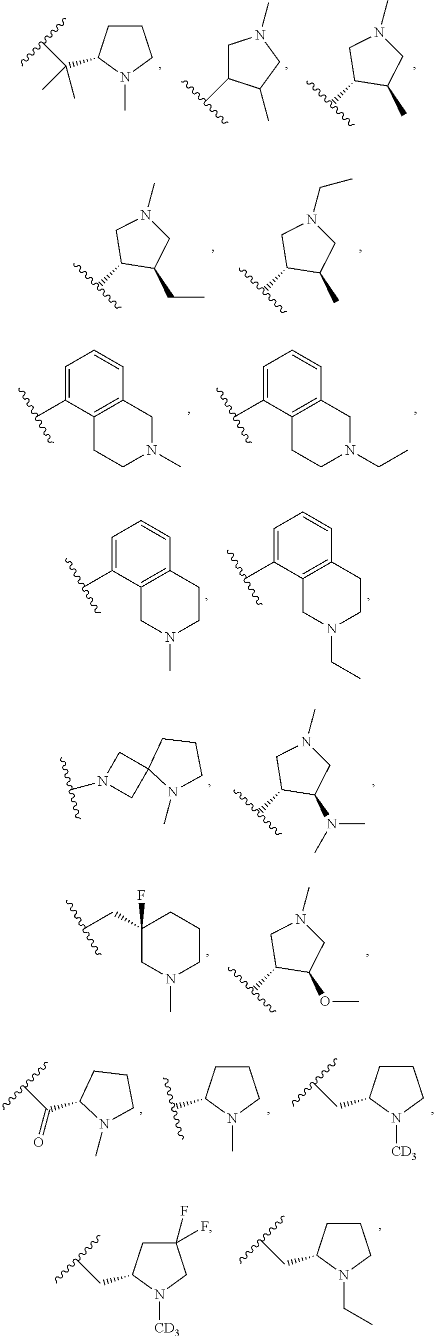

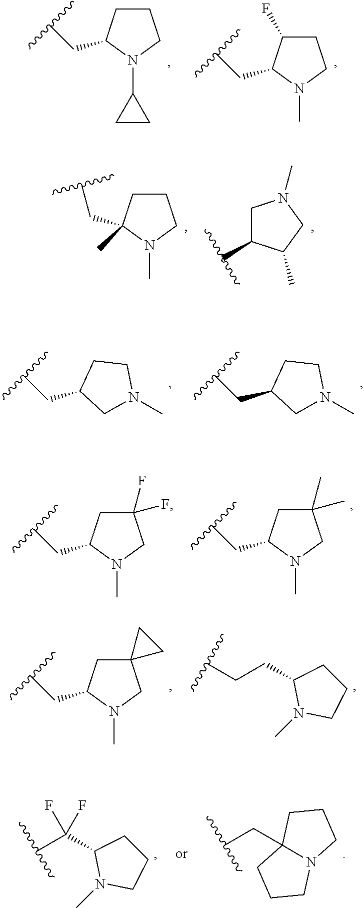

In some embodiments, R¹ is

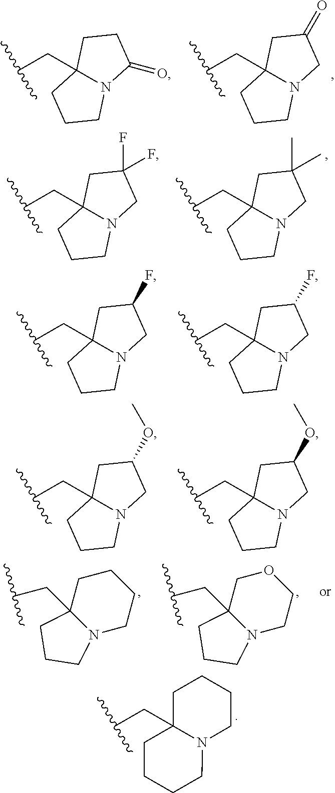

In some embodiments the compound of formula (I) or any related formula where applicable, or a pharmaceutically acceptable salt, stereoisomer, mixture of stereoisomers, solvate, or tautomer thereof, has a compound of formula (VI-g),

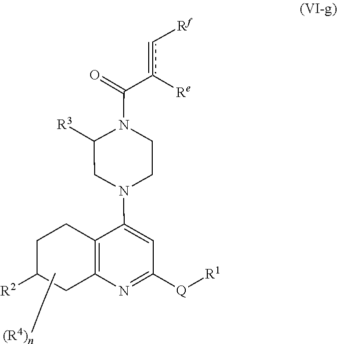

wherein, R¹ is

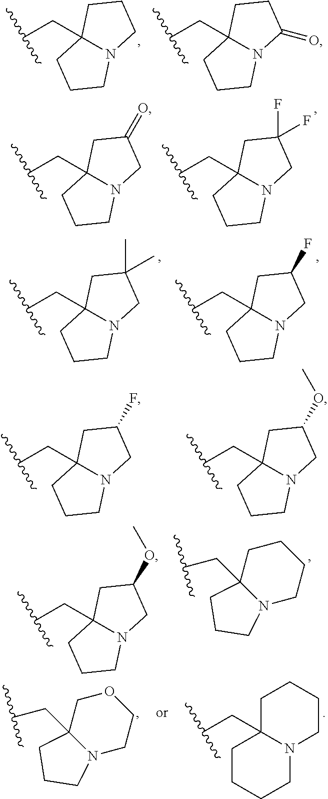

In some such embodiments, Q is —O—. In other such embodiments, R¹ is

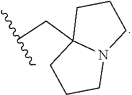 [SMILES: *CC12CCCN1CCC2]

In other such embodiments, Q is —O— and R¹ is

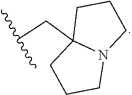 [SMILES: *CC12CCCN1CCC2]

In other such embodiments,

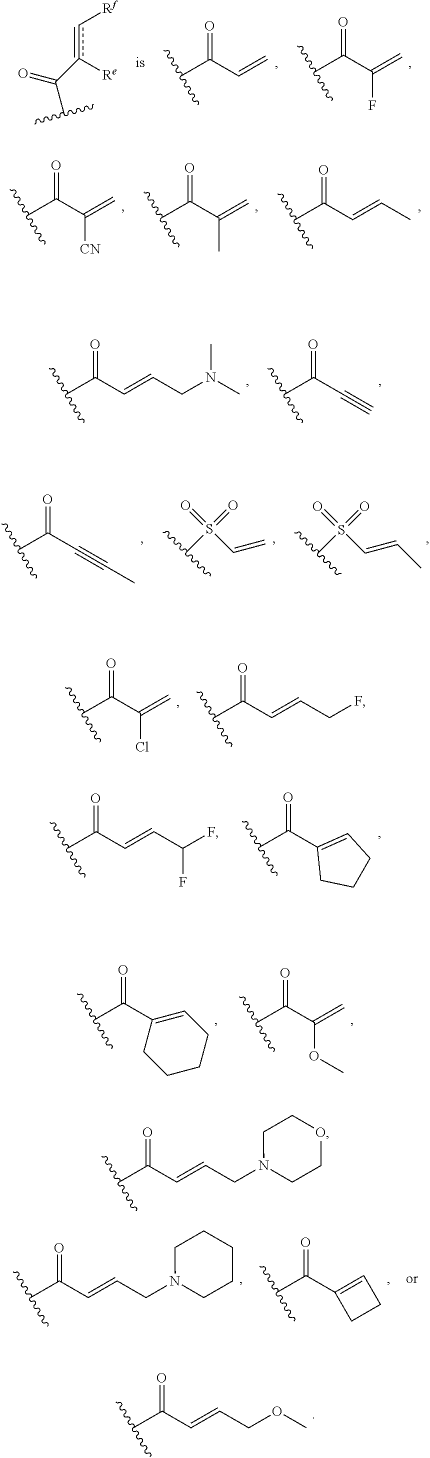

In other such embodiments,

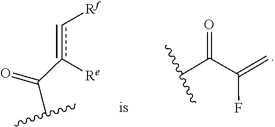

In other such embodiments,

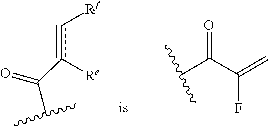

### and R¹ is

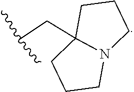 [SMILES: *CC12CCCN1CCC2]

In other such embodiments, R² is

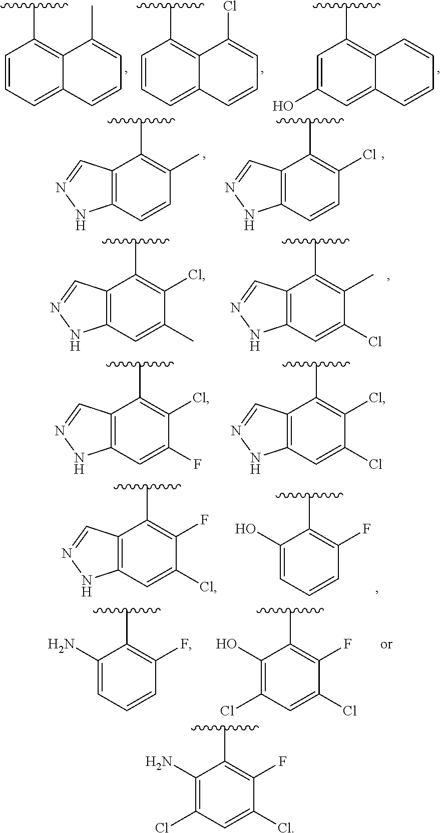

In other such embodiments,

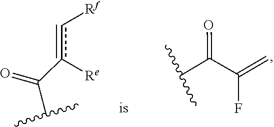

### R¹ is

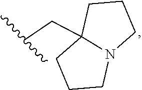 [SMILES: *CC12CCCN1CCC2]

### and R² is

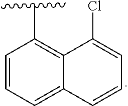 [SMILES: *c1cccc2cccc(Cl)c12]

In some embodiments the compound of formula (I) or any related formula where applicable, or a pharmaceutically acceptable salt, stereoisomer, mixture of stereoisomers, solvate, or tautomer thereof, has a compound of formula (VI-g′),

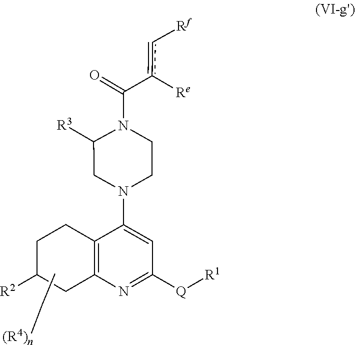

wherein, R¹ is

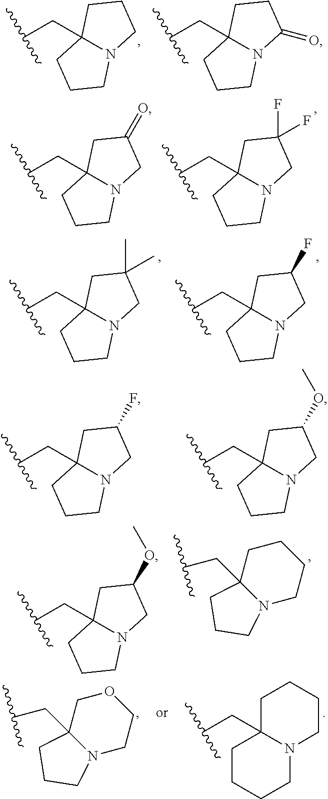

In some such embodiments, R³ is —CH₂CN. In some such embodiments, R⁴ is H, alkyl, halogen, n is 0, 1, 2 or 3. In some such embodiments, Q is —O—. In other such embodiments, R¹ is

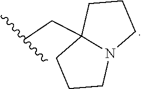 [SMILES: *CC12CCCN1CCC2]

In other such embodiments, Q is —O—and R¹ is

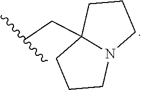 [SMILES: *CC12CCCN1CCC2]

In other such embodiments, Q is —O—, R¹ is

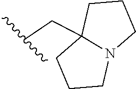 [SMILES: *CC12CCCN1CCC2]

R³ is —CH₂CN, and R⁴ is H, alkyl, halogen, n is 0, 1, 2 or 3. In other such embodiments,

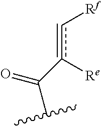

is

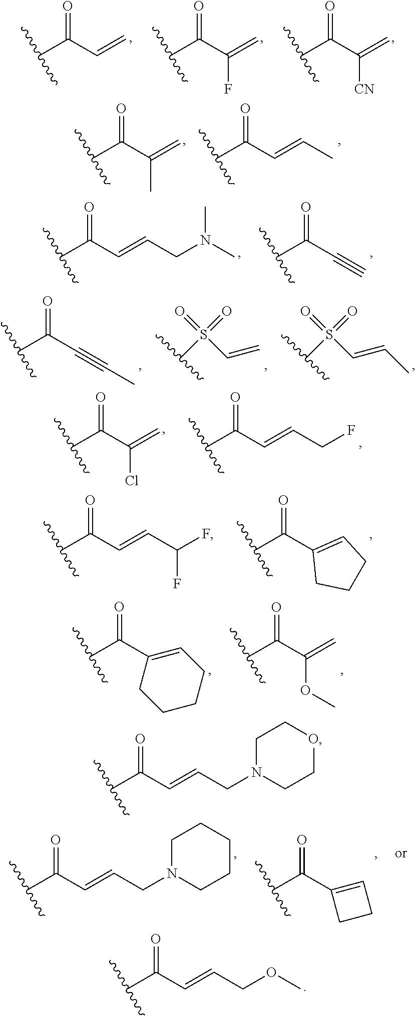

In other such embodiments,

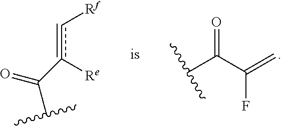

In other such embodiments,

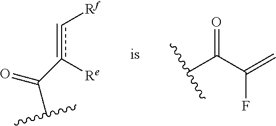

### and R¹ is

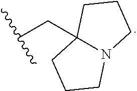 [SMILES: *CC12CCCN1CCC2]

In other such embodiments, R² is

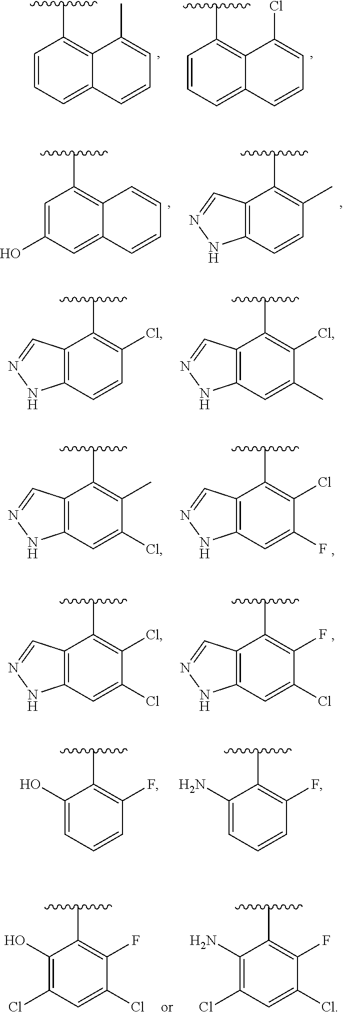

In other such embodiments,

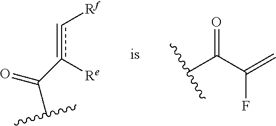

### R¹ is

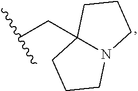 [SMILES: *CC12CCCN1CCC2]

### and R² is

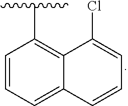 [SMILES: *c1cccc2cccc(Cl)c12]

In other such embodiments, a compound of formula (VI-g′) is provided wherein the carbon atom to which the R³ is connected is in the (S) stereochemical configuration. In other such embodiments, a compound of formula (VI-g′) is provided wherein the carbon atom to which the R³ is connected is in the (R) stereochemical configuration.

In some embodiments of a compound of formula (VI-g′) a pharmaceutically acceptable salt, stereoisomer, mixture of stereoisomers, solvate, or tautomer thereof, is provided wherein the carbon atom to which the R¹ is connected is in the (S) stereochemical configuration. In other such embodiments, a compound of formula (VI-g′), or a pharmaceutically acceptable salt, stereoisomer, mixture of stereoisomers, solvate, or tautomer thereof, is provided wherein the carbon atom to which the R¹ is connected is in the (R) stereochemical configuration. In some embodiments of a compound of formula (VI-g′), or a pharmaceutically acceptable salt, stereoisomer, mixture of stereoisomers, solvate, or tautomer thereof, is provided wherein the carbon atom to which the R² is connected is in the (S) stereochemical configuration. In other such embodiments, a compound of formula (VI-g′), or a pharmaceutically acceptable salt, stereoisomer, mixture of stereoisomers, solvate, or tautomer thereof, is provided wherein the carbon atom to which the R² is connected is in the (R) stereochemical configuration. In some embodiments of a compound of formula (VI-g′), or a pharmaceutically acceptable salt, stereoisomer, mixture of stereoisomers, solvate, or tautomer thereof, is provided wherein the carbon atom to which the R³ is connected is in the (S) stereochemical configuration. In other such embodiments, a compound of formula (VI-g′), or a pharmaceutically acceptable salt, stereoisomer, mixture of stereoisomers, solvate, or tautomer thereof, is provided wherein the carbon atom to which the R³ is connected is in the (R) stereochemical configuration.

In some embodiments of a compound of formula (I) or any related formula where applicable, or a pharmaceutically acceptable salt, stereoisomer, mixture of stereoisomers, solvate, or tautomer thereof, R^{a} is hydrogen. In some embodiments, R^{a} is —CN. In some embodiments, R^{a} is C₁-C₆ alkoxy, which is optionally substituted with one or more R³. In some embodiments, R^{a} is C₁-C₆ haloalkoxy, which is optionally substituted with one or more R³. In some embodiments, R^{a} is —C(O)R^{c}, which is optionally substituted with one or more R³. In some embodiments, R^{a} is —S(O)₂R^{b}, which is optionally substituted with one or more R³. In some embodiments, R^{a} is —C(O)OR^{c}, which is optionally substituted with one or more R³. In some embodiments, R^{a} is —C(O)N(R^{c}R^{d}), which is optionally substituted with one or more R³. In some embodiments, R^{a} is —N(R^{c}R^{d}), which is optionally substituted with one or more R³. In some embodiments, R^{a} is C₆-C₁₂ aryl, which is optionally substituted with one or more R³. In some embodiments, R^{a} is 3-12 membered heterocyclyl, which is optionally substituted with one or more R³. In some embodiments, R^{a} is 5-12 membered heteroaryl, which is optionally substituted with one or more R³. In some embodiments, R^{a} is C₁-C₆ alkoxy, which is optionally substituted with one or more R³. In some embodiments, R^{a} is halogen. In some embodiments, R^{a} is —N(R^{c})C(O)N(R^{c}R^{d}), which is optionally substituted with one or more R³. In some embodiments, R^{a} is —N(R^{c})C(O)R^{d}, which is optionally substituted with one or more R³.

In some embodiments of a compound of formula (I) or any related formula where applicable, or a pharmaceutically acceptable salt, stereoisomer, mixture of stereoisomers, solvate, or tautomer thereof, R^{e} is H. In some embodiments, R^{e} is halogen. In some embodiments, R^{e} is —CN. In some embodiments, R^{e} is —C(O)OR^{g}. In some embodiments, R^{e} is C₁-C₆ haloalkyl. In some embodiments, R^{e} is C₁-C₆ alkyl. In some embodiments, R^{e} is C₁-C₆ heteroalkyl. In some embodiments, R^{e} is —C(O)N(R^{g}R^{h}). In some embodiments, R^{e} is C₆-C₁₂ aryl. In some embodiments, R^{e} is 5-12 membered heteroaryl. In some embodiments, R^{e} is 3-12 membered heterocyclyl. In some embodiments, R^{e} is —(C₁-C₆ alkylene)OR^{g}. In some embodiments, R^{e} is —(C₁-C₆ alkylene)N(R^{g}R^{h}). In some embodiments, R^{e} is H, halogen, —CN, or C₁-C₆ alkyl.

In some embodiments of a compound of formula (I) or any related formula where applicable, or a pharmaceutically acceptable salt, stereoisomer, mixture of stereoisomers, solvate, or tautomer thereof, R^{f} is H. In some embodiments, R^{f} is halogen. In some embodiments, R^{f} is —CN. In some embodiments, R^{f} is —C(O)OR^{g}. In some embodiments, R^{f} is C₁-C₆ haloalkyl. In some embodiments, R^{f} is C₁-C₆ alkyl. In some embodiments, R^{f} is C₁-C₆ heteroalkyl. In some embodiments, R^{f} is —C(O)N(R^{g}R^{h}). In some embodiments, R^{f} is C₆-C₁₂ aryl. In some embodiments, R^{f} is 5-12 membered heteroaryl. In some embodiments, R^{f} is 3-12 membered heterocyclyl. In some embodiments, R^{f} is —(C₁-C₆ alkylene)OR^{g}. In some embodiments, R^{f} is —(C₁-C₆ alkylene)N(R^{g}R^{h}). In some embodiments, R^{f} is H, C₁-C₆ alkyl, or —(C₁-C₆ alkylene)N(R^{g}R^{h}).

In some embodiments of a compound of formula (I) or any related formula where applicable, or a pharmaceutically acceptable salt, stereoisomer, mixture of stereoisomers, solvate, or tautomer thereof, R^{e} and R^{f} are each independently H, halogen, —CN, C₁-C₆ alkyl, or —(C₁-C₆ alkylene)N(R^{g}R^{h}). In some embodiments,

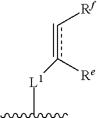

is

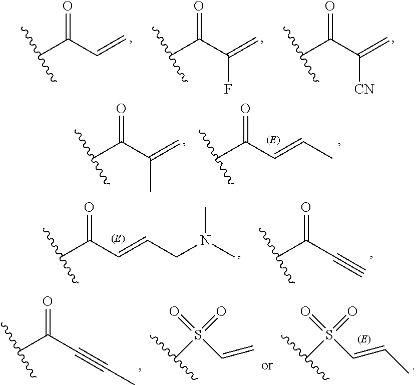

In some embodiments of a compound of formula (I) or any related formula where applicable, or a pharmaceutically acceptable salt, stereoisomer, mixture of stereoisomers, solvate, or tautomer thereof, R^{e} and R^{f} are each independently H, halogen, —CN, C₁-C₆ alkyl, or —(C₁-C₆ alkylene)N(R^{g}R^{h}). In some embodiments,

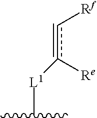

is

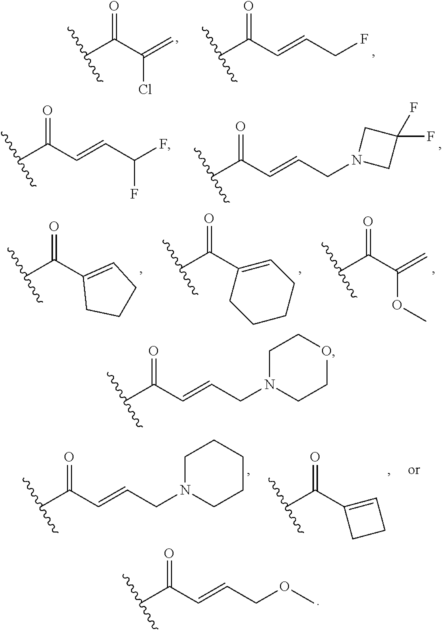

In some embodiments, R^{e} and R^{f} are each independently H, halogen, —CN, C₁-C₆ alkyl, or —(C₁-C₆ alkylene)N(R^{g}R^{h}). In some embodiments,

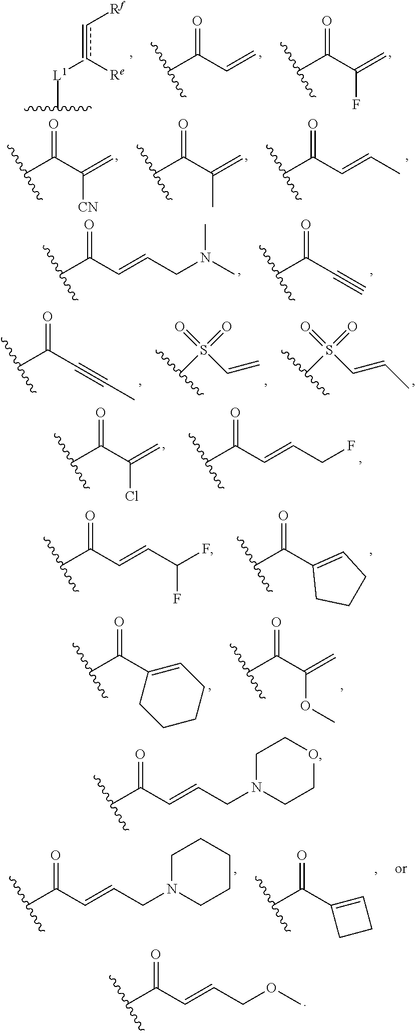

In some embodiments of a compound of formula (I) or any related formula where applicable, or a pharmaceutically acceptable salt, stereoisomer, mixture of stereoisomers, solvate, or tautomer thereof, R² is C₃-C₁₂ cycloalkyl optionally substituted with one or more R^{2a}. In some embodiments, R² is 3-12 membered heterocyclyl optionally substituted with one or more R^{2a}. In some embodiments, R² is 5-6 membered heterocyclyl optionally substituted with R^{2a}. In some embodiments, R² is C₆-C₁₂ aryl optionally substituted with one or more R^{2a}. In some embodiments, R² is phenyl optionally substituted with one or more R^{2a}. In some embodiments, R² is 5-12 membered heteroaryl optionally substituted with one or more R^{2a}. In some embodiments, R² is 5-6 membered heteroaryl optionally substituted with one or more R^{2a}.

In some embodiments of a compound of formula (I) or any related formula where applicable, or a pharmaceutically acceptable salt, stereoisomer, mixture of stereoisomers, solvate, or tautomer thereof, R² is

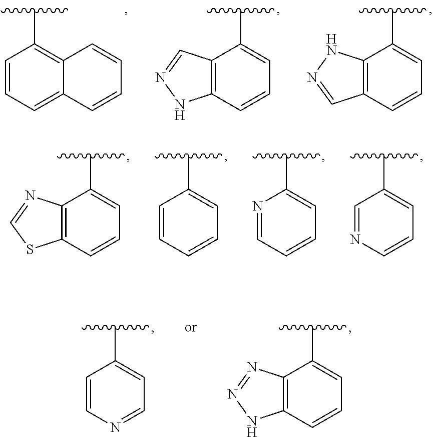

each of which is optionally substituted with one or more R^{2a}. In some embodiments, R² is

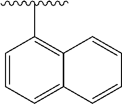 [SMILES: *c1cccc2ccccc12]

which is optionally substituted with one or more R^{2a}. In some embodiments, R² is

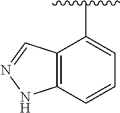 [SMILES: *c1cccc2[nH]ncc12]

which is optionally substituted with one or more R^{2a}. In some embodiments, R² is

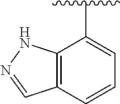 [SMILES: *c1cccc2cn[nH]c12]

which is optionally substituted with one or more R^{2a}. In some embodiments, R² is

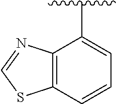 [SMILES: *c1cccc2scnc12]

which is optionally substituted with one or more R^{2a}. In some embodiments, R² is

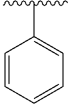 [SMILES: *c1ccccc1]

which is optionally substituted with one or more R^{2a}. In some embodiments, R² is

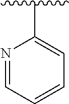 [SMILES: *c1ccccn1]

which is optionally substituted with one or more R^{2a}. In some embodiments, R² is

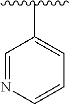 [SMILES: *c1cccnc1]

which is optionally substituted with one or more R^{2a}. In some embodiments, R² is

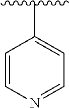 [SMILES: *c1ccncc1]

which is optionally substituted with one or more R^{2a}. In some embodiments, R^{2a} is

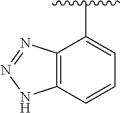 [SMILES: *c1cccc2[nH]nnc12]

which is optionally substituted with one or more R^{2a}. In some embodiments each R^{2a} is independently hydroxyl. C₁-C₆ alkyl, halogen, or —N(R^{g}R^{h}). In some embodiments R² is

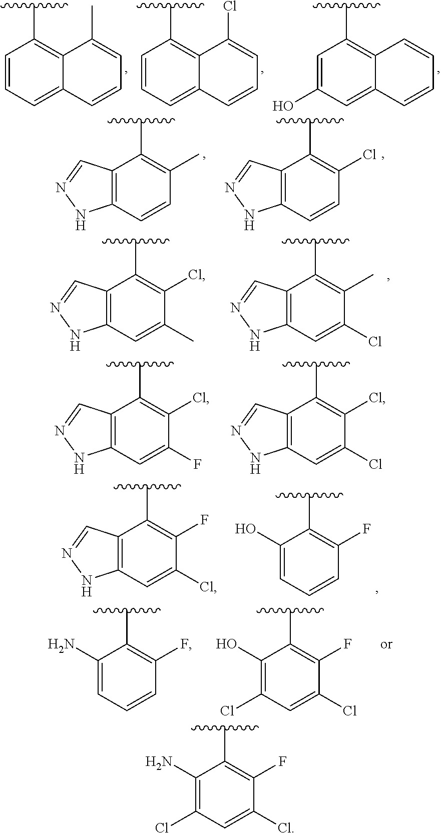

In some embodiments of a compound of formula (I) or any related formula where applicable, or a pharmaceutically acceptable salt, stereoisomer, mixture of stereoisomers, solvate, or tautomer thereof, R² is

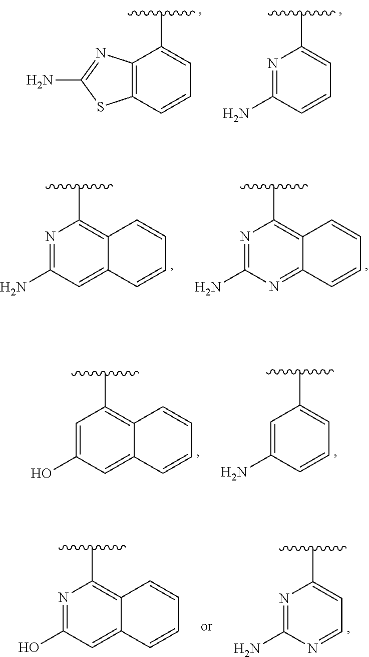

each of which is optionally substituted with one or more R^{2a}. In some embodiments, R² is

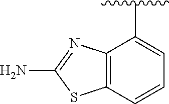 [SMILES: *c1cccc2sc(N)nc12]

which is optionally substituted with one or more R^{2a}. In some embodiments, R² is

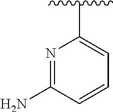 [SMILES: *c1cccc(N)n1]

which is optionally substituted with one or more R^{2a}. In some embodiments, R² is

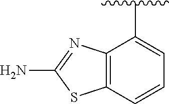 [SMILES: *c1cccc2sc(N)nc12]

which is optionally substituted with one or more R^{2a}. In some embodiments, R² is

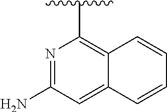 [SMILES: *c1nc(N)cc2ccccc12]

which is optionally substituted with one or more R^{2a}. In some embodiments, R² is

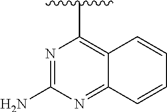 [SMILES: *c1nc(N)nc2ccccc12]

which is optionally substituted with one or more R^{2a}. In some embodiments, R² is

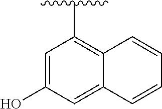 [SMILES: *c1cc(O)cc2ccccc12]

which is optionally substituted with one or more R^{2a}. In some embodiments, R² is

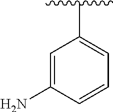 [SMILES: *c1cccc(N)c1]

which is optionally substituted with one or more R^{2a}. In some embodiments, R² is

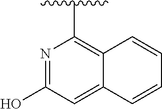 [SMILES: *c1nc(O)cc2ccccc12]

which is optionally substituted with one or more R^{2a}. In some embodiments, R² is

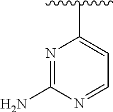 [SMILES: *c1ccnc(N)n1]

which is optionally substituted with one or more R^{2a}. In some embodiments, each R^{2a} is independently hydroxyl, C₁-C₆ alkyl, C₁-C₆ haloalkyl, halogen, or —N(R^{g}R^{h}). In some embodiments, R² is

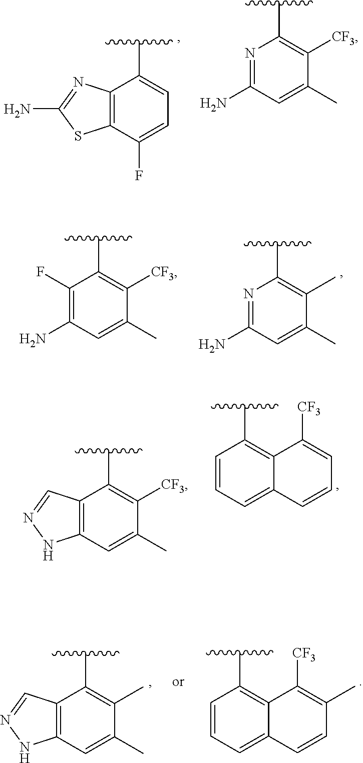

In some embodiments of a compound of formula (I) or any related formula where applicable, or a pharmaceutically acceptable salt, stereoisomer, mixture of stereoisomers, solvate, or tautomer thereof, n is 0. In some embodiments, n is 1. In some embodiments, n is 2. In some embodiments, n is 3. In some embodiments, n is 0, 1, or 2. In some embodiments, n is 0 or 1. In some embodiments, n is 1 or 2.

In some embodiments of a compound of formula (I) or any related formula where applicable, or a pharmaceutically acceptable salt, stereoisomer, mixture of stereoisomers, solvate, or tautomer thereof, when R⁴ is present, each R⁴ is independently oxo. In some embodiments, when R⁴ is present, each R⁴ is independently —C(O)R^{g} which is optionally substituted with one or more substituents selected from —ORⁱ, C₃-C₁₂ cycloalkyl, and —CN. In some embodiments, when R⁴ is present, each R⁴ is independently hydroxyl. In some embodiments, when R⁴ is present, each R⁴ is independently —(C₁-C₆ alkylene)OR^{g}, which is optionally substituted with one or more substituents selected from —ORⁱ, C₃-C₁₂ cycloalkyl, and —CN. In some embodiments, when R⁴ is present, each R⁴ is independently —CN. In some embodiments, when R⁴ is present, each R⁴ is independently halogen. In some embodiments, when R⁴ is present, each R⁴ is independently C₁-C₆ alkyl, which is optionally substituted with one or more substituents selected from —ORⁱ, C₃-C₁₂ cycloalkyl, and —CN. In some embodiments, when R⁴ is present, each R⁴ is independently —(C₁-C₆ alkylene) C₆-C₁₂ aryl, which is optionally substituted with one or more substituents selected from —ORⁱ, C₃-C₁₂ cycloalkyl, and —CN. In some embodiments, when R⁴ is present, each R⁴ is independently C₁-C₆ haloalkyl, which is optionally substituted with one or more substituents selected from —ORⁱ, C₃-C₁₂ cycloalkyl, and —CN. In some embodiments, when R⁴ is present, each R⁴ is independently C₃-C₈ cycloalkyl, which is optionally substituted with one or more substituents selected from —ORⁱ, C₃-C₁₂ cycloalkyl, and —CN. In some embodiments, when R⁴ is present, each R⁴ is independently 3-12 membered heterocyclyl, which is optionally substituted with one or more substituents selected from —ORⁱ, C₃-C₁₂ cycloalkyl, and —CN. In some embodiments, when R⁴ is present, each R⁴ is independently —(C₁-C₆ alkylene) 3-12 membered heterocyclyl, which is optionally substituted with one or more substituents selected from —ORⁱ, C₃-C₁₂ cycloalkyl, and —CN. In some embodiments, when R⁴ is present, each R⁴ is independently C₁-C₆ alkoxy, which is optionally substituted with one or more substituents selected from —ORⁱ, C₃-C₁₂ cycloalkyl, and —CN. In some embodiments, when R⁴ is present, each R⁴ is independently —(C₁-C₆ alkylene)C(O)N(R^{g}R^{h}), which is optionally substituted with one or more substituents selected from —ORⁱ, C₃-C₁₂ cycloalkyl, and —CN. In some embodiments, when R⁴ is present, each R⁴ is independently —(C₁-C₆ alkylene)N(R^{g}R^{h}), which is optionally substituted with one or more substituents selected from —ORⁱ, C₃-C₁₂ cycloalkyl, and —CN. In some embodiments, when R⁴ is present, each R⁴ is independently —S(O)₂R^{g}, which is optionally substituted with one or more substituents selected from —ORⁱ, C₃-C₁₂ cycloalkyl, and —CN. In some embodiments, when R⁴ is present, each R⁴ is independently —C(O)OR^{g}, which is optionally substituted with one or more substituents selected from —ORⁱ, C₃-C₁₂ cycloalkyl, and —CN. In some embodiments, when R⁴ is present, each R⁴ is independently —C(O)N(R^{g}R^{h}), which is optionally substituted with one or more substituents selected from —ORⁱ, C₃-C₁₂ cycloalkyl, and —CN. In some embodiments, when R⁴ is present, each R⁴ is independently —N(R^{g}R^{h}), which is optionally substituted with one or more substituents selected from —ORⁱ, C₃-C₁₂ cycloalkyl, and —CN. In some embodiments, each R⁴ is independently C₁-C₆ alkyl, which is optionally substituted with one or more substituents selected from —ORⁱ, C₃-C₁₂ cycloalkyl, and —CN.

In some embodiments of a compound of formula (I) or any related formula where applicable, or a pharmaceutically acceptable salt, stereoisomer, mixture of stereoisomers, solvate, or tautomer thereof, R⁶ is H. In some embodiments, R⁶ is C₁-C₆ haloalkyl, which is optionally substituted with one or more R³. In some embodiments, R⁶ is C₁-C₆ alkyl, which is optionally substituted with one or more R³. In some embodiments, R⁶ is C₃-C₈ cycloalkyl, which is optionally substituted with one or more R³. In some embodiments, R⁶ is C₁-C₆ haloalkoxy, which is optionally substituted with one or more R³. In some embodiments, R⁶ is C₁-C₆ alkoxy, which is optionally substituted with one or more R³. In some embodiments, R⁶ is —C(O)R^{c}, which is optionally substituted with one or more R³. In some embodiments, R⁶ is —S(O)₂R^{b}, which is optionally substituted with one or more R³. In some embodiments, R⁶ is —C(O)OR^{c}, which is optionally substituted with one or more R³. In some embodiments, R⁶ is —C(O)N(R^{c}R^{d}), which is optionally substituted with one or more R³. In some embodiments, R⁶ is C₆-C₁₂ aryl, which is optionally substituted with one or more R³. In some embodiments, R⁶ is 3-12 membered heterocyclyl, which is optionally substituted with one or more R³. In some embodiments, R⁶ is 5-12 membered heteroaryl, which is optionally substituted with one or more R³. In some embodiments, R⁶ is H or C₁-C₆ alkyl, which is optionally substituted with one or more R³. In some embodiments, R⁶ is H or C₁-C₆ alkyl. In some embodiments, R⁶ is H or methyl.

In some embodiments, the compounds of Formula I or any related formula where applicable, selectively react with the G12C mutant KRAS, HRAS or NRAS proteins to form a covalent bond. In some embodiments, the compounds react with the cysteine at position 12 of a G12C mutant KRAS, HRAS or NRAS protein to form a covalent bond.

Exemplary compounds provided by the present disclosure include, but are not limited to, a compound, shown in Table 1, or a stereoisomer, mixture of stereoisomers, hydrate, solvate, isotope or pharmaceutically acceptable salt thereof. In some embodiments, the compound is selected from the group consisting of Compound Nos. 1-227. In some embodiments, the compound is selected from the group consisting of Compound Nos. 1-271.

TABLE 1

| No. | Structure |
|---|---|
| 1 | 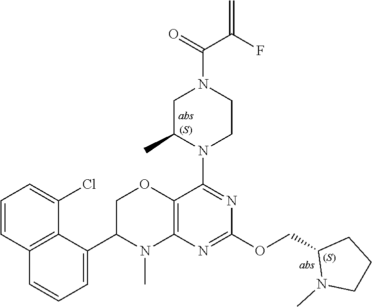 |
| 2 | 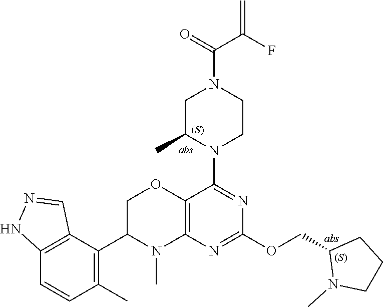 |
| 3 | 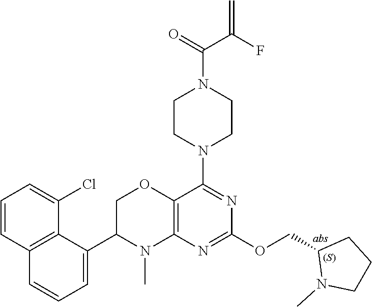 |
| 4 | 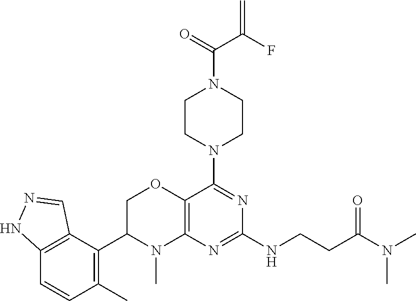 [SMILES: C=C(F)C(=O)N1CCN(c2nc(NCCC(=O)N(C)C)nc3c2OCC(c2c(C)ccc4[nH]ncc24)N3C)CC1] |
| 5 | 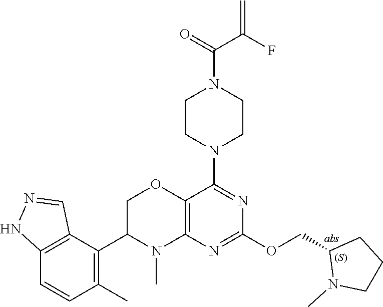 |
| 6 | 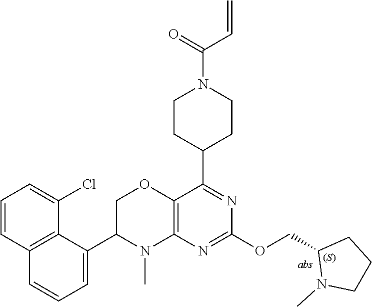 |
| 7 | 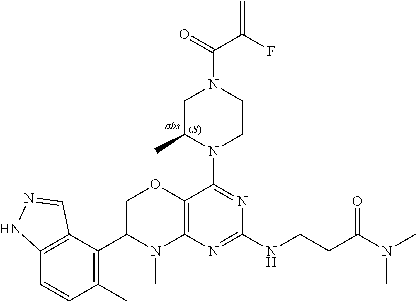 |
| 8 | 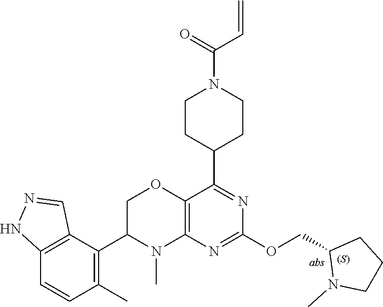 |
| 9 | 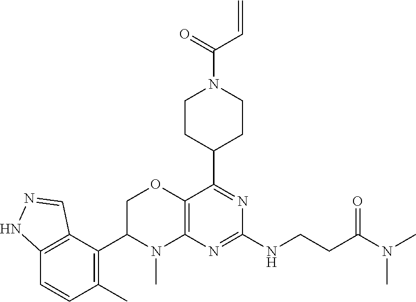 [SMILES: C=CC(=O)N1CCC(c2nc(NCCC(=O)N(C)C)nc3c2OCC(c2c(C)ccc4[nH]ncc24)N3C)CC1] |
| 10 | 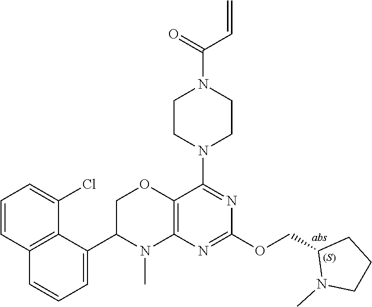 |
| 11 | 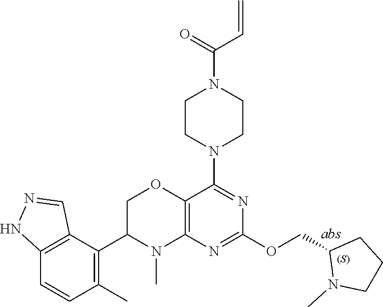 |
| 12 | 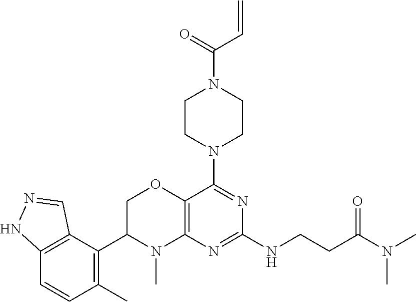 [SMILES: C=CC(=O)N1CCN(c2nc(NCCC(=O)N(C)C)nc3c2OCC(c2c(C)ccc4[nH]ncc24)N3C)CC1] |
| 13 | 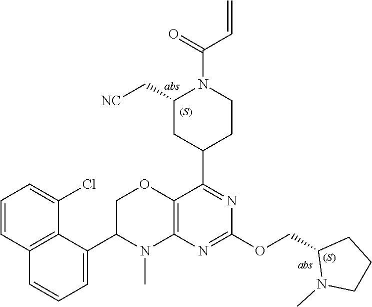 |
| 14 | 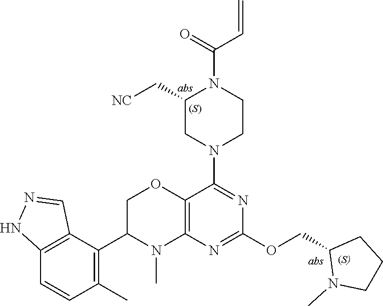 |
| 15 | 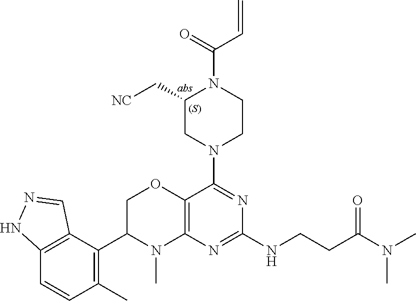 |
| 16 | 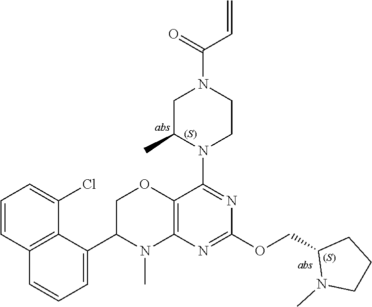 |
| 17 | 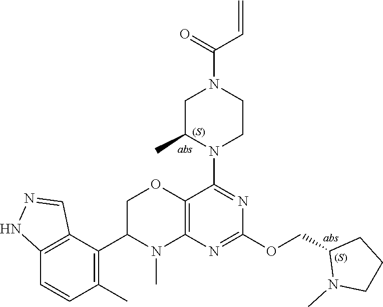 |
| 18 | 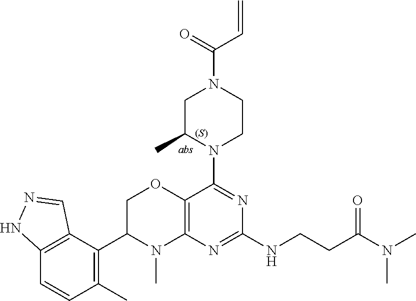 |
| 19 | 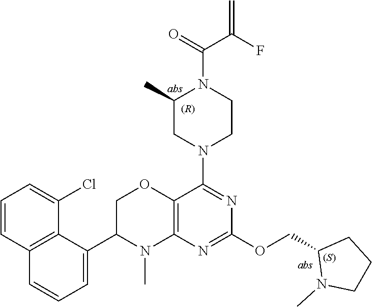 |
| 20 | 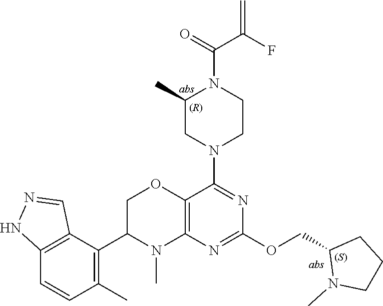 |
| 21 | 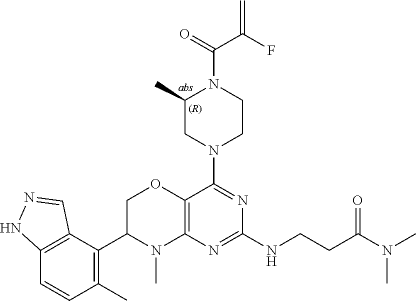 |
| 22 | 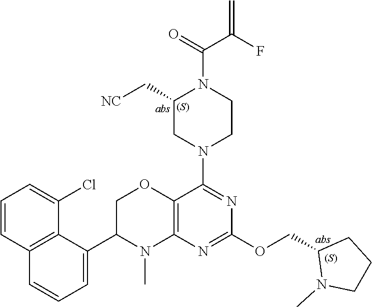 |
| 23 | 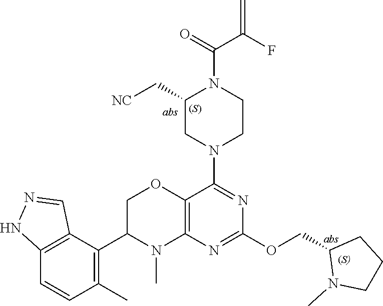 |
| 24 | 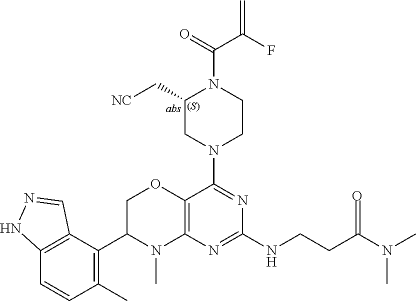 |
| 25 | 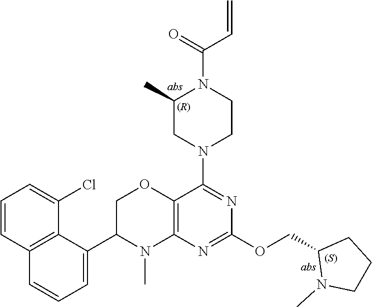 |
| 26 | 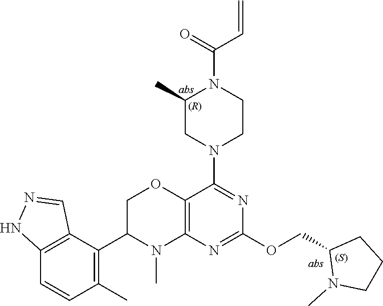 |
| 27 | 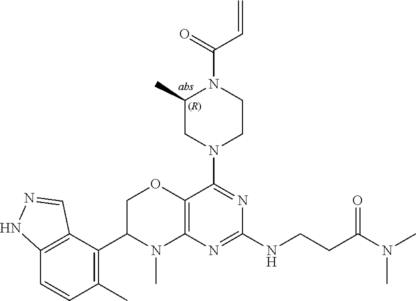 |
| 28 | 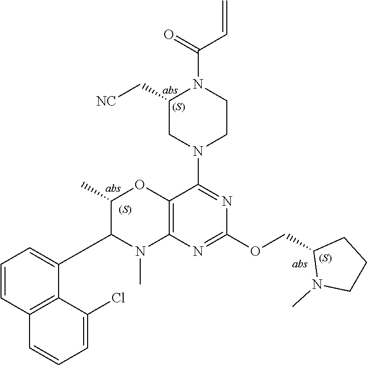 |
| 29 | 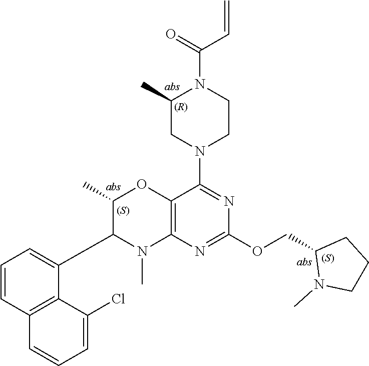 |
| 30 | 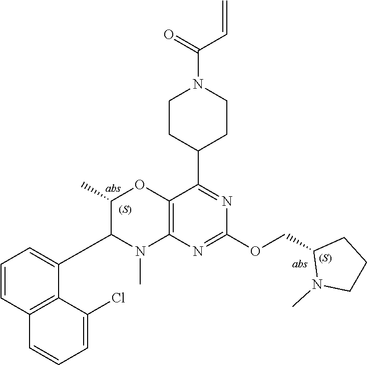 |
| 31 | 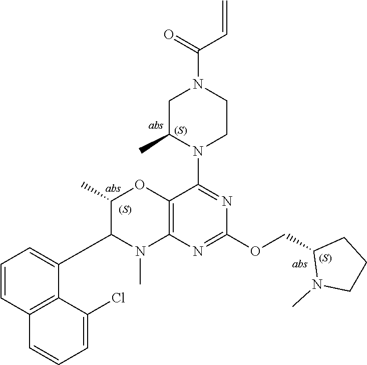 |
| 32 | 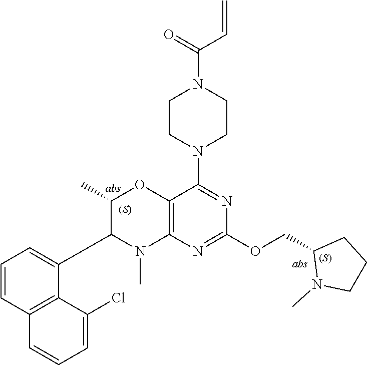 |
| 33 | 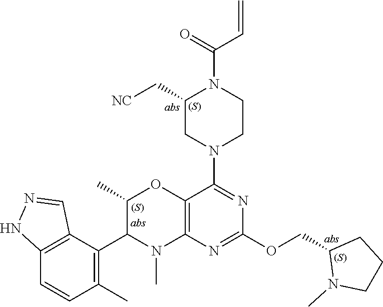 |
| 34 | 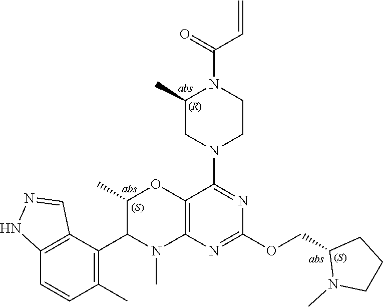 |
| 35 | 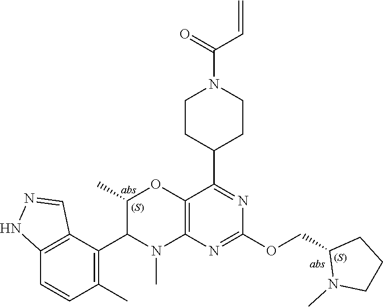 |
| 36 | 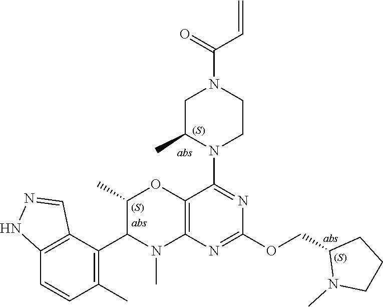 |
| 37 | 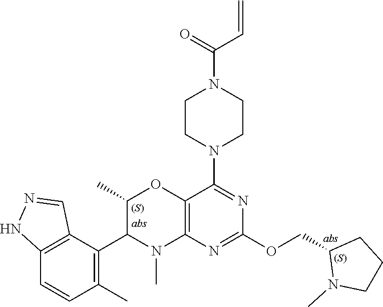 |
| 38 | 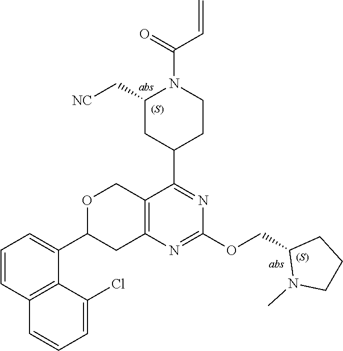 |
| 39 | 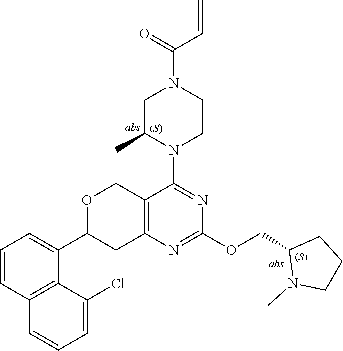 |
| 40 | 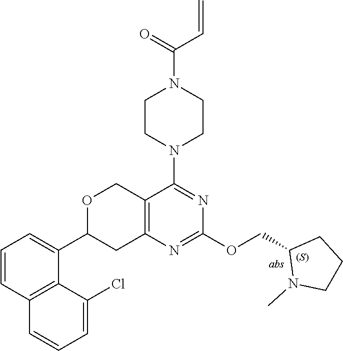 |
| 41 | 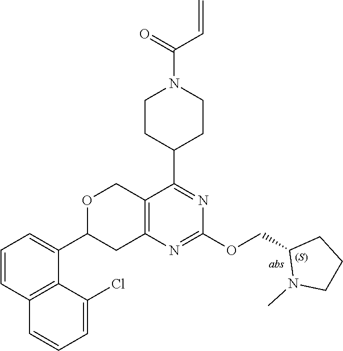 |
| 42 | 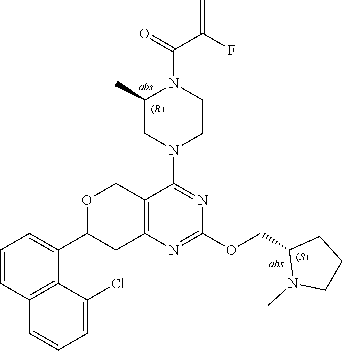 |
| 43 | 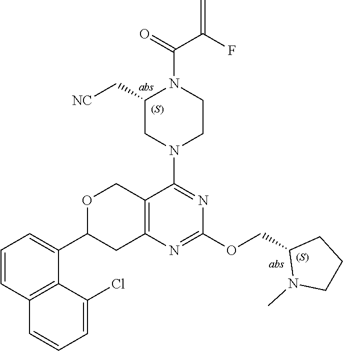 |
| 44 | 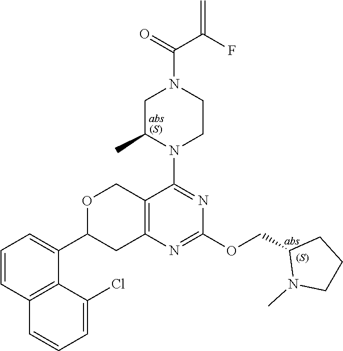 |
| 45 | 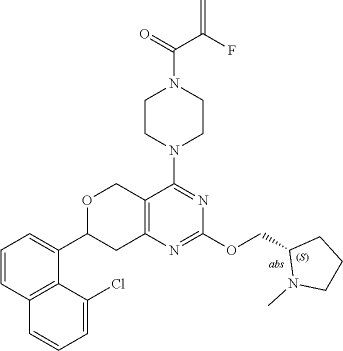 |
| 46 | 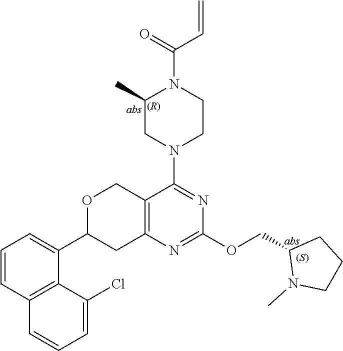 |
| 47 | 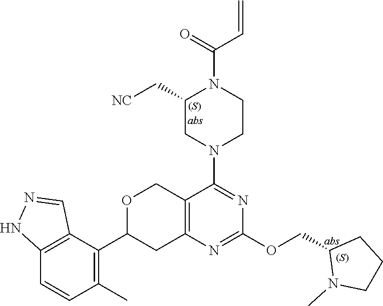 |
| 48 | 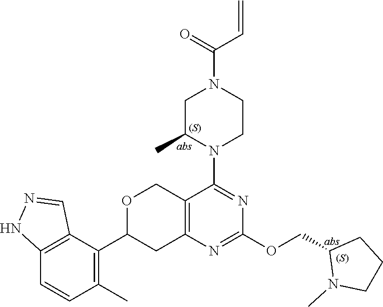 |
| 49 | 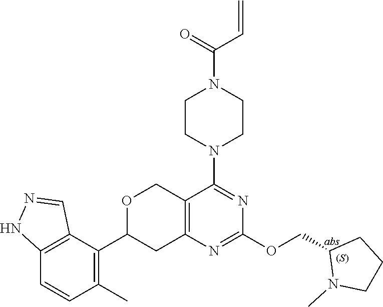 |
| 50 | 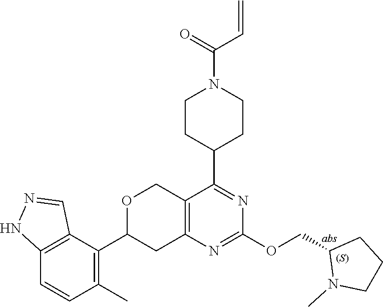 |
| 51 | 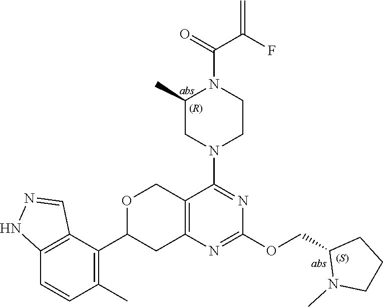 |
| 52 | 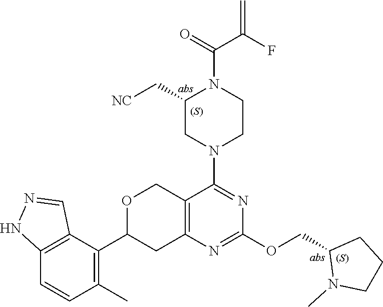 |
| 53 | 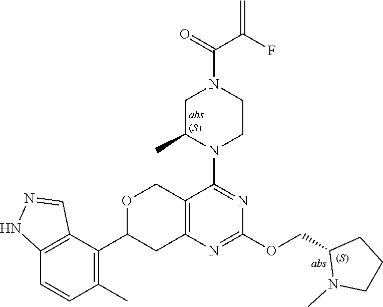 |
| 54 | 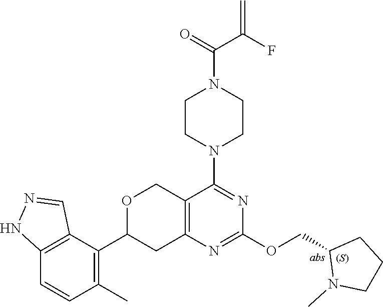 |
| 55 | 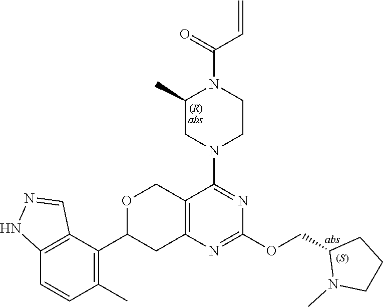 |
| 56 | 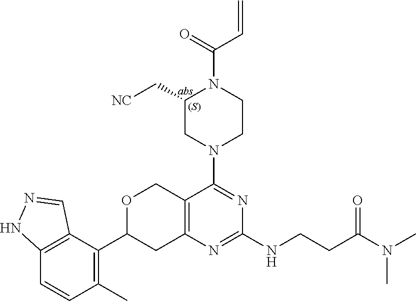 |
| 57 | 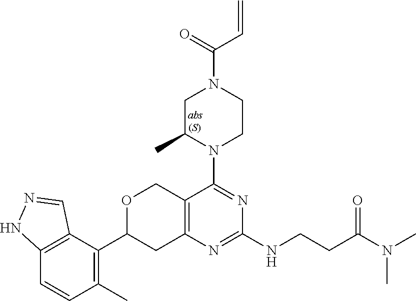 |
| 58 | 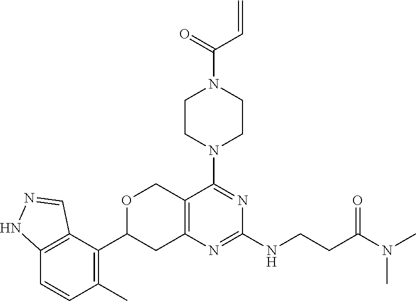 [SMILES: C=CC(=O)N1CCN(c2nc(NCCC(=O)N(C)C)nc3c2COC(c2c(C)ccc4[nH]ncc24)C3)CC1] |
| 59 | 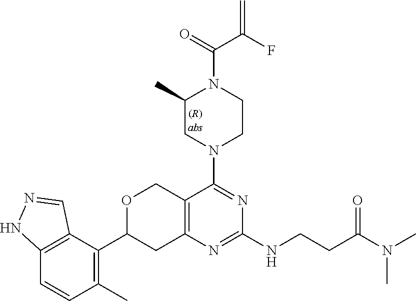 |
| 60 | 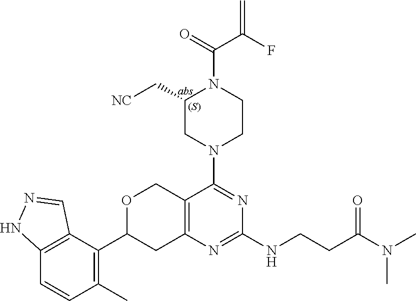 |
| 61 | 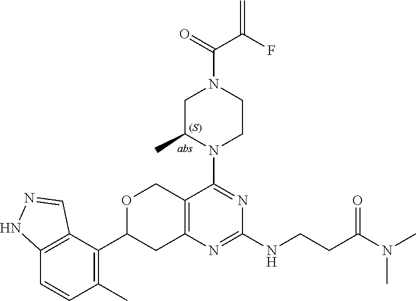 |
| 62 | 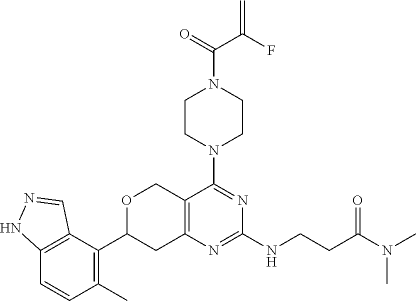 [SMILES: C=C(F)C(=O)N1CCN(c2nc(NCCC(=O)N(C)C)nc3c2COC(c2c(C)ccc4[nH]ncc24)C3)CC1] |
| 63 | 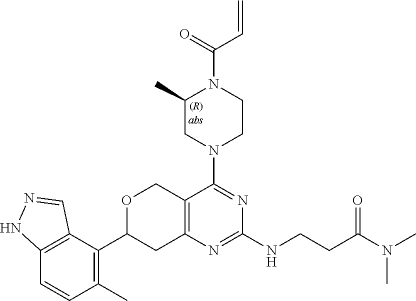 |
| 64 | 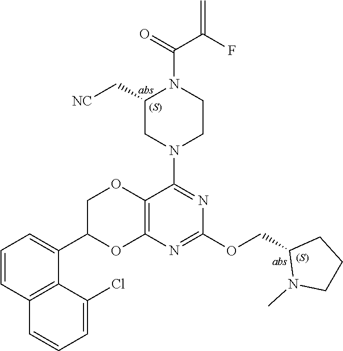 |
| 65 | 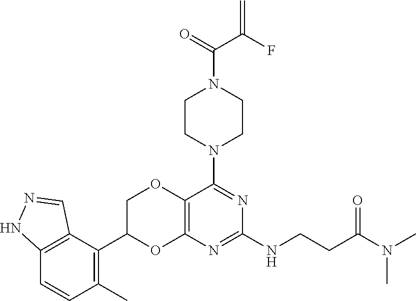 [SMILES: C=C(F)C(=O)N1CCN(c2nc(NCCC(=O)N(C)C)nc3c2OCC(c2c(C)ccc4[nH]ncc24)O3)CC1] |
| 66 | 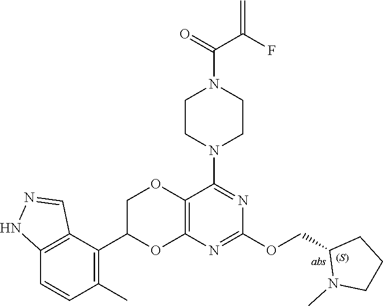 |
| 67 | 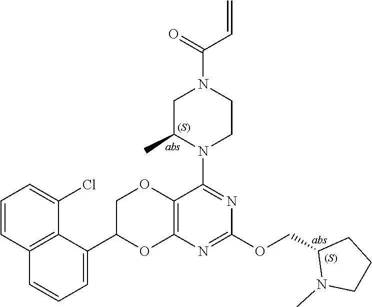 |
| 68 | 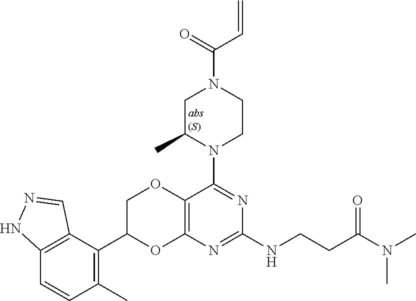 |
| 69 | 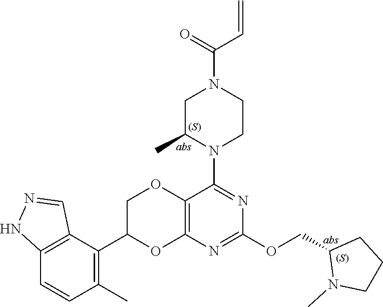 |
| 70 | 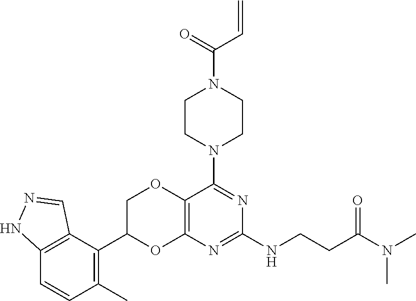 [SMILES: C=CC(=O)N1CCN(c2nc(NCCC(=O)N(C)C)nc3c2OCC(c2c(C)ccc4[nH]ncc24)O3)CC1] |
| 71 | 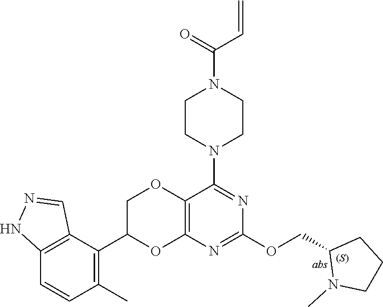 |
| 72 | 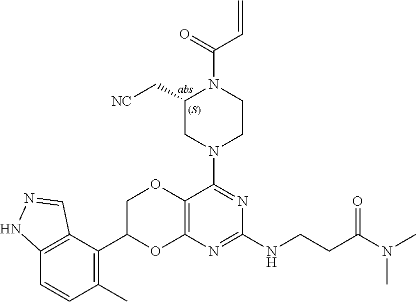 |
| 73 | 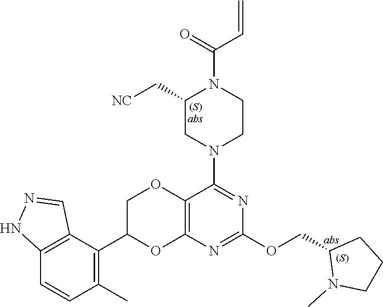 |
| 74 | 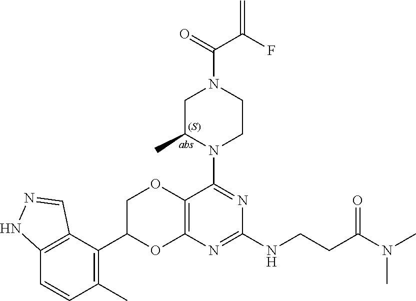 |
| 75 | 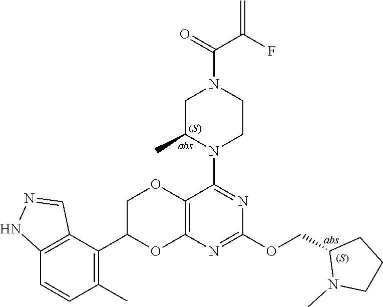 |
| 76 | 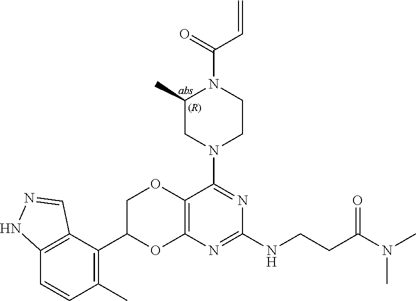 |
| 77 | 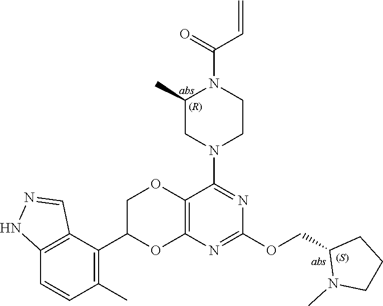 |
| 78 | 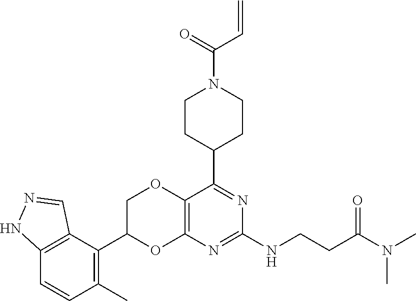 [SMILES: C=CC(=O)N1CCC(c2nc(NCCC(=O)N(C)C)nc3c2OCC(c2c(C)ccc4[nH]ncc24)O3)CC1] |
| 79 | 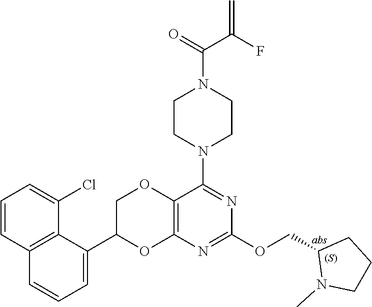 |
| 80 | 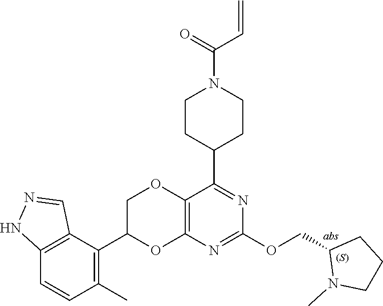 |
| 81 | 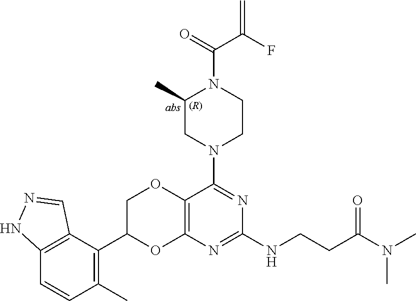 |
| 82 | 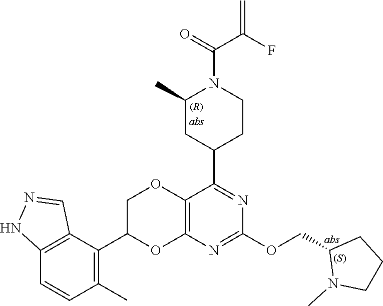 |
| 83 | 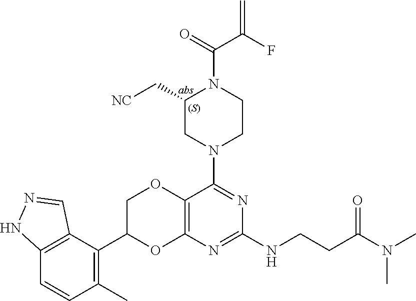 |
| 84 | 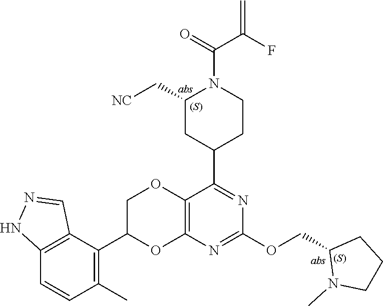 |
| 85 | 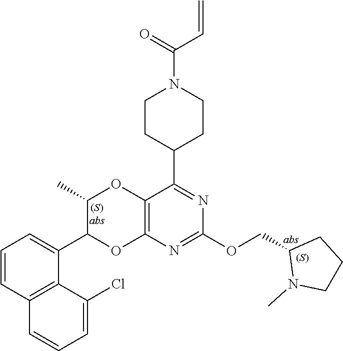 |
| 86 | 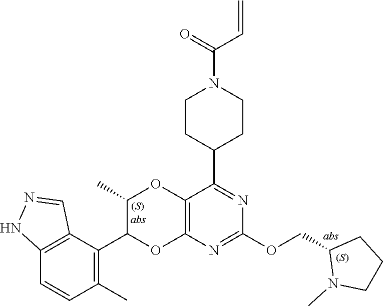 |
| 87 | 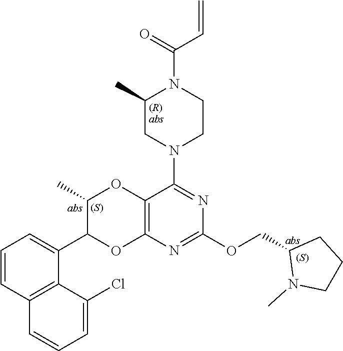 |
| 88 | 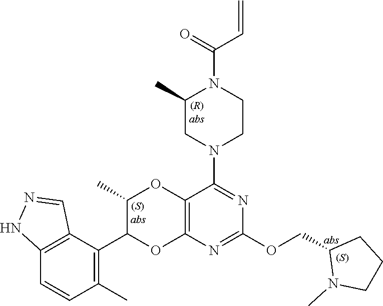 |
| 89 | 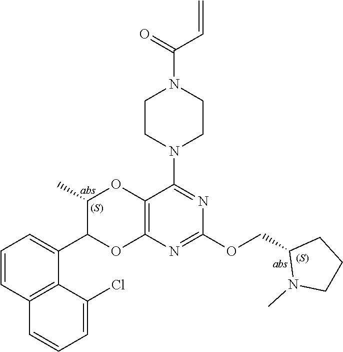 |
| 90 | 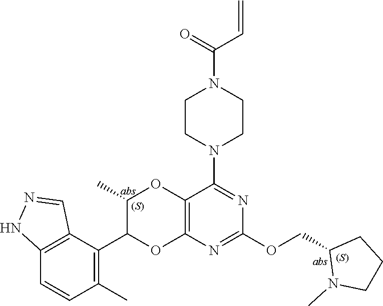 |
| 91 | 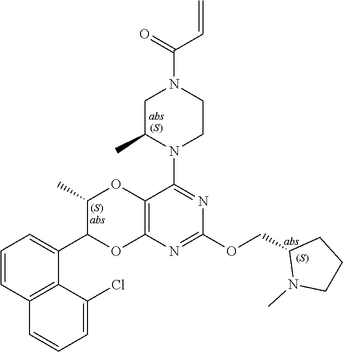 |
| 92 | 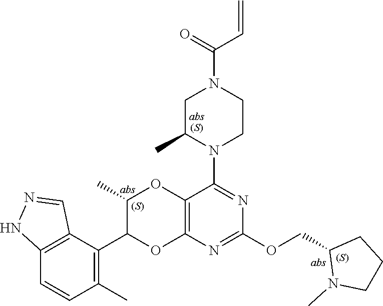 |
| 93 | 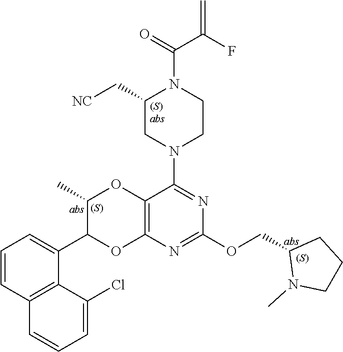 |
| 94 | 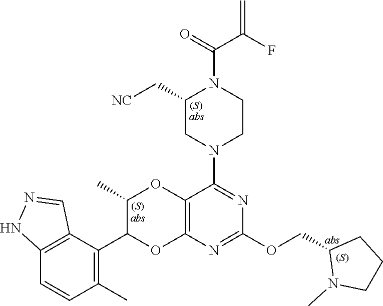 |
| 95 | 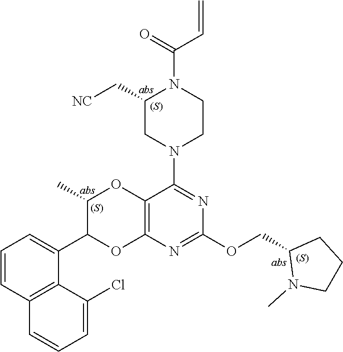 |
| 96 | 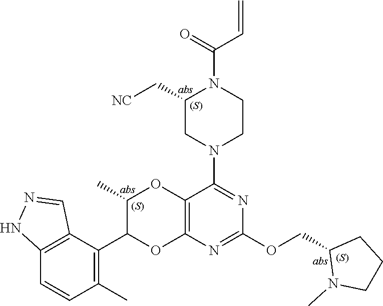 |
| 97 | 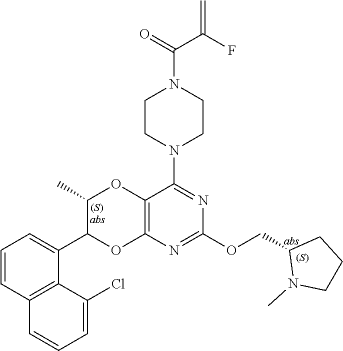 |
| 98 | 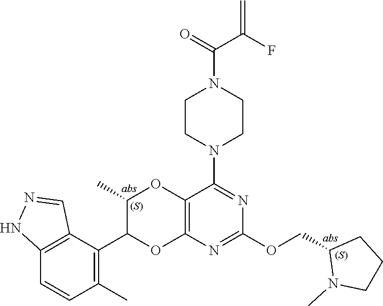 |
| 99 | 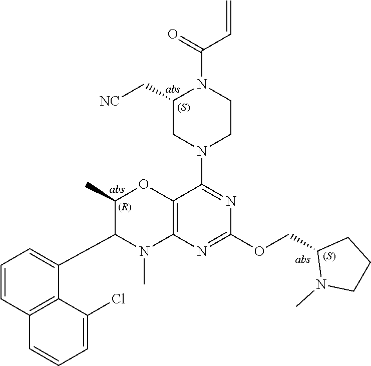 |
| 100 | 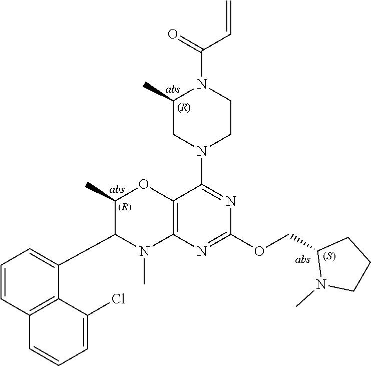 |
| 101 | 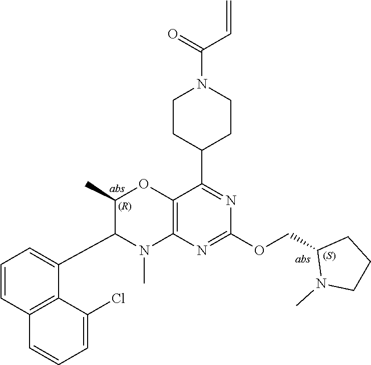 |
| 102 | 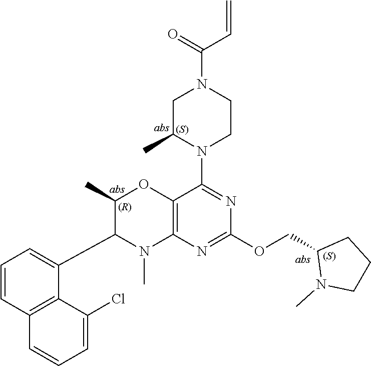 |
| 103 | 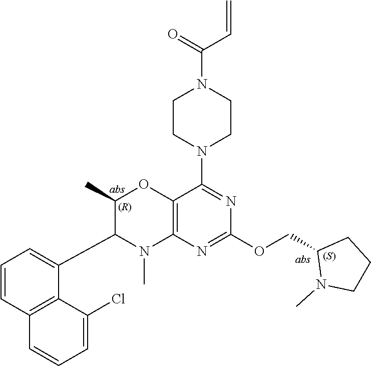 |
| 104 | 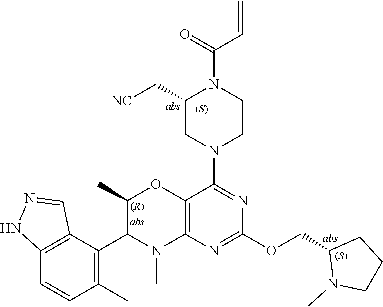 |
| 105 | 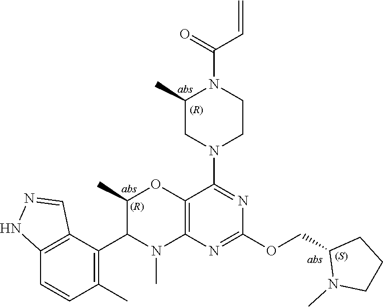 |
| 106 | 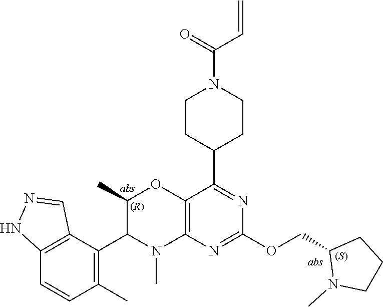 |
| 107 | 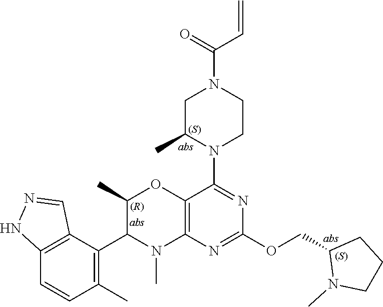 |
| 108 | 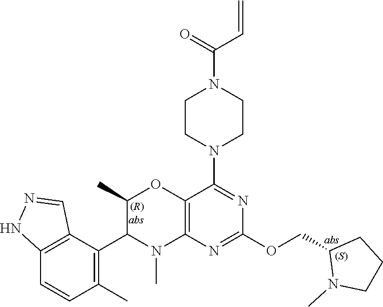 |
| 109 | 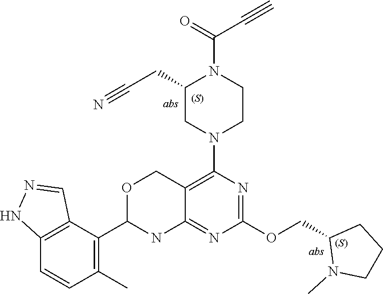 |
| 110 | 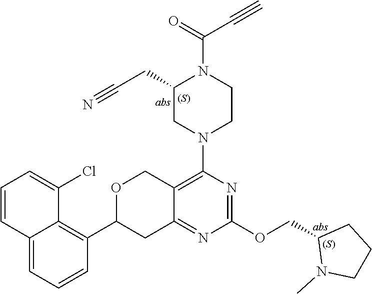 |
| 111 | 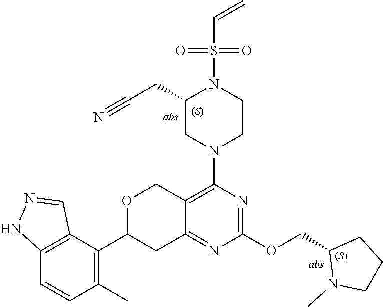 |
| 112 | 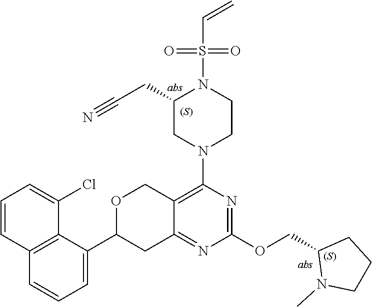 |
| 113 | 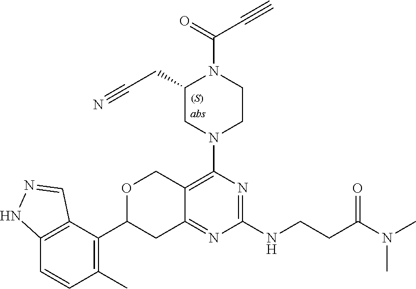 |
| 114 | 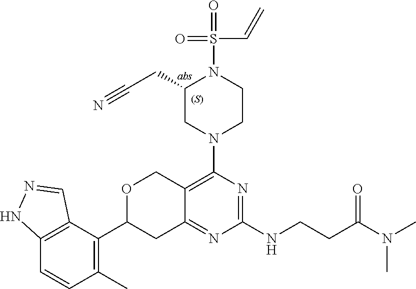 |
| 115 | 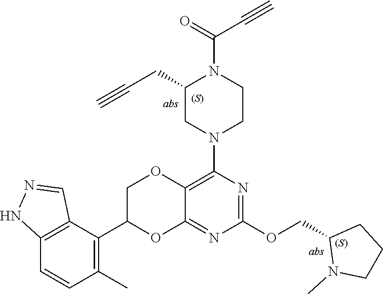 |
| 116 | 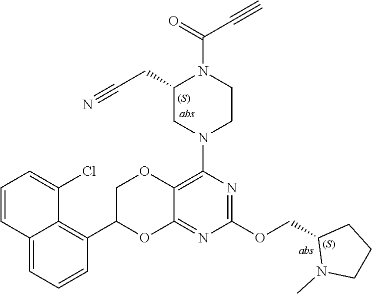 |
| 117 | 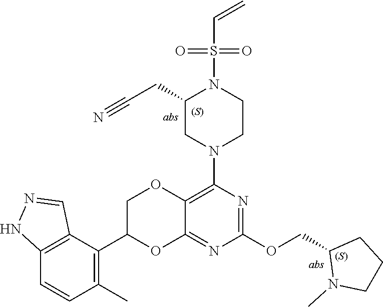 |
| 118 | 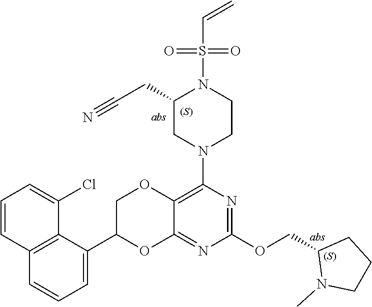 |
| 119 | 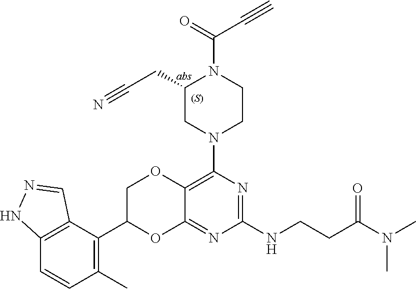 |
| 120 | 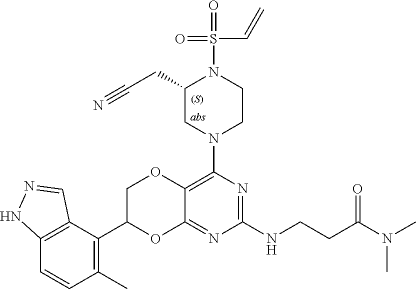 |
| 121 | 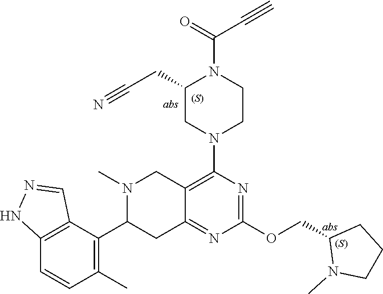 |
| 122 | 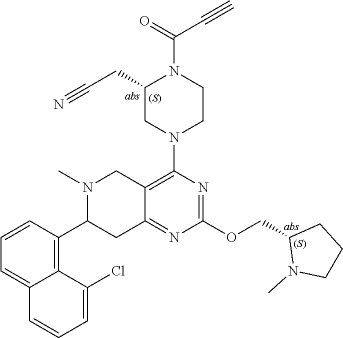 |
| 123 | 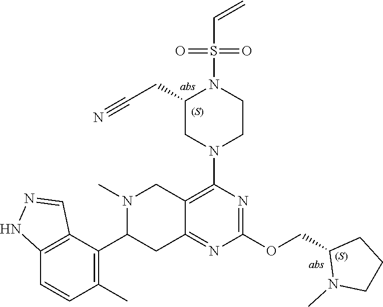 |
| 124 | 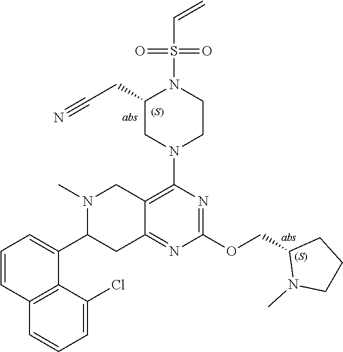 |
| 125 | 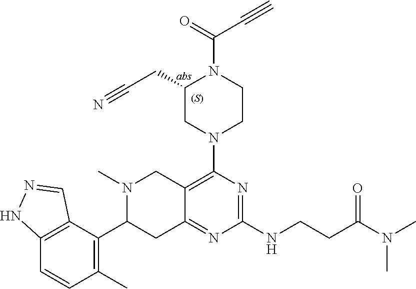 |
| 126 | 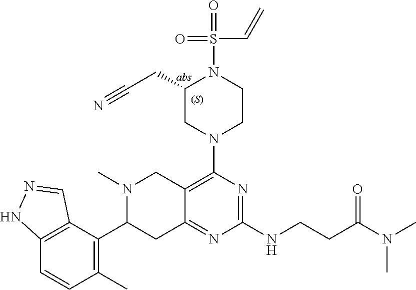 |
| 127 | 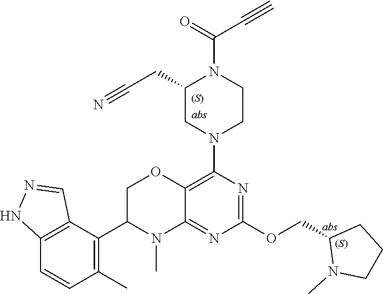 |
| 128 | 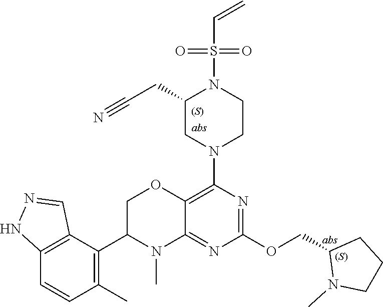 |
| 129 | 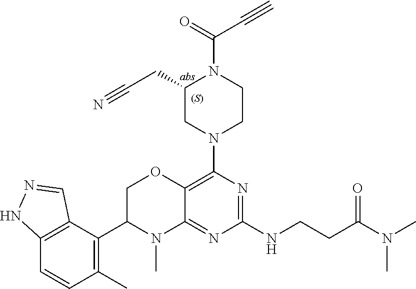 |
| 130 | 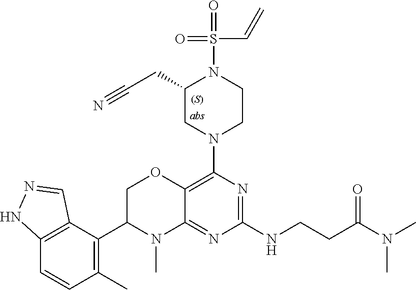 |
| 131 | 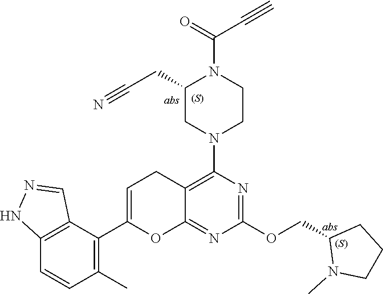 |
| 132 | 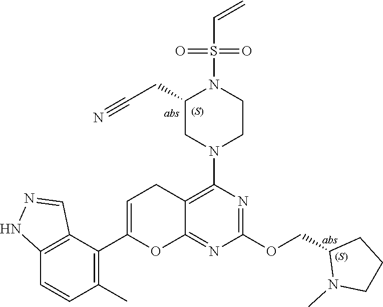 |
| 133 | 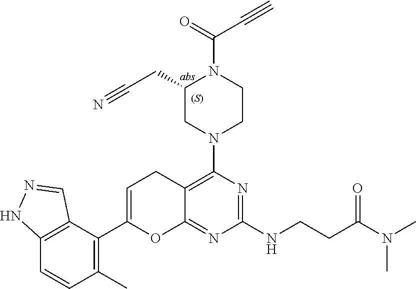 |
| 134 | 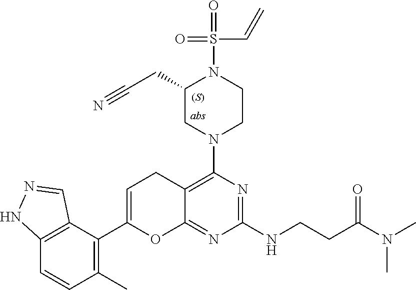 |
| 135 | 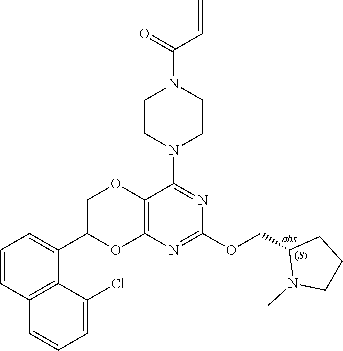 |
| 136 | 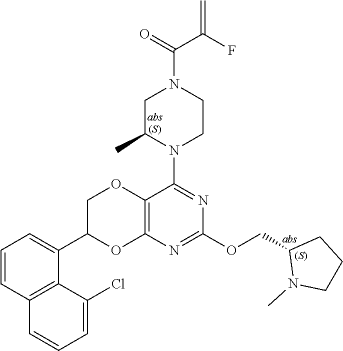 |
| 137 | 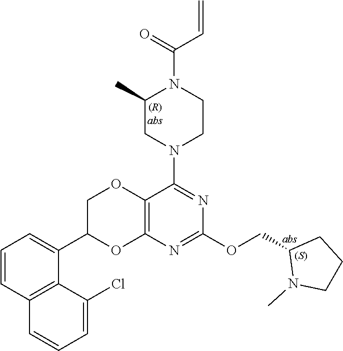 |
| 138 | 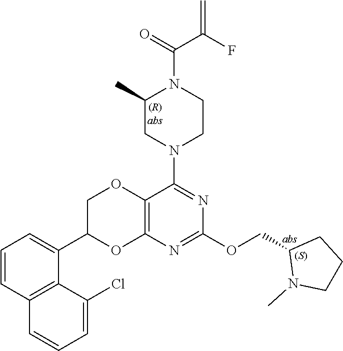 |
| 139 | 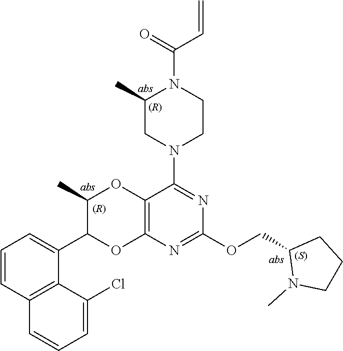 |
| 140 | 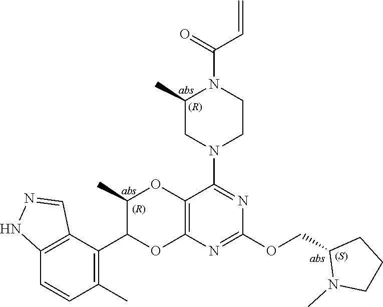 |
| 141 | 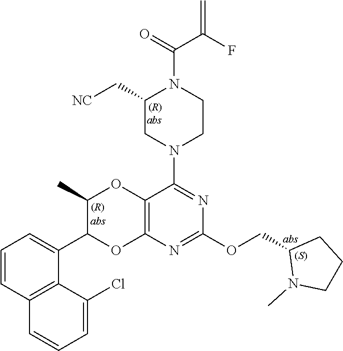 |
| 142 | 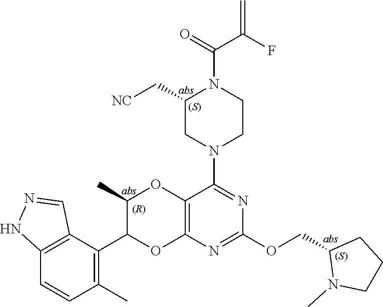 |
| 143 | 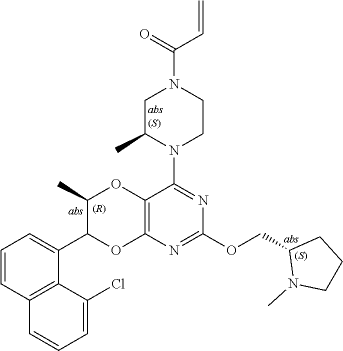 |
| 144 | 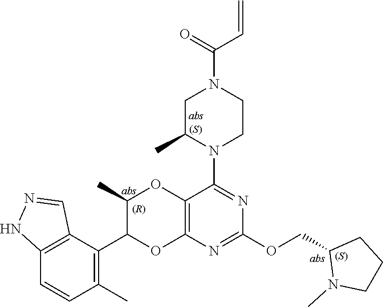 |
| 145 | 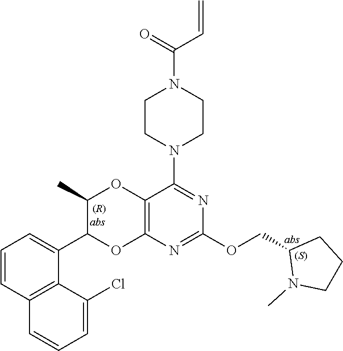 |
| 146 | 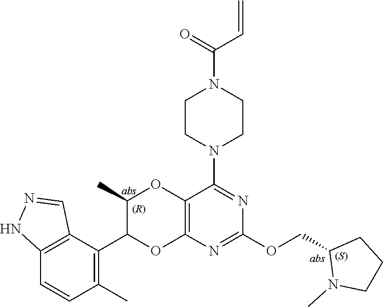 |
| 147 | 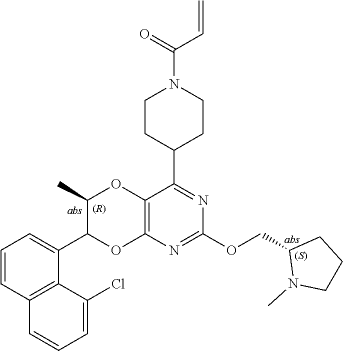 |
| 148 | 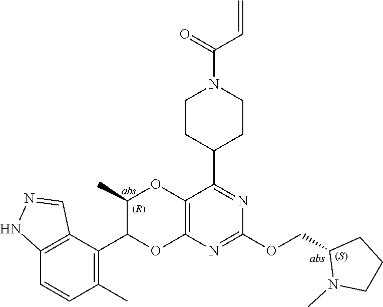 |
| 149 | 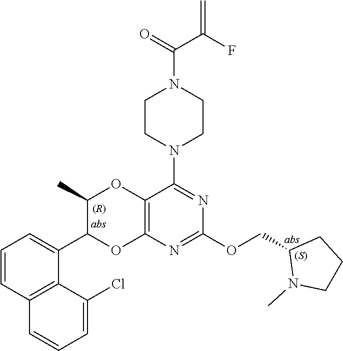 |
| 150 | 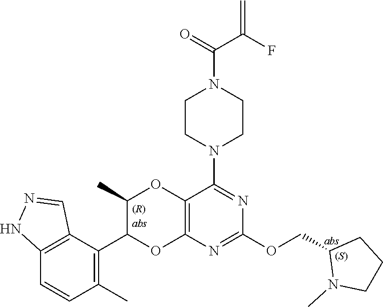 |
| 151 | 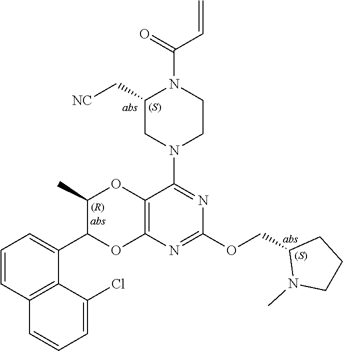 |
| 152 | 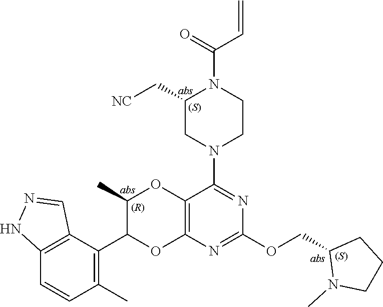 |
| 153 | 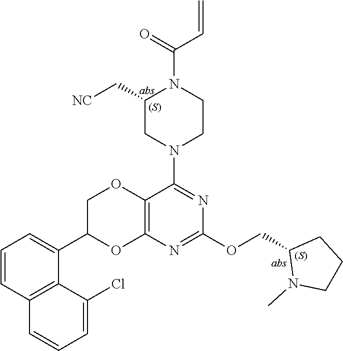 |
| 154 | 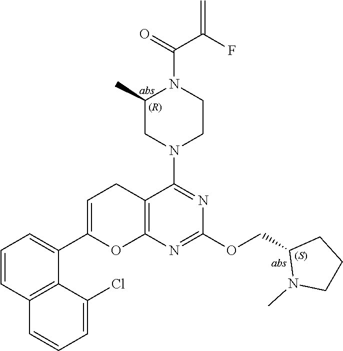 |
| 155 | 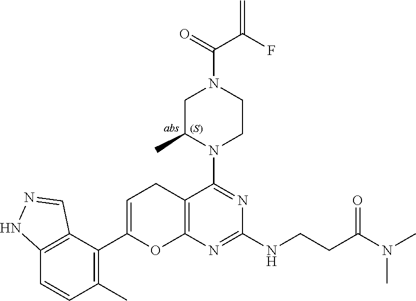 |
| 156 | 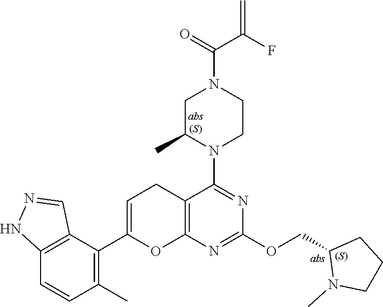 |
| 157 | 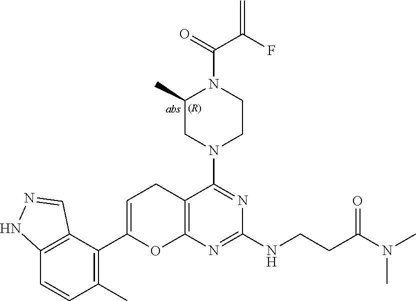 |
| 158 | 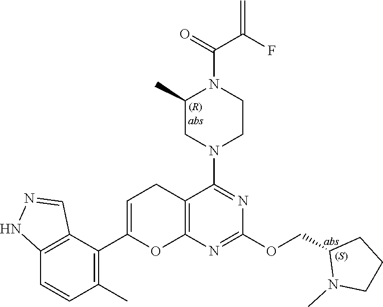 |
| 159 | 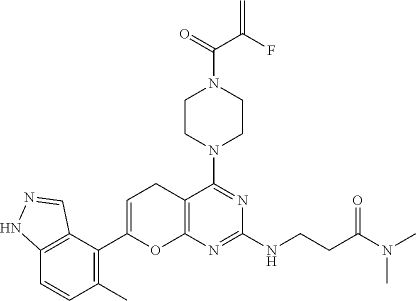 [SMILES: C=C(F)C(=O)N1CCN(c2nc(NCCC(=O)N(C)C)nc3c2CC=C(c2c(C)ccc4[nH]ncc24)O3)CC1] |
| 160 | 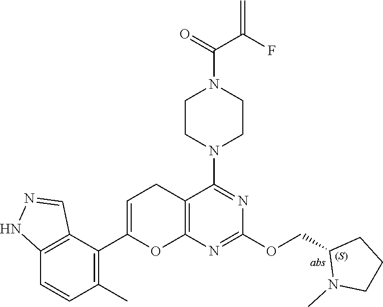 |
| 161 | 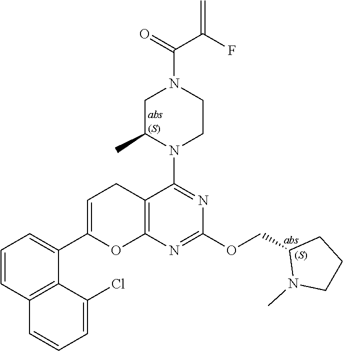 |
| 162 | 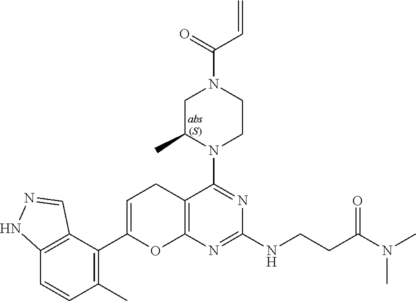 |
| 163 | 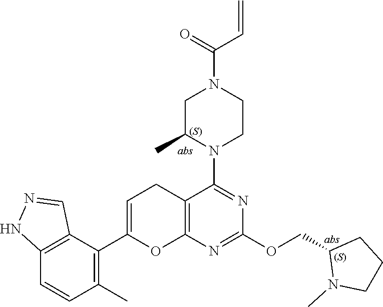 |
| 164 | 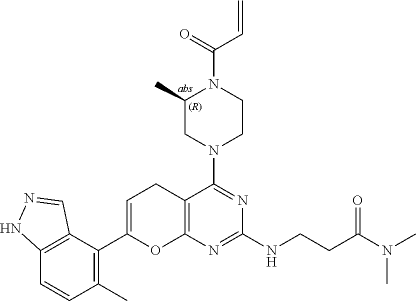 |
| 165 | 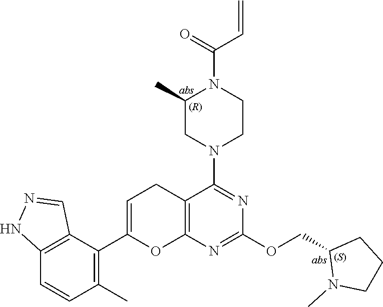 |
| 166 | 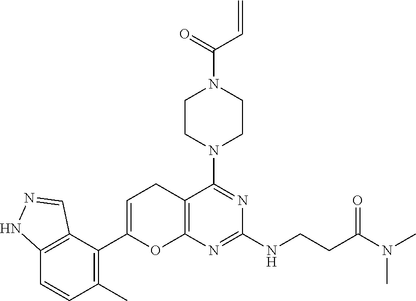 [SMILES: C=CC(=O)N1CCN(c2nc(NCCC(=O)N(C)C)nc3c2CC=C(c2c(C)ccc4[nH]ncc24)O3)CC1] |
| 167 | 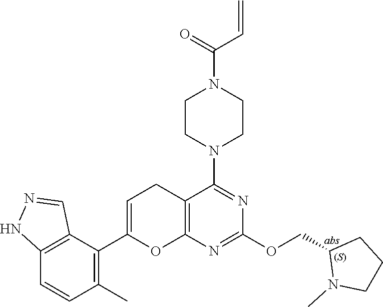 |
| 168 | 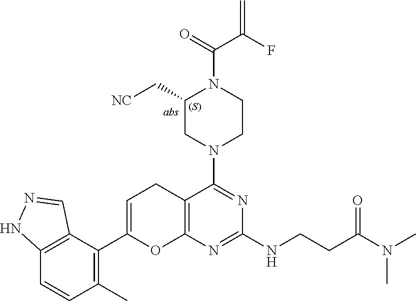 |
| 169 | 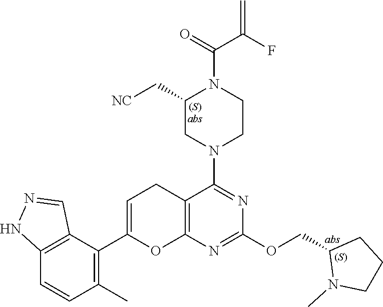 |
| 170 | 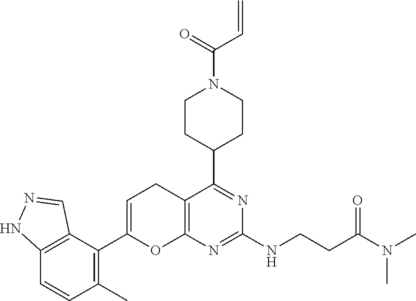 [SMILES: C=CC(=O)N1CCC(c2nc(NCCC(=O)N(C)C)nc3c2CC=C(c2c(C)ccc4[nH]ncc24)O3)CC1] |
| 171 | 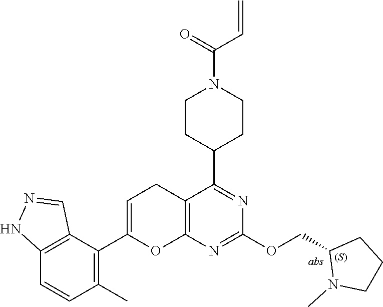 |
| 172 | 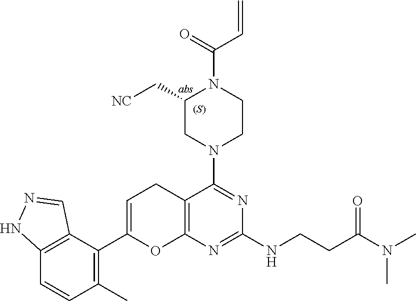 |
| 173 | 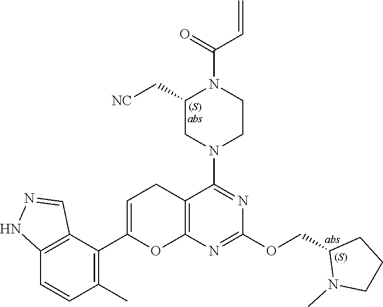 |
| 174 | 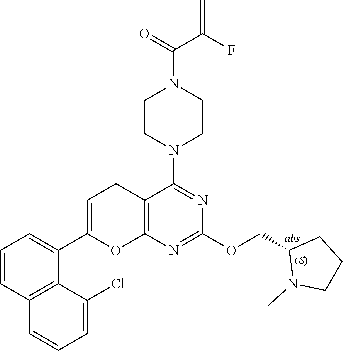 |
| 175 | 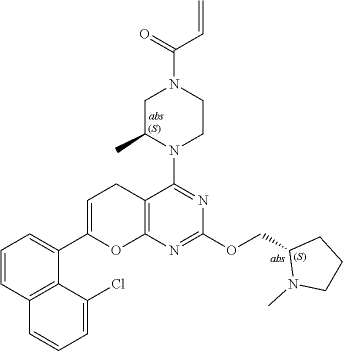 |
| 176 | 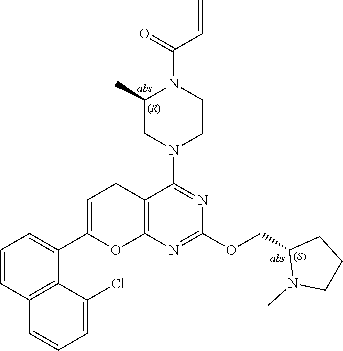 |
| 177 | 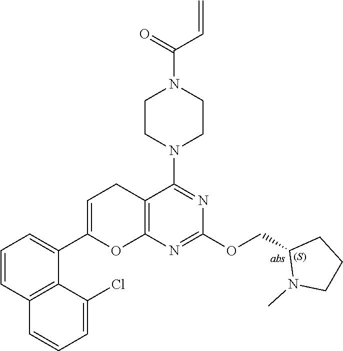 |
| 178 | 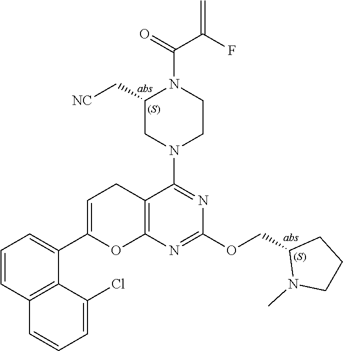 |
| 179 | 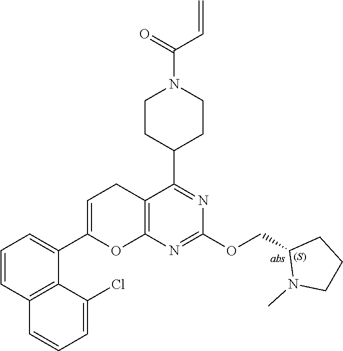 |
| 180 | 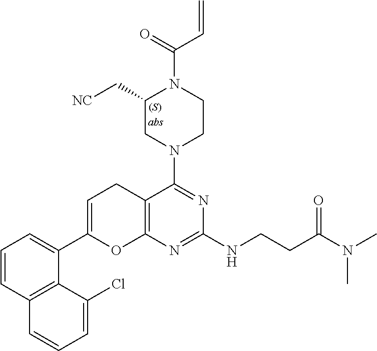 |
| 181 | 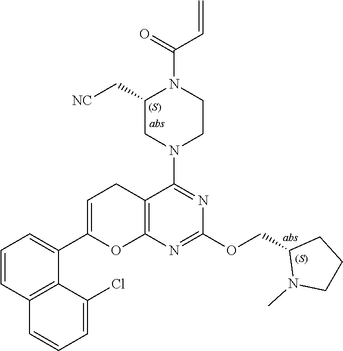 |
| 182 | 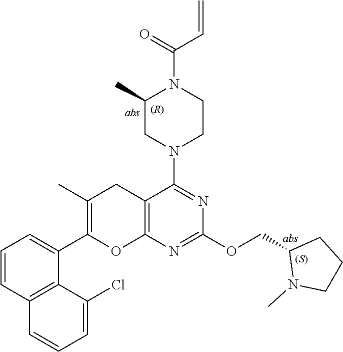 |
| 183 | 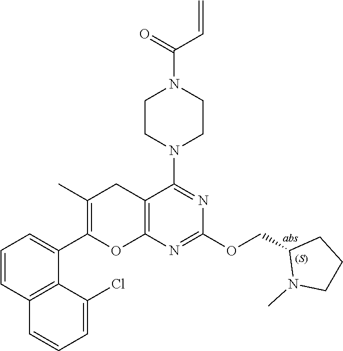 |
| 184 | 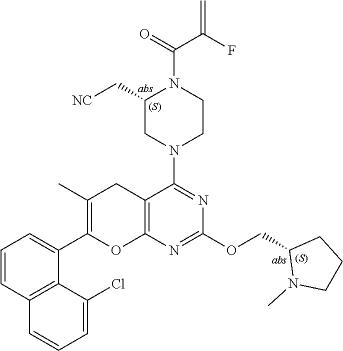 |
| 185 | 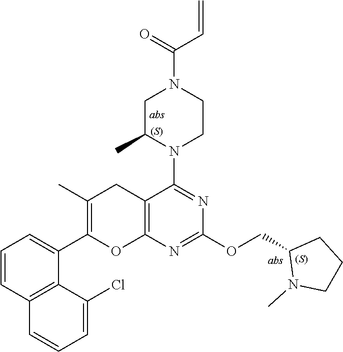 |
| 186 | 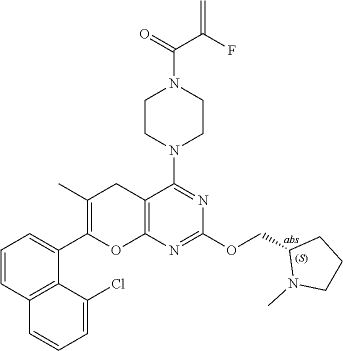 |
| 187 | 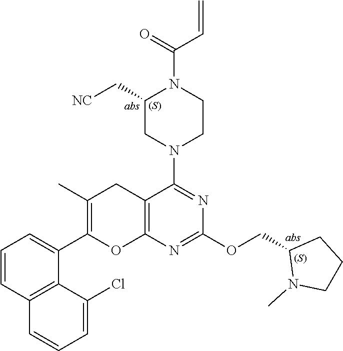 |
| 188 | 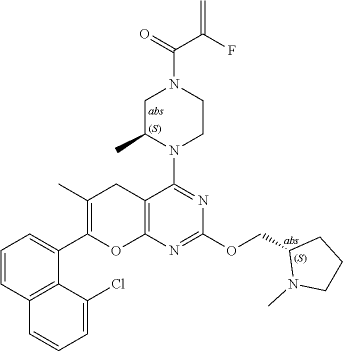 |
| 189 | 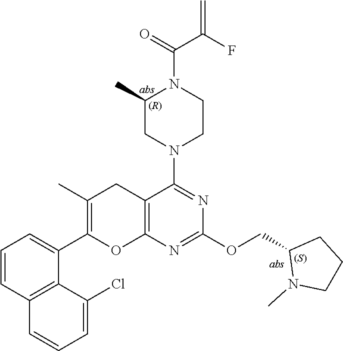 |
| 190 | 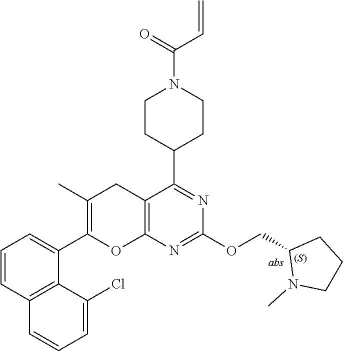 |
| 191 | 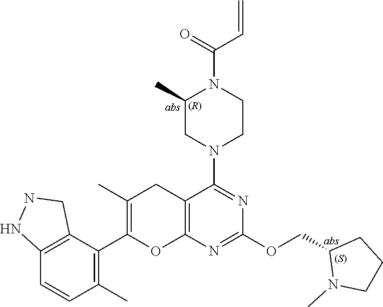 |
| 192 | 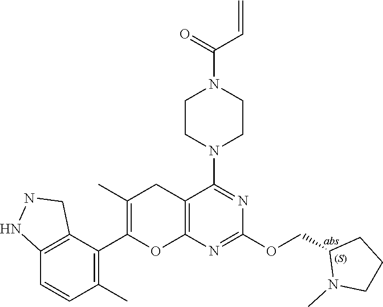 |
| 193 | 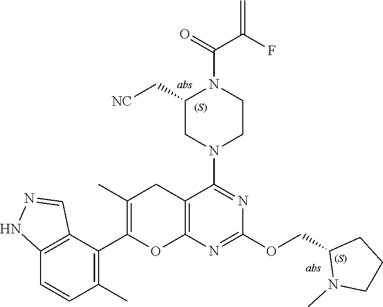 |
| 194 | 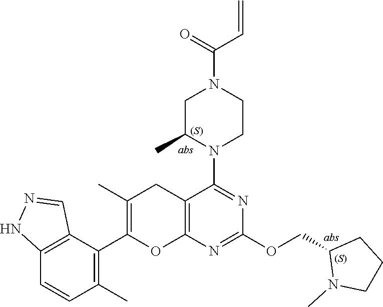 |
| 195 | 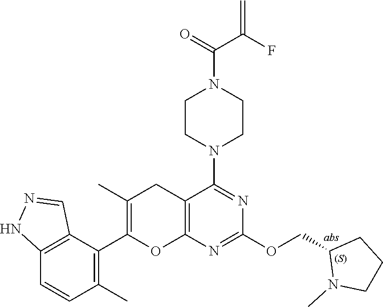 |
| 196 | 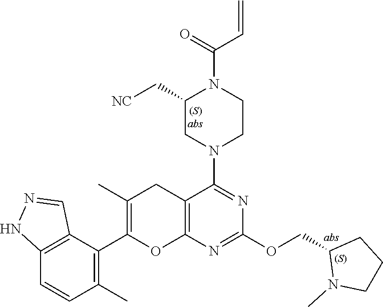 |
| 197 | 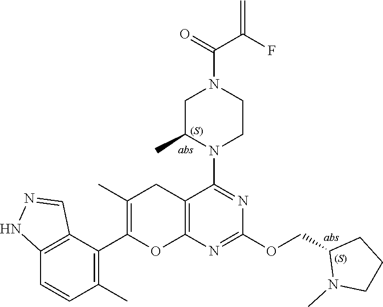 |
| 198 | 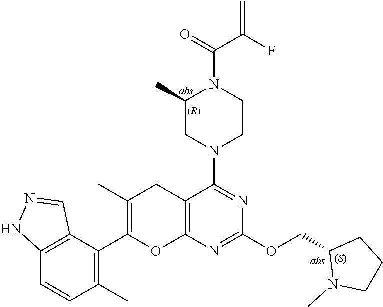 |
| 199 | 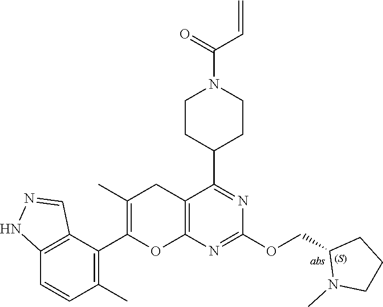 |
| 200 | 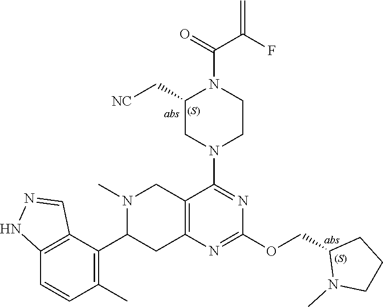 |
| 201 | 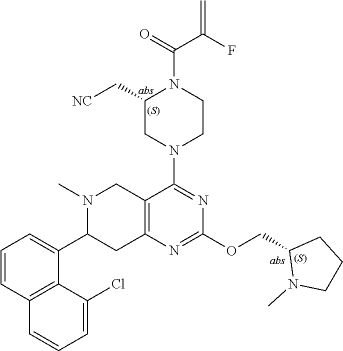 |
| 202 | 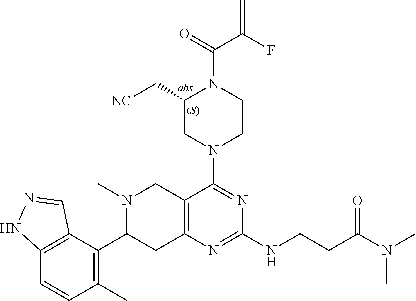 |
| 203 | 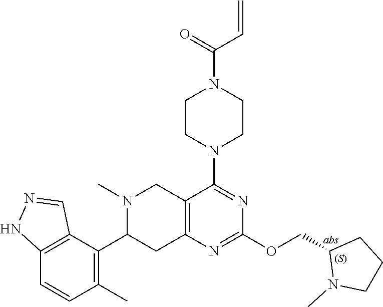 |
| 204 | 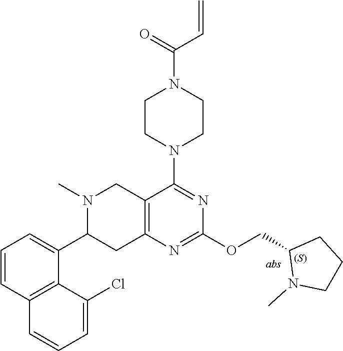 |
| 205 | 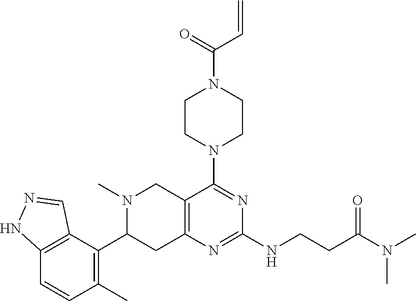 [SMILES: C=CC(=O)N1CCN(c2nc(NCCC(=O)N(C)C)nc3c2CN(C)C(c2c(C)ccc4[nH]ncc24)C3)CC1] |
| 206 | 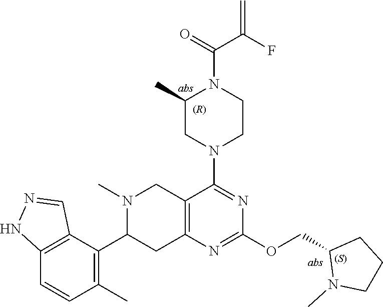 |
| 207 | 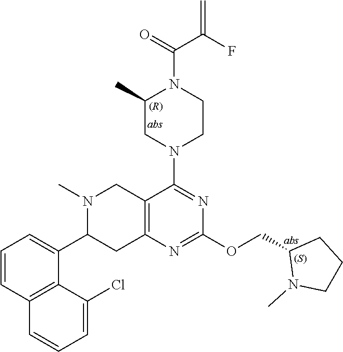 |
| 208 | 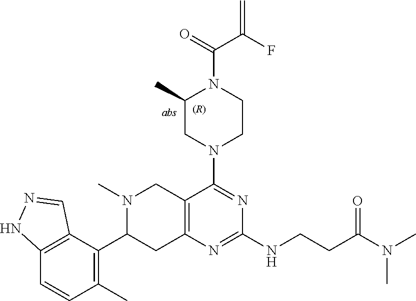 |
| 209 | 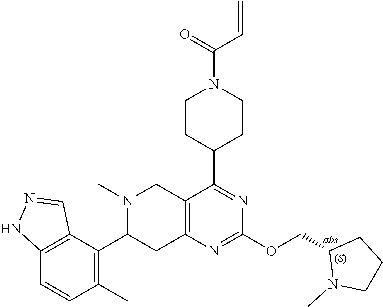 |
| 210 | 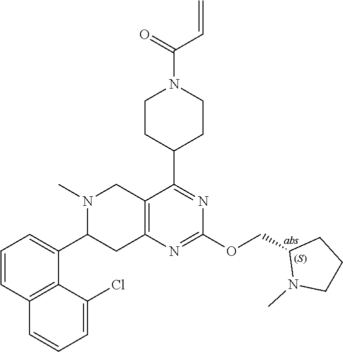 |
| 211 | 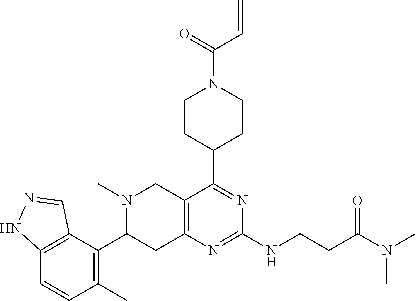 [SMILES: C=CC(=O)N1CCC(c2nc(NCCC(=O)N(C)C)nc3c2CN(C)C(c2c(C)ccc4[nH]ncc24)C3)CC1] |
| 212 | 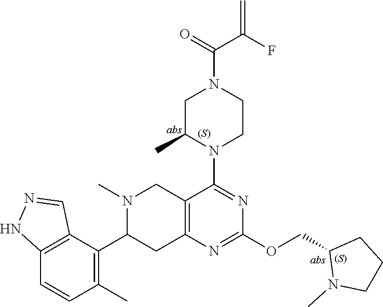 |
| 213 | 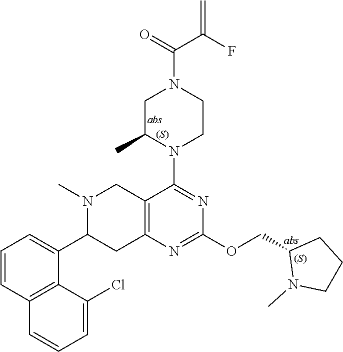 |
| 214 | 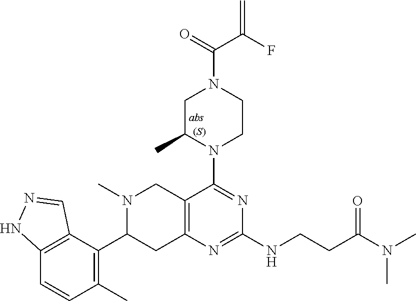 |
| 215 | 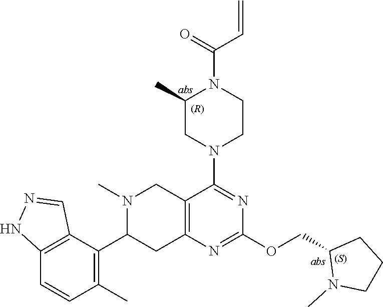 |
| 216 | 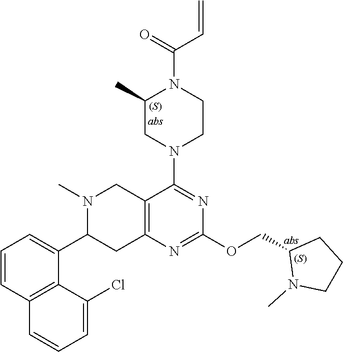 |
| 217 | 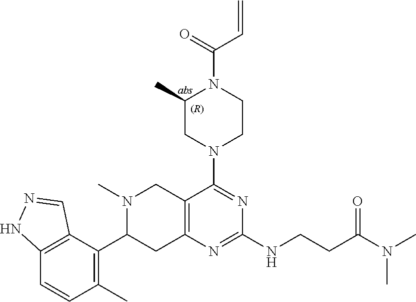 |
| 218 | 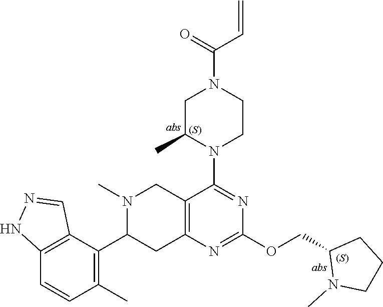 |
| 219 | 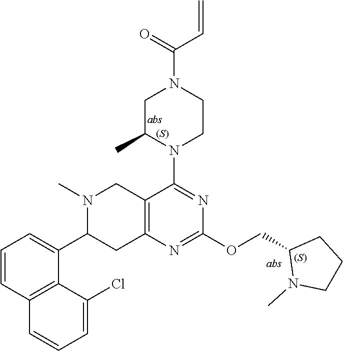 |
| 220 | 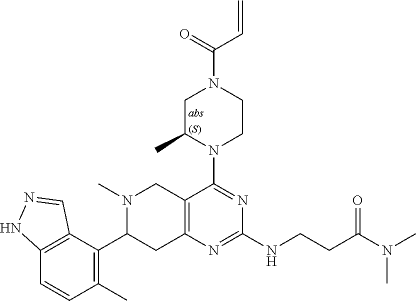 |
| 221 | 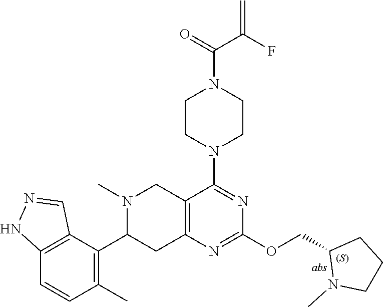 |
| 222 | 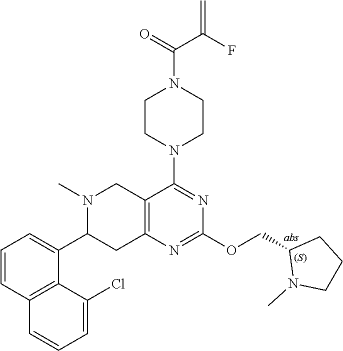 |
| 223 | 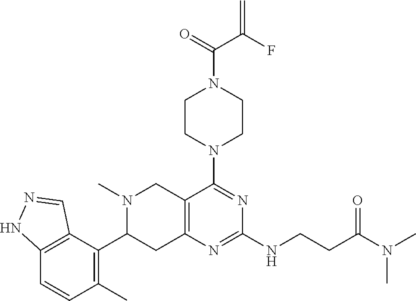 [SMILES: C=C(F)C(=O)N1CCN(c2nc(NCCC(=O)N(C)C)nc3c2CN(C)C(c2c(C)ccc4[nH]ncc24)C3)CC1] |
| 224 | 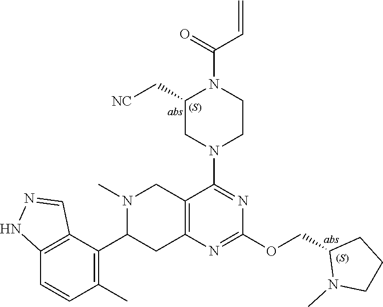 |
| 225 | 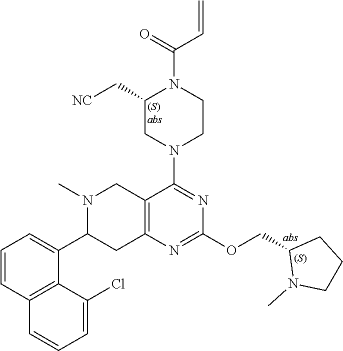 |
| 226 | 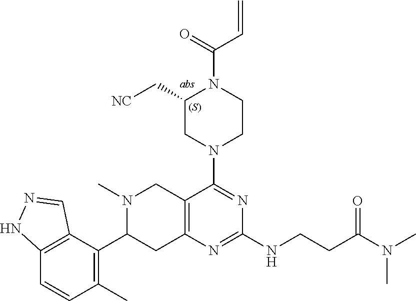 |
| 227 | 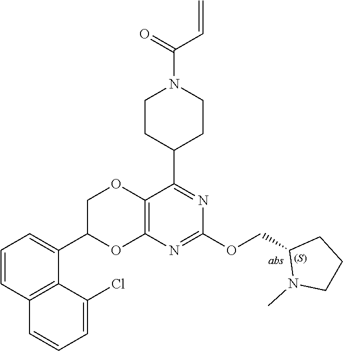 |
| 228 | 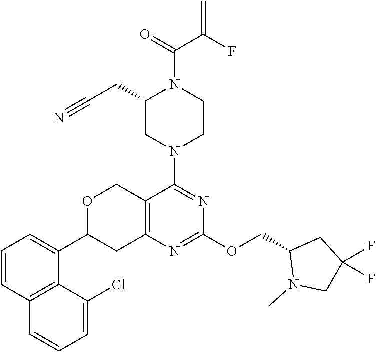 [SMILES: C=C(F)C(=O)N1CCN(c2nc(OC[C@@H]3CC(F)(F)CN3C)nc3c2COC(c2cccc4cccc(Cl)c24)C3)C[C@@H]1CC#N] |
| 229 | 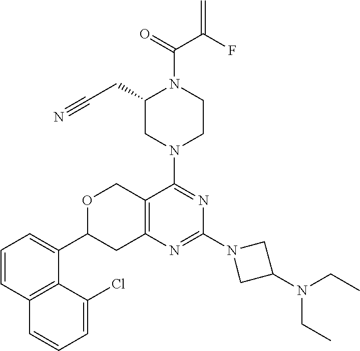 [SMILES: C=C(F)C(=O)N1CCN(c2nc(N3CC(N(CC)CC)C3)nc3c2COC(c2cccc4cccc(Cl)c24)C3)C[C@@H]1CC#N] |
| 230 | 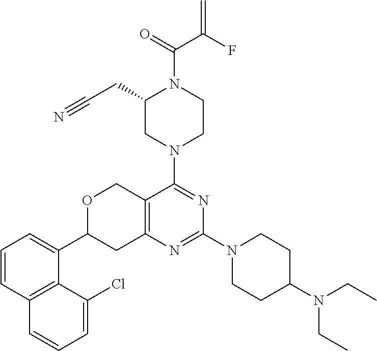 [SMILES: C=C(F)C(=O)N1CCN(c2nc(N3CCC(N(CC)CC)CC3)nc3c2COC(c2cccc4cccc(Cl)c24)C3)C[C@@H]1CC#N] |
| 231 | 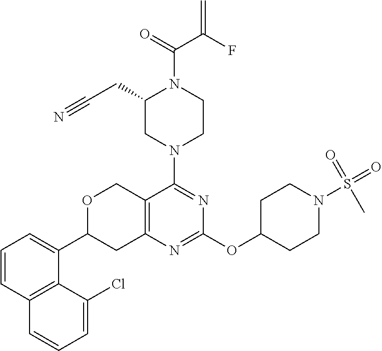 [SMILES: C=C(F)C(=O)N1CCN(c2nc(OC3CCN(S(C)(=O)=O)CC3)nc3c2COC(c2cccc4cccc(Cl)c24)C3)C[C@@H]1CC#N] |
| 232 | 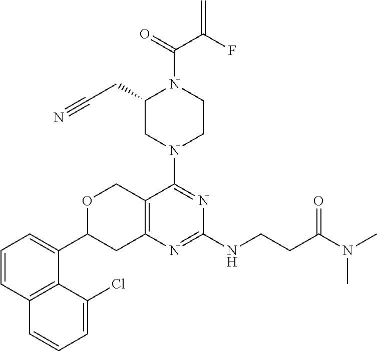 [SMILES: C=C(F)C(=O)N1CCN(c2nc(NCCC(=O)N(C)C)nc3c2COC(c2cccc4cccc(Cl)c24)C3)C[C@@H]1CC#N] |
| 233 | 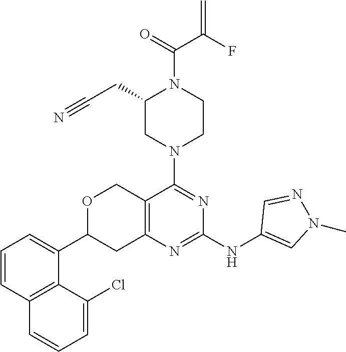 [SMILES: C=C(F)C(=O)N1CCN(c2nc(Nc3cnn(C)c3)nc3c2COC(c2cccc4cccc(Cl)c24)C3)C[C@@H]1CC#N] |
| 234 | 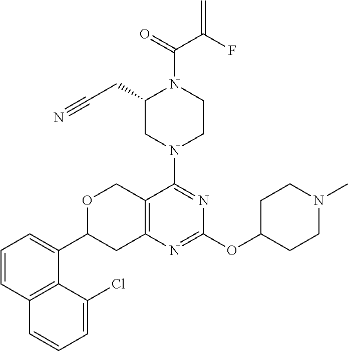 [SMILES: C=C(F)C(=O)N1CCN(c2nc(OC3CCN(C)CC3)nc3c2COC(c2cccc4cccc(Cl)c24)C3)C[C@@H]1CC#N] |
| 235 | 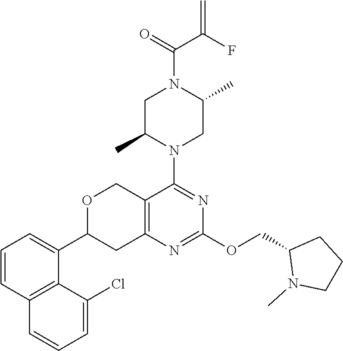 [SMILES: C=C(F)C(=O)N1C[C@H](C)N(c2nc(OC[C@@H]3CCCN3C)nc3c2COC(c2cccc4cccc(Cl)c24)C3)C[C@H]1C] |
| 236 | 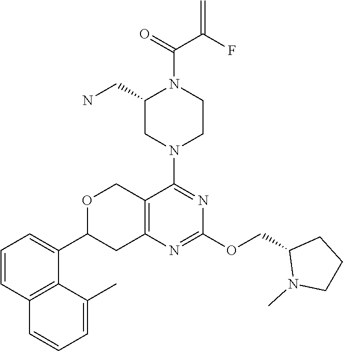 [SMILES: C=C(F)C(=O)N1CCN(c2nc(OC[C@@H]3CCCN3C)nc3c2COC(c2cccc4cccc(C)c24)C3)C[C@@H]1CN] |
| 237 | 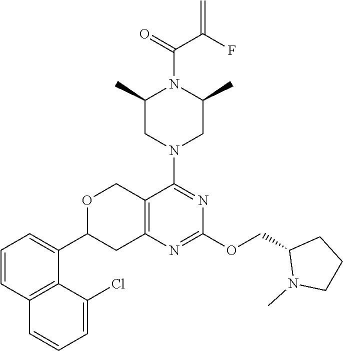 [SMILES: C=C(F)C(=O)N1[C@H](C)CN(c2nc(OC[C@@H]3CCCN3C)nc3c2COC(c2cccc4cccc(Cl)c24)C3)C[C@@H]1C] |
| 238 | 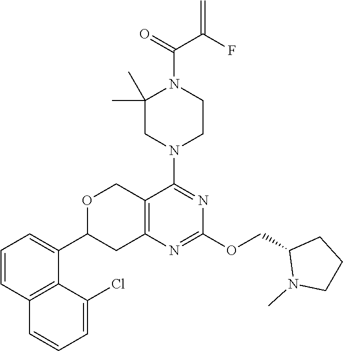 [SMILES: C=C(F)C(=O)N1CCN(c2nc(OC[C@@H]3CCCN3C)nc3c2COC(c2cccc4cccc(Cl)c24)C3)CC1(C)C] |
| 239 | 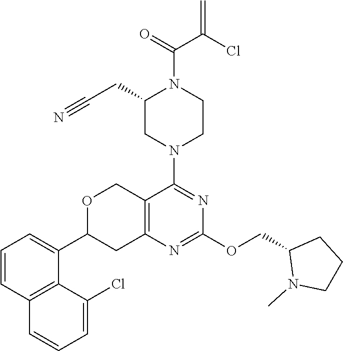 [SMILES: C=C(Cl)C(=O)N1CCN(c2nc(OC[C@@H]3CCCN3C)nc3c2COC(c2cccc4cccc(Cl)c24)C3)C[C@@H]1CC#N] |
| 240 | 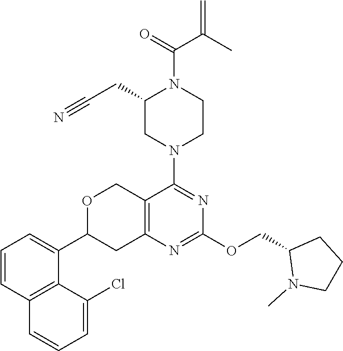 [SMILES: C=C(C)C(=O)N1CCN(c2nc(OC[C@@H]3CCCN3C)nc3c2COC(c2cccc4cccc(Cl)c24)C3)C[C@@H]1CC#N] |
| 241 | 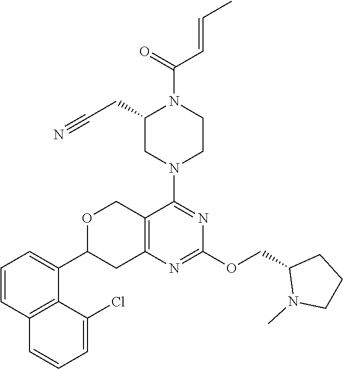 [SMILES: C/C=C/C(=O)N1CCN(c2nc(OC[C@@H]3CCCN3C)nc3c2COC(c2cccc4cccc(Cl)c24)C3)C[C@@H]1CC#N] |
| 242 | 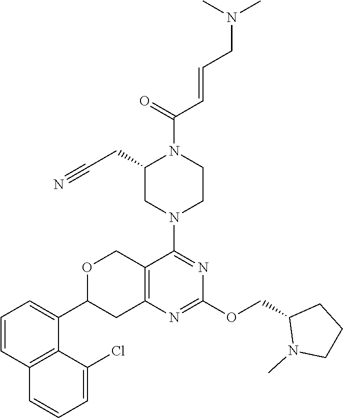 [SMILES: CN(C)C/C=C/C(=O)N1CCN(c2nc(OC[C@@H]3CCCN3C)nc3c2COC(c2cccc4cccc(Cl)c24)C3)C[C@@H]1CC#N] |
| 243 | 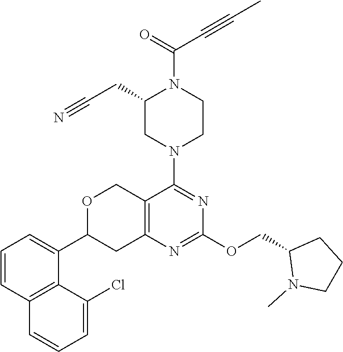 [SMILES: CC#CC(=O)N1CCN(c2nc(OC[C@@H]3CCCN3C)nc3c2COC(c2cccc4cccc(Cl)c24)C3)C[C@@H]1CC#N] |
| 244 | 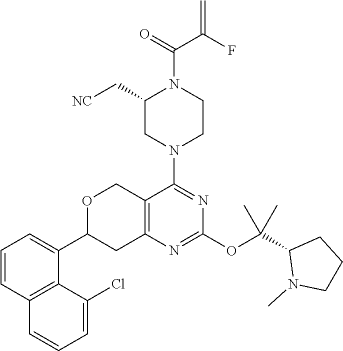 [SMILES: C=C(F)C(=O)N1CCN(c2nc(OC(C)(C)[C@@H]3CCCN3C)nc3c2COC(c2cccc4cccc(Cl)c24)C3)C[C@@H]1CC#N] |
| 245 | 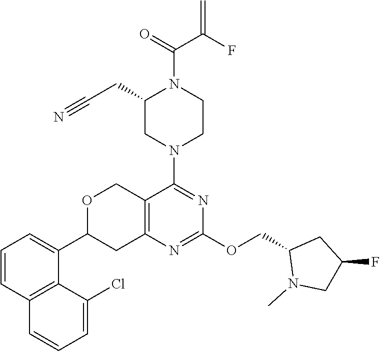 [SMILES: C=C(F)C(=O)N1CCN(c2nc(OC[C@@H]3C[C@@H](F)CN3C)nc3c2COC(c2cccc4cccc(Cl)c24)C3)C[C@@H]1CC#N] |
| 246 | 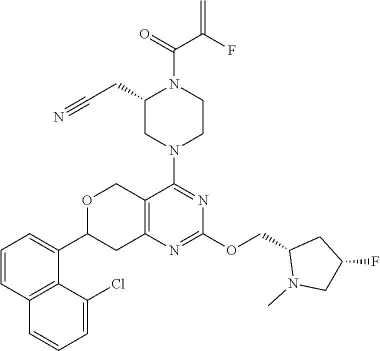 [SMILES: C=C(F)C(=O)N1CCN(c2nc(OC[C@@H]3C[C@H](F)CN3C)nc3c2COC(c2cccc4cccc(Cl)c24)C3)C[C@@H]1CC#N] |
| 247 | 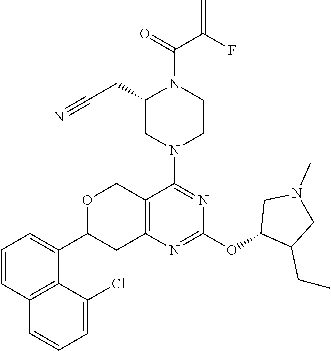 [SMILES: C=C(F)C(=O)N1CCN(c2nc(O[C@@H]3CN(C)CC3CC)nc3c2COC(c2cccc4cccc(Cl)c24)C3)C[C@@H]1CC#N] |
| 248 | 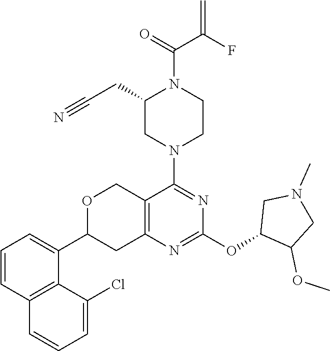 [SMILES: C=C(F)C(=O)N1CCN(c2nc(O[C@@H]3CN(C)CC3OC)nc3c2COC(c2cccc4cccc(Cl)c24)C3)C[C@@H]1CC#N] |
| 249 | 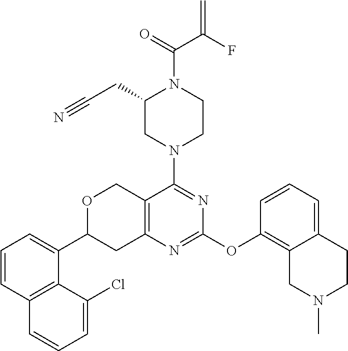 [SMILES: C=C(F)C(=O)N1CCN(c2nc(Oc3cccc4c3CN(C)CC4)nc3c2COC(c2cccc4cccc(Cl)c24)C3)C[C@@H]1CC#N] |
| 250 | 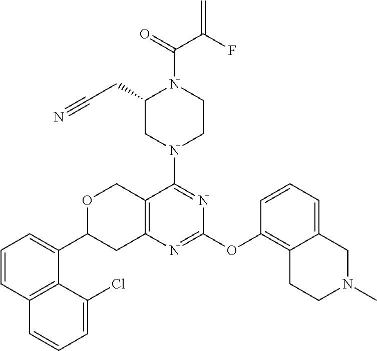 [SMILES: C=C(F)C(=O)N1CCN(c2nc(Oc3cccc4c3CCN(C)C4)nc3c2COC(c2cccc4cccc(Cl)c24)C3)C[C@@H]1CC#N] |
| 251 | 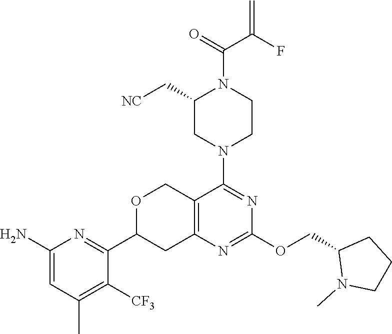 [SMILES: C=C(F)C(=O)N1CCN(c2nc(OC[C@@H]3CCCN3C)nc3c2COC(c2nc(N)cc(C)c2C(F)(F)F)C3)C[C@@H]1CC#N] |
| 252 | 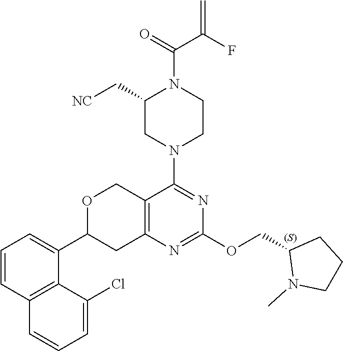 |
| 253 | 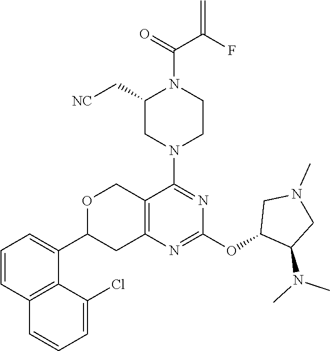 [SMILES: C=C(F)C(=O)N1CCN(c2nc(O[C@@H]3CN(C)C[C@H]3N(C)C)nc3c2COC(c2cccc4cccc(Cl)c24)C3)C[C@@H]1CC#N] |
| 254 | 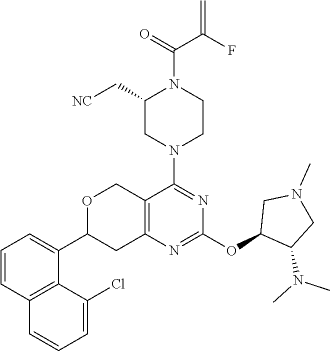 [SMILES: C=C(F)C(=O)N1CCN(c2nc(O[C@H]3CN(C)C[C@@H]3N(C)C)nc3c2COC(c2cccc4cccc(Cl)c24)C3)C[C@@H]1CC#N] |
| 255 | 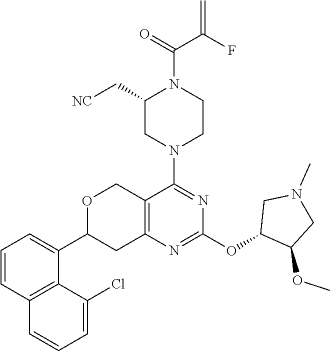 [SMILES: C=C(F)C(=O)N1CCN(c2nc(O[C@@H]3CN(C)C[C@H]3OC)nc3c2COC(c2cccc4cccc(Cl)c24)C3)C[C@@H]1CC#N] |
| 256 | 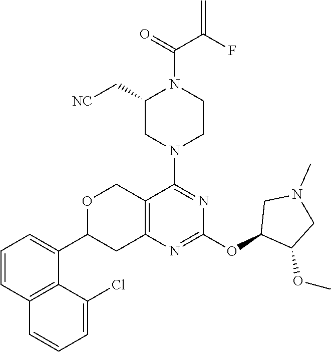 [SMILES: C=C(F)C(=O)N1CCN(c2nc(O[C@H]3CN(C)C[C@@H]3OC)nc3c2COC(c2cccc4cccc(Cl)c24)C3)C[C@@H]1CC#N] |
| 257 | 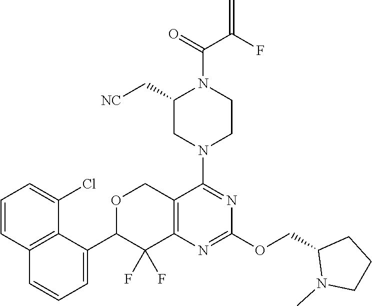 [SMILES: C=C(F)C(=O)N1CCN(c2nc(OC[C@@H]3CCCN3C)nc3c2COC(c2cccc4cccc(Cl)c24)C3(F)F)C[C@@H]1CC#N] |
| 258 | 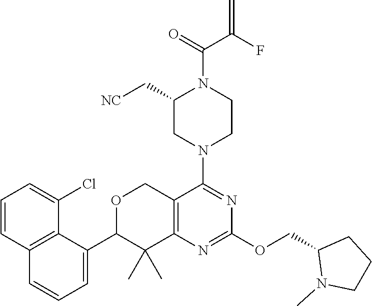 [SMILES: C=C(F)C(=O)N1CCN(c2nc(OC[C@@H]3CCCN3C)nc3c2COC(c2cccc4cccc(Cl)c24)C3(C)C)C[C@@H]1CC#N] |
| 259 | 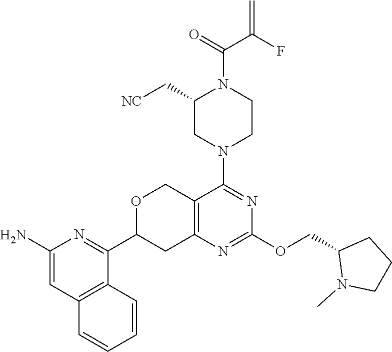 [SMILES: C=C(F)C(=O)N1CCN(c2nc(OC[C@@H]3CCCN3C)nc3c2COC(c2nc(N)cc4ccccc24)C3)C[C@@H]1CC#N] |
| 260 | 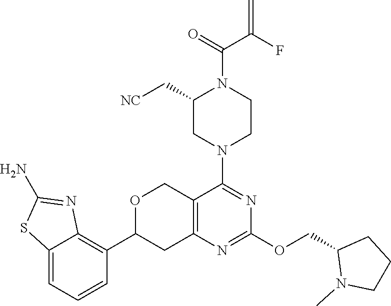 [SMILES: C=C(F)C(=O)N1CCN(c2nc(OC[C@@H]3CCCN3C)nc3c2COC(c2cccc4sc(N)nc24)C3)C[C@@H]1CC#N] |
| 261 | 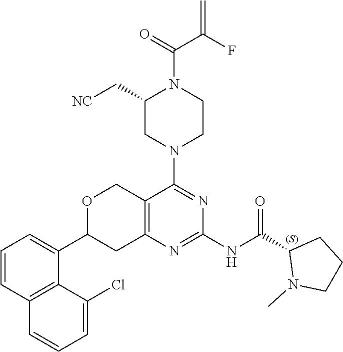 |
| 262 | 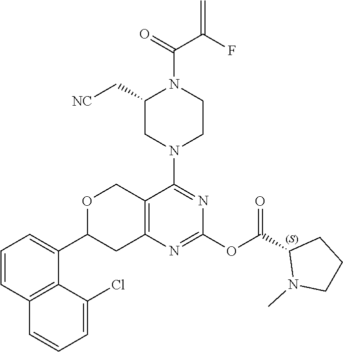 |
| 263 | 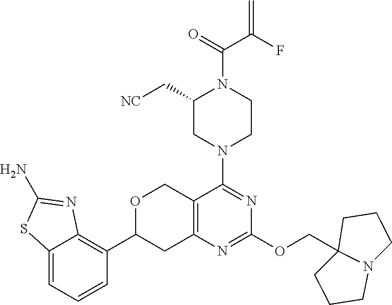 [SMILES: C=C(F)C(=O)N1CCN(c2nc(OCC34CCCN3CCC4)nc3c2COC(c2cccc4sc(N)nc24)C3)C[C@@H]1CC#N] |
| 264 | 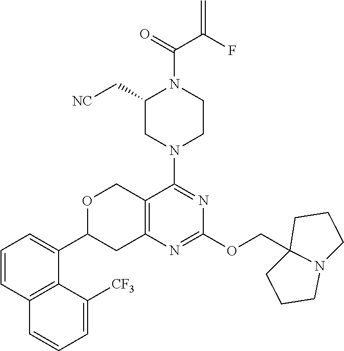 [SMILES: C=C(F)C(=O)N1CCN(c2nc(OCC34CCCN3CCC4)nc3c2COC(c2cccc4cccc(C(F)(F)F)c24)C3)C[C@@H]1CC#N] |
| 265 | 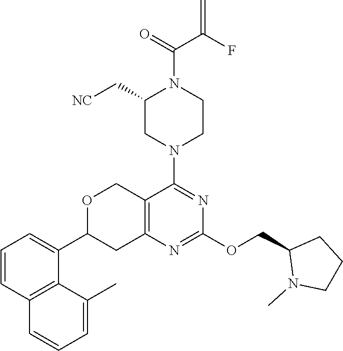 [SMILES: C=C(F)C(=O)N1CCN(c2nc(OC[C@H]3CCCN3C)nc3c2COC(c2cccc4cccc(C)c24)C3)C[C@@H]1CC#N] |
| 266 | 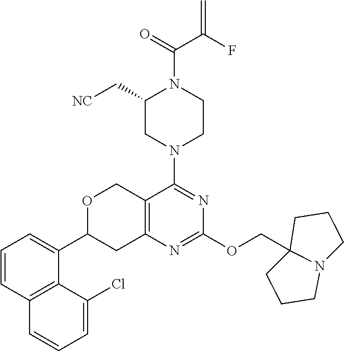 [SMILES: C=C(F)C(=O)N1CCN(c2nc(OCC34CCCN3CCC4)nc3c2COC(c2cccc4cccc(Cl)c24)C3)C[C@@H]1CC#N] |
| 267 | 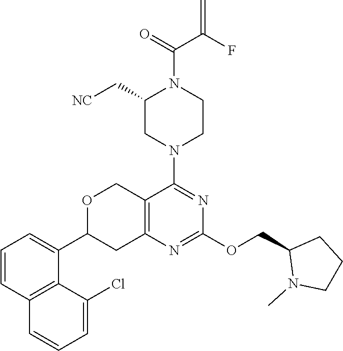 [SMILES: C=C(F)C(=O)N1CCN(c2nc(OC[C@H]3CCCN3C)nc3c2COC(c2cccc4cccc(Cl)c24)C3)C[C@@H]1CC#N] |
| 268 | 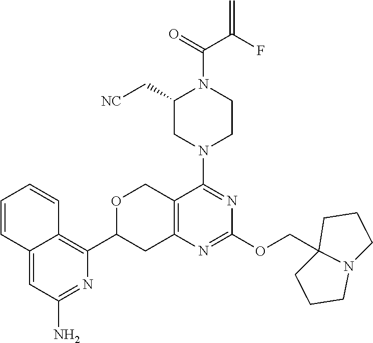 [SMILES: C=C(F)C(=O)N1CCN(c2nc(OCC34CCCN3CCC4)nc3c2COC(c2nc(N)cc4ccccc24)C3)C[C@@H]1CC#N] |
| 269 | 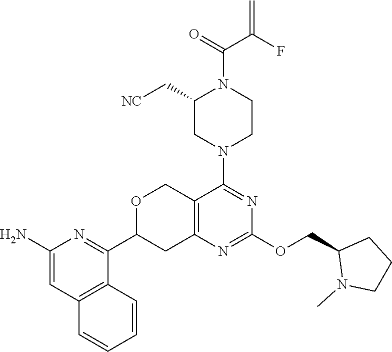 [SMILES: C=C(F)C(=O)N1CCN(c2nc(OC[C@H]3CCCN3C)nc3c2COC(c2nc(N)cc4ccccc24)C3)C[C@@H]1CC#N] |
| 270 | 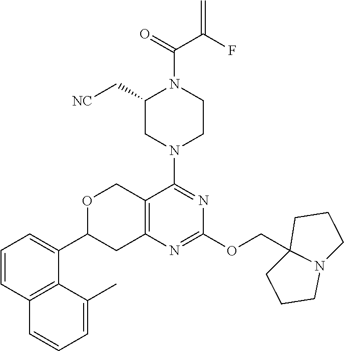 [SMILES: C=C(F)C(=O)N1CCN(c2nc(OCC34CCCN3CCC4)nc3c2COC(c2cccc4cccc(C)c24)C3)C[C@@H]1CC#N] |
| 271 | 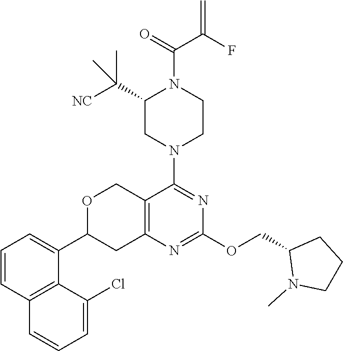 [SMILES: C=C(F)C(=O)N1CCN(c2nc(OC[C@@H]3CCCN3C)nc3c2COC(c2cccc4cccc(Cl)c24)C3)C[C@@H]1C(C)(C)C#N] |
| 272 | 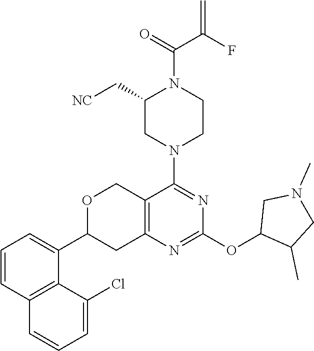 [SMILES: C=C(F)C(=O)N1CCN(c2nc(OC3CN(C)CC3C)nc3c2COC(c2cccc4cccc(Cl)c24)C3)C[C@@H]1CC#N] |
| 273 | 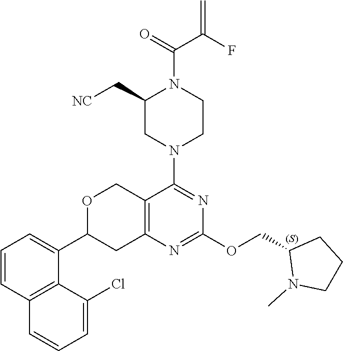 |
| 274 | 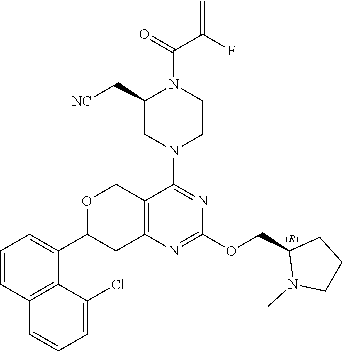 |
| 275 | 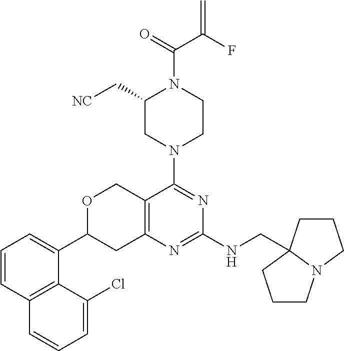 [SMILES: C=C(F)C(=O)N1CCN(c2nc(NCC34CCCN3CCC4)nc3c2COC(c2cccc4cccc(Cl)c24)C3)C[C@@H]1CC#N] |
| 276 | 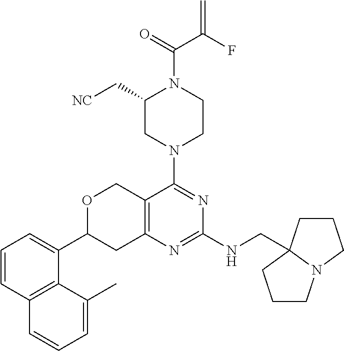 [SMILES: C=C(F)C(=O)N1CCN(c2nc(NCC34CCCN3CCC4)nc3c2COC(c2cccc4cccc(C)c24)C3)C[C@@H]1CC#N] |
| 277 | 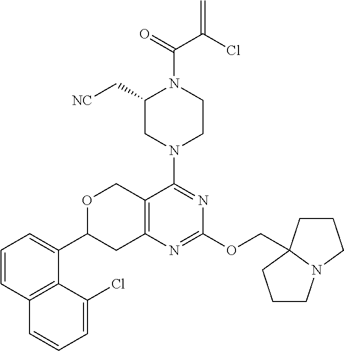 [SMILES: C=C(Cl)C(=O)N1CCN(c2nc(OCC34CCCN3CCC4)nc3c2COC(c2cccc4cccc(Cl)c24)C3)C[C@@H]1CC#N] |
| 278 | 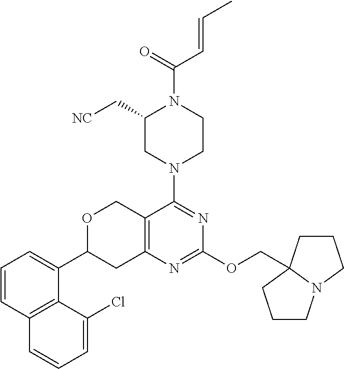 [SMILES: C/C=C/C(=O)N1CCN(c2nc(OCC34CCCN3CCC4)nc3c2COC(c2cccc4cccc(Cl)c24)C3)C[C@@H]1CC#N] |
| 279 | 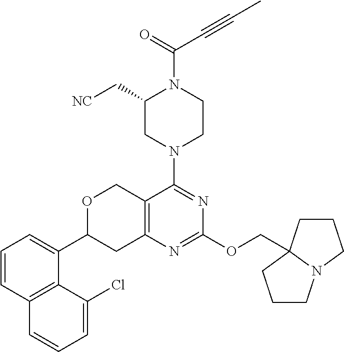 [SMILES: CC#CC(=O)N1CCN(c2nc(OCC34CCCN3CCC4)nc3c2COC(c2cccc4cccc(Cl)c24)C3)C[C@@H]1CC#N] |
| 280 | 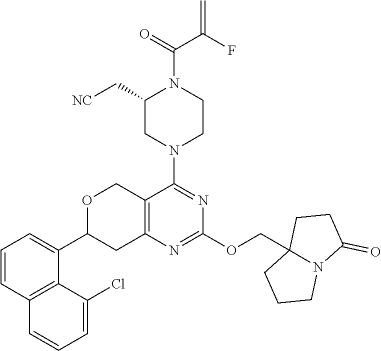 [SMILES: C=C(F)C(=O)N1CCN(c2nc(OCC34CCCN3C(=O)CC4)nc3c2COC(c2cccc4cccc(Cl)c24)C3)C[C@@H]1CC#N] |
| 281 | 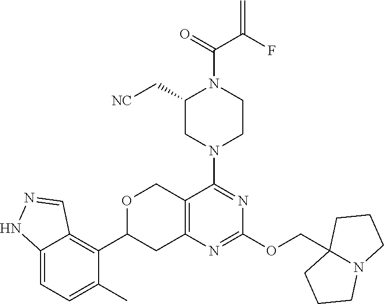 [SMILES: C=C(F)C(=O)N1CCN(c2nc(OCC34CCCN3CCC4)nc3c2COC(c2c(C)ccc4[nH]ncc24)C3)C[C@@H]1CC#N] |
| 282 | 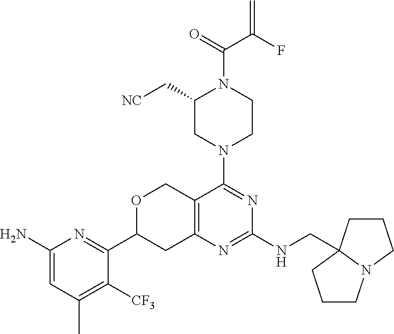 [SMILES: C=C(F)C(=O)N1CCN(c2nc(NCC34CCCN3CCC4)nc3c2COC(c2nc(N)cc(C)c2C(F)(F)F)C3)C[C@@H]1CC#N] |
| 283 | 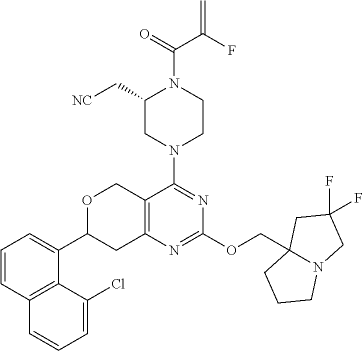 [SMILES: C=C(F)C(=O)N1CCN(c2nc(OCC34CCCN3CC(F)(F)C4)nc3c2COC(c2cccc4cccc(Cl)c24)C3)C[C@@H]1CC#N] |
| 284 | 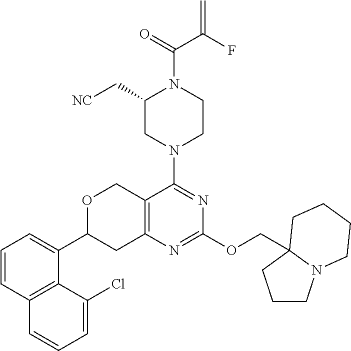 [SMILES: C=C(F)C(=O)N1CCN(c2nc(OCC34CCCCN3CCC4)nc3c2COC(c2cccc4cccc(Cl)c24)C3)C[C@@H]1CC#N] |
| 285 | 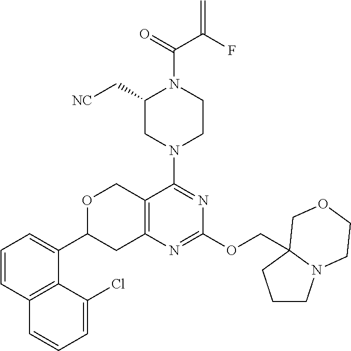 [SMILES: C=C(F)C(=O)N1CCN(c2nc(OCC34CCCN3CCOC4)nc3c2COC(c2cccc4cccc(Cl)c24)C3)C[C@@H]1CC#N] |
| 286 | 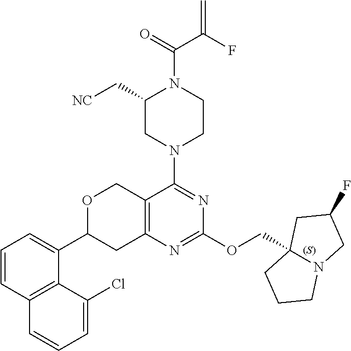 |
| 287 | 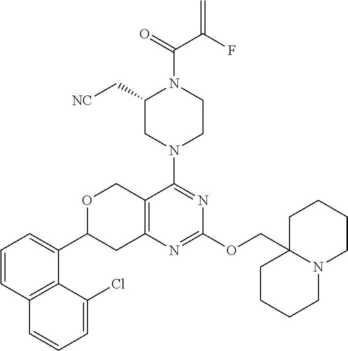 [SMILES: C=C(F)C(=O)N1CCN(c2nc(OCC34CCCCN3CCCC4)nc3c2COC(c2cccc4cccc(Cl)c24)C3)C[C@@H]1CC#N] |
| 288 | 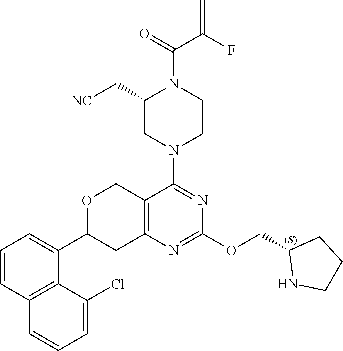 |
| 289 | 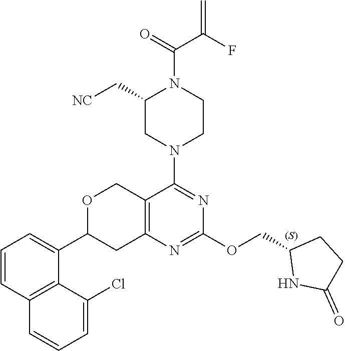 |
| 290 | 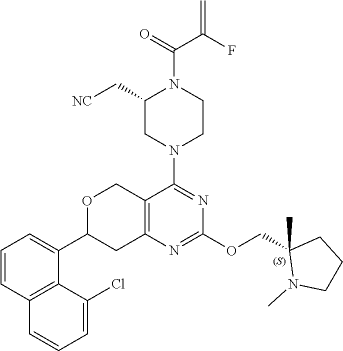 |
| 291 | 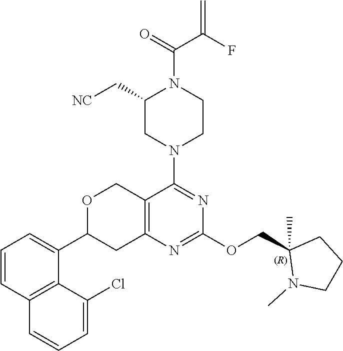 |
| 292 | 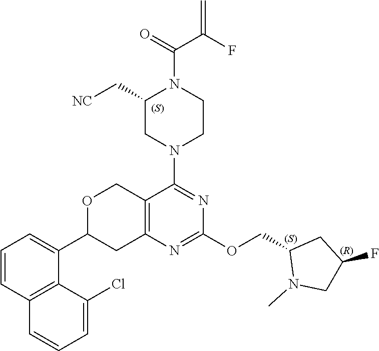 |
| 293 | 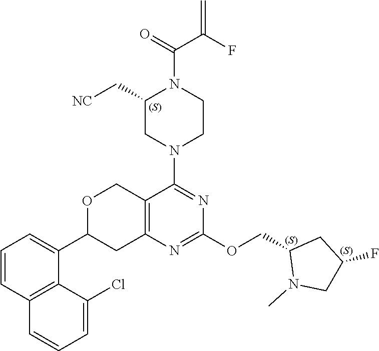 |
| 294 | 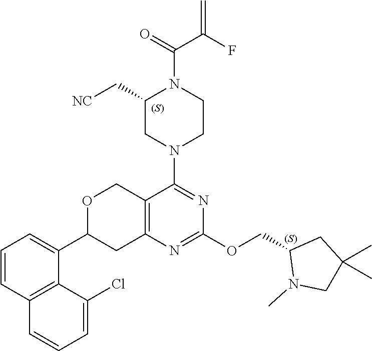 |
| 295 | 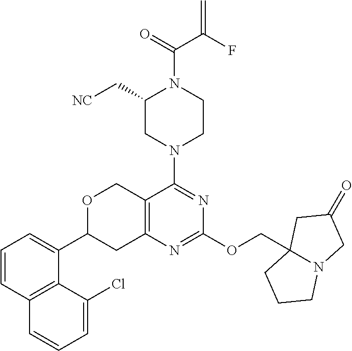 [SMILES: C=C(F)C(=O)N1CCN(c2nc(OCC34CCCN3CC(=O)C4)nc3c2COC(c2cccc4cccc(Cl)c24)C3)C[C@@H]1CC#N] |
| 296 | 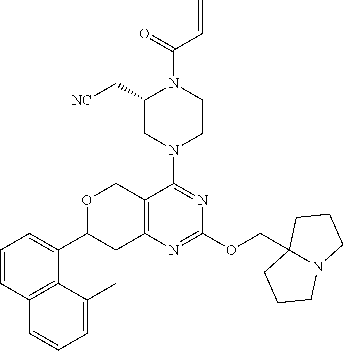 [SMILES: C=CC(=O)N1CCN(c2nc(OCC34CCCN3CCC4)nc3c2COC(c2cccc4cccc(C)c24)C3)C[C@@H]1CC#N] |
| 297 | 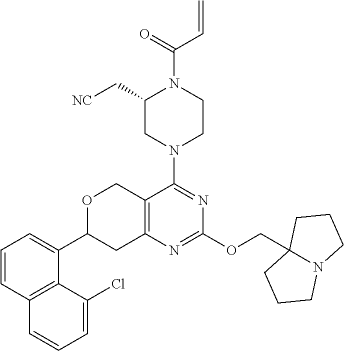 [SMILES: C=CC(=O)N1CCN(c2nc(OCC34CCCN3CCC4)nc3c2COC(c2cccc4cccc(Cl)c24)C3)C[C@@H]1CC#N] |
| 298 | 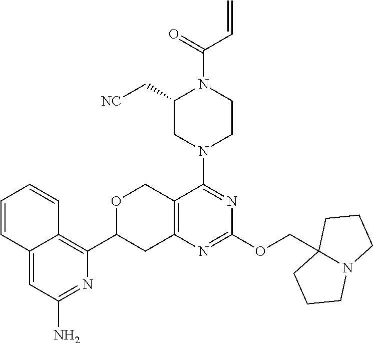 [SMILES: C=CC(=O)N1CCN(c2nc(OCC34CCCN3CCC4)nc3c2COC(c2nc(N)cc4ccccc24)C3)C[C@@H]1CC#N] |
| 299 | 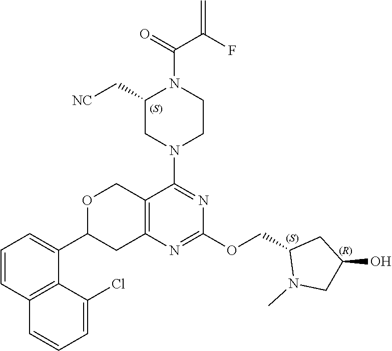 |
| 300 | 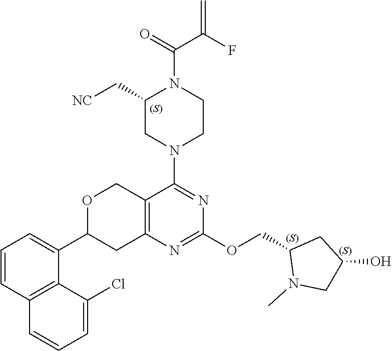 |
| 301 | 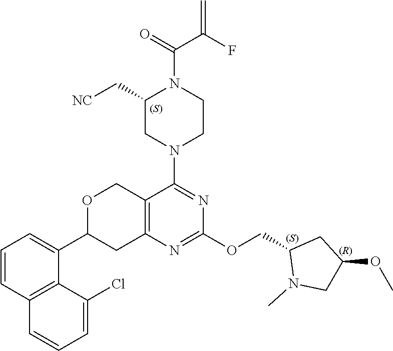 |
| 302 | 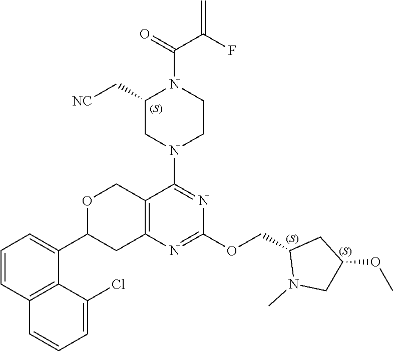 |
| 303 | 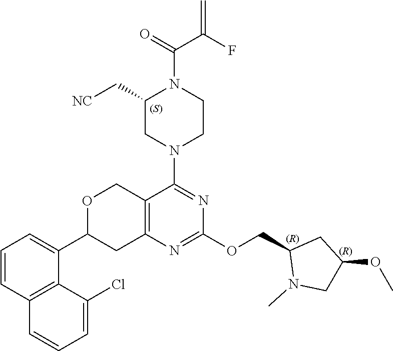 |
| 304 | 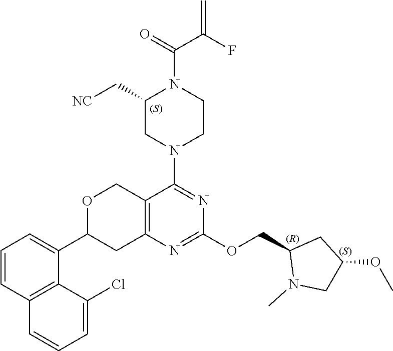 |
| 305 | 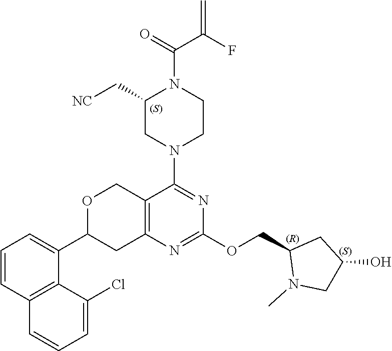 |
| 306 | 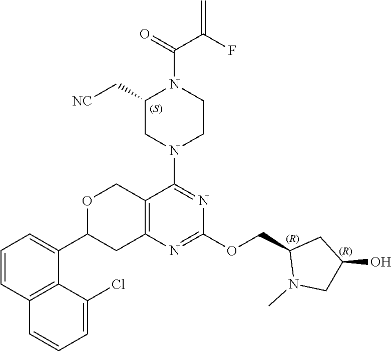 |
| 307 | 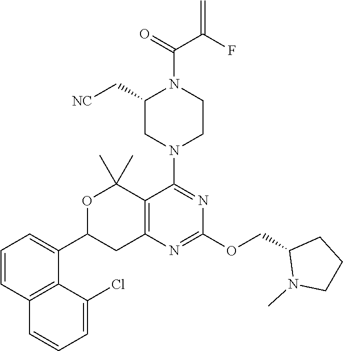 [SMILES: C=C(F)C(=O)N1CCN(c2nc(OC[C@@H]3CCCN3C)nc3c2C(C)(C)OC(c2cccc4cccc(Cl)c24)C3)C[C@@H]1CC#N] |
| 308 | 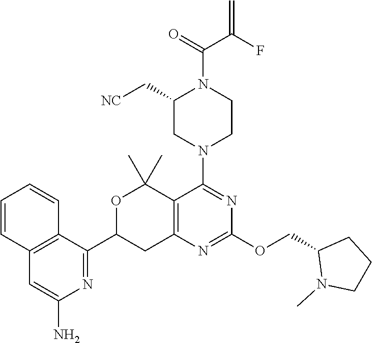 [SMILES: C=C(F)C(=O)N1CCN(c2nc(OC[C@@H]3CCCN3C)nc3c2C(C)(C)OC(c2nc(N)cc4ccccc24)C3)C[C@@H]1CC#N] |
| 309 | 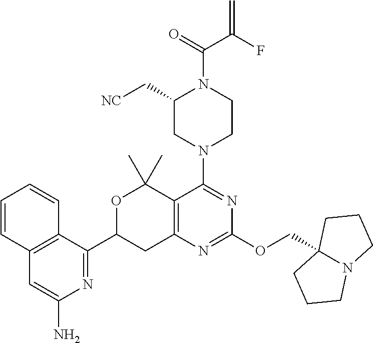 |
| 310 | 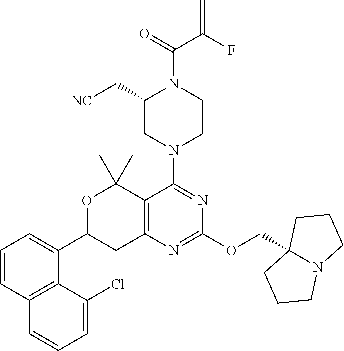 |
| 311 | 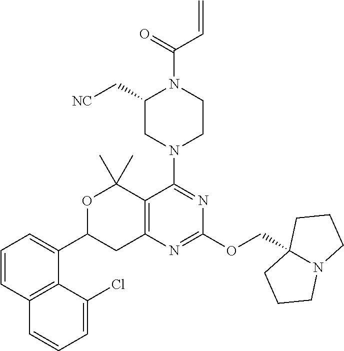 |
| 312 | 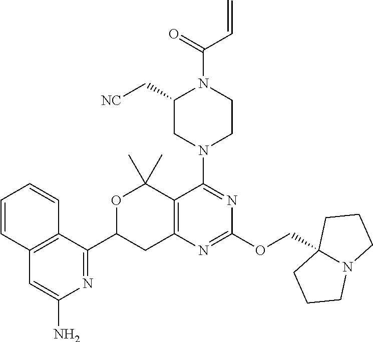 |
| 313 | 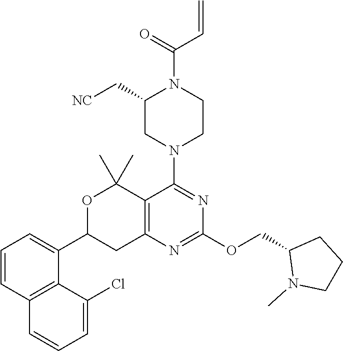 [SMILES: C=CC(=O)N1CCN(c2nc(OC[C@@H]3CCCN3C)nc3c2C(C)(C)OC(c2cccc4cccc(Cl)c24)C3)C[C@@H]1CC#N] |
| 314 | 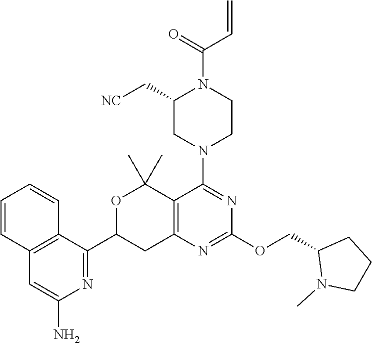 [SMILES: C=CC(=O)N1CCN(c2nc(OC[C@@H]3CCCN3C)nc3c2C(C)(C)OC(c2nc(N)cc4ccccc24)C3)C[C@@H]1CC#N] |
| 315 | 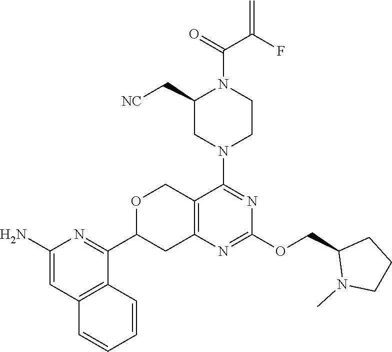 [SMILES: C=C(F)C(=O)N1CCN(c2nc(OC[C@H]3CCCN3C)nc3c2COC(c2nc(N)cc4ccccc24)C3)C[C@H]1CC#N] |
| | 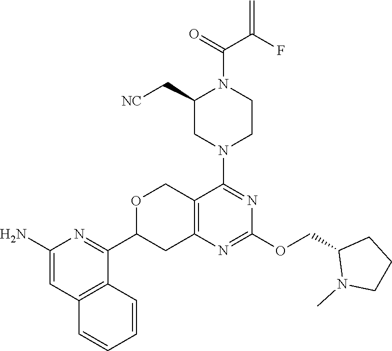 [SMILES: C=C(F)C(=O)N1CCN(c2nc(OC[C@@H]3CCCN3C)nc3c2COC(c2nc(N)cc4ccccc24)C3)C[C@H]1CC#N] |
| 317 | 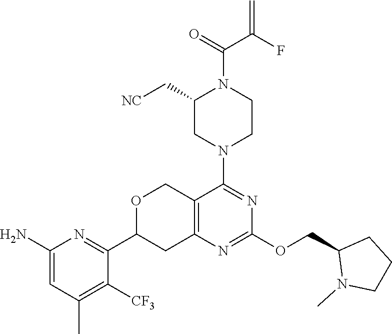 [SMILES: C=C(F)C(=O)N1CCN(c2nc(OC[C@H]3CCCN3C)nc3c2COC(c2nc(N)cc(C)c2C(F)(F)F)C3)C[C@@H]1CC#N] |
| 318 | 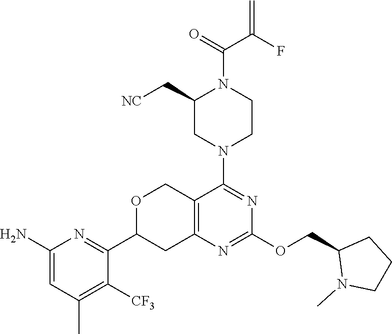 [SMILES: C=C(F)C(=O)N1CCN(c2nc(OC[C@H]3CCCN3C)nc3c2COC(c2nc(N)cc(C)c2C(F)(F)F)C3)C[C@H]1CC#N] |
| 319 | 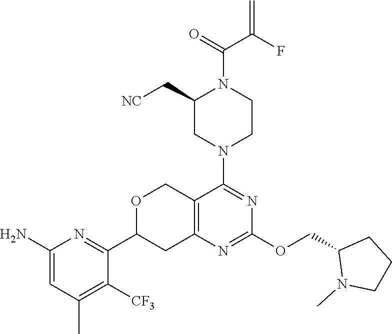 [SMILES: C=C(F)C(=O)N1CCN(c2nc(OC[C@@H]3CCCN3C)nc3c2COC(c2nc(N)cc(C)c2C(F)(F)F)C3)C[C@H]1CC#N] |
| 320 | 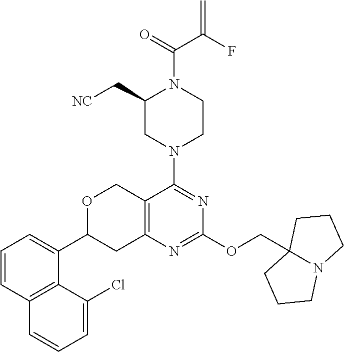 [SMILES: C=C(F)C(=O)N1CCN(c2nc(OCC34CCCN3CCC4)nc3c2COC(c2cccc4cccc(Cl)c24)C3)C[C@H]1CC#N] |
| 321 | 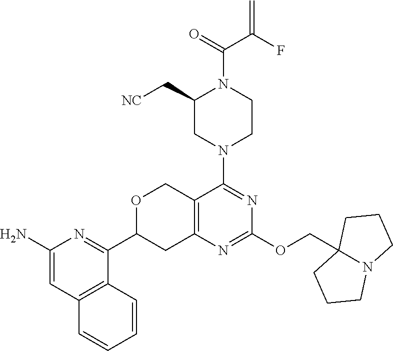 [SMILES: C=C(F)C(=O)N1CCN(c2nc(OCC34CCCN3CCC4)nc3c2COC(c2nc(N)cc4ccccc24)C3)C[C@H]1CC#N] |
| 322 | 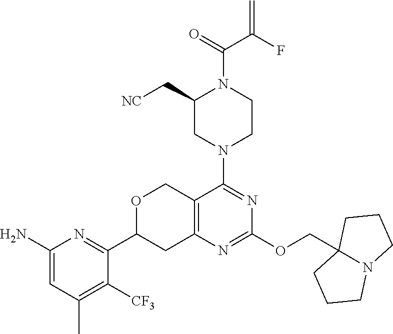 [SMILES: C=C(F)C(=O)N1CCN(c2nc(OCC34CCCN3CCC4)nc3c2COC(c2nc(N)cc(C)c2C(F)(F)F)C3)C[C@H]1CC#N] |

In some embodiments, a compound of Table 1 is provided wherein the carbon atom to which the moiety corresponding to L-R² in formula (I) is connected is in the (S) stereochemical configuration. In some embodiments, a compound of Table 1 is provided wherein the carbon atom to which the moiety corresponding to L-R² in formula (I) is connected is in the (R) stereochemical configuration. Pharmaceutically acceptable salts of such compounds are also provided herein.

### Methods of Treatment

It is contemplated that the compounds described herein antagonize activity of one or more RAS isoforms, such as KRAS, HRAS, NRAS, or a mutant thereof. As such, also provided is a method of treating diseases or conditions that are mediated by KRAS, HRAS, NRAS, or a mutant thereof. In some embodiments, the method comprises treating the diseases or conditions. In one embodiment, the disease is cancer and the treatment comprises administering an effective amount of a compound, pharmaceutically acceptable salt thereof, or composition as described herein to an individual in need thereof. In some embodiments, the compounds provided herein reduce tumor volume. In some embodiments, the compounds provided herein reduce cell proliferation. In some embodiments, the compounds provided herein prevent tumor metastasis. For purposes of this disclosure, beneficial or desired clinical results include, but are not limited to, alleviation of a symptom and/or diminishment of the extent of a symptom and/or preventing a worsening of a symptom associated with a disease or condition. In one variation, beneficial or desired clinical results include, but are not limited to, alleviation of a symptom and/or diminishment of the extent of a symptom and/or preventing a worsening of a symptom associated with a cancer. Preferably, treatment of a disease or condition with a compound of the disclosure or a pharmaceutically acceptable salt thereof is accompanied by no or fewer side effects than are associated with currently available therapies for the disease or condition and/or improves the quality of life of the individual.

In one embodiment, the present disclosure provides a method of treating a disease or condition mediated by KRAS, HRAS, NRAS, or a mutant thereof, comprising administering to an individual in need thereof a compound provided herein or a pharmaceutically acceptable salt thereof. In some embodiments, the disease or condition is a cancer. In some embodiments, the disease or condition is pancreatic cancer, lung adenocarcinoma or colorectal cancer. In some embodiments, the disease or condition is MYH-associated polyposis, biliary tract cancer or a hematologic malignancy. The methods of treatment in some embodiments comprise administering a compound provided herein or a pharmaceutically acceptable salt thereof as part of a combination therapy to treat the disease or condition.

In some embodiments, the method of treatment comprises inhibiting or antagonizing a mutant KRAS, such as KRAS G12D, KRAS G12V, KRAS G13D or KRAS G12C. In some embodiments, the method comprises inhibiting an oncogenic form or oncogenic mutant of KRAS. In some embodiments, the method comprises inhibiting KRAS G12C.

In some embodiments, the method of treatment comprises inhibiting or antagonizing a mutant HRAS, such as HRAS G12D, HRAS G12V, HRAS G13D or HRAS G12C. In some embodiments, the method comprises inhibiting an oncogenic form or oncogenic mutant of HRAS. In some embodiments, the method comprises inhibiting HRAS G12C.

In some embodiments, the method of treatment comprises inhibiting or antagonizing a mutant NRAS, such as NRAS G12D, NRAS G12V, NRAS G13D or NRAS G12C. In some embodiments, the method comprises inhibiting an oncogenic form or oncogenic mutant of NRAS. In some embodiments, the method comprises inhibiting NRAS G12C.

In some embodiments, the method of treatment comprises inhibiting or antagonizing a mutant RAS, such as a RAS G12D, RAS G12V, RAS G13D or RAS G12C. In some embodiments, the method comprises inhibiting an oncogenic form or oncogenic mutant of RAS. In some embodiments, the method comprises inhibiting RAS G12C, RAS G12D or RAS G12V.

In some embodiments, the compounds provided herein reduce cell proliferation. In some embodiments, cell proliferation is reduced by at least 10%, at least 20%, at least 30%, at least 40%, or at least 50%.

As used herein, the term “cancer” refers to a class of diseases of mammals characterized by uncontrolled cellular growth. The term “cancer” is used interchangeably with the terms “tumor,” “solid tumor,” “malignancy,” “hyperproliferation” and “neoplasm.” Cancer includes all types of hyperproliferative growth, hyperplasic growth, neoplastic growth, cancerous growth or oncogenic processes, metastatic tissues or malignantly transformed cells, tissues, or organs, irrespective of histopathologic type or stage of invasiveness. Illustrative examples include, lung, prostate, head and neck, pancreatic, breast and colorectal cancer, melanomas and gliomas (such as a high-grade glioma, including glioblastoma multiforme (GBM), the most common and deadliest of malignant primary brain tumors in adult humans).

The phrase “solid tumor” includes, for example, lung cancer, head and neck cancer, brain cancer, oral cancer, colorectal cancer, breast cancer, prostate cancer, pancreatic cancer and liver cancer. Other types of solid tumors are named for the particular cells that form them, for example, sarcomas formed from connective tissue cells (for example, bone cartilage, fat), carcinomas formed from epithelial tissue cells (for example, breast, colon, pancreas) and lymphomas formed from lymphatic tissue cells (for example, lymph nodes, spleen, thymus). Treatment of all types of solid tumors regardless of naming convention is within the scope of this disclosure.

The cancer can be a blood cancer, lung cancer, breast cancer, colorectal cancer, fallopian tube cancer, brain cancer, head and neck cancer, esophageal cancer, ovarian cancer, pancreatic cancer, peritoneal cancer, prostate cancer or skin cancer, such as, but not limited to, liver cancer, melanoma, Hodgkin's disease, non-Hodgkin's lymphomas, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, chronic lymphocytic leukemia, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, malignant melanoma, choriocarcinoma, mycosis fungoide, head neck carcinoma, osteogenic sarcoma, pancreatic carcinoma, acute granulocytic leukemia, hairy cell leukemia, rhabdomyosarcoma, Kaposi's sarcoma, genitourinary carcinoma, appendix cancer, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, or prostatic carcinoma.

Also provided is a method of treating bladder cancer, breast cancer, colorectal cancer, fallopian tube cancer, ovarian cancer, prostate cancer, non-small cell lung cancer, pancreatic cancer, peritoneal cancer, testicular cancer, endometrial cancer, or uterine cancer, comprising administering an effective amount of a compound or composition as described herein, or a pharmaceutically acceptable salt or solvate thereof, to an individual in need thereof. In some embodiments, the method comprises treating bladder cancer, breast cancer, colorectal cancer, fallopian tube cancer, ovarian cancer, prostate cancer, non-small cell lung cancer, pancreatic cancer, peritoneal cancer, testicular cancer, endometrial cancer, or uterine cancer.

Also provided is a method of treating cancer, comprising administering an effective amount of a compound or composition as described herein, or a pharmaceutically acceptable salt or solvate thereof, in combination with an additional chemotherapeutic agent, to an individual in need thereof. In some embodiments, the method comprises treating cancer.

The compounds provided herein also modulate the function of KRAS, HRAS or NRAS and include compounds that are, for example, selective antagonists of KRAS, HRAS or NRAS. Thus, the present compounds are useful in the treatment of RAS-associated conditions. A “RAS-associated condition,” as used herein, denotes a condition or disorder which can be treated by modulating the function or activity of KRAS, HRAS or NRAS in a subject, wherein treatment comprises partial alleviation or cure of the condition or disorder. Modulation can occur locally, for example, within certain tissues of the subject, or more extensively throughout a subject being treated for such a condition or disorder.

In some embodiments, the compounds provided herein reduce tumor volume. In some embodiments, the compounds reduce tumor volume by at least 10%, at least 20%, at least 30%, at least 40%, or at least 50%.

In some embodiments, the compounds provided herein reduce KRAS signaling. In some embodiments, the compounds provided herein reduce the level of phosphorylated extracellular signal-regulated kinase (ERK). In some embodiments, the level of phosphorylated ERK is reduced at least 10%, at least 20%, at least 30%, at least 40%, or at least 50%.

In some embodiments, the compounds provided herein reduce the level of activated KRAS. In some embodiments, the compounds provided herein reduce the level of KRAS-bound to GTP. In some embodiments, the level of KRAS-GTP is reduced by at least 10%, at least 20%, at least 30%, at least 40%, or at least 50%.

The compounds with potent antagonistic activity are used for the treatment of KRAS related lung cancer, colorectal cancer, or pancreatic cancer.

### Compositions

Compositions, including pharmaceutical compositions, of any of the compounds detailed herein are embraced by this disclosure. Thus, provided herein are pharmaceutical compositions comprising a compound of the disclosure, or a pharmaceutically acceptable salt thereof and a pharmaceutically acceptable carrier or excipient. The pharmaceutical compositions provided herein may take a form suitable for oral, buccal, parenteral (e.g., intravenous, intramuscular, infusion or subcutaneous injection), nasal, topical or rectal administration, or a form suitable for administration by inhalation.

A compound as described herein may, in one aspect, be in a purified form. Compositions comprising a compound as described herein, or a salt thereof, are provided, such as compositions of substantially pure compounds. In some embodiments, a composition comprising a compound as described herein, or a salt thereof, is in substantially pure form. Unless otherwise stated, “substantially pure” refers to a composition which contains no more than 35% impurity, wherein the impurity denotes a compound other than the desired compound, or a salt thereof, which comprises the majority of the composition. In one variation, a composition of substantially pure compound, or a salt thereof, is provided wherein the composition contains no more than 25% impurity. In another variation, a composition of substantially pure compound, or a salt thereof, is provided wherein the composition contains or no more than 20% impurity. In still another variation, a composition of substantially pure compound, or a salt thereof, is provided wherein the composition contains or no more than 10% impurity. In a further variation, a composition of substantially pure compound, or a salt thereof, is provided wherein the composition contains or no more than 5% impurity. In another variation, a composition of substantially pure compound, or a salt thereof, is provided wherein the composition contains or no more than 3% impurity. In still another variation, a composition of substantially pure compound, or a salt thereof, is provided wherein the composition contains or no more than 1% impurity. In a further variation, a composition of substantially pure compound, or a salt thereof, is provided wherein the composition contains or no more than 0.5% impurity.

In certain embodiments, pharmaceutical compositions are formulated in any manner, including using one or more physiologically acceptable carriers comprising excipients and/or auxiliaries which facilitate processing of the active compounds into pharmaceutical compositions. In some embodiments, proper formulation is dependent upon the route of administration chosen. In various embodiments, any techniques, carriers and excipients are used as suitable.

Provided herein are pharmaceutical compositions that include a compound described herein and a pharmaceutically acceptable diluent(s), excipient(s) and/or carrier(s). In addition, in some embodiments, the compounds described herein are administered as pharmaceutical compositions in which compounds described herein are mixed with other active ingredients, as in combination therapy.

A pharmaceutical composition, as used herein, refers to a mixture of a compound described herein with other chemical components, such as carriers, stabilizers, diluents, dispersing agents, suspending agents, thickening agents and/or excipients. In certain embodiments, a pharmaceutical composition facilitates administration of the compound to an organism. In some embodiments, practicing the methods of treatment or use provided herein, includes administering or using a pharmaceutical composition comprising a therapeutically effective amount of a compound provided herein. In specific embodiments, the methods of treatment provided for herein include administering such a pharmaceutical composition to a mammal having a disease or condition to be treated. In one embodiment, the mammal is a human. In some embodiments, the therapeutically effective amount varies widely depending on the severity of the disease, the age and relative health of the subject, the potency of the compound used and other factors. In various embodiments, the compounds described herein are used singly or in combination with one or more therapeutic agents as components of mixtures.

In certain embodiments, the pharmaceutical compositions provided herein are formulated for intravenous injections. In certain aspects, the intravenous injection formulations provided herein are formulated as aqueous solutions and, in some embodiments, in physiologically compatible buffers such as Hank's solution, Ringer's solution, or physiological saline buffer. In certain embodiments, the pharmaceutical compositions provided herein are formulated for transmucosal administration. In some aspects, transmucosal formulations include penetrants appropriate to the barrier to be permeated. In certain embodiments, the pharmaceutical compositions provided herein are formulated for other parenteral injections, appropriate formulations include aqueous or nonaqueous solutions and in one embodiment, with physiologically compatible buffers or excipients.

In certain embodiments, the pharmaceutical compositions provided herein are formulated for oral administration. In certain aspects, the oral formulations provided herein comprise compounds described herein that are formulated with pharmaceutically acceptable carriers or excipients. Such carriers enable the compounds described herein to be formulated as tablets, powders, pills, dragees, capsules, liquids, gels, syrups, elixirs, slurries, suspensions and the like, for oral ingestion by a patient to be treated.

In some embodiments, pharmaceutical compositions for oral use are obtained by mixing one or more solid excipient with one or more of the compounds described herein, optionally grinding the resulting mixture and processing the mixture of granules, after adding suitable auxiliaries, if desired, to obtain tablets or dragee cores. Suitable excipients include, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are optionally added, such as the cross-linked croscarmellose sodium, polyvinylpyrrolidone, agar, or alginic acid or a salt thereof such as sodium alginate.

In certain embodiments, provided herein is a pharmaceutical composition formulated as dragee cores with suitable coatings. In certain embodiments, concentrated sugar solutions are used in forming the suitable coating and optionally contain gum arabic, talc, polyvinylpyrrolidone, carbopol gel, polyethylene glycol and/or titanium dioxide, lacquer solutions and suitable organic solvents or solvent mixtures. In some embodiments, dyestuffs and/or pigments are added to tablets, dragees and/or the coatings thereof for, e.g., identification or to characterize different combinations of active compound doses.

In certain embodiments, pharmaceutical compositions which are used include orally include push-fit capsules made of gelatin, as well as soft, sealed capsules made of gelatin and a plasticizer, such as glycerol or sorbitol. In some embodiments, the push-fit capsules contain the active ingredients in admixture with filler such as lactose, binders such as starches and/or lubricants such as talc or magnesium stearate and, optionally, stabilizers. In certain embodiments, in soft capsules, the active compounds are dissolved or suspended in suitable liquids, such as fatty oils, liquid paraffin, or liquid polyethylene glycols. In addition, stabilizers are optionally added. In certain embodiments, the formulations for oral administration are in dosages suitable for such administration.

In certain embodiments, the pharmaceutical compositions provided herein are formulated for buccal or sublingual administration. In certain embodiments, buccal or sublingual compositions take the form of tablets, lozenges, or gels formulated in a conventional manner. In certain embodiments, parenteral injections involve bolus injection or continuous infusion. In some embodiments, formulations for injection are presented in unit dosage form, e.g., in ampoules or in multi-dose containers, with an added preservative. In some embodiments, the pharmaceutical composition described herein is in a form suitable for parenteral injection as a sterile suspensions, solutions or emulsions in oily or aqueous vehicles and optionally contains formulatory agents such as suspending, stabilizing and/or dispersing agents. Pharmaceutical formulations for parenteral administration include aqueous solutions of the active compounds in water-soluble form. In some embodiments, suspensions of the active compounds are prepared as appropriate oily injection suspensions. Suitable lipophilic solvents or vehicles include fatty oils such as sesame oil, or synthetic fatty acid esters, such as ethyl oleate or triglycerides, or liposomes. In certain embodiments, aqueous injection suspensions contain substances which increase the viscosity of the suspension, such as sodium carboxymethyl cellulose, sorbitol, or dextran. Optionally, the suspensions also contain suitable stabilizers or agents which increase the solubility of the compounds to allow for the preparation of highly concentrated solutions. In alternative embodiments, the active ingredient is in powder form for constitution with a suitable vehicle, e.g., sterile pyrogen-free water, before use.

In some embodiments, the compounds described herein are administered topically. In specific embodiments, the compounds described herein are formulated into a variety of topically administrable compositions, such as solutions, suspensions, lotions, gels, pastes, medicated sticks, balms, creams or ointments. Such pharmaceutical compounds optionally contain solubilizers, stabilizers, tonicity enhancing agents, buffers and/or preservatives.

In certain embodiments, the pharmaceutical compositions provided herein are formulated for transdermal administration of compounds described herein. In some embodiments, administration of such compositions employs transdermal delivery devices and transdermal delivery patches. In certain embodiments, the compositions are lipophilic emulsions or buffered, aqueous solutions, dissolved and/or dispersed in a polymer or an adhesive. Such patches include those constructed for continuous, pulsatile, or on demand delivery of pharmaceutical agents. In some embodiments, transdermal delivery of the compounds described herein is accomplished by use of iontophoretic patches and the like. In certain embodiments, the rate of absorption is slowed by using rate-controlling membranes or by trapping the compound within a polymer matrix or gel. Conversely, absorption enhancers are optionally used to increase absorption. Absorption enhancer and carrier include absorbable pharmaceutically acceptable solvents that assist in passage of the compound through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time and means to secure the device to the skin.

In certain embodiments, the pharmaceutical compositions provided herein are formulated for administration by inhalation. In certain embodiments, in such pharmaceutical compositions formulated for inhalation, the compounds described herein are in a form as an aerosol, a mist or a powder. In some embodiments, pharmaceutical compositions described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In certain aspects of a pressurized aerosol, the dosage unit is determined by providing a valve to deliver a metered amount. In certain embodiments, capsules and cartridges of, such as, by way of example only, gelatin for use in an inhaler or insufflator is formulated containing a powder mix of the compound described herein and a suitable powder base such as lactose or starch.

In some embodiments, the compounds described herein are formulated in rectal compositions such as enemas, rectal gels, rectal foams, rectal aerosols, suppositories, jelly suppositories, or retention enemas. In certain embodiments, rectal compositions optionally contain conventional suppository bases such as cocoa butter or other glycerides, as well as synthetic polymers such as polyvinylpyrrolidone, PEG and the like. In certain suppository forms of the compositions, a low-melting wax such as, but not limited to, a mixture of fatty acid glycerides, optionally in combination with cocoa butter is first melted.

In various embodiments provided herein, the pharmaceutical compositions are formulated in a conventional manner using one or more physiologically acceptable carriers comprising excipients and auxiliaries which facilitate processing of the active compounds into pharmaceutically acceptable preparations. In certain embodiments, proper formulation is dependent upon the route of administration chosen. In various embodiments, any of the techniques, carriers and excipients is used as suitable. In some embodiments, pharmaceutical compositions comprising a compound described herein are manufactured in a conventional manner, such as, by way of example only, by means of conventional mixing, dissolving, granulating, dragee-making, levigating, emulsifying, encapsulating, entrapping or compression processes.

In certain embodiments, the pharmaceutical compositions include at least one pharmaceutically acceptable carrier, diluent or excipient and a compound described herein described herein as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds described herein exist as tautomers. All tautomers are included within the scope of the compounds presented herein. Additionally, included herein are the solvated and unsolvated forms of the compounds described herein. Solvated compounds include those that are solvated with pharmaceutically acceptable solvents such as water, ethanol and the like. The solvated forms of the compounds presented herein are also considered to be disclosed herein. In some embodiments, the pharmaceutical compositions described herein include other medicinal or pharmaceutical agents, carriers, adjuvants, such as preserving, stabilizing, wetting or emulsifying agents, solution promoters, salts for regulating the osmotic pressure and/or buffers. In additional embodiments, the pharmaceutical compositions described herein also contain other therapeutically valuable substances.

Methods for the preparation of compositions containing the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. In various embodiments, the compositions are in liquid solutions or suspensions, solid forms suitable for solution or suspension in a liquid prior to use, or as emulsions. These compositions optionally contain minor amounts of nontoxic, auxiliary substances, such as wetting or emulsifying agents, pH buffering agents and so forth.

In some embodiments, a composition comprising a compound described herein takes the form of a liquid where the agents are present in solution, in suspension or both. In some embodiments, when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

Useful aqueous suspension optionally contain one or more polymers as suspending agents. Useful polymers include water-soluble polymers such as cellulosic polymers, e.g., hydroxypropyl methylcellulose and water-insoluble polymers such as cross-linked carboxyl-containing polymers. Useful compositions optionally comprise an mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful compositions optionally include solubilizing agents to aid in the solubility of a compound described herein. The term “solubilizing agent” generally includes agents that result in formation of a micellar solution or a true solution of the agent. Solubilizing agents include certain acceptable nonionic surfactants, for example polysorbate 80 and ophthalmologically acceptable glycols, polyglycols, e.g., polyethylene glycol 400 and glycol ethers.

Useful compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Useful compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Certain useful compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Some useful compositions optionally include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Certain useful compositions optionally one or more antioxidants to enhance chemical stability where required. Suitable antioxidants include, by way of example only, ascorbic acid and sodium metabisulfite.

In some embodiments, aqueous suspension compositions are packaged in single-dose non-reclosable containers. In alternative embodiments, multiple-dose reclosable containers are used, in which case it is typical to include a preservative in the composition.

In various embodiments, any delivery system for hydrophobic pharmaceutical compounds is employed. Liposomes and emulsions are examples of delivery vehicles or carriers for hydrophobic drugs. In certain embodiments, certain organic solvents such as N-methylpyrrolidone are employed. In some embodiments, the compounds are delivered using a sustained-release system, such as semipermeable matrices of solid hydrophobic polymers containing the therapeutic agent. Various sustained-release materials are utilized in the embodiments herein. In certain embodiments, sustained-release capsules release the compounds for a few weeks up to over 100 days. In some embodiments, depending on the chemical nature and the biological stability of the therapeutic reagent, additional strategies for protein stabilization are employed.

In certain embodiments, the formulations or compositions described herein benefit from and/or optionally comprise antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

### Dosing and Treatment Regimens

In certain embodiments, the compounds described herein are used in the preparation or manufacture of medicaments for the treatment of diseases or conditions that are mediated through RAS mutations, such as G12D, G12V, G13D and G12C mutant KRAS, HRAS or NRAS. In some embodiments, a method for treating any of the diseases or conditions described herein in a subject in need of such treatment, involves administration of pharmaceutical compositions containing at least one compound described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

In certain embodiments, the compositions containing the compound(s) described herein are administered for therapeutic treatments. In certain therapeutic applications, the compositions are administered to a patient already suffering from a disease or condition, in an amount sufficient to cure or at least partially arrest the symptoms of the disease or condition. In some embodiments, amounts effective for this use will depend on the severity and course of the disease or condition, previous therapy, the patient's health status, weight and response to the drugs and the judgment of the treating physician.

In certain embodiments, the amount of a given agent that corresponds to an effective amount varies depending upon factors such as the particular compound, disease or condition and its severity, the identity (e.g., weight) of the subject or host in need of treatment. In some embodiments, the effective amount is, nevertheless, determined according to the particular circumstances surrounding the case, including, e.g., the specific agent that is administered, the route of administration, the condition being treated and the subject or host being treated. In certain embodiments, however, doses employed for adult human treatment is in the range of about 0.02 to about 5000 mg per day, in a specific embodiment about 1 to about 1500 mg per day. In various embodiments, the desired dose is conveniently presented in a single dose or as divided doses administered simultaneously (or over a short period of time) or at appropriate intervals, for example as two, three, four or more sub-doses per day.

In some embodiments, the pharmaceutical compositions described herein are in a unit dosage form suitable for single administration of precise dosages. In some instances, in unit dosage form, the formulation is divided into unit doses containing appropriate quantities of one or more compound. In certain embodiments, the unit dosage is in the form of a package containing discrete quantities of the formulation. Non-limiting examples are packaged tablets or capsules and powders in vials or ampoules. In some embodiments, aqueous suspension compositions are packaged in single-dose non-reclosable containers. In alternative embodiments, multiple-dose reclosable containers are used, in which case it is typical to include a preservative in the composition. By way of example only, formulations for parenteral injection are, in some embodiments, presented in unit dosage form, which include, but are not limited to ampoules, or in multi-dose containers, with an added preservative.

In certain embodiments, the daily dosages appropriate for the compounds described herein are from about 0.01 to about 20 mg/kg per body weight. In some embodiments, an indicated daily dosage in the larger subject, including, but not limited to, humans, is in the range from about 0.5 mg to about 1500 mg, conveniently administered in divided doses, including, but not limited to, up to four times a day or in extended release form. In certain embodiments, suitable unit dosage forms for oral administration comprise from about 1 to about 500 mg active ingredient. The foregoing ranges are merely suggestive, as the number of variables in regard to an individual treatment regime is large and considerable excursions from these recommended values are not uncommon. In certain embodiments, the dosages are altered depending on a number of variables, not limited to the activity of the compound used, the disease or condition to be treated, the mode of administration, the requirements of the individual subject, the severity of the disease or condition being treated and the judgment of the practitioner.

In certain embodiments, toxicity and therapeutic efficacy of such therapeutic regimens are determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. In certain embodiments, compounds exhibiting high therapeutic indices are preferred. In some embodiments, the data obtained from cell culture assays and animal studies is used in formulating a range of dosage for use in human. In specific embodiments, the dosage of such compounds lies within a range of circulating concentrations that include the ED₅₀ with minimal toxicity. In certain embodiments, the dosage varies within this range depending upon the dosage form employed and the route of administration utilized.

### Combination Therapy

Compounds described herein can also be used in combination with other active ingredients, therapeutic agents or treatment modalities. Such combinations are selected based on the condition to be treated, cross-reactivities of ingredients and pharmaco-properties of the combination. In one embodiment, provided is a compound as described herein, or pharmaceutically acceptable salt thereof, used in combination with another anti-cancer therapy, such as a chemotherapeutic agent, an immunotherapeutic agent, a gene therapeutic agent or a combination thereof. For example, when treating cancer, the compounds and compositions provided herein can be combined with other anti-cancer therapeutic agents, surgical procedures, radiation procedures or a combination of any of the foregoing. The treatment methods described herein also contemplate combination therapy.

It is also possible to combine a compound of the disclosure with one or more other active ingredients in a unitary dosage form for simultaneous or sequential administration to a patient. The combination therapy may be administered as a simultaneous or sequential regimen. When administered sequentially, the combination may be administered in two or more administrations.

### Kits

Also provided herein are kits for treating cancer comprising a compound or composition described herein are provided. In certain embodiments, the kit comprises a unit dose of a compound or composition described herein and instructions for administering the same. In certain aspects, the kit further comprises a second drug suitable for anti-cancer therapy, or instructions for co-administering an additional anti-cancer therapy (such as radiation or gene therapy). In another aspect, kits for use to achieve anti-cancer effects comprise less than about 500 mg/day, or less than about 400 mg/day, or less than about 300 mg/day, or less than about 200 mg/day of a compound or composition described herein and a second drug suitable for anti-cancer therapy. In yet another variation, kits for use to achieve anti-cancer effects comprise a greater than about 500 mg/day of a compound or composition as described herein and a second drug suitable for anti-cancer therapy.

### Methods of Manufacturing a Medicament

In a further aspect of the disclosure, use of the compounds and compositions described herein in the manufacture of a medicament is provided. In particular, the manufacture of a medicament for use in the treatment of cancer are provided.

### EXAMPLES

The disclosure is further illustrated by the following examples. The examples below are non-limiting are merely representative of various aspects of the disclosure. Solid and dotted wedges within the structures herein disclosed illustrate relative stereochemistry, with absolute stereochemistry depicted only when specifically stated or delineated.

Compounds having the structure of Formula I, or any sub-formula described herein can be synthesized using standard synthetic techniques known to those of skill in the art. Compounds of the present disclosure can be synthesized using the general synthetic procedures set forth in the examples that follow.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g. a racemate, and an appropriate chiral compound. The diastereomers may then be separated by any convenient means, for example by crystallization and the desired enantiomer recovered. In another resolution process, a racemate may be separated using chiral High-Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

Compounds as described herein may be prepared according to the process outlined in Schemes 1-8 below.

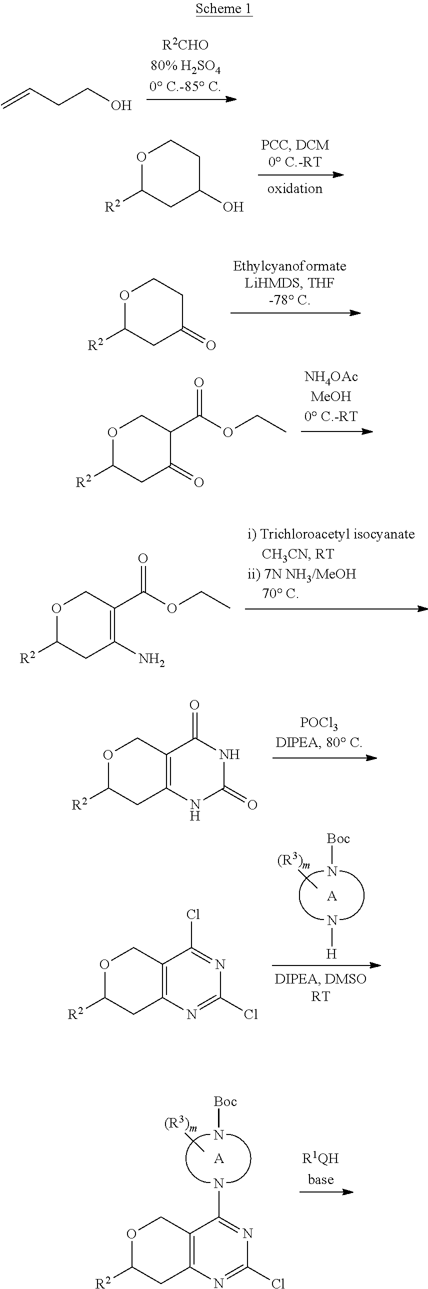

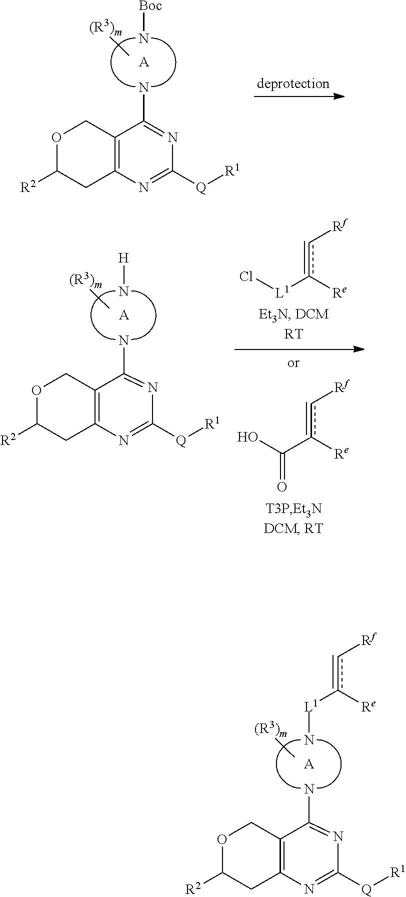

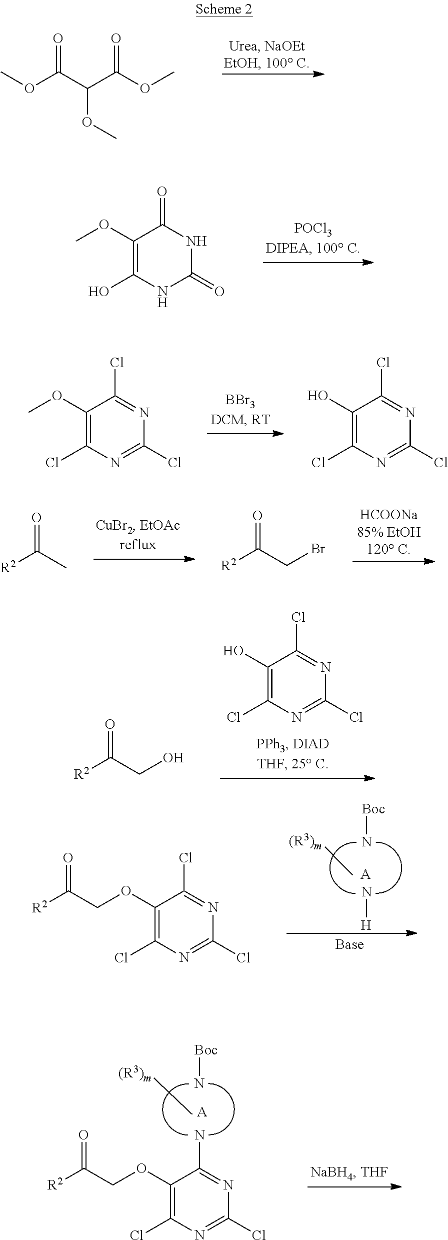

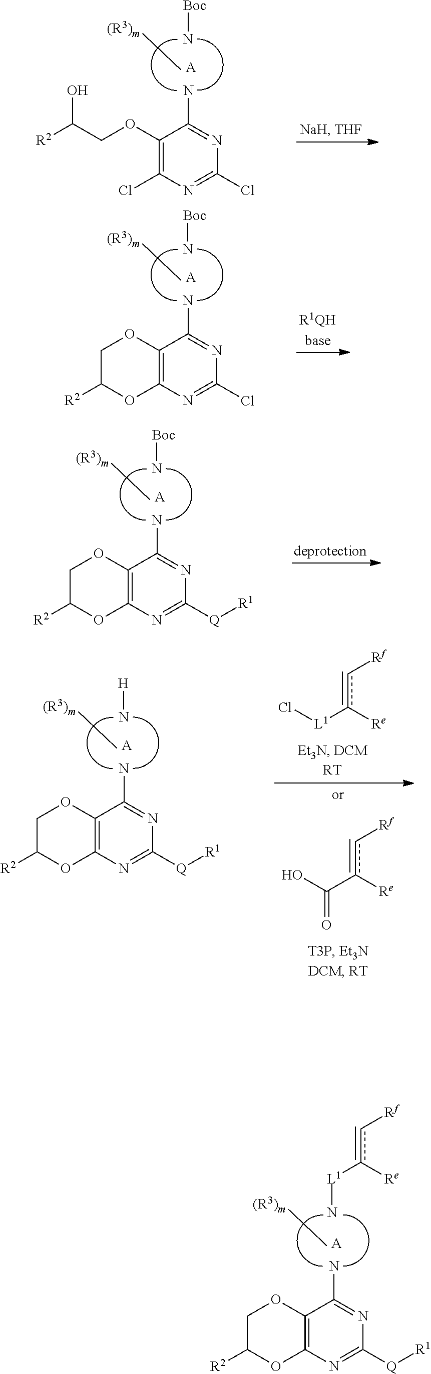

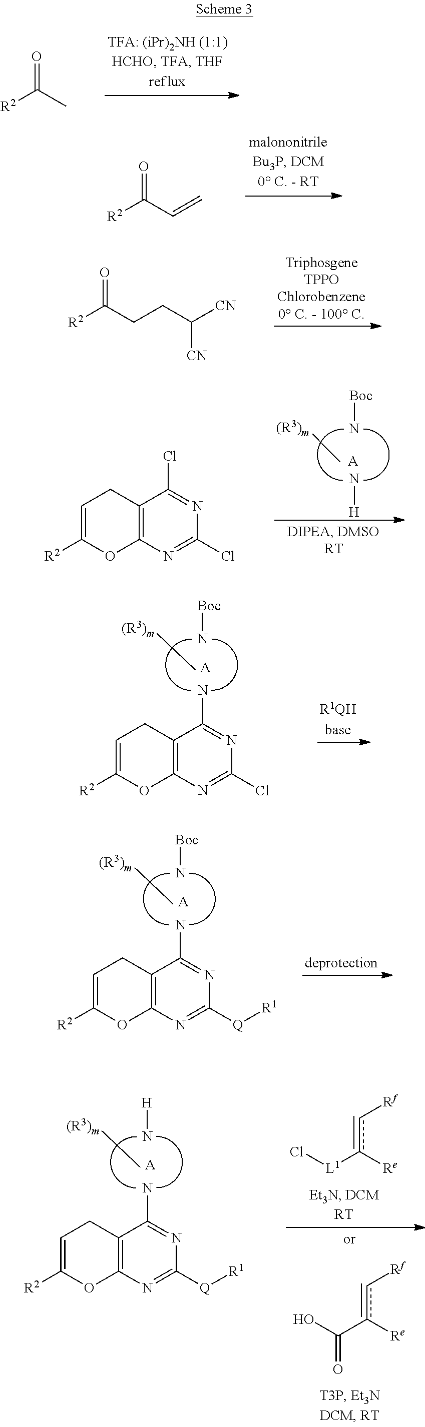

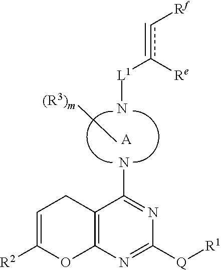

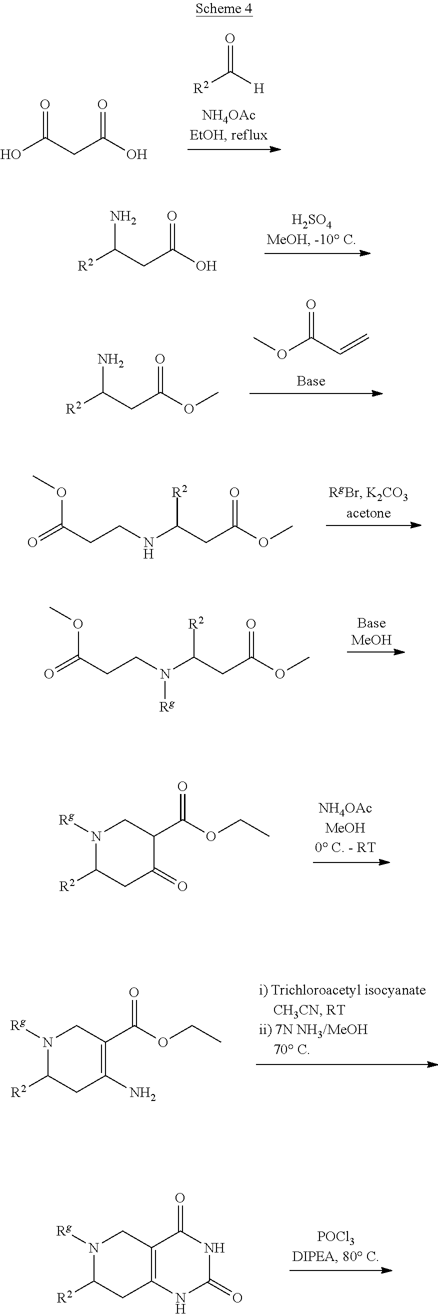

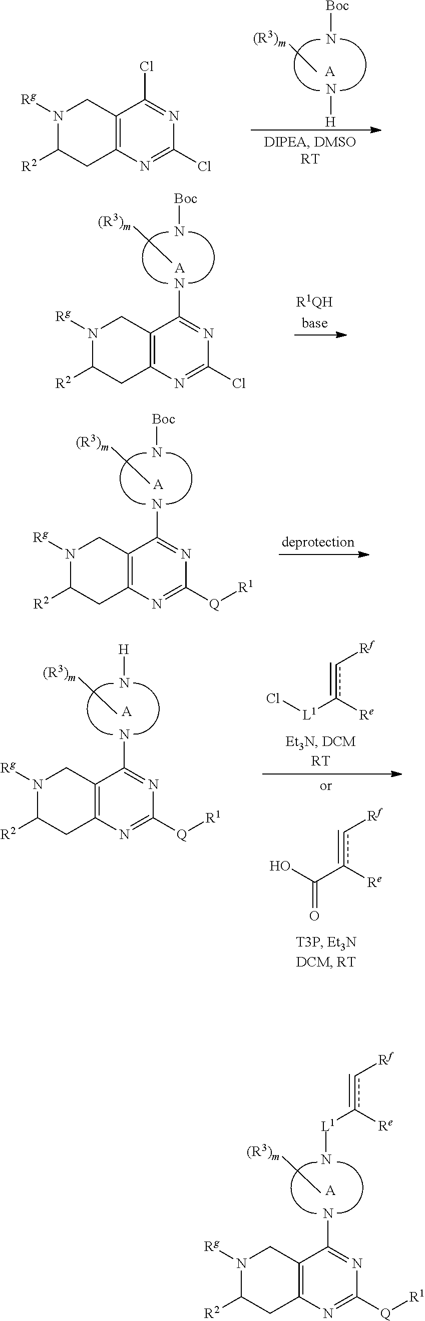

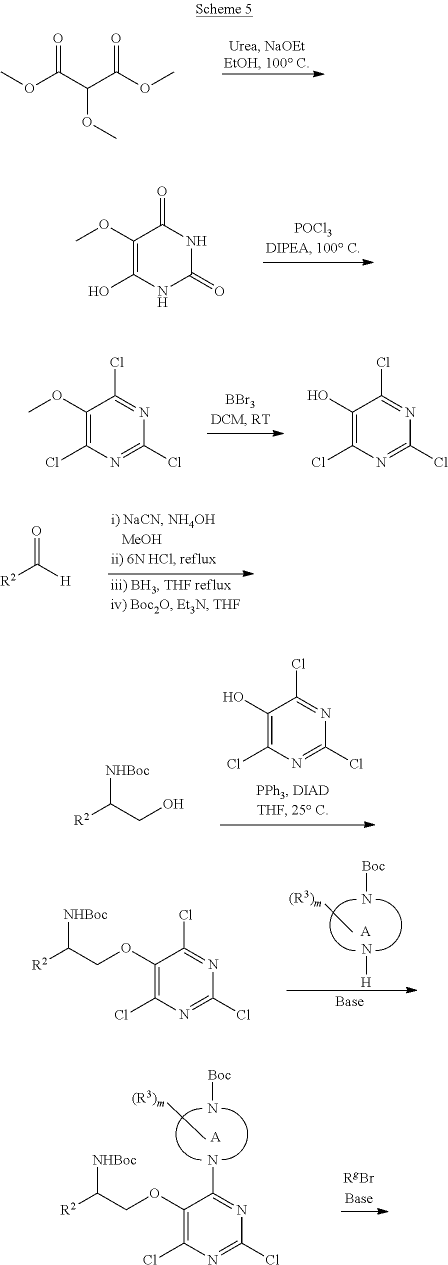

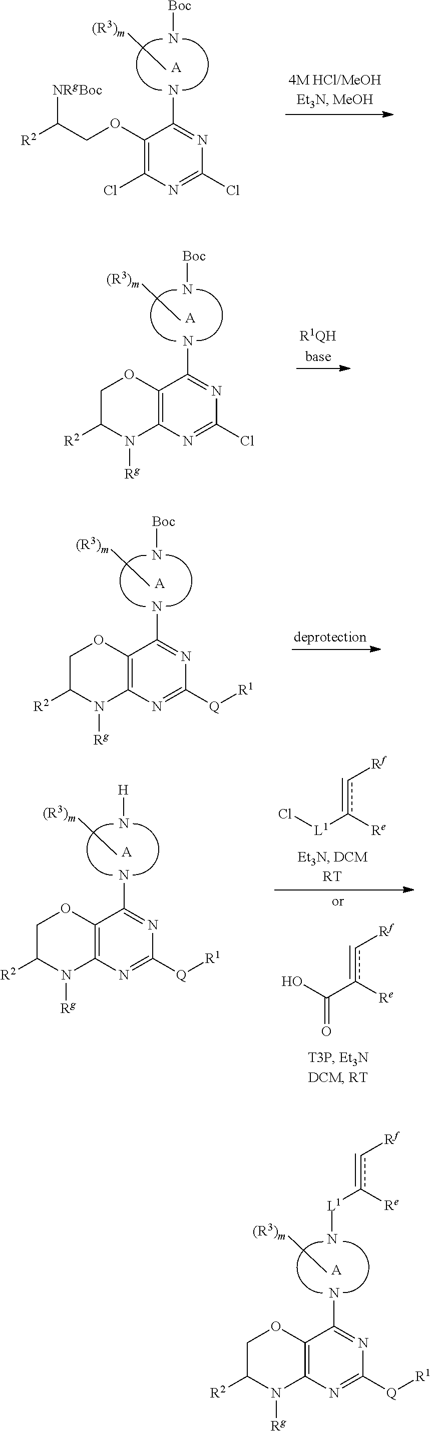

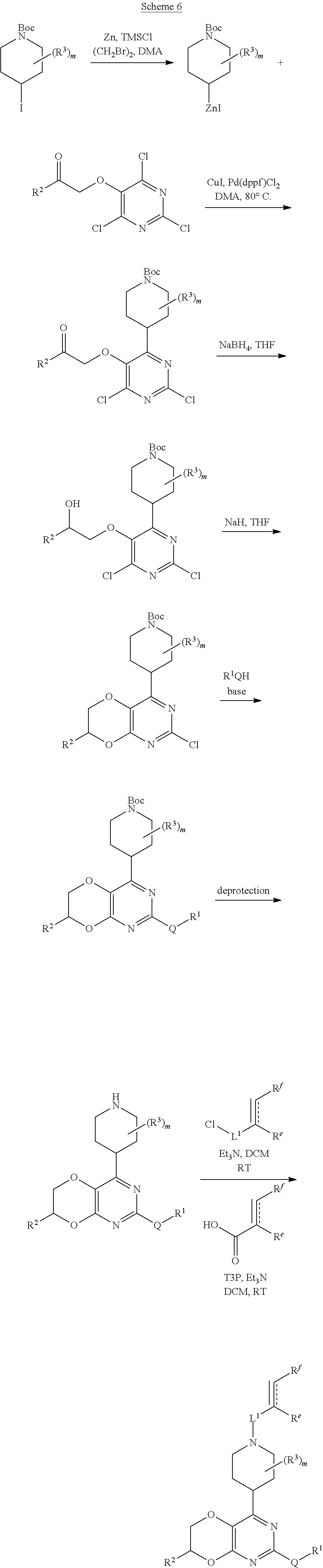

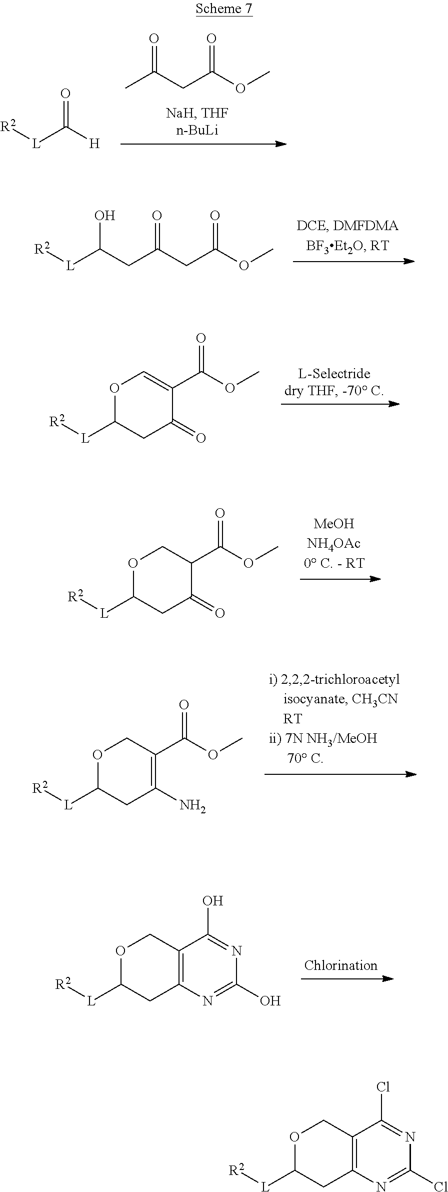

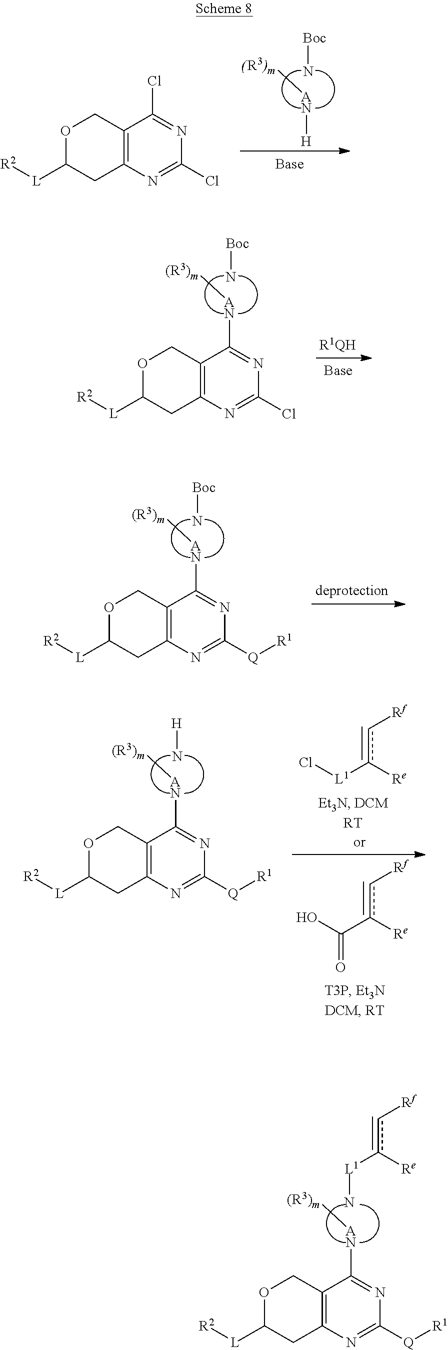

### SYNTHETIC EXAMPLES

### Example S-1: Synthesis of 1-(4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-1-yl)prop-2-en-1-one (Compound 40, Isomer A and Isomer B)

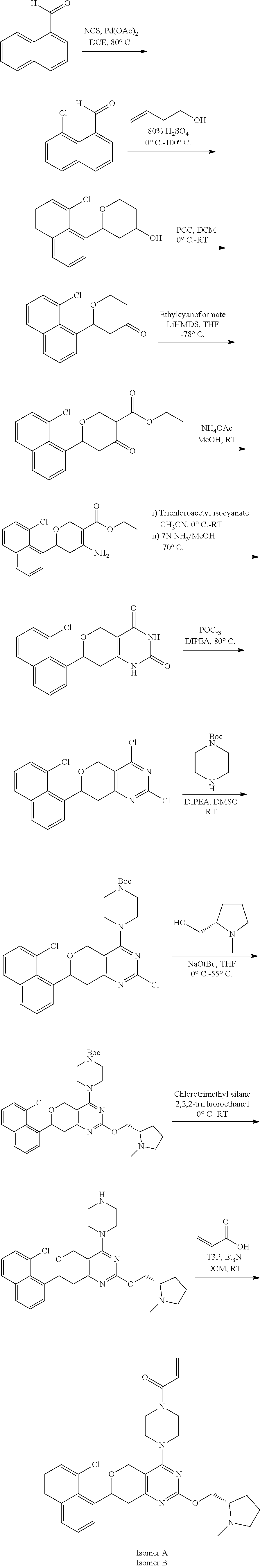

Step 1: Synthesis of 8-chloro-1-naphthaldehyde. To a solution of naphthaldehyde (5.0 g, 32.01 mmol, 1.0 eq) in DCE (50 mL) at 0° C., was added NCS (4.70 g, 35.21 mmol, 1.1 eq) followed by Pd(OAc)₂ (0.72 g, 3.20 mmol, 0.1 eq). TfOH (2.8 mL, 32.01 mmol, 1.0 eq) was added to the reaction mixture dropwise and stirred at 80° C. for 18 h. The reaction mixture was cooled to RT and concentrated under reduced pressure. The obtained material was purified by silica gel chromatography to afford the title compound.

Step 2: Synthesis of 2-(8-chloronaphthalen-1-yl)tetrahydro-2H-pyran-4-ol. To a solution of 8-chloro-1-naphthaldeyde (1.00 g, 5.25 mmol, 1.0 eq) in 80% H₂SO₄ (10.0 mL) was added 3-butene-1-ol (0.9 mL, 10.49 mmol, 2.0 eq) at 0° C. and stirred at 100° C. for 4 h. The reaction mixture was cooled to RT and poured into ice cold water, pH of the mixture was adjusted to 5 using 6N NaOH solution (15 mL) and extracted with CH₂Cl₂ (3×10 mL). The combined organic layers were dried over Na₂SO₄, filtered, and concentrated under reduced pressure. The obtained material was purified by silica gel chromatography to afford the title compound.

Step 3: Synthesis of 2-(8-chloronaphthalen-1-yl)tetrahydro-4H-pyran-4-one. To a solution of 2-(8-chloronaphthalen-1-yl)tetrahydro-2H-pyran-4-ol (0.50 g, 1.90 mmol, 1.0 eq) in dry CH₂Cl₂ (10 mL) was added PCC (0.82 g, 3.81 mmol, 2.0 eq) portion wise at 0° C. and allowed to stir at RT for 4 h. The reaction mixture was diluted with CH₂Cl₂ (5 mL) filtered through a pad of Celite and washed with CH₂Cl₂ (10 mL). The obtained filtrate was washed with brine (10 mL) and water (20 mL). The combined organic layers were dried over Na₂SO₄, filtered, and concentrated under reduced pressure. The obtained crude material was purified by silica gel chromatography to afford the title compound.

Step 4: Synthesis of ethyl-6-(8-chloronaphthalen-1-yl)-4-oxotetrahydro-2H-pyran-3-carboxylate. To a solution of 2-(8-chloronaphthalen-1-yl)tetrahydro-4H-pyran-4-one (0.20 g, 0.77 mmol, 1.0 eq) in dry THF (5 mL) was added LiHMDS (1.0 M in THF; 0.84 mL, 0.84 mmol, 1.1 eq) at −78° C., stirred for 1 h. ethyl cyanoformate (0.08 mL, 0.846 mmol, 1.1 eq) was added at same temperature and stirred for 2 h. The reaction mixture was quenched with sat. NH₄Cl solution (5 mL) and extracted with EtOAc (3×10 mL). The combined organic layers were dried over Na₂SO₄, filtered, and concentrated under reduced pressure. The obtained crude material was purified by silica gel chromatography to afford the title compound.

Step 5: Synthesis of ethyl-4-amino-6-(8-chloronaphthalen-1-yl)-5,6-dihydro-2H-pyran-3-carboxylate. To a solution of ethyl-6-(8-chloronaphthalen-1-yl)-4-oxotetrahydro-2H-pyran-3-carboxylate (0.10 g, 0.30 mmol, 1.0 eq) in methanol (5 mL) was added NFLOAc (0.07 g, 0.90 mmol, 3.0 eq) at RT and stirred for 18 h. The reaction mixture was concentrated under reduced pressure and diluted with H₂O (10 mL) and extracted with EtOAc (3×10 mL). The combined organic layers were dried over Na₂SO₄, filtered, and concentrated under reduced pressure to afford the title compound. The crude material was taken to the next step without any further purification.

Step 6: Synthesis of 7-(8-chloronaphthalen-1-yl)-1,5,7,8-tetrahydro-2H-pyrano[4,3-d]pyrimidine-2,4(3H)-dione. To a solution of ethyl-4-amino-6-(8-chloronaphthalen-1-yl)-5,6-dihydro-2H-pyran-3-carboxylate (0.10 g, 0.30 mmol, 1.0 eq) in dry acetonitrile (5 mL) was added trichloro acetyl isocyanate (0.07 mL, 0.60 mmol, 2.0 eq) at 0° C. and allowed to stir at RT for 30 min. The obtained solid was collected through filtration, dissolved in 7N ammonia in methanol and stirred at 70° C. for 2 h. The reaction mixture was cooled to RT. The obtained solid was collected through filtration to afford the title compound.

Step 7: Synthesis of 2,4-dichloro-7-(8-chloronaphthalen-1-yl)-7,8-dihydro-5H-pyrano[4,3-d]pyrimidine. To a stirred solution of 7-(8-chloronaphthalen-1-yl)-1,5,7,8-tetrahydro-2H-pyrano[4,3-d]pyrimidine-2,4(3H)-dione (0.30 g, 0.91 mmol, 1.0 eq) in dry POCl₃ (10 mL) was added DIPEA (0.3 mL, 1.82 mmol, 2.0 eq) and stirred at 80° C. for 18 h. The reaction mixture was cooled to RT, poured in ice cold water and basified to pH=8-10 using aqueous sat. NaHCO₃ solution, and extracted with CH₂Cl₂ (3×10 mL). The combined organic layers were dried over Na₂SO₄, filtered, and concentrated under reduced pressure. The resulted crude material was purified by silica gel chromatography to afford the title compound.

Step 8: Synthesis of tert-butyl-4-(2-chloro-7-(8-chloronaphthalen-1-yl)-7,8-dihydro-5H-pyrano[4,3-d]pyrimidin-4-yl)piperazine-1-carboxylate. A solution of 2,4-dichloro-7-(8-chloronaphthalen-1-yl)-7,8-dihydro-5H-pyrano[4,3-d]pyrimidine (0.05 g, 0.14 mmol, 1.0 eq) in dry DMSO (2.0 mL) was added 1-Boc-piperazine (0.029 g, 0.15 mmol, 1.1 eq) and DIPEA (0.07 mL, 0.42 mmol, 3.0 eq) at RT and stirred for 4 h. The reaction mixture was diluted with cold H₂O (10 mL) and extracted with EtOAc (3×10 mL). The combined organic layers were dried over Na₂SO₄, filtered, and concentrated under reduced pressure to afford the title compound.

Step 9: Synthesis of tert-butyl-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazine-1-carboxylate. A solution of N-methyl-L-prolinol (0.07 mL, 0.58 mmol, 1.5 eq) in dry THE (2.0 mL) was added sodium tert-butoxide (0.08 g, 0.78 mmol, 2.0 eq) at 0° C. and stirred for 1 h at the same temperature then the reaction mixture was added a solution of tert-butyl-4-(2-chloro-7-(8-chloronaphthalen-1-yl)-7,8-dihydro-5H-pyrano[4,3-d]pyrimidin-4-yl)piperazine-1-carboxylate (0.21 g, 0.39 mmol, 1.0 eq) in THE (0.5 mL) and stirred at 55° C. for 24 h. The reaction mixture was cooled to RT, quenched with ice cold water (10 mL) and extracted with EtOAc (2×10 mL). The combined organic layers were dried over Na₂SO₄, filtered, and concentrated under reduced pressure to afford the title compound.

Step 10: Synthesis of 7-(8-chloronaphthalen-1-yl)-2-(((S)-1-methylpyrrolidin-2-yl)methoxy)-4-(piperazin-1-yl)-7,8-dihydro-5H-pyrano[4,3-d]pyrimidine. To a solution of tert-butyl-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazine-1-carboxylate (0.27 g, 0.45 mmol, 1.0 eq) in 2,2,2-trifluoroethanol (2.0 mL) was cooled to 0° C. and chlorotrimethylsilane (0.5 mL) was added dropwise at 0° C. The reaction mixture was stirred at RT for 2 h. The volatiles were removed under reduced pressure to obtain the crude material which was triturated with n-pentane and MTBE (5.0 mL) to afford the title compound.

Step 11: Synthesis of 1-(4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-1-yl)prop-2-en-1-one. To a solution of 7-(8-chloronaphthalen-1-yl)-2-(((5)-1-methylpyrrolidin-2-yl)methoxy)-4-(piperazin-1-yl)-7,8-dihydro-5H-pyrano[4,3-d]pyrimidine (0.27 g, 0.51 mmol, 1.0 eq) in dry dichloromethane (3.0 mL) was added triethylamine (0.2 mL, 1.53 mmol, 3.0 eq), acrylic acid (0.04 mL, 0.61 mmol, 1.2 eq). T3P (50% in EtOAc; 0.70 mL, 1.02 mmol, 2.0 eq) was added dropwise at 0° C. and stirred at RT for 4 h. The reaction mixture was quenched with saturated NaHCO₃ (5.0 mL) and extracted with dichloromethane (2×10 mL). The combined organic layers were dried over anhydrous Na₂SO₄ and concentrated under reduced pressure to obtain the crude material. The obtained crude material was purified by preparative HPLC. Pure fractions were collected and concentrated under reduced pressure to afford the title compound as a mixture of isomers which were further separated by SLC chromatography to afford the title compound as Isomer A and Isomer B.

Isomer A: LCMS (m/z): 548.3 [M+H]⁺; ¹H NMR (400 MHz, CDCl₃): δ 7.89 (d, J=7.2 Hz, 1H), 7.82 (t, J=8.0 Hz, 2H), 7.61-7.53 (m, 2H), 7.36 (t, J=8.0 Hz, 1H), 6.56-6.49 (m, 1H), 6.42 (dd, J=3.6 Hz and 11.2 Hz, 1H), 6.30-6.26 (m, 1H), 5.69 (dd, J=2.0 Hz and 10.8 Hz, 1H), 4.87-4.70 (m, 2H), 4.35-4.31 (m, 1H) 4.07-4.03 (m, 1H), 3.73 (br s, 3H), 3.57-3.21 (m, 6H), 3.01 (t, J=7.2 Hz, 1H), 2.83-2.76 (m, 1H), 2.61-2.58 (m, 1H), 2.41 (s, 3H), 2.23-2.16 (m, 1H), 2.00-1.93 (m, 1H), 1.78-1.70 (m, 3H).

Isomer B: LCMS (m/z): 548.4 [M+H]⁺; ¹H NMR (400 MHz, CDCl₃): δ 7.97 (d, J=7.2 Hz, 1H), 7.75 (t, J=8.0 Hz, 2H), 7.54-7.46 (m, 2H), 7.29 (t, J=8.0 Hz, 1H), 6.62-6.56 (m, 1H), 6.49 (dd, J=3.2 Hz and 11.2 Hz, 1H), 6.36-6.32 (m, 1H), 5.75 (dd, J=2.0 Hz and 10.4 Hz, 1H), 4.94-4.77 (m, 2H), 4.42 (br s, 1H), 4.20 (br s, 1H), 3.80 (br s, 3H), 3.64-3.37 (m, 7H), 3.07 (br s, 1H), 2.89-2.82 (m, 1H), 2.53 (br s, 3H), 2.34 (s, 1H), 2.08 (br s, 1H), 1.79 (br s, 3H).

### Example S-2: Synthesis of 2-((2S)-1-acryloyl-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile (Compound 38, Isomer A and Isomer B)

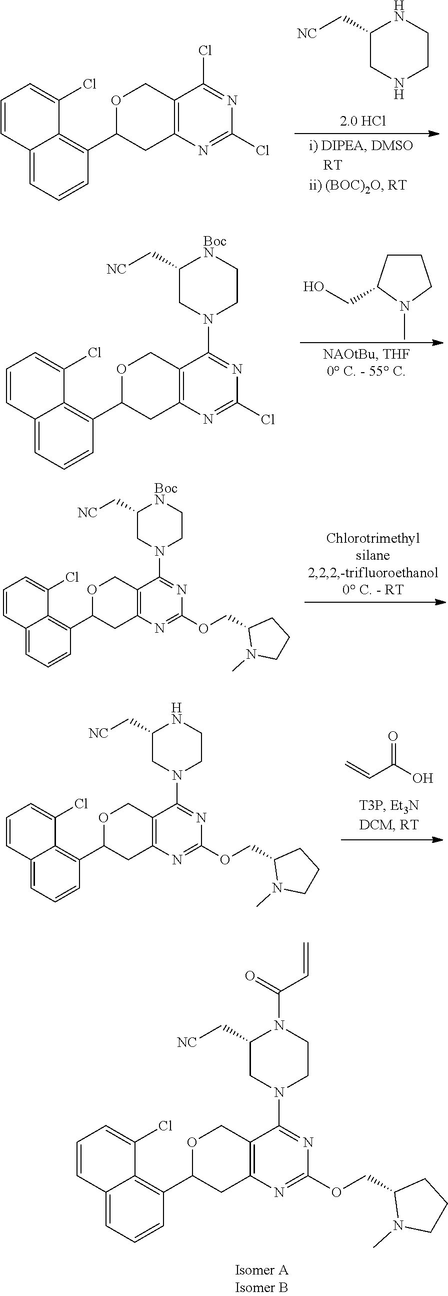

Step 1: Synthesis of tert-butyl-(2S)-4-(2-chloro-7-(8-chloronaphthalen-1-yl)-7,8-dihydro-5H-pyrano[4,3-d]pyrimidin-4-yl)-2-(cyanomethyl)piperazine-1-carboxylate. To a solution of 2,4-dichloro-7-(8-chloronaphthalen-1-yl)-7,8-dihydro-5H-pyrano[4,3-d]pyrimidine (0.15 g, 0.41 mmol, 1.0 eq) in dry DMSO (3.0 mL) was added 2[(2S)-piperazine-2-yl]acetonitrile dihydrochloride (0.089 g, 0.45 mmol, 1.1 eq) and DIPEA (0.34 mL, 2.05 mmol, 5.0 eq) at RT and stirred for 4 h. To the resulting mixture, Boc anhydride (0.14 mL, 0.60 mmol, 1.5 eq) was added and stirred at RT for 10 h. The reaction mixture was diluted with cold water (20 mL) and extracted with EtOAc (2×5 mL). The combined organic layers were washed with ice water (2×2 mL), brine (5 mL), dried over Na₂SO₄ and concentrated under reduced pressure. The crude material was purified by silica gel chromatography to afford the title compound.

Step 2: Synthesis of tert-butyl-(2S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)-2-(cyanomethyl)piperazine-1-carboxylate. To a solution of N-methyl-L-prolinol (0.08 g, 0.67 mmol, 1.5 eq) in dry THE (1.0 mL) was added sodium tert-butoxide (0.09 mg, 0.90 mmol, 2.0 eq) at 0° C. and stirred for 1 h at the same temperature followed by the addition of tert-butyl-(2S)-4-(2-chloro-7-(8-chloronaphthalen-1-yl)-7,8-dihydro-5H-pyrano[4,3-d]pyrimidin-4-yl)-2-(cyanomethyl)piperazine-1-carboxylate (0.25 g, 0.45 mmol, 1.0 eq) in THF (0.5 mL) and stirred at 55° C. for 24 h. The reaction mixture was cooled to RT, quenched with ice cold water (5 mL) and extracted with EtOAc (2×5 mL). The combined organic layers were dried over Na₂SO₄, filtered, and concentrated under reduced pressure to afford the title compound.

Step 3: Synthesis of 2-((2S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile. To a solution of tert-butyl-(2S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)-2-(cyanomethyl)piperazine-1-carboxylate (0.25 g, 0.39 mmol, 1.0 eq) in 2,2,2-trifluoroethanol (3 mL) was cooled to 0° C. and chlorotrimethylsilane (0.11 mL, 0.78 mmol, 2.0 eq) was added dropwise at 0° C. The reaction mixture was stirred at RT for 2 h. The volatiles were removed under reduced pressure to obtain the crude material which was triturated with n-pentane and MTBE (5 mL) to afford the title compound.

Step 4: Synthesis of 2-((2S)-1-acryloyl-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile. To a solution of 2-((2S)-4-(7-(8-chloronaphthalen-1-yl)-2-(((5)-1-methylpyrrolidin-2-yl)methoxy)-7,8-dihydro-5H-pyrano[4,3-d]pyrimidin-4-yl)piperazin-2-yl)acetonitrile (0.15 g, 0.26 mmol, 1.0 eq) in dry dichloromethane (2.0 mL) was added triethylamine (0.11 mL, 0.78 mmol, 3.0 eq), acrylic acid (0.022 g, 0.31 mmol, 1.2 eq). T3P (50% in EtOAc; 0.31 mL, 0.52 mmol, 2.0 eq) was added dropwise at 0° C. and stirred at RT for 2 h. The reaction mixture was quenched with sat. NaHCO₃ (5.0 mL) and extracted with dichloromethane (2×10 mL). The combined organic layers were dried over anhydrous Na₂SO₄ and concentrated under reduced pressure to obtain the crude material. The obtained crude material was purified by preparative-HPLC. Pure fractions were collected and concentrated under reduced pressure to afford the title compound as Isomer A and Isomer B.

Isomer A: LCMS (m/z): 587.3 [M+H]⁺; ¹H NMR (400 MHz, CDCl₃): δ 7.96 (d, J=7.2 Hz, 1H), 7.82 (t, J=8.0 Hz, 2H), 7.61-7.59 (m, 1H), 7.54 (t, J=7.6 Hz, 1H), 7.36 (t, J=7.6 Hz, 1H), 6.62-6.56 (m, 1H), 6.47 (dd, J=3.2 Hz and 10.8 Hz, 1H), 6.39 (dd, J=1.2 Hz and 16.4 Hz, 1H), 5.82 (d, J=11.6 Hz, 1H), 4.99-4.83 (m, 3H), 4.37 (dd, J=5.2 Hz and J=10.8 Hz, 1H), 4.17 (dd, J=6.4 Hz and J=10.4 Hz, 1H), 3.79-3.56 (m, 4H), 3.22-2.85 (m, 6H), 2.69-2.66 (m, 2H), 2.43 (s, 3H), 2.29-2.25 (m, 1H), 2.07-2.05 (m, 1H), 1.83-1.74 (m, 3H).

Isomer B: LCMS (m/z): 587.3 [M+H]+; ¹H NMR (400 MHz, CDCl₃): δ 7.97 (d, J=7.6 Hz, 1H), 7.82 (t, J=9.2 Hz, 2H), 7.60 (d, J=6.4 Hz, 1H), 7.55 (t, J=8.0 Hz, 1H), 7.36 (t, J=7.6 Hz, 1H), 6.58-6.57 (m, 1H), 6.51 (dd, J=3.2 Hz and 11.2 Hz, 1H), 6.41-6.37 (m, 1H), 5.82 (d, J=10.4 Hz, 1H), 5.02-4.81 (m, 3H), 4.43-4.40 (m, 1H), 4.19-4.14 (m, 1H), 4.01-3.48 (m, 6H) 3.12-3.10 (m, 2H), 2.91-2.71 (m, 4H), 2.50 (s, 3H), 2.39-2.20 (m, 1H), 2.14-2.01 (m, 1H), 1.84-1.83 (m, 3H).

### Example S-3: Synthesis of 2-((2S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)-1-(2-fluoroacryloyl)piperazin-2-yl)acetonitrile (Compound 43, Isomer A and Isomer B)

### 2-((S)-4-((S)-7-(8-chloronaphthalen-1-yl)-2-(((S)-1-methylpyrrolidin-2-yl)methoxy)-7,8-dihydro-5H-pyrano[4,3-d]pyrimidin-4-yl)-1-(2-fluoroacryloyl)piperazin-2-yl)acetonitrile (Compound 43, Isomer A) and 2-((S)-4-((R)-7-(8-chloronaphthalen-1-yl)-2-(((S)-1-methylpyrrolidin-2-yl)methoxy)-7,8-dihydro-5H-pyrano[4,3-d]pyrimidin-4-yl)-1-(2-fluoroacryloyl)piperazin-2-yl)acetonitrile (Compound 43, Isomer B)

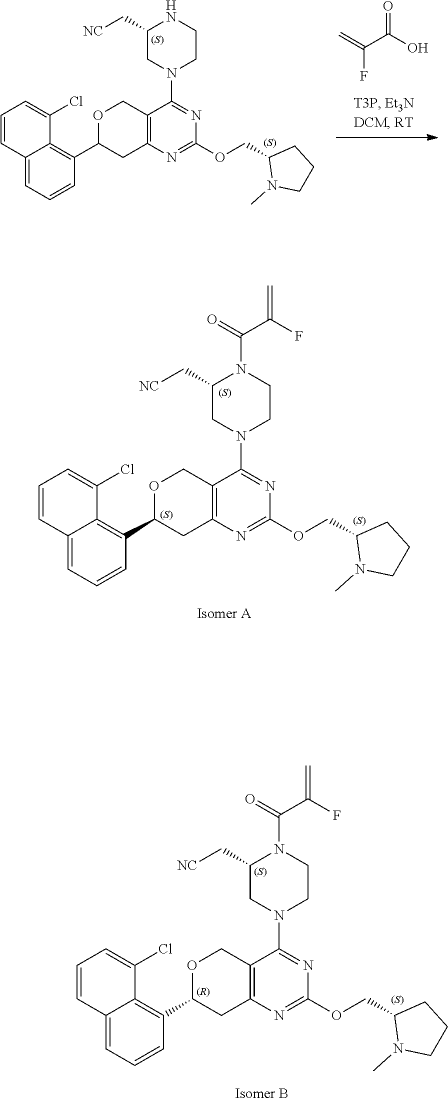

To a solution of 2-((2S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile (0.10 g, 0.17 mmol, 1.0 eq) in CH₂Cl₂ (2 mL) was added triethylamine (0.075 mL, 0.51 mmol, 3.0 eq), 2-fluoroacrylic acid (0.019 g, 0.20 mmol, 1.2 eq). T3P (50% in ethyl acetate; 0.2 mL, 0.34 mmol, 2.0 eq) was added dropwise at 0° C. The reaction mixture was stirred at RT for 2 h. The reaction mixture was quenched with sat. NaHCO₃ (5 mL) and extracted with dichloromethane (2×10 mL). The combined organic layers were dried over Na₂SO₄ and concentrated under reduced pressure to obtain the crude material. The crude material was purified by preparative-HPLC. Pure fractions were collected and concentrated under reduced pressure to afford the title compound as Isomer A and Isomer B.

The absolute configuration of compound 43 (Isomer A) was determined to be (S,S,S) from a single crystal X-ray structure and the absolute configuration of compound 43 (Isomer B) was inferred to be (S,R,S) from the stereochemistry of compound 43 (Isomer A).

Isomer A: LCMS (m/z): 605.4 [M+H]+; ¹H NMR (400 MHz, CDCl₃): δ 7.96 (d, J=6.8 Hz, 1H), 7.82 (t, J=8.0 Hz, 2H), 7.60 (d, 1H), 7.55 (t, J=8.0 Hz, 1H), 7.36 (t, J=7.6 Hz, 1H), 6.47 (dd, J=3.2 Hz and 10.8 Hz, 1H), 5.47-5.36 (m, 1H), 5.25 (dd, J=3.2 Hz and 16.4 Hz, 1H), 4.97 (d, J=14.0 Hz, 1H), 4.84 (d, J=13.6 Hz, 1H), 4.43-4.38 (m, 1H), 4.19-4.13 (m, 2H), 4.06-4.00 (m, 2H), 3.78-3.76 (m, 2H), 3.61-3.56 (m, 2H), 3.26-3.03 (m, 4H), 2.92-2.85 (m, 2H), 2.68-2.40 (m, 3H), 2.31-2.29 (m, 1H), 2.05-2.00 (m, 1H), 1.85-1.77 (m, 3H).

Isomer B: LCMS (m/z): 605.3 [M+H]⁺; ¹H NMR (400 MHz, CDCl₃): δ 7.97 (d, J=7.2 Hz, 1H), 7.82 (t, J=8.8 Hz, 2H), 7.60 (d, J=6.8 Hz, 1H), 7.55 (t, J=7.2 Hz, 1H), 7.36 (t, J=7.6 Hz, 1H), 6.52 (dd, J=3.2 Hz and 10.8 Hz, 1H), 5.47-5.35 (m, 1H), 5.25 (dd, J=3.6 Hz and 17.2 Hz, 1H), 4.99 (d, J=13.6 Hz, 1H), 4.82 (d, J=13.6 Hz, 1H), 4.58-4.50 (m, 1H), 4.23 (s, 1H), 4.02 (d, J=14.4 Hz, 1H), 3.81 (d, J=11.2 Hz, 2H), 3.62-3.49 (m, 4H), 3.07 (br s, 2H), 2.91-2.75 (m, 4H), 2.58 (br s, 3H), 2.35 (br s, 1H), 2.10-2.04 (m, 1H), 1.83-1.75 (m, 3H).

### Example S-4: Synthesis of(S)-1-(4-(7-(8-chloronaphthalen-1-yl)-2-((1-methylpyrrolidin-2-yl)methoxy)-5H-pyrano[2,3-d]pyrimidin-4-yl)piperazin-1-yl)-2-fluoroprop-2-en-1-one (Compound 174)

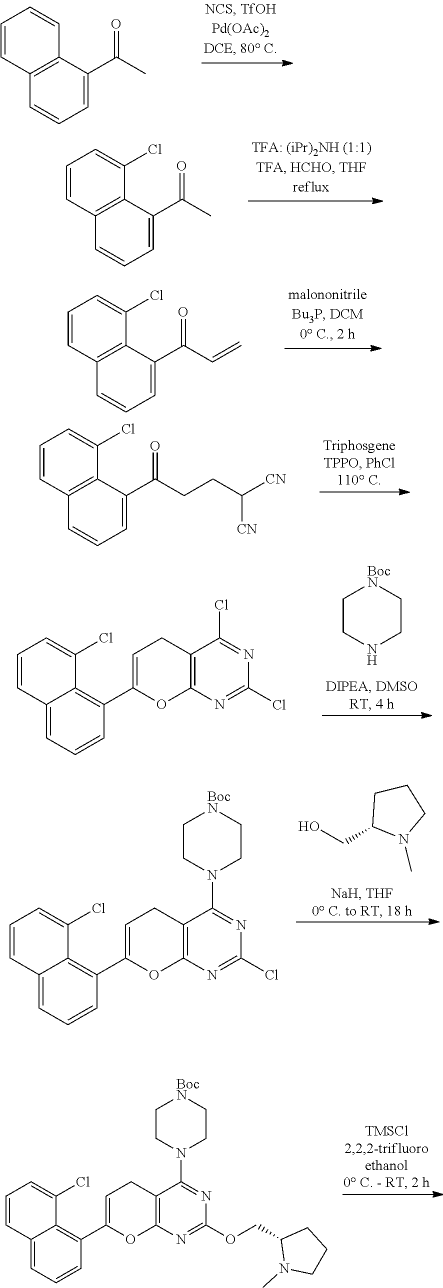

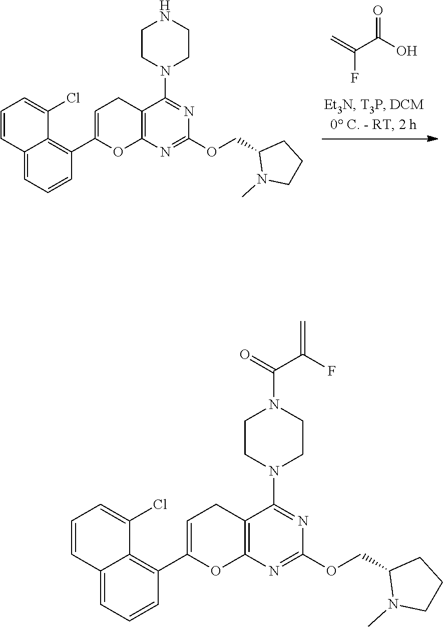

Step 1: Synthesis of 1-(8-chloronaphthalen-1-yl)ethan-1-one. To a solution of 1-(naphthalen-1-yl)ethan-1-one (5.0 g, 29.38 mmol, 1.0 eq) in DCE (100 mL) was cooled 0° C. and added NCS (4.30 g, 32.31 mmol, 1.1 eq) followed by Pd(OAc)₂ (0.66 g, 2.94 mmol, 0.1 eq). To the resulting reaction mixture, TfOH (1.96 mL, 29.38 mmol, 1.0 eq) was added dropwise and heated to 80° C. for 18 h. The reaction mixture was cooled to RT and concentrated under reduced pressure. The crude material was purified by silica gel chromatography to afford the title compound.

Step 2: Synthesis of 1-(8-chloronaphthalen-1-yl)prop-2-en-1-one. To a solution of l-(8-chloronaphthalen-1-yl)ethan-1-one (2.0 g, 9.77 mmol, 1.0 eq) in dry THF (20 mL) was added TFA:(iPr₂NH) salt (2.31 g, 10.74 mmol, 1.0 eq), followed by formaldehyde (1.46 g, 48.78 mmol, 5.0 eq) and catalytic amount of TFA (0.1 mL) at RT and heated to reflux for 18 h. The reaction mixture was cooled to RT and the solvent was removed under reduced pressure. The obtained crude material was diluted with EtOAc (100 mL) and washed with 1N HCl solution (50 mL), followed by sat. NaHCO₃ (50 mL) and brine (20 mL). The combined organic layers were dried over Na₂SO₄, filtered, and concentrated under reduced pressure. The crude material was purified by silica gel chromatography to afford the title compound.

Step 3: Synthesis of 2-(3-(8-chloronaphthalen-1-yl)-3-oxopropyl)malononitrile. To a solution of 1-(8-chloronaphthalen-1-yl)prop-2-en-1-one (0.30 g, 1.38 mmol, 1.0 eq) in dry CH₂Cl₂ (10 mL) at 0° C. was added malononitrile (0.09 g, 1.38 mmol, 1.0 eq) followed by dropwise addition of tri-n-butylphosphine (0.03 mL g, 0.14 mmol, 0.1 eq) and the resulting mixture was stirred at 0° C. for 2 h. The reaction mixture was concentrated under reduced pressure to provide crude material which was purified by silica gel chromatography to afford the title compound.

Step 4: Synthesis of 2,4-dichloro-7-(8-chloronaphthalen-1-yl)-5H-pyrano[2,3-d]pyrimidine. A solution of TPPO (0.295 g, 1.05 mmol, 3.0 eq) in chlorobenzene (5 mL) was cooled to 0° C. and charged with triphosgene (0.10 g in 3 mL, 0.35 mmol, 1.0 eq) in chlorobenzene solution drop wise at 0° C. and allowed to stirred at RT for 30 min. Then 2-(3-(8-chloronaphthalen-1-yl)-3-oxopropyl)malononitrile (0.10 g, 0.35 mmol, 1.0 eq) in chlorobenzene solution was added at RT and heated to 110° C. for 8 h. The reaction mixture was cooled to RT and poured in ice cold water, basified by using sat. NaHCO₃ solution (15 mL) and extracted with CH₂Cl₂ (3×10 mL). The combined organic layers were dried over Na₂SO₄, filtered, and concentrated under reduced pressure to provide crude material which was purified by silica gel chromatography to afford the title compound.

Step 5: Synthesis of tert-butyl-4-(2-chloro-7-(8-chloronaphthalen-1-yl)-5H-pyrano[2,3-d]pyrimidin-4-yl)piperazine-1-carboxylate. To a solution of 2,4-dichloro-7-(8-chloronaphthalen-1-yl)-5H-pyrano[2,3-d]pyrimidine (0.05 g, 0.14 mmol, 1.0 eq) in dry DMSO (5 mL) was added 1-Boc-piperazine (0.028 g, 0.15 mmol, 1.1 eq) and DIPEA (0.07 mL, 0.41 mmol, 3.0 eq) at RT and stirred for 4 h. The reaction mixture was diluted with cold H₂O (10 mL) and extracted with EtOAc (3×10 mL). The combined organic layers were dried over Na₂SO₄, filtered and concentrated under reduced pressure to afford the title compound.

Step 6: Synthesis of tert-butyl (S)-4-(7-(8-chloronaphthalen-1-yl)-2-((1-methylpyrrolidin-2-yl)methoxy)-5H-pyrano[2,3-d]pyrimidin-4-yl)piperazine-1-carboxylate. To a stirred solution of NaH (60%, 0.018 g, 0.75 mmol, 3.0 eq) in dry THF (10 mL) was added N-methyl-L-prolinol (0.029 g, 0.25 mmol, 1.0 eq) dropwise at 0° C. and stirred for 1 h. Then a solution of tert-butyl-4-(2-chloro-7-(8-chloronaphthalen-1-yl)-5H-pyrano[2,3-d]pyrimidin-4-yl)piperazine-1-carboxylate (0.13 g, 0.25 mmol, 1.0 eq) in THF was added dropwise to reaction mixture and stirred at RT for 18 h. The reaction mixture was quenched with ice cold water (10 mL) and extracted with EtOAc (2×10 mL). The combined organic layers were dried over Na₂SO₄, filtered, and concentrated under reduced pressure. The crude mixture was purified by silica gel chromatography to afford the title compound.

Step 7: Synthesis of (S)-7-(8-chloronaphthalen-1-yl)-2-((1-methylpyrrolidin-2-yl)methoxy)-4-(piperazin-1-yl)-5H-pyrano[2,3-d]pyrimidine. To a solution of tert-butyl (S)-4-(7-(8-chloronaphthalen-1-yl)-2-((1-methylpyrrolidin-2-yl)methoxy)-5H-pyrano[2,3-d]pyrimidin-4-yl)piperazine-1-carboxylate (0.035 g, 0.06 mmol, 1.0 eq) in 2,2,2-trifluoroethanol (5 mL) at 0° C. was added chlorotrimethylsilane (0.015 mL, 0.12 mmol, 2.0 eq) dropwise and the reaction mixture was stirred at RT for 2 h. The volatiles were removed under reduced pressure to obtain the crude material which was triturated with n-pentane and hexane to afford the title compound.

Step 8: Synthesis of (S)-1-(4-(7-(8-chloronaphthalen-1-yl)-2-((1-methylpyrrolidin-2-yl)methoxy)-5H-pyrano[2,3-d]pyrimidin-4-yl)piperazin-1-yl)-2-fluoroprop-2-en-1-one. To a solution of (S)-7-(8-chloronaphthalen-1-yl)-2-((1-methylpyrrolidin-2-yl)methoxy)-4-(piperazin-1-yl)-5H-pyrano[2,3-d]pyrimidine (0.03 g, 0.056 mmol, 1.0 eq) in CH₂Cl₂ (3 mL) at 0° C., was added Et₃N (0.023 mL, 0.168 mmol, 3.0 eq) followed by the addition of 2-fluoroacrylic acid (6.60 mg, 0.073 mmol, 1.3 eq) and T3P (50% in EtOAc; 0.07 mL, 0.112 mmol, 2.0 eq). The reaction mixture was stirred at RT for 2 h. The reaction mixture was quenched with aqueous sat. NaHCO₃ (5 mL) and extracted with CH₂Cl₂ (2×10 mL). The combined organic layers were dried over Na₂SO₄ and concentrated under reduced pressure to obtain the crude material. The crude material was purified by preparative-HPLC to afford the title compound. LCMS (m/z): 564.3 [M+H]⁺; ¹H NMR (400 MHz, CDCl₃): δ 7.82 (d, J=8.0 Hz, 1H), 7.71 (d, J=8.0 Hz, 1H), 7.52-7.47 (m, 2H), 7.41-7.37 (m, 1H), 7.33-7.29 (m, 1H), 5.30-5.08 (m, 3H), 4.27-4.19 (m, 1H), 4.13-4.04 (m, 1H), 3.68 (br s, 4H), 3.41 (br s, 4H), 3.36 (d, J=3.6 Hz, 2H), 3.02-2.98 (m, 1H), 2.57-2.54 (m, 1H), 2.37 (s, 3H), 2.22-2.15 (m, 1H), 1.97-1.87 (m, 1H), 1.76-1.57 (m, 3H).

### Example S-5: Synthesis of 2-((S)-4-(7-(8-chloronaphthalen-1-yl)-2-(((S)-1-methylpyrrolidin-2-yl)methoxy)-5H-pyrano[2,3-d]pyrimidin-4-yl)-1-(2-fluoroacryloyl)piperazin-2-yl)acetonitrile (Compound 178)

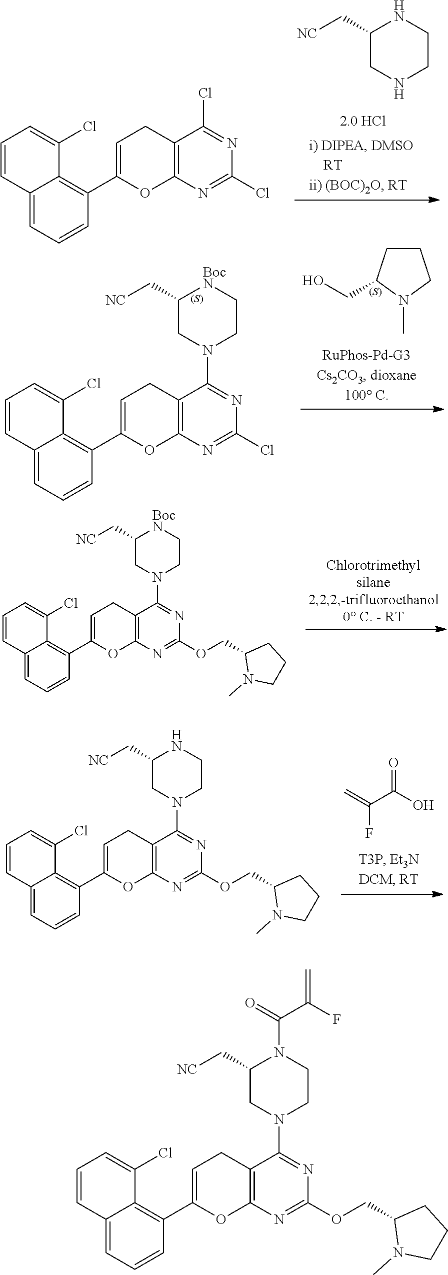

Step 1: Synthesis of tert-butyl (S)-4-(2-chloro-7-(8-chloronaphthalen-1-yl)-5H-pyrano[2,3-d]pyrimidin-4-yl)-2-(cyanomethyl)piperazine-1-carboxylate. To a solution of 2,4-dichloro-7-(8-chloronaphthalen-1-yl)-5H-pyrano[2,3-d]pyrimidine (0.10 g, 0.27 mmol, 1.0 eq) in dry DMSO (5 mL) was added 2[(2S)-piperazin-2-yl]acetonitrile dihydrochloride (0.065 g, 0.33 mmol, 1.2 eq) and DIPEA (0.24 mL, 1.37 mmol, 5.0 eq) and stirred at RT for 4 h. To the resulting mixture, Boc anhydride (0.09 mL, 0.41 mmol, 1.5 eq) was added and stirred at RT for 10 h. The reaction mixture was diluted with cold H₂O (10 mL) and extracted with EtOAc (2×20 mL). The combined organic layers were dried over Na₂SO₄, filtered, and concentrated under reduced pressure to afford the title compound.

Step 2: Synthesis of tert-butyl-(S)-4-(7-(8-chloronaphthalen-1-yl)-2-(((S)-1-methylpyrrolidin-2-yl)methoxy)-5H-pyrano[2,3-d]pyrimidin-4-yl)-2-(cyanomethyl)piperazine-1-carboxylate. To a stirred solution of tert-butyl (S)-4-(2-chloro-7-(8-chloronaphthalen-1-yl)-5H-pyrano[2,3-d]pyrimidin-4-yl)-2-(cyanomethyl)piperazine-1-carboxylate (0.20 g, 0.36 mmol, 1.0 eq) in dry dioxane (5 mL), was added N-methyl-L-prolinol (0.13 g, 1.08 mmol, 3.0 eq) under argon atmosphere followed by RuPhos-Pd-G3 (0.015 g, 0.018 mmol, 0.05 eq) and Cs₂CO₃ (0.35 g, 1.08 mmol, 3.0 eq) and the resulting reaction mixture was heated to 100° C. for 48 h. The reaction mixture was cooled to RT, filtered through Celite bed and the filtrate was concentrated under reduced pressure. The crude mixture was purified by silica gel chromatography to afford the title compound.

Step 3: Synthesis of 2-((S)-4-(7-(8-chloronaphthalen-1-yl)-2-(((S)-1-methylpyrrolidin-2-yl)methoxy)-5H-pyrano[2,3-d]pyrimidin-4-yl)piperazin-2-yl)acetonitrile. To a solution of tert-butyl-(S)-4-(7-(8-chloronaphthalen-1-yl)-2-(((S)-1-methylpyrrolidin-2-yl)methoxy)-5H-pyrano[2,3-d]pyrimidin-4-yl)-2-(cyanomethyl)piperazine-1-carboxylate H-14 (0.085 g, 0.13 mmol, 1.0 eq) in 2,2,2-trifluoroethanol (10 mL) at 0° C., was added chlorotrimethylsilane (0.03 mL, 0.26 mmol, 2.0 eq) and the reaction mixture was stirred at RT for 2 h. The volatiles were removed under reduced pressure to obtain the crude material. The obtained crude material was triturated with n-pentane and hexane to afford the title compound.

Step 4: Synthesis of 2-((S)-4-(7-(8-chloronaphthalen-1-yl)-2-(((S)-1-methylpyrrolidin-2-yl)methoxy)-5H-pyrano[2,3-d]pyrimidin-4-yl)-1-(2-fluoroacryloyl)piperazin-2-yl)acetonitrile. To a solution of 2-((S)-4-(7-(8-chloronaphthalcn-1-yl)-2-(((S)-1-methylpyrrolidin-2-yl)methoxy)-5H-pyrano[2,3-d]pyrimidin-4-yl)piperazin-2-yl)acetonitrile (0.03 g, 0.06 mmol, 1.0 eq) in CH₂Cl₂ (5 mL) at 0° C., was added Et₃N (0.02 mL, 0.17 mmol, 3.0 eq) followed by the addition of 2-fluoroacrylic acid (6.1 mg, 0.067 mmol, 1.2 eq) and T3P (50% in EtOAc; 0.06 mL, 0.11 mmol, 2.0 eq) and the reaction mixture was stirred at RT for 2 h. The reaction mixture was quenched with aqueous sat. NaHCO₃ (5 mL) and extracted with CH₂Cl₂ (2×10 mL). The combined organic layers were dried over Na₂SO₄ and concentrated under reduced pressure to obtain the crude material. The crude material was further purified by preparative-HPLC to afford the title compound. LCMS (m/z): 603.3 [M+H]⁺; ¹H NMR (400 MHz, CDCl₃): δ 7.90 (d, J=7.2 Hz, 1H), 7.78 (d, J=8.4 Hz, 1H), 7.60-7.55 (m, 2H), 7.49-7.45 (m, 1H), 7.40-7.36 (m, 1H), 5.47-5.35 (m, 1H), 5.35-5.22 (m, 2H), 4.88 (br s, 4H), 4.29-4.24 (m, 1H), 4.21-4.15 (m, 1H), 3.96-3.93 (m, 1H), 3.85-3.83 (m, 1H), 3.49 (br s, 2H), 3.44-3.27 (m, 2H), 3.08-2.95 (m, 3H), 2.85-2.82 (m, 1H), 2.61-2.60 (m, 1H), 2.43 (s, 3H), 2.28-2.22 (m, 1H), 2.03-1.94 (m, 1H), 1.85-1.66 (m, 3H).

### Example S-6: Synthesis of 2-((S)-1-acryloyl-4-(7-(8-chloronaphthalen-1-yl)-2-(((S)-1-methylpyrrolidin-2-yl)methoxy)-5H-pyrano[2,3-d]pyrimidin-4-yl)piperazin-2-yl)acetonitrile (Compound 181)

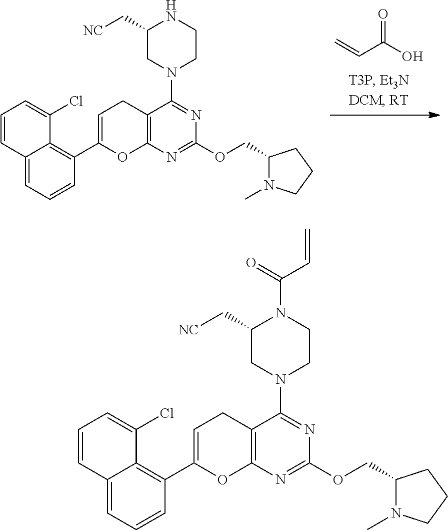

To a solution of 2-((S)-4-(7-(8-chloronaphthalen-1-yl)-2-(((S)-1-methylpyrrolidin-2-yl)methoxy)-5H-pyrano[2,3-d]pyrimidin-4-yl)piperazin-2-yl)acetonitrile (0.05 g, 0.094 mmol, 1.0 eq) in CH₂Cl₂ (5 mL)) at 0° C., was added Et₃N (0.04 mL, 0.28 mmol, 3.0 eq) followed by the addition of acrylic acid (8.1 mg, 0.11 mmol, 1.2 eq) and T3P (50% in EtOAc; 0.11 mL, 0.18 mmol, 2.0 eq) and the reaction mixture was stirred at RT for 2 h. The reaction mixture was quenched with aqueous sat. NaHCO₃ (5 mL) and extracted with CH₂Cl₂ (2×10 mL). The combined organic layers were dried over Na₂SO₄ and concentrated under reduced pressure to obtain the crude material which was purified by preparative-HPLC to afford the title compound. LCMS (m/z): 585.4 [M+H]⁺; ¹H NMR (400 MHz, CDCl₃): δ 7.91-7.89 (m, 1H), 7.80-7.78 (m, 1H), 7.60-7.55 (m, 2H), 7.79-7.45 (m, 1H), 7.40-7.36 (m, 1H), 6.59 (br s, 1H), 6.41-6.37 (m, 1H), 5.84-5.81 (m, 1H), 5.29 (t, 7=3.6, Hz, 1H), 5.07 (br s, 1H), 4.34-4.28 (m, 1H), 4.22-4.17 (m, 1H), 3.96-3.81 (m, 3H), 3.62-3.50 (m, 2H), 3.35-3.25 (m, 1H), 3.11-3.01 (m, 2H), 2.99-2.92 (m, 2H), 2.78-2.67 (m, 2H), 2.46 (s, 3H), 2.32-2.26 (m, 1H), 2.05-1.93 (m, 1H), 1.91-1.73 (m, 3H).

### Example 5-7: Synthesis of 2,4,6-trichloropyrimidin-5-ol

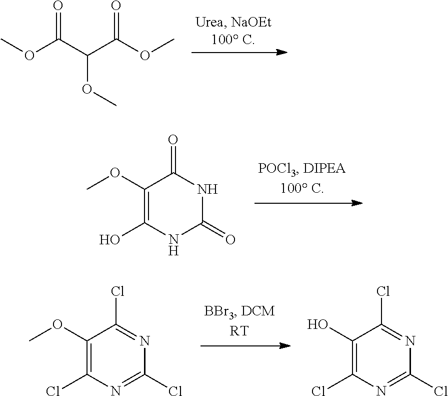

Step 1: Synthesis of 6-hydroxy-5-methoxypyrimidine-2,4(1H,3H)-dione. Under an atmosphere of nitrogen, sodium metal (3.5 g, 0.15 mol, 3.75 eq) was added portion wise to ethanol (100 mL) at 40° C., and the mixture was stirred until a solution was formed. Urea (2.5 g, 0.04 mol, 1.0 eq) was added and the mixture heated at 100° C. for 30 min. The reaction mixture was cooled to 60° C., dimethyl 2-methoxymalonate (10 g, 0.06 mol, 1.5 eq) was added and the resulting mixture was stirred at 100° C. for 4 h. The reaction mixture was cooled to RT and concentrated under reduced pressure. Water was added to the mixture and the pH was adjusted to 3 with cone. HCl (12N). The precipitate was filtered and dried to produce the title compound which was used in the next step without any further purification.

Step 2: Synthesis of 2,4,6-trichloro-5-methoxypyrimidine. To a solution of 6-hydroxy-5-methoxypyrimidine-2,4(1H,3H)-dione (17 g, crude) in POCl₃ (50 mL) at 5° C. was added DIPEA (5.7 g, 44 mmol) and the reaction mixture was stirred at 100° C. overnight. The reaction mixture was cooled to RT and concentrated under reduced pressure. The residue was poured into ice-water and extracted with DCM. The combined organic layers were washed with brine, dried over Na₂SO₄, filtered, and concentrated under reduced pressure to obtain crude material. The crude material was purified by silica gel chromatography to afford the title compound.

Step 3: Synthesis of Synthesis of 2,4,6-trichloropyrimidin-5-ol. To a solution of 2,4,6-trichloro-5-methoxypyrimidine (3.8 g, 17.8 mmol, 1.0 eq) in DCM (40 mL) was added BBr₃ (15.6 g, 62.3 mmol, 3.5 eq) at 0° C. and the resulting mixture was stirred at RT overnight. The reaction was quenched with water at 0° C. and extracted with DCM. The combined organic layers were washed with brine, dried over Na₂SO₄, filtered, and concentrated under reduced pressure to afford the title compound.

### Example S-8: Synthesis of 2-((2S)-4-(7-(8-chloronaphthalen-1-yl)-2-(((S)-1-methylpyrrolidin-2-yl)methoxy)-6,7-dihydro-[1,4]dioxino[2,3-d]pyrimidin-4-yl)-1-(2-fluoroacryloyl)piperazin-2-yl)acetonitrile (Compound 64, Isomer A and Isomer B)

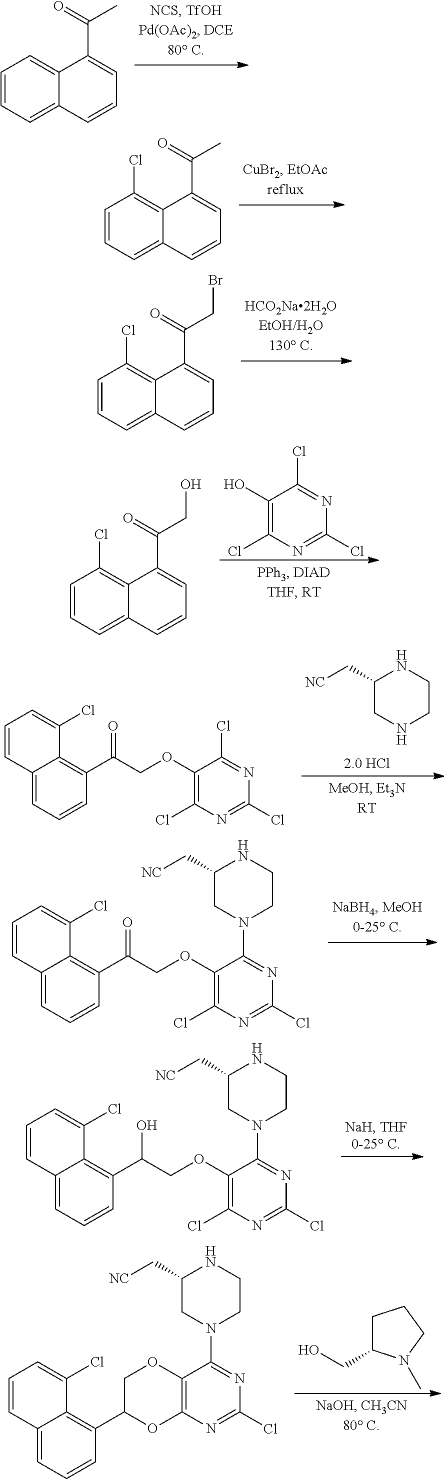

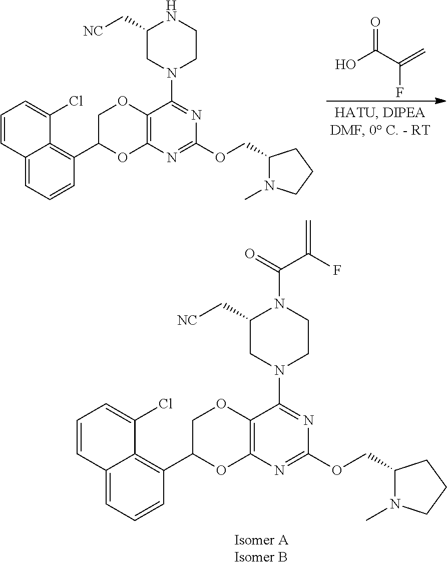

Step 1: Synthesis of 1-(8-chloronaphthalen-1-yl)ethan-1-one. To a solution of 1-(naphthalen-1-yl)ethan-1-one (10.0 g, 58.7 mmol, 1.0 eq) in DCE (250 mL) was added NCS (8.3 g, 62.1 mmol, 1.05 eq), TfOH (8.8 g, 58.7 mmol, 1.0 eq), Pd(OAc)₂ (1.3 g, 5.8 mmol, 0.1 eq) and the reaction mixture was degassed and purged with N₂ (3 x). Then the mixture was stirred at 80° C. for 4 h. The reaction mixture was filtered through a pad of Celite. The filtrate was diluted with DCM, washed with brine, dried over Na₂SO₄, filtered, and concentrated under reduced pressure to obtain crude material. The crude material was purified by silica gel chromatography to afford the title compound.

Step 2: Synthesis of 2-bromo-1-(8-chloronaphthalen-1-yl)ethan-1-one. To a solution of 1-(8-chloronaphthalen-1-yl)ethan-1-one (8.5 g, 41.5 mmol, 1.0 eq) in EtOAc (100 mL) was added CuBr₂ (19.0 g, 85.0 mmol, 2.05 eq) and stirred at 80° C. overnight. The reaction mixture was filtered, and the filtrate was concentrated under reduced pressure to produce the title compound which was used in the next step without any further purification.

Step 3: Synthesis of 1-(8-chloronaphthalen-1-yl)-2-hydroxyethan-1-one. To a solution of 2-bromo-1-(8-chloronaphthalen-1-yl)ethan-1-one (12 g, crude) in EtOH/H₂O (85 mL/15 mL) was added sodium formate (26.5 g, 0.25 mol) and stirred at 130° C. in a sealed tube. TLC indicated the disappearance of starting materials and the reaction mixture was diluted with water, extracted with EtOAc. The combined organic layers were washed with brine, dried over Na₂SO₄, filtered, and concentrated under reduced pressure to obtain crude material. The crude material was purified by silica gel chromatography to afford the title compound.

Step 4: Synthesis of 1-(8-chloronaphthalen-1-yl)-2-((2,4,6-trichloropyrimidin-5-yl)oxy) ethan-1-one. To a solution of 1-(8-chloronaphthalen-1-yl)-2-hydroxyethan-1-one (3.90 g, 17.6 mmol, 1.2 eq) in THF (40 mL) was added 2,4,6-trichloropyrimidin-5-ol (3.0 g, 14.7 mmol, 1.0 eq), PPh₃ (4.60 g, 17.6 mmol, 1.2 eq), DIAD (3.60 g, 17.6 mmol, 1.2 eq) and the reaction mixture was stirred at RT overnight. The solvent was evaporated under reduced pressure and the crude material was purified by silica gel chromatography to afford the title compound.

Step 5: Synthesis of (S)-2-(4-(2,6-dichloro-5-(2-(8-chloronaphthalen-1-yl)-2-oxoethoxy)pyrimidin-4-yl)piperazin-2-yl)acetonitrile. To a solution of 1-(8-chloronaphthalen-1-yl)-2-((2,4,6-trichloropyrimidin-5-yl)oxy)ethan-1-one (0.97 g, 2.4 mmol, 1.0 eq) in MeOH (20 mL) was added (S)-2-(piperazin-2-yl)acetonitrile hydrochloride (0.48 g, 2.4 mmol, 1.0 eq), Et₃N (1.3 g, 12.0 mmol, 5.0 eq) and the reaction mixture was stirred at RT. The reaction mixture was diluted with EtOAc, washed with 1N HCl. The combined organic layers were washed with brine, dried over Na₂SO₄, filtered, and concentrated under reduced pressure to produce the title compound. LCMS (m/z): 490.1 [M+H]⁺

Step 6: Synthesis of 2-((2S)-4-(2,6-dichloro-5-(2-(8-chloronaphthalen-1-yl)-2-hydroxy-ethoxy)pyrimidin-4-yl)piperazin-2-yl)acetonitrile. To a solution of (S)-2-(4-(2,6-dichloro-5-(2-(8-chloronaphthalen-1-yl)-2-oxoethoxy)pyrimidin-4-yl)piperazin-2-yl)acetonitrile (0.9 g, 1.8 mmol, 1.0 eq) in MeOH (20 mL) was added NaBH₄ (0.35 g, 9.0 mmol, 5.0 eq) portion wise at 0° C. and the reaction mixture was stirred at RT for 5 h. The reaction mixture was quenched with water, extracted with EtOAc. The combined organic layers were washed with brine, dried over Na₂SO₄, filtered, and concentrated under reduced pressure to produce the title compound. LCMS (m/z): 492.1 [M+H]⁺

Step 7: Synthesis of 2-((2S)-4-(2-chloro-7-(8-chloronaphthalen-1-yl)-6,7-dihydro-[1.4]dioxino[2,3-d]pyrimidin-4-yl)piperazin-2-yl)acetonitrile. To a solution of 2-((2S)-4-(2,6-dichloro-5-(2-(8-chloronaphthalen-1-yl)-2-hydroxyethoxy)pyrimidin-4-yl)piperazin-2-yl)acetonitrile (0.93 g, crude) in THF (20 mL) was added NaH (60% dispersion in oil, 0.22 g, 9.1 mmol) at 0° C. and the reaction mixture was stirred at RT for 3 h. The reaction mixture was quenched with water, extracted with EtOAc. The combined organic layers were washed with brine, dried over Na₂SO₄, filtered, and concentrated under reduced pressure to produce the crude compound. The crude material was purified by silica gel chromatography to afford the title compound. LCMS (m/z): 456.3 [M+H]⁺

Step 8: Synthesis of 2-((2S)-4-(7-(8-chloronaphthalen-1-yl)-2-(((S)-1-methylpyrrolidin-2-yl) methoxy)-6,7-dihydro-[1,4]dioxino[2,3-d]pyrimidin-4-yl)piperazin-2-yl)acetonitrile. To a solution of 2-((2S)-4-(2-chloro-7-(8-chloronaphthalen-1-yl)-6,7-dihydro-[1.4]dioxino[2,3-d]pyrimidin-4-yl)piperazin-2-yl)acetonitrile (700 mg, 1.5 mmol, 1.0 eq) in acetonitrile (30 mL) was added N-methyl-L-prolinol (3.60 g, 30 mmol, 20 eq), NaOH (0.61 g, 15 mmol, 10 eq) and the reaction mixture was stirred at 80° C. The reaction mixture was diluted with water, extracted with DCM. The combined organic layers were dried over Na₂SO₄, filtered, and concentrated under reduced pressure to produce crude material. The crude material was purified by reversed-phase chromatography followed by prep-TLC to afford the title compound. LCMS (m/z): 535.3 [M+H]⁺

Step 9: Synthesis of 2-((2S)-4-(7-(8-chloronaphthalen-1-yl)-2-(((S)-1-methyl pyrrolidin-2-yl)methoxy)-6,7-dihydro-[1,4]dioxino[2,3-d]pyrimidin-4-yl)-1-(2-fluoro acryloyl)piperazin-2-yl)acetonitrile. To a solution of 2-((2S)-4-(7-(8-chloronaphthalen-1-yl)-2-(((S)-1-methylpyrrolidin-2-yl)methoxy)-6,7-dihydro-[1,4]dioxino[2,3-d]pyrimidin-4-yl)piperazin-2-yl)acetonitrile (100 mg, 0.18 mmol, 1.0 eq) in DMF (3 mL) was added 2-fluoroacrylic acid (17 mg, 0.18 mmol, 1.0 eq), DIPEA (0.1 mL, 0.56 mmol, 3.1 eq), HATU (76 mg, 0.20 mmol, 1.1 eq) at 0° C. and the resulting mixture was stirred at RT for 10 min. Water was added to the reaction mixture and the precipitate formed was filtered and washed with excess water to afford the title compound as a mixture of isomers. The mixture of isomers was separated by chiral SFC to provide the title compound as Isomer A and Isomer B.

Isomer A: LCMS (m/z): 607.4 [M+H]⁺; ¹H NMR (400 MHz, DMSO-d₆) δ 8.08 (dd, J=21.8, 8.2 Hz, 2H), 7.80 (dd, J=21.4, 7.4 Hz, 2H), 7.69 (t, J=7.8 Hz, 1H), 7.55 (t, J=7.8 Hz, 1H), 6.96 (dd, J=8.0, 2.4 Hz, 1H), 5.38 (dd, J=17.2, 4.4 Hz, 1H), 5.26 (d, J=49.6 Hz, 1H), 4.84 (dd, J=11.6, 2.4 Hz, 1H), 4.59 (d, J=13.6 Hz, 1H), 4.49 (d, J=13.6 Hz, 1H), 4.18 (dd, J=10.4, 5.2 Hz, 1H), 3.96 (m, 2H), 3.20 (dd, J=13.8, 3.4 Hz, 1H), 3.11-2.85 (m, 4H), 2.49 (m, 4H), 2.33 (s, 3H), 2.16 (m, 1H), 2.04-1.85 (m, 1H), 1.72-1.51 (m, 3H).

Isomer B: LCMS (m/z): 607.3 [M+H]⁺; ¹H NMR (400 MHz, DMSO-d₆) δ 8.08 (m, 2H), 7.80 (m, 2H), 7.69 (t, J=7.8 Hz, 1H), 7.55 (t, J=7.8 Hz, 1H), 6.97 (dd, J=7.6, 2.4 Hz, 1H), 5.37 (dd, J=18.2, 4.2 Hz, 1H), 5.26 (d, J=47.2 Hz, 1H), 4.82 (dd, J=11.8, 2.2 Hz, 1H), 4.59-4.45 (m, 2H), 4.17-4.16 (m, 1H), 4.03-4.01 (m, 2H), 3.22 (dd, J=13.6, 3.6 Hz, 1H), 2.97-2.95 (m, 4H), 2.50-2.49 (m, 4H), 2.36 (s, 3H), 2.25-2.22 (m, 1H), 2.03-1.87 (m, 1H), 1.72-1.68 (m, 2H), 1.62-1.49 (m, 1H).

### Example S-9: Synthesis of 7-(8-chloronaphthalen-1-yl)-7,8-dihydro-5H-pyrano[4,3-d]pyrimidine-2,4-diol

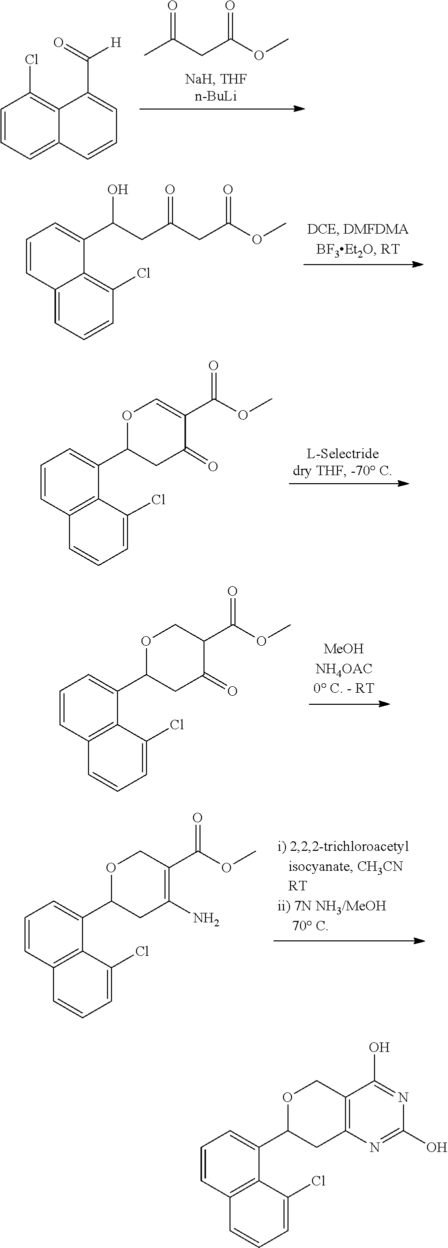

Step 1: Synthesis of methyl-5-(8-chloronaphthalen-1-yl)-5-hydroxy-3-oxopentanoate. To a solution of methylacetoacetate (5.8 g, 0.05 mol, 1.0 eq) in THF (120 mL) was added NaH (2.5 g, 0.06 mol, 1.2 eq) portionwise at 0° C. and the mixture was stirred at 0° C. for 1 h. Then n-BuLi (25 mL, 2.4 M in n-hexane) was added and the mixture was stirred for another 1 h. To the reaction mixture, chloronaphthaldehyde (19.6 g, 0.06 mol, 1.2 eq) was added and the mixture was stirred overnight. The reaction mixture was diluted with EtOAc (200 mL), then poured into ice-cooled saturated aqueous NH₄Cl (100 mL) and extracted with EtOAc (2×50 mL). The combined organic layers were washed with brine, dried over Na₂SO₄, filtered, and concentrated under reduced pressure to produce the crude compound. The crude material was purified by silica gel chromatography to afford the title compound. LCMS (m/z): 307.0 [M+H]⁺

Step 2: Synthesis of methyl-2-(8-chloronaphthalen-1-yl)-4-oxo-3,4-dihydro-2H-pyran-5-carboxylate. To a solution of methyl-5-(8-chloronaphthalen-1-yl)-5-hydroxy-3-oxopentanoate (14.0 g, 0.045 mol, 1.0 eq) in 1,2-dichloroethane (140 mL) was added dropwise a solution of DML-DMA (5.5 g, 0.045 mol, 1.0 eq) in 1,2-dichloroethane (10 mL) and the mixture was stirred at RT for 1 h. Then was added dropwise BF₃·Et₂O (6.8 g, 0.045 mol, 1.0 eq) and the mixture was stirred at RT for 2 h. The reaction mixture was diluted with EtOAc (300 mL) and washed with saturated aqueous NaHCO₃ (100 mL), brine (2×100 mL). The organic layer was dried over anhydrous Na₂SO₄, filtered and concentrated under reduced pressure to afford the title product as a crude material which was used directly in the next step without any further purification. LCMS (m/z): 317.0 [M+H]⁺

Step 3: Synthesis of methyl 6-(8-chloronaphthalen-1-yl)-4-oxotetrahydro-2H-pyran-3-carboxylate. To a solution of methyl-2-(8-chloronaphthalen-1-yl)-4-oxo-3,4-dihydro-2H-pyran-5-carboxylate (15.0 g, 0.040 mol, 1.0 eq) in dry THE (300 mL) at −70° C. was added L-selectride (48 mL, 0.048 mol, 1M in THE, 1.2 eq) dropwise and the mixture was stirred at −70° C. for 2 h. Then the reaction mixture was diluted with EtOAc (300 mL), poured into ice-cooled saturated aqueous NH₄Cl (100 mL) and the organic phase was washed with brine (2×100 mL), dried over anhydrous Na₂SO₄. After filtration, the filtrate was concentrated under reduced pressure produced the crude title product which was used directly in the next step without any further purification. LCMS (m/z): 319.0 [M+H]⁺

Step 4: Synthesis of methyl-4-amino-6-(8-chloronaphthalen-1-yl)-5,6-dihydro-2H-pyran-3-carboxylate. To a solution of methyl-6-(8-chloronaphthalen-1-yl)-4-oxotetrahydro-2H-pyran-3-carboxylate (15.0 g, 0.047 mol, 1.0 eq) in MeOH (300 mL) was added NH₄OAc (5.5 g, 0.094 mol, 2.0 eq) and the reaction mixture was stirred at RT overnight. The reaction mixture was concentrated under reduced pressure and diluted with H₂O (100 mL) and extracted with EtOAc (3×100 mL). The combined organic layers were washed with brine, dried over Na₂SO₄, filtered, and concentrated under reduced pressure to produce the crude compound. The crude material was purified by silica gel chromatography to afford the title compound. LCMS (m/z): 318.0 [M+H]⁺

Step 5: Synthesis of 7-(8-chloronaphthalen-1-yl)-7,8-dihydro-5H-pyrano[4,3-d]pyrimidine-2,4-diol. To a solution of methyl-4-amino-6-(8-chloronaphthalen-1-yl)-5,6-dihydro-2H-pyran-3-carboxylate (8.0 g, 0.025 mol, 1.0 eq) in acetonitrile (15 mL) was added 2,2,2-trichloroacetyl isocyanate (9.45 g, 0.05 mol, 2.0 eq) at 0° C. and the mixture was stirred at RT for 30 min. The obtained solid was collected through filtration, dissolved in 7N ammonia in methanol (120 mL) and stirred in a 250 mL sealed tube at 70° C. for 12 h. The reaction mixture was cooled to RT and the precipitated solid was filtered and washed with MeOH (150 mL) to afford the title compound. LCMS (m/z): 329.0 [M+H]⁺

### Example S-10: Synthesis of 2-((2S)-4-(7-(8-chloronaphthalen-1-yl)-2-(3-(diethylamino)azetidin-1-yl)-7,8-dihydro-5H-pyrano[4,3-d]pyrimidin-4-yl)-1-(2-fluoroacryloyl)piperazin-2-yl)acetonitrile (Compound 229, Isomer A and Isomer B)

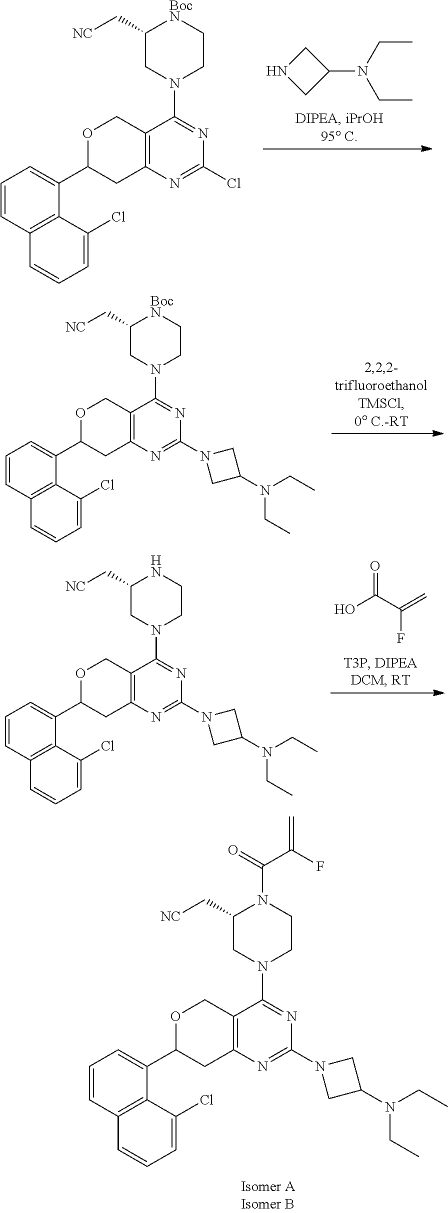

Step 1: Synthesis of tert-butyl (2S)-4-(7-(8-chloronaphthalen-1-yl)-2-(3-(diethyl amino)-azetidin-1-yl)-7,8-dihydro-5H-pyrano[4,3-d]pyrimidin-4-yl)-2-(cyano methyl) piperazine-1-carboxylate. To a solution of tert-butyl (2S)-4-(2-chloro-7-(8-chloronaphthalen-1-yl)-7,8-dihydro-5H-pyrano[4,3-d]pyrimidin-4-yl)-2-(cyanomethyl)piperazine-1-carboxylate (0.55 g, 1.0 mmol, 1.0 eq) in iPrOH (10 mL) was added N,N-diethylazetidin-3-amine (0.15 g, 1.19 mmol, 1.2 eq), DIPEA (0.52 mL, 2.98 mmol, 3.0 eq) and stirred at 95° C. for 4 h. The reaction solution was quenched with water (10 mL) and extracted with EtOAc (3×50 mL). The combined organic layers were washed with brine (3×50 mL), dried over Na₂SO₄, filtered, and concentrated under reduced pressure to produce the crude compound. The crude material was purified by silica gel chromatography to afford the title compound. LCMS (m/z): 646.0 [M+H]⁺

Step 2: Synthesis of 2-((2S)-4-(7-(8-chloronaphthalen-1-yl)-2-(3-(diethylamino)azetidin-1-yl)-7,8-dihydro-5H-pyrano[4,3-d]pyrimidin-4-yl)piperazin-2-yl)acetonitrile. To a solution of tert-butyl (2S)-4-(7-(8-chloronaphthalen-1-yl)-2-(3-(diethylamino)-azetidin-1-yl)-7,8-dihydro-5H-pyrano[4,3-d]pyrimidin-4-yl)-2-(cyanomethyl)piperazine-1-carboxylate (0.46 g, 0.71 mmol, 1.0 eq) in 2,2,2-trifluoroethanol (5 mL) at 0° C. was added TMSCl (0.18 mL, 1.43 mmol, 2.0 eq) dropwise and stirred at RT for 40 min. The reaction mixture was concentrated under reduced pressure to give the crude material. The crude was triturated with petroleum ether/MTBE (80 mL, 1:1) and filtered to afford the title compound. LCMS (m/z): 546.1 [M+H]⁺

Step 3: Synthesis of 2-((2S)-4-(7-(8-chloronaphthalen-1-yl)-2-(3-(diethylamino) azetidin-1-yl)-7,8-dihydro-5H-pyrano[4,3-d]pyrimidin-4-yl)-1-(2-fluoroacryloyl)piperazin-2-yl)acetonitrile. To a solution of 2-((2S)-4-(7-(8-chloronaphthalen-1-yl)-2-(3-(diethylamino)-azetidin-1-yl)-7,8-dihydro-5H-pyrano[4,3-d]pyrimidin-4-yl)piperazin-2-yl)acetonitrile (0.27 g, 0.50 mmol, 1.0 eq) in DCM (10 mL) was added 2-fluoroacrylic acid (0.09 g, 0.99 mmol, 2.0 eq), DIPEA (0.26 mL, 1.49 mmol, 3.0 eq), T3P (0.44 mL, 1.49 mmol, 3.0 eq) dropwise and stirred at RT for 12 h. The reaction solution was diluted with water (10 mL) and extracted with DCM (3×30 mL). The combined organic layers were washed with brine (3×30 mL), dried over Na₂SO₄, filtered, and concentrated under reduced pressure to produce the crude compound as a mixture of isomers. The mixture of isomers was purified and separated by chiral SEC to afford the title compound as Isomer A and Isomer B.

Isomer A: LCMS (m/z): 618.2 [M+H]⁺; ¹H NMR (400 MHz, CDCl₃): δ 7.96 (dd, J=7.4, 0.8 Hz, 1H), 7.80 (td, J=8.1, 1.2 Hz, 2H), 7.60-7.52 (m, 2H), 7.37-7.30 (m, 1H), 6.47 (dd, J=11.0, 3.4 Hz, 1H), 5.39 (d, J=48.4 Hz, 1H), 5.22 (dd, J=16.9, 3.4 Hz, 1H), 4.93-4.88 (d, J=13.2 Hz, 1H), 4.76 (d, J=13.4 Hz, 1H), 4.13-4.05 (m, 2H), 4.02-3.81 (m, 4H), 3.64 (m, 2H), 3.53-3.47 (m, 1H), 3.41-3.29 (m, 1H), 3.01-2.88 (m, 1H), 2.85-2.71 (m, 4H), 2.63-2.51 (m, 4H), 1.90-1.56 (s, 1H), 1.02 (t, J=7.1 Hz, 6H).

Isomer B: LCMS (m/z): 618.2 [M+H]⁺; ¹H NMR (400 MHz, CDCl₃): δ 7.94 (dd, J=7.4, 0.8 Hz, 1H), 7.82-7.76 (m, 2H), 7.59-7.51 (m, 2H), 7.37-7.32 (m, 1H), 6.43 (dd, J=11.0, 3.4 Hz, 1H), 5.47-5.30 (m, 1H), 5.27-5.17 (m, 1H), 4.91 (d, J=12.4 Hz, 1H), 4.76-4.69 (m, 1H), 4.49-4.28 (m, 2H), 4.26-4.08 (m, 4H), 3.84 (s, 1H), 3.63 (d, J=10.1 Hz, 1H), 3.50 (dd, J=18.1, 2.7 Hz, 1H), 3.27-3.08 (m, 4H), 2.92-2.74 (m, 6H), 1.23-1.13 (m, 6H).

### Example S-11: Synthesis of 2-((2S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)-1-methacryloylpiperazin-2-yl)acetonitrile (Compound 240, Isomer A and Isomer B)

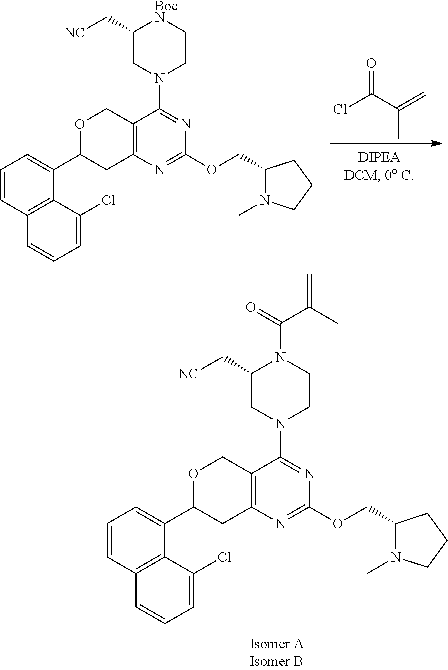

To a solution of 2-((2S)-4-(7-(8-chloronaphthalen-1-yl)-2-(((S)-1-methylpyrrolidin-2-yl)-methoxy)-7,8-dihydro-5H-pyrano[4,3-d]pyrimidin-4-yl)piperazin-2-yl)acetonitrile (300 mg, 0.56 mmol, 1.0 eq), DIPEA (0.39 mL, 2.25 mmol, 4.0 eq) in DCM (10 mL) at 0° C. was added methacryloyl chloride (0.07 mL, 0.67 mmol, 1.2 eq) and the reaction mixture was stirred at 0° C. for 10 min. The reaction mixture was diluted with H₂O (30 mL) and extracted with DCM (3×30 mL). The combined organic layers were washed with brine (3×30 mL), dried over anhydrous Na₂SO₄, filtered and concentrated under reduced pressure to yield the crude compound as a mixture of isomers. The crude was purified by prep-HPLC followed by chiral SLC to afford the title compound as Isomer A and Isomer B.

Isomer A: LCMS (m/z): 600.7 [M+H]⁺; ¹H NMR (400 MHz, CDCl₃): δ 7.95 (d, J=6.8 Hz, 1H), 7.85-7.79 (m, 2H), 7.61-7.52 (m, 2H), 7.38-7.33 (m, 1H), 6.46 (dd, J=10.9, 3.3 Hz, 1H), 5.31 (s, 1H), 5.15 (s, 1H), 4.97-4.79 (m, 3H), 4.30-4.01 (m, 2H), 3.74 (d, J=10.4 Hz, 1H), 3.64-3.53 (m, 1H), 3.19-3.15 (m, 4H), 3.06-2.81 (m, 4H), 2.75-2.64 (m, 4H), 2.12-2.01 (m, 5H), 1.68-1.87 (m, 4H).

Isomer B: LCMS (m/z): 600.7 [M+H]⁺; tH NMR (400 MHz, CDCl₃): δ 7.95 (d, J=7.4 Hz, 1H), 7.85-7.80 (m, 2H), 7.59-7.51 (m, 2H), 7.38-7.33 (m, 1H), 6.50 (dd, J=11.1, 3.4 Hz, 1H), 5.31 (s, 1H), 5.14 (s, 1H), 5.01-4.77 (m, 3H), 4.39-4.35 (m, 1H), 3.98 (d, J=13.7 Hz, 1H), 3.79-3.55 (m, 3H), 3.45-3.41 (m, 2H), 3.28-3.09 (m, 1H), 2.99-2.69 (m, 8H), 2.26-2.04 (m, 2H), 2.00-1.98 (m, 7H).

### Example S-12: Synthesis of 1-((3S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)-3-methylpiperazin-1-yl)-2-fluoroprop-2-en-1-one (Compound 44, Isomer A and Isomer B)

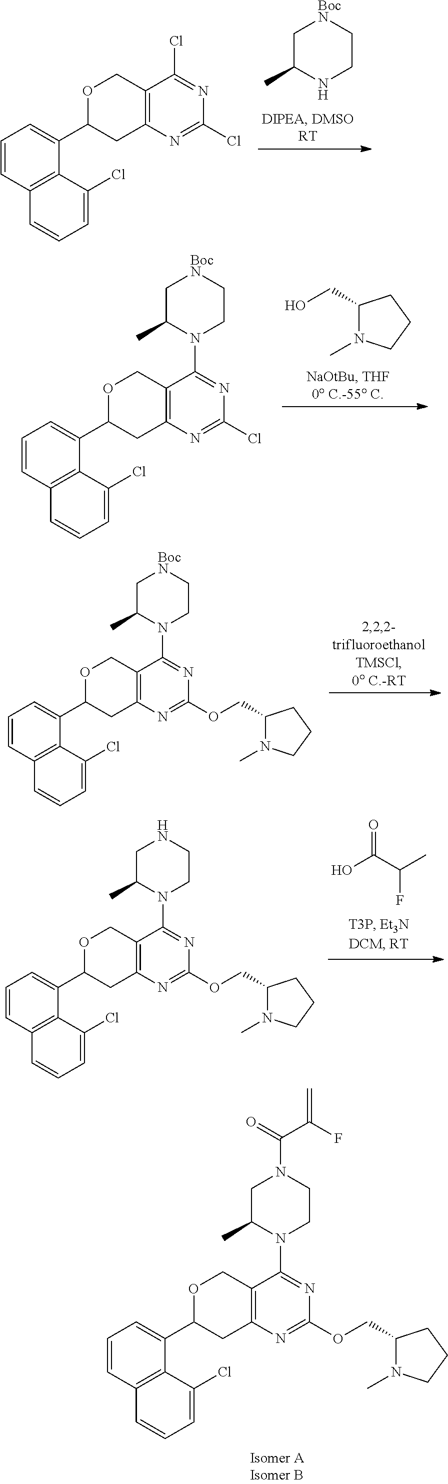

Step 1: Synthesis of tert-butyl (3S)-4-(2-chloro-7-(8-chloronaphthalen-1-yl)-7,8-dihydro-5H-pyrano[4,3-d]pyrimidin-4-yl)-3-methylpiperazine-1-carboxylate. To a solution of 2,4-dichloro-7-(8-chloronaphthalen-1-yl)-7,8-dihydro-5H-pyrano[4,3-d]pyrimidine (0.5 g, 1.37 mmol, 1.0 eq), tert-butyl (S)-3-methylpiperazine-1-carboxylate (0.27 g, 1.37 mmol, 1.0 eq) in DMSO (10 mL) was added DIPEA (0.71 mmol, 4.10 mmol, 3.0 eq) and stirred at RT for 12 h. The reaction mixture was diluted with H₂O (30 mL) and extracted with EtOAc (3×30 mL). The combined organic layers were washed with brine (30 mL), dried over anhydrous Na₂SO₄, filtered and concentrated under reduced pressure to yield the crude compound. The crude material was purified by silica gel chromatography to afford the title compound. LCMS (m/z): 529.2 [M+H]⁺

Step 2: Synthesis of tert-butyl (3S)-4-(7-(8-chloronaphthalen-1-yl)-2-(((S)-1-methyl-pyrrolidin-2-yl)methoxy)-7,8-dihydro-5H-pyrano[4,3-d]pyrimidin-4-yl)-3-methyl-piperazine-1-carboxylate. To a stirred solution of tert-butyl (3S)-4-(2-chloro-7-(8-chloronaphthalen-1-yl)-7,8-dihydro-5H-pyrano[4,3-d]pyrimidin-4-yl)-3-methylpiperazine-1-carboxylate (0.20 g, 0.38 mmol, 1.0 eq), N-methyl-L-prolinol (0.054 g, 0.46 mmol, 1.2 eq) in DMSO (5 mL) at 0° C. under nitrogen, was added NaOtBu (0.090 g, 0.94 mmol, 2.5 eq) and the reaction mixture was heated to 55° C. and stirred for 3 h. The reaction mixture was diluted with DCM (30 mL), washed with brine (30 mL), dried over anhydrous Na₂SO₄, filtered and concentrated under reduced pressure to yield the crude compound. The crude material was purified by silica gel chromatography to afford the title compound. LCMS (m/z): 608.1 [M+H]⁺

Step 3: Synthesis of 7-(8-chloronaphthalen-1-yl)-4-((S)-2-methylpiperazin-1-yl)-2-(((S)-1-methylpyrrolidin-2-yl)methoxy)-7,8-dihydro-5H-pyrano[4,3-d]pyrimidine. The title compound was synthesized following the procedure as described in Example S-10, Step 2 using tert-butyl (3S)-4-(7-(8-chloronaphthalen-1-yl)-2-(((S)-1-methyl-pyrrolidin-2-yl)methoxy)-7,8-dihydro-5H-pyrano[4,3-d]pyrimidin-4-yl)-3-methyl-piperazine-1-carboxylate (0.17 g, 0.28 mmol, 1.0 eq). LCMS (m/z): 508.0 [M+H]⁺

Step 4: Synthesis of 1-((3S)-4-(7-(8-chloronaphthalen-1-yl)-2-(((S)-1-methyl pyrrolidin-2-yl)methoxy)-7,8-dihydro-5H-pyrano[4,3-d]pyrimidin-4-yl)-3-methylpiperazin-1-yl)-2-fluoroprop-2-en-1-one. The title compound was synthesized following the procedure as described in Example S-10, Step 3 using 7-(8-chloronaphthalen-1-yl)-4-((S)-2-methylpiperazin-1-yl)-2-(((S)-1-methylpyrrolidin-2-yl)methoxy)-7,8-dihydro-5H-pyrano[4,3-d]pyrimidine (0.17 g, 0.33 mmol, 1.0 eq) and Et₃N (0.14 mL, 1 mmol, 3.0 eq). The crude compound was purified by silica gel chromatography and chiral SFC to afford the title compound as Isomer A and Isomer B.

Isomer A: LCMS (m/z): 580.1 [M+H]⁺; ¹H NMR (400 MHz, CD₃OD): δ 8.01 (d, J=7.4 Hz, 1H), 7.91 (d, J=8.2 Hz, 2H), 7.65 (dd, J=7.4, 1.1 Hz, 1H), 7.57 (t, J=7.8 Hz, 1H), 7.42 (t, J=7.8 Hz, 1H), 6.51 (dd, J=11.0, 3.2 Hz, 1H), 5.33-5.21 (m, 2H), 5.03 (d, J=14.0 Hz, 1H), 4.82 (d, J=13.8 Hz, 1H), 4.40-4.26 (m, 3H), 4.07 (s, 1H), 3.90 (s, 1H), 3.66 (d, J=13.9 Hz, 1H), 3.59-3.44 (m, 2H), 3.17-2.99 (m, 1H), 2.84-2.59 (m, 2H), 2.49 (s, 3H), 2.32-2.28 (m, 1H), 2.15-2.00 (m, 1H), 1.87-1.76 (m, 2H), 1.75-1.63 (m, 1H), 1.28 (s, 2H), 1.18 (d, J=6.6 Hz, 3H).

Isomer B: LCMS (m/z): 580.2 [M+H]⁺; ¹H NMR (400 MHz, CD₃OD): δ 8.01 (d, J=7.6 Hz, 1H), 7.91 (d, J=8.1 Hz, 2H), 7.65 (d, J=7.4 Hz, 1H), 7.58 (t, J=7.8 Hz, 1H), 7.43 (t, J=7.8 Hz, 1H), 6.50 (dd, J=10.8, 3.1 Hz, 1H), 5.36-5.17 (m, 2H), 5.09 (d, J=13.9 Hz, 1H), 4.78 (d, J=13.7 Hz, 1H), 4.39 (d, J=5.6 Hz, 2H), 4.23 (s, 1H), 4.00 (d, J=13.5 Hz, 1H), 3.58-3.37 (m, 5H), 2.99-3.17 (m, 1H), 2.70-2.58 (m, 4H), 2.49 (m, 1H), 2.21-2.06 (m, 2H), 1.90-1.78 (m, 4H), 1.42 (d, J=6.6 Hz, 3H).

### Example S-13: Synthesis of 1-((2R)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)-2-methylpiperazin-1-yl)-2-fluoroprop-2-en-1-one (Compound 42, Isomer A and Isomer B)

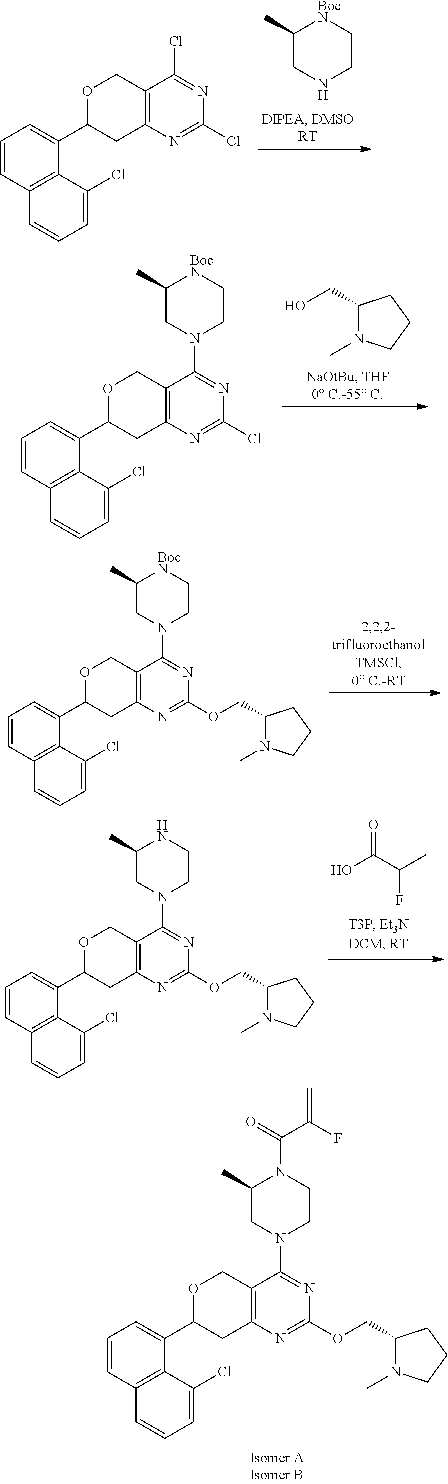

Step 1: Synthesis of tert-butyl (2R)-4-(2-chloro-7-(8-chloronaphthalen-1-yl)-7,8-dihydro-5H-pyrano[4,3-d]pyrimidin-4-yl)-2-methylpiperazine-1-carboxylate. The title compound was synthesized following the procedure as described in Example S-12, Step 1 using tert-butyl (R)-2-methylpiperazine-1-carboxylate (0.36 g, 1.78 mmol, 1.3 eq). LCMS (m/z): 529.1 [M+H]⁺

Step 2: Synthesis of tert-butyl (2R)-4-(7-(8-chloronaphthalen-1-yl)-2-(((S)-1-methyl-pyrrolidin-2-yl)methoxy)-7,8-dihydro-5H-pyrano[4,3-d]pyrimidin-4-yl)-2-methyl-piperazine-1-carboxylate. The title compound was synthesized following the procedure as described in Example S-12, Step 2 using tert-butyl (2R)-4-(2-chloro-7-(8-chloronaphthalen-1-yl)-7,8-dihydro-5H-pyrano[4,3-d]pyrimidin-4-yl)-2-methylpiperazine-1-carboxylate (0.30 g, 0.56 mmol, 1.0 eq). LCMS (m/z): 608.1 [M+H]⁺

Step 3: Synthesis of 7-(8-chloronaphthalen-1-yl)-4-((R)-3-methylpiperazin-1-yl)-2-(((S)-1-methylpyrrolidin-2-yl)methoxy)-7,8-dihydro-5H-pyrano[4,3-d]pyrimidine. The title compound was synthesized following the procedure as described in Example S-10, Step 2 using tert-butyl (2R)-4-(7-(8-chloronaphthalen-1-yl)-2-(((S)-1-methyl-pyrrolidin-2-yl)methoxy)-7,8-dihydro-5H-pyrano[4,3-d]pyrimidin-4-yl)-2-methyl-piperazine-1-carboxylate (0.24 g, 0.39 mmol, 1.0 eq). LCMS (m/z): 508.1 [M+H]⁺

Step 4: Synthesis of 1-((2R)-4-(7-(8-chloronaphthalen-1-yl)-2-(((S)-1-methyl pyrrolidin-2-yl)methoxy)-7,8-dihydro-5H-pyrano[4,3-d]pyrimidin-4-yl)-2-methylpiperazin-1-yl)-2-fluoroprop-2-en-1-one. The title compound was synthesized following the procedure as described in Example S-10, Step 3 using 7-(8-chloronaphthalen-1-yl)-4-((R)-3-methylpiperazin-1-yl)-2-(((S)-1-methylpyrrolidin-2-yl)methoxy)-7,8-dihydro-5H-pyrano[4,3-d]pyrimidine (0.26 g, 0.51 mmol, 1.0 eq) and Et₃N (0.22 mL, 1.59 mmol, 3.1 eq). The crude compound was purified by prep TLC and chiral SFC to afford the title compound as Isomer A and Isomer B.

Isomer A: LCMS (m/z): 580.2 [M+H]⁺; ¹H NMR (400 MHz, CDCl₃): δ 7.95 (d, J=7.2 Hz, 1H), 7.82 (t, J=8.4 Hz, 2H), 7.59-7.51 (m, 2H), 7.36 (t, J=7.8 Hz, 1H), 6.48 (dd, J=11.0, 3.3 Hz, 1H), 5.34-5.12 (m, 2H), 4.93 (d, J=13.3 Hz, 1H), 4.80 (d, J=13.6 Hz, 1H), 4.48 (s, 1H), 4.19 (d, J=4.5 Hz, 1H), 3.86 (d, J=12.2 Hz, 1H), 3.70 (d, J=13.3 Hz, 1H), 3.59-3.54 (m, 2H), 3.36 (dd, J=13.3, 3.3 Hz, 1H), 3.17-3.03 (m, 2H), 2.86-2.82 (m, 2H), 2.67-2.29 (m, 4H), 2.10-1.81 (m, 6H), 1.30 (d, J=6.5 Hz, 3H).

Isomer B: LCMS (m/z): 580.2 [M+H]⁺; ¹H NMR (400 MHz, CDCl₃): δ 7.97 (d, J=7.2 Hz, 1H), 7.82 (t, J=9.0 Hz, 2H), 7.60-7.54 (m, 2H), 7.36 (t, J=7.8 Hz, 1H), 6.47 (dd, J=10.9, 3.2 Hz, 1H), 5.34-5.13 (m, 2H), 4.95-4.82 (m, 2H), 4.55 (dd, J=12.4, 2.8 Hz, 1H), 4.06-3.85 (m, 2H), 3.70-3.33 (m, 4H), 3.13-3.03 (m, 2H), 2.99 (s, 3H), 2.92-2.87 (m, 2H), 2.33-2.08 (m, 4H), 1.86-1.58 (m, 3H), 1.47 (d, J=6.7 Hz, 3H).

### Example S-14: Synthesis of 1-((2P5S)-4-(7-(8-chloronaphthalen-1-yl)-2-(((S)-1-methyl-pyrrolidin-2-yl)methoxy)-7,8-dihydro-5H-pyrano[4,3-d]pyrimidin-4-yl)-2,5-dimethylpiperazin-1-yl)-2-fluoroprop-2-en-1-one (Compound 235, Isomer A and Isomer B)

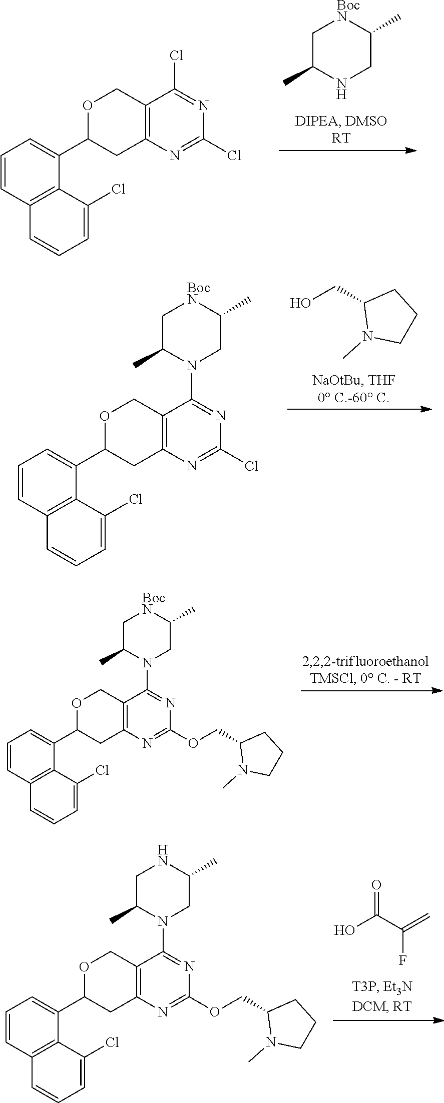

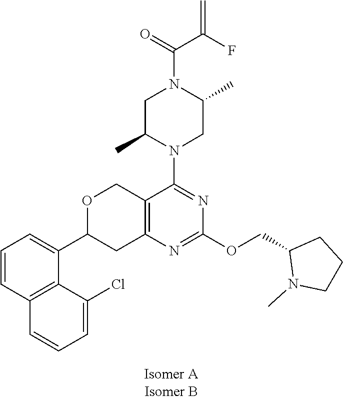 [SMILES: C=C(F)C(=O)N1C[C@H](C)N(c2nc(OC[C@@H]3CCCN3C)nc3c2COC(c2cccc4cccc(Cl)c24)C3)C[C@H]1C]

Step 1: Synthesis of tert-butyl (2R,5S)-4-(2-chloro-7-(8-chloronaphthalen-1-yl)-7,8-dihydro-5H-pyrano[4,3-d]pyrimidin-4-yl)-2,5-dimethylpiperazine-1-carboxylate. The title compound was synthesized following the procedure as described in Example S-12, Step 1 using tert-butyl (2R,5S)-2,5-dimethylpiperazine-1-carboxylate (0.38 g, 1.78 mmol, 1.3 eq). LCMS (m/z): 543.1 [M+H]⁺

Step 2: Synthesis of tert-butyl (2R,5S)-4-(7-(8-chloronaphthalen-1-yl)-2-(((S)-1-methyl-pyrrolidin-2-yl)methoxy)-7,8-dihydro-5H-pyrano[4,3-d]pyrimidin-4-yl)-2,5-dimethyl-piperazine-1-carboxylate. The title compound was synthesized following the procedure as described in Example S-12, Step 2 using tert-butyl (2R,5S)-4-(2-chloro-7-(8-chloronaphthalen-1-yl)-7,8-dihydro-5H-pyrano[4,3-d]pyrimidin-4-yl)-2,5-dimethylpiperazine-1-carboxylate (0.50 g, 0.92 mmol, 1.0 eq) and N-methyl-L-prolinol (0.17 g, 1.47 mmol, 1.6 eq). LCMS (m/z): 622.0 [M+H]⁺

Step 3: Synthesis of 7-(8-chloronaphthalen-1-yl)-4-((2S,5R)-2,5-dimethyl piperazin-1-yl)-2-(((S)-1-methylpyrrolidin-2-yl)methoxy)-7,8-dihydro-5H-pyrano[4,3-d]pyrimidine. The title compound was synthesized following the procedure as described in Example S-10, Step 2 using tert-butyl (2R,5S)-4-(7-(8-chloronaphthalen-1-yl)-2-(((S)-1-methyl-pyrrolidin-2-yl)methoxy)-7,8-dihydro-5H-pyrano[4,3-d]pyrimidin-4-yl)-2,5-dimethyl-piperazine-1-carboxylate (0.29 g, 0.46 mmol, 1.0 eq). LCMS (m/z): 522 [M+H]⁺

Step 4: Synthesis of 1-((2R,5S)-4-(7-(8-chloronaphthalen-1-yl)-2-(((S)-1-methyl-pyrrolidin-2-yl)methoxy)-7,8-dihydro-5H-pyrano[4,3-d]pyrimidin-4-yl)-2,5-dimethyl piperazin-1-yl)-2-fluoroprop-2-en-1-one. The title compound was synthesized following the procedure as described in Example S-10, Step 3 using 7-(8-chloronaphthalen-1-yl)-4-((2S,5R)-2,5-dimethylpiperazin-1-yl)-2-(((S)-1-methylpyrrolidin-2-yl)methoxy)-7,8-dihydro-5H-pyrano[4,3-d]pyrimidine (0.28 g, 0.54 mmol, 1.0 eq) and Et₃N (0.23 mL, 1.66 mmol, 3.1 eq). The crude compound was purified by prep TLC and chiral SFC to afford the title compound as Isomer A and Isomer B.

Isomer A: LCMS (m/z): 594.2 [M+H]⁺; ¹H NMR (400 MHz, CDCl₃): δ 7.96 (d, J=7.2 Hz, 1H), 7.81 (t, J=8.2 Hz, 2H), 7.62-7.52 (m, 2H), 7.36 (t, J=7.8 Hz, 1H), 6.49 (dd, J=11.0, 3.4 Hz, 1H), 5.34-5.12 (m, 2H), 4.89 (d, J=13.5 Hz, 1H), 4.75 (d, J=13.5 Hz, 1H), 4.38-4.20 (m, 4H), 3.79-3.56 (m, 3H), 3.39-3.12 (m, 2H), 2.84 (dd, J=18.2, 11.0 Hz, 1H), 2.69-2.31 (m, 5H), 2.05-1.66 (m, 5H), 1.32-1.24 (m, 3H), 1.17 (s, 3H).

Isomer B: LCMS (m/z): 594.2 [M+H]⁺; ¹H NMR (400 MHz, CDCl₃): δ 7.94 (d, J=7.2 Hz, 1H), 7.82 (t, J=8.2 Hz, 2H), 7.61-7.50 (m, 2H), 7.35 (t, J=7.8 Hz, 1H), 6.46 (dd, J=10.9, 3.3 Hz, 1H), 5.35-5.15 (m, 2H), 4.95 (d, J=13.4 Hz, 1H), 4.81 (d, J=13.4 Hz, 1H), 4.47-4.11 (m, 3H), 3.78-3.42 (m, 4H), 3.27-3.03 (m, 1H), 2.91-2.84 (m, 1H), 2.57-2.49 (m, 3H), 2.29-2.09 (m, 2H), 1.77-1.57 (m, 6H), 1.41-1.36 (m, 6H).

### Example S-15: Synthesis of 1-((2S,6R)-4-(7-(8-chloronaphthalen-1-yl)-2-(((S)-1-methyl-pyrrolidin-2-yl)methoxy)-7,8-dihydro-5H-pyrano[4,3-d]pyrimidin-4-yl)-2,6-dimethylpiperazin-1-yl)-2-fluoroprop-2-en-1-one (Compound 237, Isomer A and Isomer B)

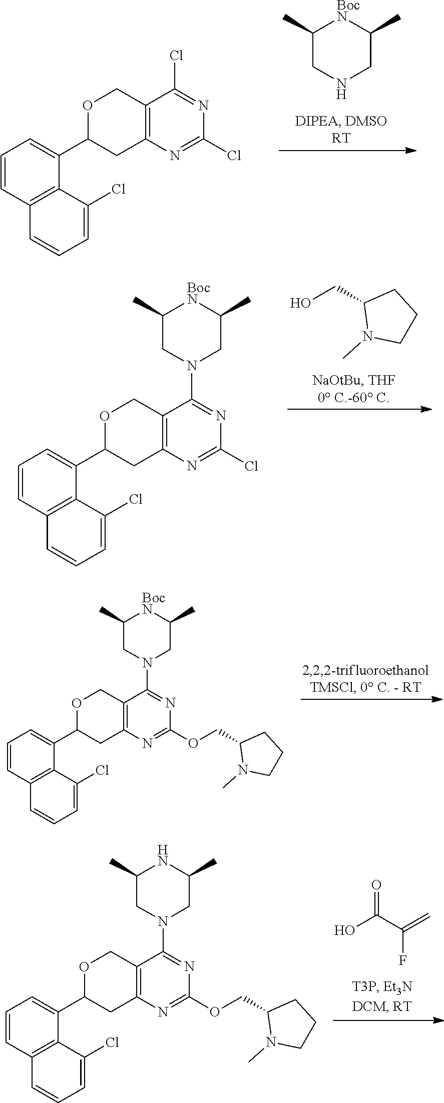

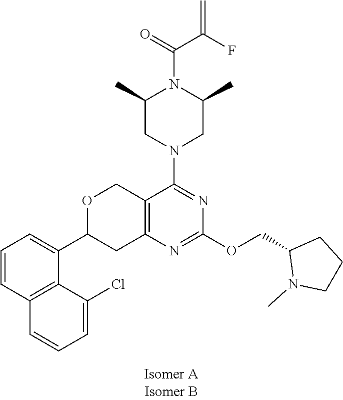 [SMILES: C=C(F)C(=O)N1[C@H](C)CN(c2nc(OC[C@@H]3CCCN3C)nc3c2COC(c2cccc4cccc(Cl)c24)C3)C[C@@H]1C]

Step 1: Synthesis of tert-butyl (2S,6R)-4-(2-chloro-7-(8-chloronaphthalen-1-yl)-7,8-dihydro-5H-pyrano[4,3-d]pyrimidin-4-yl)-2,6-dimethylpiperazine-1-carboxylate. The title compound was synthesized following the procedure as described in Example S-12, Step 1 using tert-butyl (2R,6S)-2,6-dimethylpiperazine-1-carboxylate (0.29 g, 1.36 mmol, 1.0 eq). LCMS (m/z): 543.5 [M+H]⁺

Step 2: Synthesis of tert-butyl (2S,6R)-4-(7-(8-chloronaphthalen-1-yl)-2-(((S)-1-methyl-pyrrolidin-2-yl)methoxy)-7,8-dihydro-5H-pyrano[4,3-d]pyrimidin-4-yl)-2,6-di methyl-piperazine-1-carboxylate. The title compound was synthesized following the procedure as described in Example S-12, Step 2 using tert-butyl (2S,6R)-4-(2-chloro-7-(8-chloronaphthalen-1-yl)-7,8-dihydro-5H-pyrano[4,3-d]pyrimidin-4-yl)-2,6-dimethylpiperazine-1-carboxylate (0.54 g, 1.0 mmol, 1.0 eq)N-methyl-L-prolinol (0.17 g, 1.49 mmol, 1.5 eq). LCMS (m/z): 622.0 [M+H]⁺

Step 3: Synthesis of 7-(8-chloronaphthalen-1-yl)-4-((3S,5R)-3,5-dimethyl piperazin-1-yl)-2-(((S)-1-methylpyrrolidin-2-yl)methoxy)-7,8-dihydro-5H-pyrano[4,3-d]pyrimidine. The title compound was synthesized following the procedure as described in Example S-10, Step 2 using tert-butyl (2S,6R)-4-(7-(8-chloronaphthalen-1-yl)-2-(((S)-1-methyl-pyrrolidin-2-yl)methoxy)-7,8-dihydro-5H-pyrano[4,3-d]pyrimidin-4-yl)-2,6-dimethyl-piperazine-1-carboxylate (0.20 g, 0.32 mmol, 1.0 eq). LCMS (m/z): 522.3 [M+H]⁺

Step 4: Synthesis of 1-((2S,6R)-4-(7-(8-chloronaphthalen-1-yl)-2-(((S)-1-methyl-pyrrolidin-2-yl)methoxy)-7,8-dihydro-5H-pyrano[4,3-d]pyrimidin-4-yl)-2,6-dimethyl piperazin-1-yl)-2-fluoroprop-2-en-1-one. The title compound was synthesized following the procedure as described in Example S-10, Step 3 using 7-(8-chloronaphthalen-1-yl)-4-((3S,5R)-3,5-dimethylpiperazin-1-yl)-2-(((S)-1-methylpyrrolidin-2-yl)methoxy)-7,8-dihydro-5H-pyrano[4,3-d]pyrimidine (0.12 g, 0.23 mmol, 1.0 eq). The crude compound was purified by chiral SFC to afford the title compound as Isomer A and Isomer B.

Isomer A: LCMS (m/z): 594.2 [M+H]⁺; ¹H NMR (400 MHz, CDCl₃): δ 7.94 (d, J=7.6 Hz, 1H), 7.84-7.80 (m, 2H), 7.61-7.52 (m, 2H), 7.38-7.33 (t, J=7.8 Hz, 1H), 6.47 (dd, J=11.0, 3.3 Hz, 1H), 5.30-5.18 (m, 1H), 5.15-5.09 (m, 1H), 4.96 (s, 2H), 4.55-4.23 (m, 3H), 3.91 (d, J=13.2 Hz, 1H), 3.67-3.52 (m, 2H), 3.18-3.05 (m, 2H), 2.93-2.71 (m, 2H), 2.66-2.43 (m, 4H), 2.14-1.80 (m, 4H), 1.55 (d, J=7.1 Hz, 3H), 1.46 (d, J=7.1 Hz, 3H), 1.32-1.09 (m, 1H).

Isomer B: LCMS (m/z): 594.2 [M+H]⁺; ¹H NMR (400 MHz, CDCl₃): δ 7.96 (d, J=7.5 Hz, 1H), 7.82-7.80 (m, 2H), 7.62-7.53 (m, 2H), 7.39-7.33 (m, 1H), 6.48 (dd, J=11.0, 3.3 Hz, 1H), 5.30-5.09 (m, 2H), 4.96 (s, 2H), 4.54-4.38 (m, 2H), 4.19-4.10 (m, 1H), 3.94-3.84 (m, 1H), 3.66-3.53 (m, 2H), 3.25-3.01 (m, 3H), 2.95-2.83 (m, 1H), 2.71-2.49 (m, 4H), 2.30-2.13 (m, 2H), 1.93-1.66 (m, 4H), 1.55 (d, J=6.9 Hz, 3H), 1.41 (d, J=6.9 Hz, 3H).

### Example S-16: Synthesis of 2-((2S)-1-(2-chloroacryloyl)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile (Compound 239, Isomer A and Isomer B)

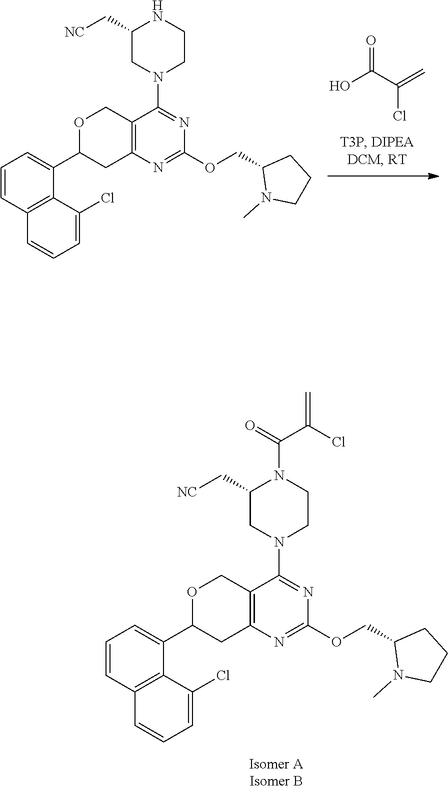

The title compound was synthesized following the procedure as described in Example S-10, Step 3 using 2-((2S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile (0.20 g, 0.37 mmol, 1.0 eq), 2-chloroacrylic acid (0.12 g, 1.12 mmol, 3.0 eq), T3P (0.45 mL, 1.50 mmol, 4.0 eq) and DIPEA (0.26 mL, 1.50 mmol, 4.0 eq). The crude compound was purified by prep HPLC and chiral SFC to afford the title compound as Isomer A and Isomer B.

Isomer A: LCMS (m/z): 620.7 [M+H]⁺; ¹H NMR (400 MHz, CDCl₃): δ 7.96 (d, J=7.1 Hz, 1H), 7.83 (t, J=8.6 Hz, 2H), 7.64-7.52 (m, 2H), 7.37 (t, J=7.8 Hz, 1H), 6.48 (dd, J=10.9, 3.2 Hz, 1H), 5.81-5.76 (m, 2H), 4.98-4.81 (m, 3H), 4.42-3.99 (m, 3H), 3.77 (d, J=10.9 Hz, 1H), 3.61-3.57 (m, 1H), 3.29-3.12 (m, 3H), 3.04-2.85 (m, 3H), 2.74-2.34 (m, 5H), 2.08-1.66 (m, 6H).

Isomer B: LCMS (m/z): 620.7 [M+H]⁺; ¹H NMR (400 MHz, CDCl₃): δ 7.97 (d, J=7.1 Hz, 1H), 7.83 (t, J=9.0 Hz, 2H), 7.63-7.51 (m, 2H), 7.37 (t, J=7.8 Hz, 1H), 6.52 (dd, J=10.9, 3.1 Hz, 1H), 5.81-5.76 (m, 2H), 5.01-4.81 (m, 3H), 4.52-4.24 (m, 2H), 3.98-3.46 (m, 4H), 3.22-3.04 (m, 2H), 2.95-2.72 (m, 4H), 2.61-2.58 (m, 3H), 2.11-1.81 (m, 7H).

### Example S-17: Synthesis of 2-((2S)-1-(but-2-ynoyl)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile (Compound 243, Isomer A and Isomer B)

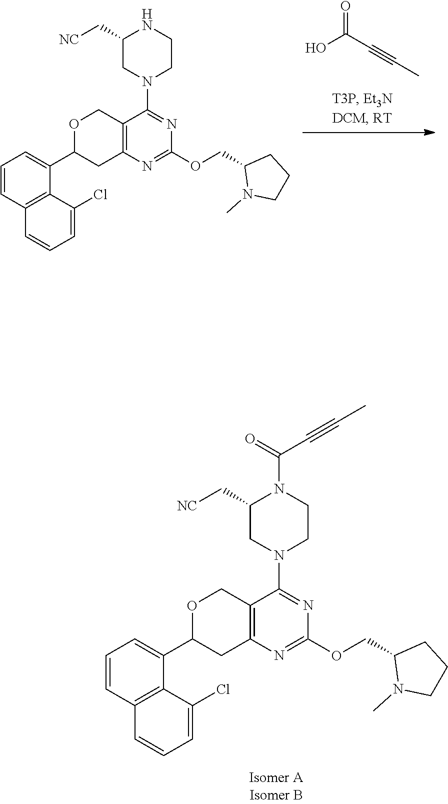

The title compound was synthesized following the procedure as described in Example S-10, Step 3 using 2-((2S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile (0.20 g, 0.37 mmol, 1.0 eq), but-2-ynoic acid (0.038 g, 0.45 mmol, 1.2 eq), T3P (0.22 mL, 0.75 mmol, 2.0 eq) and Et₃N (0.16 mL, 1.13 mmol, 3.0 eq). The crude compound was purified by silica gel chromatography and chiral SFC to afford the title compound as Isomer A and Isomer B.

Isomer A: LCMS (m/z): 599.2 [M+H]⁺; ¹H NMR (400 MHz, CD₃OD): δ 7.99 (d, J=7.4 Hz, 1H), 7.89 (d, J=8.2 Hz, 2H), 7.63 (dd, J=7.5, 1.1 Hz, 1H), 7.56 (t, J=7.8 Hz, 1H), 7.40 (t, J=7.8 Hz, 1H), 6.46 (dd, J=11.0, 3.1 Hz, 1H), 5.17-4.95 (m, 2H), 4.84-4.78 (m, 1H), 4.54-4.18 (m, 4H), 3.93-3.90 (m, 1H), 3.45-3.32 (m, 2H), 3.26-3.12 (m, 2H), 3.10-3.02 (m, 3H), 2.81-2.62 (m, 2H), 2.50-2.37 (m, 4H), 2.05 (t, J=9.6 Hz, 4H), 1.82-1.68 (m, 3H).

Isomer B: LCMS (m/z): 599.1 [M+H]⁺; ¹H NMR (400 MHz, CD₃OD): δ 8.00 (d, J=7.5 Hz, 1H), 7.94-7.83 (m, 2H), 7.63 (dd, J=7.5, 1.3 Hz, 1H), 7.56 (t, J=7.8 Hz, 1H), 7.45-7.36 (m, 1H), 6.50 (dd, J=11.0, 3.3 Hz, 1H), 5.08-4.90 (m, 3H), 4.49-4.25 (m, 3H), 4.17-4.03 (m, 1H), 3.94-3.89 (m, 1H), 3.55-3.32 (m, 2H), 3.18-3.00 (m, 3H), 2.96-2.81 (m, 2H), 2.71-2.48 (s, 5H), 2.37-2.28 (m, 1H), 2.11-1.99 (m, 4H), 1.79-1.76 (m, 2H), 1.72-1.56 (m, 1H).

### Example S-18: Synthesis of 2-((2S)-4-(7-(8-chloronaphthalen-1-yl)-2-(4-(diethylamino)-piperidin-1-yl)-7,8-dihydro-5H-pyrano[4,3-d]pyrimidin-4-yl)-1-(2-fluoroacryloyl)piperazin-2-yl)acetonitrile (Compound 230, Isomer A and Isomer B)

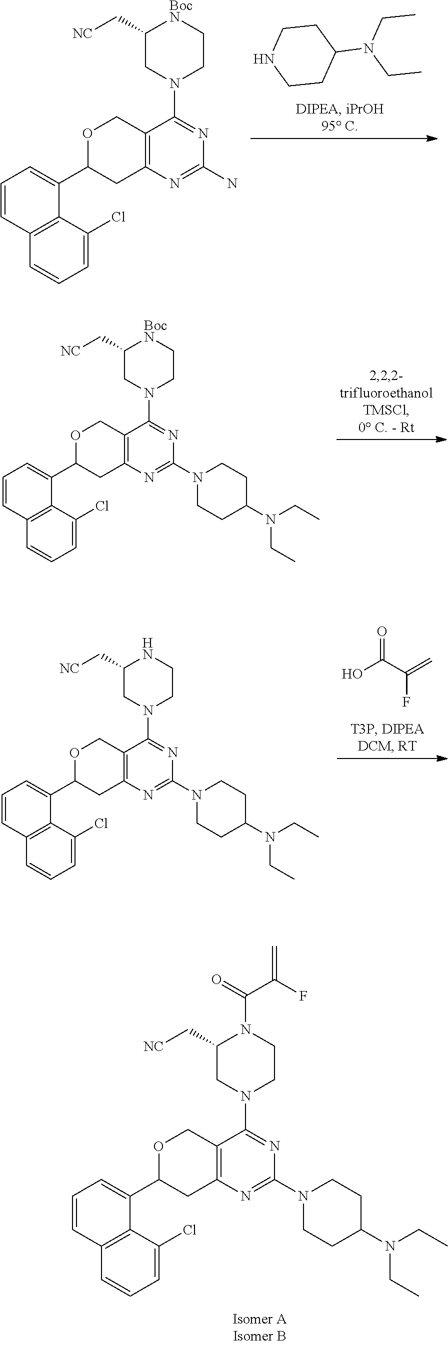

Step 1: Synthesis of tert-butyl (2S)-4-(7-(8-chloronaphthalen-1-yl)-2-(4-(diethylamino)-piperidin-1-yl)-7,8-dihydro-5H-pyrano[4,3-d]pyrimidin-4-yl)-2-(cyano methyl)piperazine-1-carboxylate. The title compound was synthesized following the procedure as described in Example S-10, Step 1 using N,N-diethylpiperidin-4-amine (0.16 g, 1.0 mmol, 1.0 eq). LCMS (m/z): 674.0 [M+H]⁺

Step 2: Synthesis of 2-((2S)-4-(7-(8-chloronaphthalen-1-yl)-2-(4-(diethylamino) piperidin-1-yl)-7,8-dihydro-5H-pyrano[4,3-d]pyrimidin-4-yl)piperazin-2-yl)acetonitrile. The title compound was synthesized following the procedure as described in Example S-10, Step 2 using tert-butyl (2S)-4-(7-(8-chloronaphthalen-1-yl)-2-(4-(diethylamino)-piperidin-1-yl)-7,8-dihydro-5H-pyrano[4,3-d]pyrimidin-4-yl)-2-(cyanomethyl)piperazine-1-carboxylate (0.37 g, 0.54 mmol, 1.0 eq). LCMS (m/z): 574.0 [M+H]⁺

Step 3: Synthesis of 2-((2S)-4-(7-(8-chloronaphthalen-1-yl)-2-(4-(diethylamino)-piperidin-1-yl)-7,8-dihydro-5H-pyrano[4,3-d]pyrimidin-4-yl)-1-(2-fluoroacryloyl)piperazin-2-yl)-acetonitrile. The title compound was synthesized following the procedure as described in Example S-10, Step 3 using 2-((2S)-4-(7-(8-chloronaphthalen-1-yl)-2-(4-(diethylamino)-piperidin-1-yl)-7,8-dihydro-5H-pyrano[4,3-d]pyrimidin-4-yl)piperazin-2-yl)acetonitrile (0.21 g, 0.36 mmol, 1.0 eq). The crude compound was purified by chiral SFC to afford the title compound as Isomer A and Isomer B.

Isomer A: LCMS (m/z): 646.2 [M+H]⁺; ¹H NMR (400 MHz, CDCl₃): δ 7.97 (d, J=7.1 Hz, 1H), 7.82 (t, J=7.1 Hz, 2H), 7.60 (d, J=7.4 Hz, 1H), 7.54 (t, J=7.8 Hz, 1H), 7.36 (t, J=7.8 Hz, 1H), 6.47-6.44 (m, 1H), 5.47-5.23 (m, 2H), 4.94-4.77 (m, 4H), 4.15-4.11 (m, 1H), 3.92-3.89 (m, 1H), 3.69-3.66 (m, 1H), 3.49-3.45 (m, 1H), 3.20-3.01 (m, 2H), 2.96-2.86 (m, 2H), 2.81-2.75 (m, 5H), 2.22-1.91 (m, 3H), 1.56-1.53 (m, 2H), 1.41-1.33 (m, 4H), 1.25-1.18 (m, 4H), 0.96-0.88 (m, 2H).

Isomer B: LCMS (m/z): 646.2 [M+H]⁺; ¹H NMR (400 MHz, CDCl₃): δ 7.99 (d, J=7.3 Hz, 1H), 7.82 (t, J=7.3 Hz, 2H), 7.64-7.53 (m, 2H), 7.35 (t, J=7.8 Hz, 1H), 6.50 (dd, J=11.0, 3.0 Hz, 1H), 5.46-5.24 (m, 2H), 4.92-4.78 (m, 3H), 3.90-3.86 (m, 1H), 3.66-3.35 (m, 3H), 2.94-2.71 (m, 5H), 2.59-2.48 (m, 3H), 1.99-1.81 (m, 3H), 1.52-1.40 (m, 2H), 1.27-1.24 (m, 6H), 1.06 (t, J=6.7 Hz, 4H), 0.88 (t, J=6.7 Hz, 2H).

### Example S-19: Synthesis of 2-((2S)-1-((E)-but-2-enoyl)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile (Compound 241, Isomer A and Isomer B)

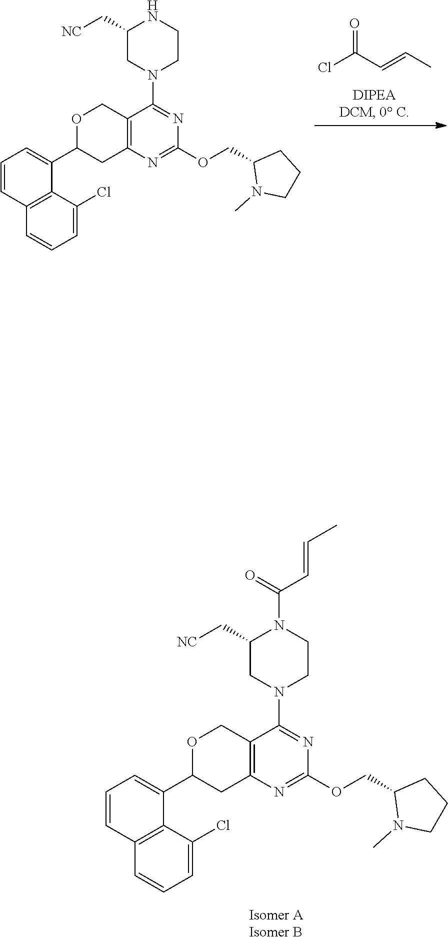

The title compound was synthesized following the procedure as described in Example S-11 using 2-((2S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile (0.30 g, 0.56 mmol, 1.0 eq), (E)-but-2-enoyl chloride (0.07 g, 0.67 mmol, 1.2 eq) and DIPEA (0.39 mL, 2.25 mmol, 4.0 eq). The crude material was purified by prep-HPLC followed by chiral SFC produced the title compound as Isomer A and Isomer B.

Isomer A: LCMS (m/z): 601.1 [M+H]⁺; ¹H NMR (400 MHz, CDCl₃): δ 7.97 (d, J=7.1 Hz, 1H), 7.83 (t, J=8.8 Hz, 2H), 7.63-7.53 (m, 2H), 7.37 (t, J=7.8 Hz, 1H), 7.02-6.95 (m, 1H), 6.52 (dd, J=11.0, 3.2 Hz, 1H), 6.29-6.26 (m, 1H), 5.01 (d, J=13.6 Hz, 1H), 4.83 (d, J=13.6 Hz, 1H), 4.47-4.20 (m, 2H), 4.02-3.98 (m, 1H), 3.66-3.43 (m, 3H), 3.13-2.64 (m, 7H), 2.51-2.35 (m, 4H), 2.09-1.93 (m, 5H), 1.83-1.79 (m, 4H).

Isomer B: LCMS (m/z): 601.1 [M+H]⁺; ¹H NMR (400 MHz, CDCl₃): δ 7.96 (d, J=7.3 Hz, 1H), 7.83 (t, J=8.3 Hz, 2H), 7.60-7.52 (m, 2H), 7.37 (t, J=7.8 Hz, 1H), 7.04-6.95 (m, 1H), 6.47 (dd, J=10.9, 3.2 Hz, 1H), 6.31-6.27 (m, 1H), 4.98-4.85 (m, 2H), 4.47-4.22 (m, 2H), 3.98-3.78 (m, 2H), 3.62-3.56 (m, 1H), 3.38-3.17 (m, 4H), 3.04-2.99 (m, 1H), 2.93-2.71 (m, 3H), 2.54 (s, 3H), 2.35-2.09 (m, 3H), 1.94 (d, J=6.8 Hz, 3H), 1.83-1.79 (s, 4H).

### Example S-20: Synthesis of 2-((2S)-4-(7-(8-chloronaphthalen-1-yl)-2-((2-methyl-1,2,3,4-tetra hydroisoquinolin-8-yl)oxy)-7,8-dihydro-5H-pyrano[4,3-d]pyrimidin-4-yl)-1-(2-fluoro acryloyl) piperazin-2-yl)acetonitrile (Compound 249, Isomer A and Isomer B)

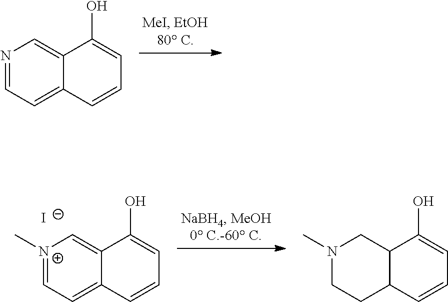

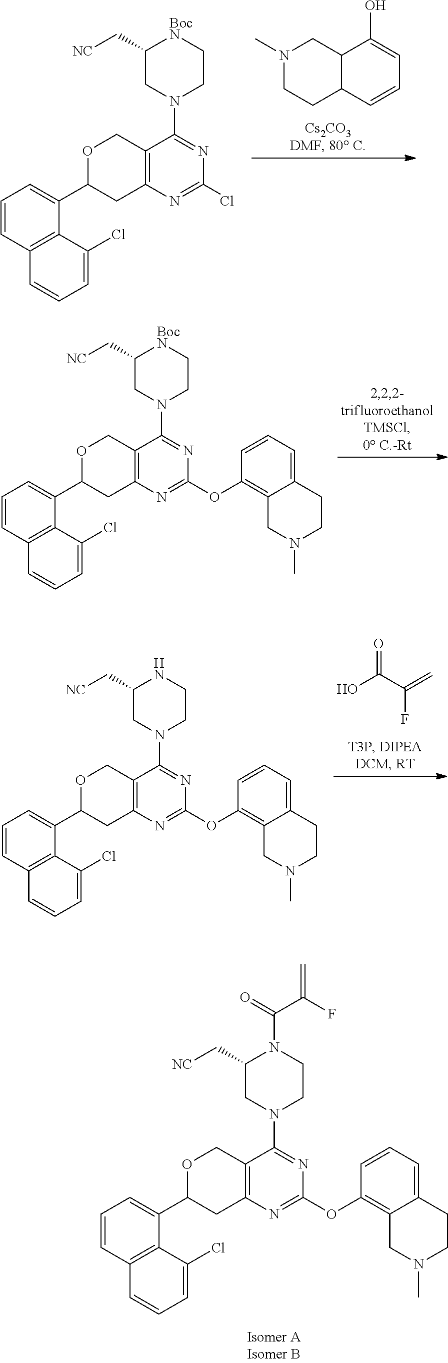

Step 1: Synthesis of 8-hydroxy-2-methylisoquinolin-2-ium iodide. To a solution of isoquinolin-8-ol (1.0 g, 6.89 mmol, 1.0 eq) in EtOH (20 mL) was added CH₃I (4.9 g, 34.52 mmol, 5.0 eq) at RT and the reaction mixture was stirred at 80° C. for 2 h. The reaction mixture was concentrated under reduced pressure to give the crude material. The crude was diluted with H₂O (30 mL) and extracted with EtOAc (3×50 mL). The combined organic layers were washed with brine (30 mL), dried over Na₂SO₄, filtered, and concentrated under reduced pressure to afford the title compound.

Step 2: Synthesis of 2-methyl-1,2,3,4-tetrahydroisoquinolin-8-ol. To a solution of 8-hydroxy-2-methylisoquinolin-2-ium iodide (1.0 g, 3.48 mmol, 1.0 eq) in MeOH, was added NaBH₄ (0.53 g, 14.01 mmol, 4.0 eq) at 0° C. over a period 15 min and the reaction mixture was stirred at 60° C. for 2 h. The reaction mixture was concentrated under reduced pressure to give the crude material. The crude was diluted with saturated aqueous NH₄Cl (50 mL) and extracted with EtOAc (3×50 mL). The combined organic layers were washed with brine (3×30 mL), dried over Na₂SO₄, filtered, and concentrated under reduced pressure to afford the title compound. LCMS (m/z): 164.3 [M+H]⁺

Step 3: Synthesis of tert-butyl (2S)-4-(7-(8-chloronaphthalen-1-yl)-2-((2-methyl-1,2,3,4-tetrahydroisoquinolin-8-yl)oxy)-7,8-dihydro-5H-pyrano[4,3-d]pyrimidin-4-yl)-2-(cyano methyl)piperazine-1-carboxylate. To a solution of tert-butyl (2S)-4-(2-chloro-7-(8-chloro naphthalen-1-yl)-7,8-dihydro-5H-pyrano[4,3-d]pyrimidin-4-yl)-2-(cyanomethyl) piperazine-1-carboxylate (0.10 g, 0.61 mmol, 1.0 eq), 2-methyl-1,2,3,4-tetrahydroisoquinolin-8-ol (0.34 g, 0.61 mmol, 1.0 eq) in DMF (10 mL) was added Cs₂CO₃ (0.40 g, 1.22 mmol, 2.0 eq) at RT and the reaction mixture was stirred at 80° C. for 2 h. The reaction mixture was diluted with H₂O (30 mL) and extracted with EtOAc (3×50 mL). The combined organic layers were washed with brine (30 mL), dried over Na₂SO₄, filtered, and concentrated under reduced pressure to afford the crude compound which was purified using silica gel chromatography to afford the title compound. LCMS (m/z): 681.0 [M+H]⁺

Step 4: Synthesis of 2-((2S)-4-(7-(8-chloronaphthalen-1-yl)-2-((2-methyl-1,2,3,4-tetra hydroisoquinolin-8-yl)oxy)-7,8-dihydro-5H-pyrano[4,3-d]pyrimidin-4-yl)piperazin-2-yl)acetonitrile. The title compound was synthesized following the procedure as described in Example S-10, Step 2 using tert-butyl (2S)-4-(7-(8-chloronaphthalen-1-yl)-2-((2-methyl-1,2,3,4-tetrahydroisoquinolin-8-yl)oxy)-7,8-dihydro-5H-pyrano[4,3-d]pyrimidin-4-yl)-2-(cyano methyl) piperazine-1-carboxylate (0.20 g, 0.29 mmol, 1.0 eq). LCMS (m/z): 581.1 [M+H]⁺

Step 5: Synthesis of 2-((2S)-4-(7-(8-chloronaphthalen-1-yl)-2-((2-methyl-1,2,3,4-tetra hydroisoquinolin-8-yl)oxy)-7,8-dihydro-5H-pyrano[4,3-d]pyrimidin-4-yl)-1-(2-fluoro acryloyl) piperazin-2-yl)acetonitrile. The title compound was synthesized following the procedure as described in Example S-10, Step 3 using 2-((2S)-4-(7-(8-chloronaphthalen-1-yl)-2-((2-methyl-1,2,3,4-tetra hydroisoquinolin-8-yl)oxy)-7,8-dihydro-5H-pyrano[4,3-d]pyrimidin-4-yl)piperazin-2-yl)acetonitrile (0.17 g, 0.29 mmol, 1.0 eq), 2-fluoroacrylic acid (0.10 g, 1.16 mmol, 4.0 eq), T3P (50% in EtOAc; 0.47 g, 0.75 mmol, 2.5 eq) and DIPEA (0.19 mg, 1.50 mmol, 5.1 eq). The crude material was purified by prep HPLC and SFC to afford the title compound as Isomer A and Isomer B.

Isomer A: LCMS (m/z): 653.6 [M+H]⁺; ¹H NMR (400 MHz, CDCl₃): δ 7.97 (d, J=7.1 Hz, 1H), 7.88-7.78 (m, 2H), 7.64-7.52 (m, 2H), 7.40-7.33 (m, 1H), 7.18 (t, J=7.7 Hz, 1H), 7.01 (d, J=7.4 Hz, 1H), 6.92 (d, J=8.1 Hz, 1H), 6.53 (dd, J=11.0, 3.3 Hz, 1H), 5.45-5.39 (m, 1H), 5.23 (dd, J=16.9, 3.7 Hz, 1H), 5.01 (d, J=13.8 Hz, 1H), 4.83 (d, J=13.7 Hz, 1H), 3.84-3.51 (m, 5H), 3.40-2.86 (m, 6H), 2.72-2.49 (m, 7H), 1.69-1.66 (m, 2H).

Isomer B: LCMS (m/z): 653.6 [M+H]⁺; ¹H NMR (400 MHz, CDCl₃): δ 7.96 (dd, J=7.4, 0.9 Hz, 1H), 7.84-7.81 (m, 2H), 7.62 (dd, J=7.5, 1.3 Hz, 1H), 7.59-7.54 (m, 1H), 7.40-7.35 (m, 1H), 7.22 (dd, J=11.6, 4.2 Hz, 1H), 7.06 (d, J=7.5 Hz, 1H), 6.93-6.87 (m, 1H), 6.51 (dd, J=11.1, 3.3 Hz, 1H), 5.45-5.33 (m, 1H), 5.22 (dd, J=16.9, 3.7 Hz, 1H), 4.95 (d, J=13.8 Hz, 1H), 4.77 (d, J=13.7 Hz, 1H), 3.92 (d, J=13.5 Hz, 1H), 3.72-3.40 (m, 4H), 3.36-2.96 (m, 5H), 2.93-2.59 (m, 5H), 2.47 (s, 3H), 2.05-1.70 (m, 2H).

### Example S-21: Synthesis of 1-(4-(7-(8-chloronaphthalen-1-yl)-2-(((S)-1-methylpyrrolidin-2-yl)methoxy)-7,8-dihydro-5H-pyrano[4,3-d]pyrimidin-4-yl)-2,2-dimethylpiperazin-1-yl)-2-fluoroprop-2-en-1-one (Compound 238, Isomer A and Isomer B)

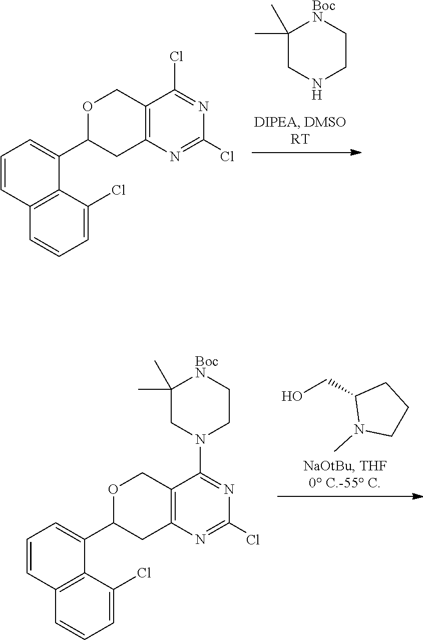

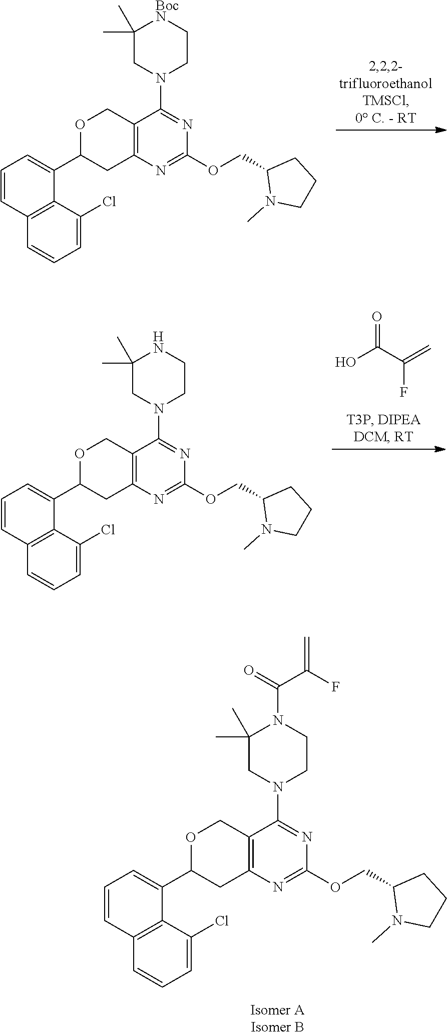

Step 1: Synthesis of tert-butyl 4-(2-chloro-7-(8-chloronaphthalen-1-yl)-7,8-dihydro-5H-pyrano[4,3-d]pyrimidin-4-yl)-2,2-dimethylpiperazine-1-carboxylate. The title compound was synthesized following the procedure as described in Example S-12, Step 1 using tert-butyl 2,2-dimethylpiperazine-1-carboxylate (0.29 g, 1.37 mmol, 1.0 eq). LCMS (m/z): 543.1 [M+H]⁺

Step 2: Synthesis of tert-butyl 4-(7-(8-chloronaphthalen-1-yl)-2-(((S)-1-methyl pyrrolidin-2-yl)methoxy)-7,8-dihydro-5H-pyrano[4,3-d]pyrimidin-4-yl)-2,2-dimethyl piperazine-1-carboxylate. The title compound was synthesized following the procedure as described in Example S-12, Step 2 using tert-butyl 4-(2-chloro-7-(8-chloronaphthalen-1-yl)-7,8-dihydro-5H-pyrano[4,3-d]pyrimidin-4-yl)-2,2-dimethylpiperazine-1-carboxylate (0.40 g, 0.73 mmol, 1.0 eq) and N-methyl-L-prolinol (0.13 g, 1.17 mmol, 1.6 eq). LCMS (m/z): 622.1 [M+H]⁺

Step 3: Synthesis of 7-(8-chloronaphthalen-1-yl)-4-(3,3-dimethylpiperazin-1-yl)-2-(((S)-1-methylpyrrolidin-2-yl)methoxy)-7,8-dihydro-5H-pyrano[4,3-d]pyrimidine. The title compound was synthesized following the procedure as described in Example S-10, Step 2 using tert-butyl 4-(7-(8-chloronaphthalen-1-yl)-2-(((S)-1-methylpyrrolidin-2-yl)methoxy)-7,8-dihydro-5H-pyrano[4,3-d]pyrimidin-4-yl)-2,2-dimethylpiperazine-1-carboxylate (0.15 g, 0.24 mmol, 1.0 eq). LCMS (m/z): 522.0 [M+H]⁺

Step 4: Synthesis of 1-(4-(7-(8-chloronaphthalen-1-yl)-2-(((S)-1-methyl pyrrolidin-2-yl)-methoxy)-7,8-dihydro-5H-pyrano[4,3-d]pyrimidin-4-yl)-2,2-dimethylpiperazin-1-yl)-2-fluoroprop-2-en-1-one. The title compound was synthesized following the procedure as described in Example S-10, Step 3 using 7-(8-chloronaphthalen-1-yl)-4-(3,3-dimethylpiperazin-1-yl)-2-(((S)-1-methylpyrrolidin-2-yl)methoxy)-7,8-dihydro-5H-pyrano[4,3-d]pyrimidine (0.14 g, 0.26 mmol, 1.0 eq), 2-fluoroacrylic acid (0.096 g, 1.07 mmol, 4.0 eq), T3P (0.45 mL, 1.50 mmol, 5.6 eq) and DIPEA (0.26 mL, 1.50 mmol, 5.6 eq). The crude compound was purified by prep HPLC and chiral SFC to afford the title compound as Isomer A and Isomer B.

Isomer A: LCMS (m/z): 594.0 [M+H]⁺; ¹H NMR (400 MHz, CDCl₃): δ 7.97 (d, J=7.4 Hz, 1H), 7.83 (t, J=8.2 Hz, 2H), 7.61-7.52 (m, 2H), 7.37 (t, J=7.8 Hz, 1H), 6.45 (dd, J=11.0, 3.2 Hz, 1H), 5.34-5.21 (m, 1H), 5.11-4.99 (m, 2H), 4.90 (d, J=13.6 Hz, 1H), 4.44-4.16 (m, 2H), 3.86-3.53 (m, 6H), 3.43 (d, J=13.6 Hz, 1H), 3.10-2.86 (m, 2H), 2.51 (s, 3H), 2.10-2.05 (m, 2H), 1.79 (s, 3H), 1.58-1.55 (m, 7H).

Isomer B: LCMS (m/z): 594.1 [M+H]⁺; ¹H NMR (400 MHz, CDCl₃): δ 7.97 (d, J=7.2 Hz, 1H), 7.83 (t, J=8.3 Hz, 2H), 7.64-7.50 (m, 2H), 7.37 (t, J=7.8 Hz, 1H), 6.45 (dd, J=11.0, 3.2 Hz, 1H), 5.34-5.21 (m, 1H), 5.13-4.99 (m, 2H), 4.91-4.88 (m, 1H), 4.48-4.23 (m, 2H), 3.76-3.43 (m, 7H), 3.17-2.86 (m, 2H), 2.58 (s, 3H), 2.20-1.66 (m, 6H), 1.57 (d, J=7.9 Hz, 6H).

### Example S-22: Synthesis of(S)-(4,4-difluoro-1-methylpyrrolidin-2-yl)methanol

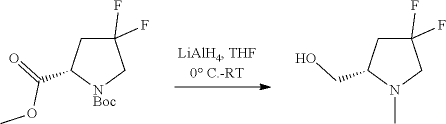

To a solution of 1-(tert-butyl) 2-methyl (S)-4,4-difluoropyrrolidine-1,2-dicarboxylate (1.0 g, 3.76 mmol, 1.0 eq) in THF (10 mL) at 0° C. under nitrogen atmosphere, was added LiAlH₄ in THF (1M; 11.0 mL, 11 mmol, 2.9 eq) and stirred at RT for 3 h. LCMS indicated the formation of the desired product. The solution was quenched by sequential dropwise addition of water (1 mL), 15% aqueous NaOH (1 mL) and water (3 mL) at −40° C. The reaction mixture was filtered through a Celite pad and the filtrate was concentrated under reduced pressure produced the desired title compound. LCMS (m/z): 152.0 [M+H]⁺

### Example S-23: Synthesis of 2-((2S)-4-(7-(8-chloronaphthalen-1-yl)-2-(((S)-4,4-difluoro-1-methyl pyrrolidin-2-yl)methoxy)-7,8-dihydro-5H-pyrano[4,3-d]pyrimidin-4-yl)-1-(2-fluoro-acryloyl) piperazin-2-yl)acetonitrile (Compound 228, Isomer A and Isomer B)

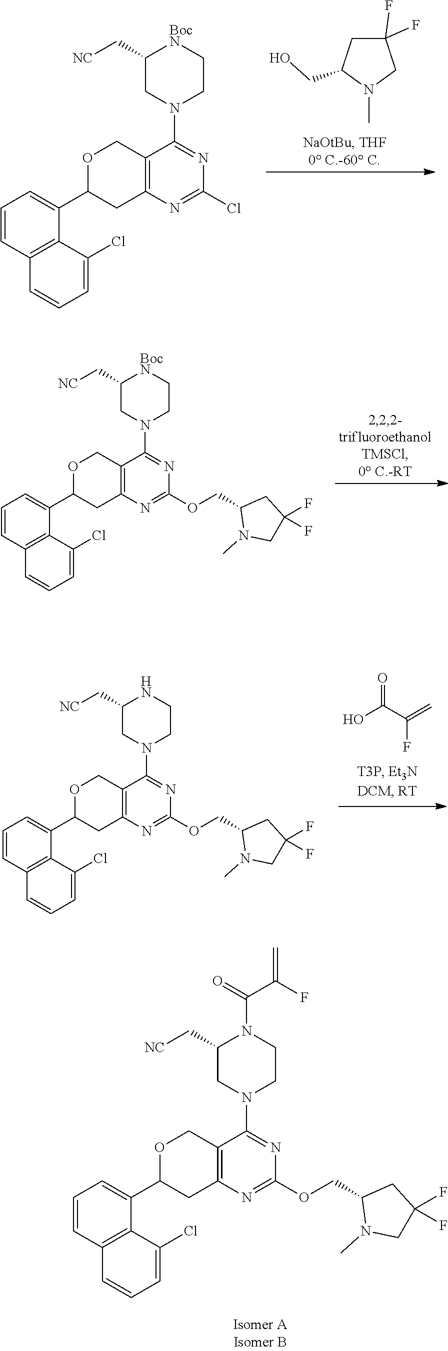

Step 1: Synthesis of tert-butyl (2S)-4-(7-(8-chloronaphthalen-1-yl)-2-(((S)-4,4-difluoro-1-methylpyrrolidin-2-yl)methoxy)-7,8-dihydro-5H-pyrano[4,3-d]pyrimidin-4-yl)-2-(cyanomethyl)piperazine-1-carboxylate. The title compound was synthesized following the procedure as described in Example S-12, Step 2 using tert-butyl (2S)-4-(2-chloro-7-(8-chloronaphthalen-1-yl)-7,8-dihydro-5H-pyrano[4,3-d]pyrimidin-4-yl)-2-(cyanomethyl)-piperazine-1-carboxylate (0.55 g, 1.0 mmol, 1.0 eq) and (S)-(4,4-difluoro-1-methylpyrrolidin-2-yl)methanol (0.30 g, 1.98 mmol, 2.0 eq). LCMS (m/z): 669.1 [M+H]⁺

Step 2: Synthesis of 2-((2S)-4-(7-(8-chloronaphthalen-1-yl)-2-(((S)-4,4-difluoro-1-methyl-pyrrolidin-2-yl)methoxy)-7,8-dihydro-5H-pyrano[4,3-d]pyrimidin-4-yl)piperazin-2-yl)acetonitrile. The title compound was synthesized following the procedure as described in Example S-10, Step 2 using tert-butyl (2S)-4-(7-(8-chloronaphthalen-1-yl)-2-(((S)-4,4-difluoro-1-methylpyrrolidin-2-yl)methoxy)-7,8-dihydro-5H-pyrano[4,3-d]pyrimidin-4-yl)-2-(cyanomethyl)piperazine-1-carboxylate (0.40 g, 0.59 mmol, 1.0 eq). LCMS (m/z): 569.0 [M+H]⁺

Step 3: Synthesis of 2-((2S)-4-(7-(8-chloronaphthalen-1-yl)-2-(((S)-4,4-difluoro-1-methyl-pyrrolidin-2-yl)methoxy)-7,8-dihydro-5H-pyrano[4,3-d]pyrimidin-4-yl)-1-(2-fluoro-acryloyl)piperazin-2-yl)acetonitrile. The title compound was synthesized following the procedure as described in Example S-10, Step 3 using 2-((2S)-4-(7-(8-chloronaphthalen-1-yl)-2-(((S)-4,4-difluoro-1-methyl-pyrrolidin-2-yl)methoxy)-7,8-dihydro-5H-pyrano[4,3-d]pyrimidin-4-yl)piperazin-2-yl)acetonitrile (0.17 g, 0.30 mmol, 1.0 eq), 2-fluoroacrylic acid (0.043 g, 0.47 mmol, 1.6 eq), T3P (0.27 mL, 0.90 mmol, 3.0 eq) and Et₃N (0.13 mL, 0.95 mmol, 3.2 eq). The crude compound was purified by prep TLC and chiral SFC to afford the title compound as Isomer A and Isomer B.

Isomer A: LCMS (m/z): 641.1 [M+H]⁺; ¹H NMR (400 MHz, CDCl₃): δ 7.97 (d, J=7.0 Hz, 1H), 7.86-7.81 (m, 2H), 7.64-7.53 (m, 2H), 7.37 (t, J=7.8 Hz, 1H), 6.52 (dd, J=11.0, 3.2 Hz, 1H), 5.49-5.37 (m, 1H), 5.29-5.23 (m, 1H), 5.01 (d, J=13.7 Hz, 1H), 4.83 (d, J=13.7 Hz, 1H), 4.56-4.32 (m, 2H), 4.00 (d, J=13.7 Hz, 1H), 3.83-3.29 (m, 6H), 3.09-2.79 (m, 6H), 2.68-2.20 (m, 6H).

Isomer B: LCMS (m/z): 641.2 [M+H]⁺; ¹H NMR (400 MHz, CDCl₃): δ 7.95 (d, J=7.0 Hz, 1H), 7.84 (t, J=8.5 Hz, 2H), 7.64-7.52 (m, 2H), 7.37 (t, J=7.8 Hz, 1H), 6.48-6.45 (m, 1H), 5.48-5.27 (m, 2H), 4.99 (d, J=13.5, 1H), 4.84 (d, J=13.5, 1H), 4.49-4.05 (m, 4H), 3.78 (d, J=9.6 Hz, 1H), 3.61-3.43 (m, 3H), 3.28-3.22 (m, 3H), 3.09-3.03 (m, 2H), 2.93-2.72 (m, 3H), 2.51-2.19 (m, 5H).

### Example S-24: Synthesis of 2-((2S)-4-(7-(8-chloronaphthalen-1-yl)-2-((1-methylpiperidin-4-yl)oxy)-7,8-dihydro-5H-pyrano[4,3-d]pyrimidin-4-yl)-1-(2-fluoroacryloyl)piperazin-2-yl)-acetonitrile (Compound 234, Isomer A and Isomer B)

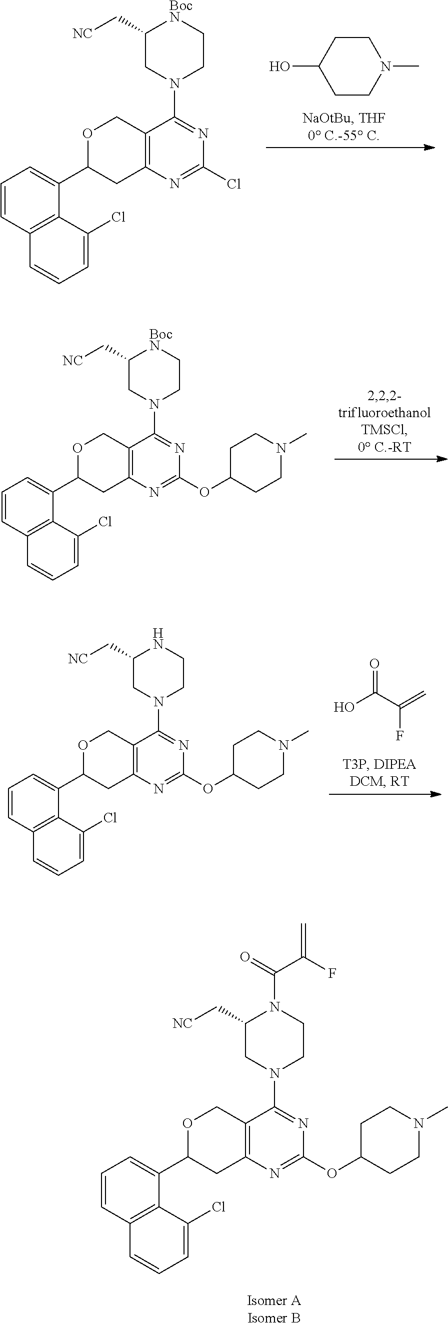

Step 1: Synthesis of tert-butyl (2S)-4-(7-(8-chloronaphthalen-1-yl)-2-((1-methylpiperidin-4-yl)oxy)-7,8-dihydro-5H-pyrano[4,3-d]pyrimidin-4-yl)-2-(cyanomethyl) piperazine-1-carboxylate. The title compound was synthesized following the procedure as described in Example S-12, Step 2 using tert-butyl (2S)-4-(2-chloro-7-(8-chloronaphthalen-1-yl)-7,8-dihydro-5H-pyrano[4,3-d]pyrimidin-4-yl)-2-(cyanomethyl)-piperazine-1-carboxylate (0.55 g, 1.0 mmol, 1.0 eq) and 1-methylpiperidin-4-ol (0.15 g, 1.29 mmol, 1.3 eq). LCMS (m/z): 633.2 [M+H]⁺

Step 2: Synthesis of 2-((2S)-4-(7-(8-chloronaphthalen-1-yl)-2-((1-methyl piperidin-4-yl)oxy)-7,8-dihydro-5H-pyrano[4,3-d]pyrimidin-4-yl)piperazin-2-yl) acetonitrile. The title compound was synthesized following the procedure as described in Example S-10, Step 2 using tert-butyl (2S)-4-(7-(8-chloronaphthalen-1-yl)-2-((1-methyl piperidin-4-yl)oxy)-7,8-dihydro-5H-pyrano-[4,3-d]pyrimidin-4-yl)-2-(cyanomethyl)piperazine-1-carboxylate (0.38 g, 0.60 mmol, 1.0 eq). LCMS (m/z): 532.0 [M+H]⁺

Step 3: Synthesis of 2-((2S)-4-(7-(8-chloronaphthalen-1-yl)-2-((1-methyl piperidin-4-yl)oxy)-7,8-dihydro-5H-pyrano[4,3-d]pyrimidin-4-yl)-1-(2-fluoroacryloyl) piperazin-2-yl)-acetonitrile. The title compound was synthesized following the procedure as described in Example S-10, Step 3 using 2-((2S)-4-(7-(8-chloronaphthalen-1-yl)-2-((1-methylpiperidin-4-yl)oxy)-7,8-dihydro-5H-pyrano[4,3-d]pyrimidin-4-yl)piperazin-2-yl)acetonitrile (0.24 g, 0.45 mmol, 1.0 eq), 2-fluoroacrylic acid (0.081 g, 0.90 mmol, 2.0 eq), T3P (0.4 mL, 1.35 mmol, 3.0 eq) and DIPEA (0.24 mL, 1.35 mmol, 3.0 eq). The crude compound was purified by chiral SFC to afford the title compound as Isomer A and Isomer B.

Isomer A: LCMS (m/z): 605.2 [M+H]⁺; ¹H NMR (400 MHz, CDCl₃): δ 7.96 (d, J=7.1 Hz, 1H), 7.83 (t, J=8.5 Hz, 2H), 7.61 (d, J=7.5 Hz, 1H), 7.55 (t, J=7.8 Hz, 1H), 7.37 (t, J=7.8 Hz, 1H), 6.48 (dd, J=10.9, 3.3 Hz, 1H), 5.49-5.27 (m, 2H), 5.14-5.08 (m, 1H), 4.97 (d, J=13.8 Hz, 1H), 4.85 (d, J=13.8 Hz, 1H), 4.12 (dd, J=14.3, 7.1 Hz, 1H), 3.93 (d, J=13.8 Hz, 1H), 3.75 (d, J=10.2 Hz, 1H), 3.62-3.57 (m, 1H), 3.42-3.17 (m, 3H), 3.06-2.89 (m, 6H), 2.53-2.42 (m, 5H), 2.11-1.99 (m, 4H).

Isomer B: LCMS (m/z): 605.2 [M+H]⁺; ¹H NMR (400 MHz, CDCl₃): δ 7.97 (d, J=7.4 Hz, 1H), 7.84 (t, J=8.9 Hz, 2H), 7.63 (d, J=8.6 Hz, 1H), 7.56 (t, J=7.8 Hz, 1H), 7.37 (t, J=7.8 Hz, 1H), 6.52 (dd, J=11.1, 3.2 Hz, 1H), 5.49-5.26 (m, 2H), 5.08-4.99 (m, 2H), 4.83 (d, J=13.6 Hz, 1H), 4.12-3.99 (m, 2H), 3.77-3.42 ((m, 5H), 3.07-2.80 (m, 8H), 2.46 (s, 3H), 2.15-2.01 (m, 4H).

### Example S-25: Synthesis of 3-((7-(8-chloronaphthalen-1-yl)-4-((S)-3-(cyanomethyl)-4-(2-fluoroacryloyl)piperazin-1-yl)-7,8-dihydro-5H-pyrano[4,3-d]pyrimidin-2-yl)amino)-N,N-dimethylpropanamide (Compound 232, Isomer A and Isomer B)

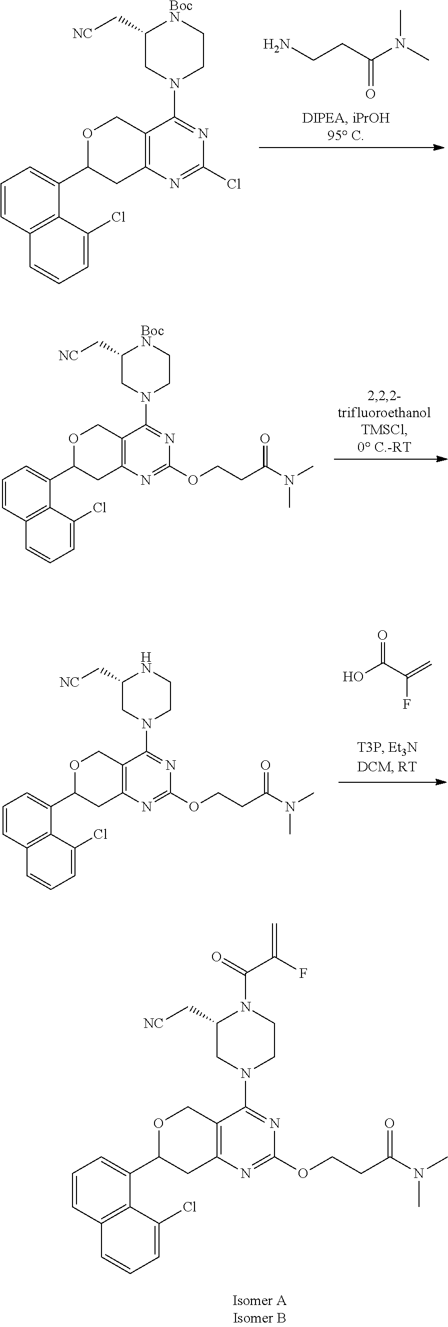

Step 1: Synthesis of tert-butyl (2S)-4-(7-(8-chloronaphthalen-1-yl)-2-((3-(dimethylamino)-3-oxopropyl)amino)-7,8-dihydro-5H-pyrano[4,3-d]pyrimidin-4-yl)-2-(cyanomethyl)-piperazine-1-carboxylate. The title compound was synthesized following the procedure as described in Example S-10, Step 1 using 3-amino-N,N-dimethylpropanamide (0.21 g, 1.35 mmol, 1.5 eq). LCMS (m/z): 634.0 [M+H]⁺

Step 2: Synthesis of 3-((7-(8-chloronaphthalen-1-yl)-4-((S)-3-(cyanomethyl) piperazin-1-yl)-7,8-dihydro-5H-pyrano[4,3-d]pyrimidin-2-yl)amino)-N,N-dimethyl propanamide. The title compound was synthesized following the procedure as described in Example S-10, Step 2 using tert-butyl-(2S)-4-(7-(8-chloronaphthalen-1-yl)-2-((3-(dimethylamino)-3-oxopropyl)amino)-7,8-dihydro-5H-pyrano[4,3-d]pyrimidin-4-yl)-2-(cyanomethyl)-piperazine-1-carboxylate (0.37 g, 0.58 mmol, 1.0 eq). LCMS (m/z): 534.0 [M+H]⁺

Step 3: Synthesis of 3-((7-(8-chloronaphthalen-1-yl)-4-((S)-3-(cyanomethyl)-4-(2-fluoro-acryloyl)piperazin-1-yl)-7,8-dihydro-5H-pyrano[4,3-d]pyrimidin-2-yl)amino)-N,N-di-methylpropanamide. The title compound was synthesized following the procedure as described in Example S-10, Step 3 using 3-((7-(8-chloronaphthalen-1-yl)-4-((S)-3-(cyanomethyl) piperazin-1-yl)-7,8-dihydro-5H-pyrano[4,3-d]pyrimidin-2-yl)amino)-N,N-dimethylpropan amide (0.17 g, 0.32 mmol, 1.0 eq), 2-fluoroacrylic acid (0.043 g, 0.47 mmol, 1.5 eq), T3P (0.28 mL, 0.95 mmol, 3.0 eq) and Et₃N (0.13 mL, 0.95 mmol, 3.0 eq). The crude compound was purified by prep TLC and chiral SFC to afford the title compound as Isomer A and Isomer B.

Isomer A: LCMS (m/z): 606.1 [M+H]⁺; ¹H NMR (400 MHz, CDCl₃): δ 7.95 (d, J=7.3 Hz, 1H), 7.83 (t, J=8.5 Hz, 2H), 7.61-7.52 (m, 2H), 7.37 (t, J=7.8 Hz, 1H), 6.49 (dd, J=10.9, 3.3 Hz, 1H), 5.71-5.09 (m, 3H), 4.95 (d, J=13.4 Hz, 1H), 4.77 (d, J=13.3 Hz, 1H), 3.99-3.50 (m, 7H), 3.14-2.72 (m, 11H), 2.63-2.57 (m, 3H).

Isomer B: LCMS (m/z): 606.1 [M+H]⁺; ¹H NMR (400 MHz, CDCl₃): δ 7.95 (d, J=7.0 Hz, 1H), 7.87-7.78 (m, 2H), 7.63-7.49 (m, 2H), 7.41 (t, J=7.8 Hz, 1H), 6.45 (dd, J=10.8, 3.3 Hz, 1H), 5.48-5.36 (m, 2H), 5.26 (dd, J=16.8, 3.6 Hz, 1H), 4.91 (d, J=13.3 Hz, 1H), 4.77 (d, J=13.3 Hz, 1H), 4.13-3.49 (m, 6H), 3.21-2.70 (m, 13H), 2.60 (t, J=6.2 Hz, 2H).

### Example S-26: Synthesis of 2-((2S)-4-(7-(8-chloronaphthalen-1-yl)-2-((1-methyl-1H-pyrazol-4-yl)amino)-7,8-dihydro-5H-pyrano[4,3-d]pyrimidin-4-yl)-1-(2-fluoroacryloyl)piperazin-2-yl)-acetonitrile (Compound 233, Isomer A and Isomer B)

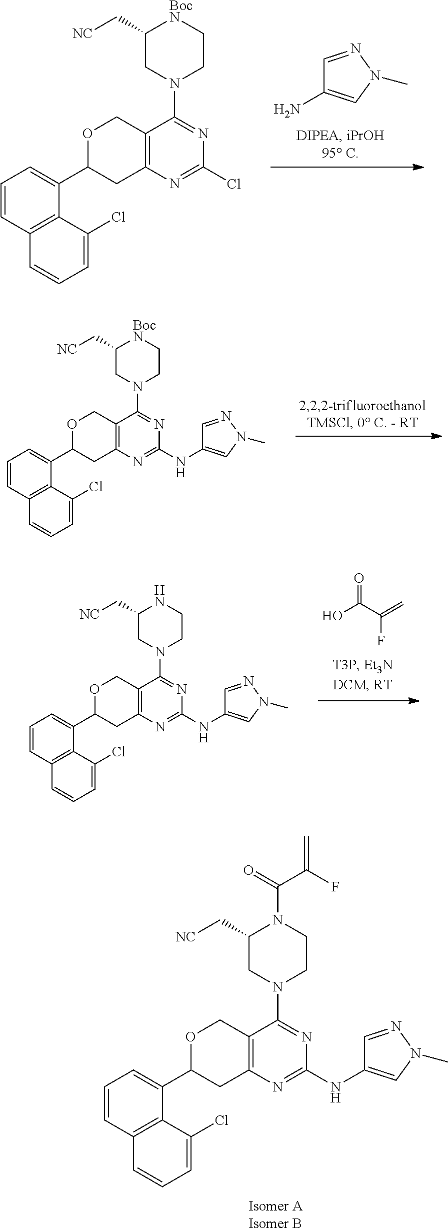

Step 1: Synthesis of tert-butyl (2S)-4-(7-(8-chloronaphthalen-1-yl)-2-((1-methyl-1H-pyrazol-4-yl)amino)-7,8-dihydro-5H-pyrano[4,3-d]pyrimidin-4-yl)-2-(cyanomethyl)-piperazine-1-carboxylate. The title compound was synthesized following the procedure as described in Example S-10, Step 1 using 1-methyl-1H-pyrazol-4-amine (0.18 g, 1.80 mmol, 2.0 eq). LCMS (m/z): 615.0 [M+H]⁺

Step 2: Synthesis of 2-((2S)-4-(7-(8-chloronaphthalen-1-yl)-2-((1-methyl-1H-pyrazol-4-yl)-amino)-7,8-dihydro-5H-pyrano[4,3-d]pyrimidin-4-yl)piperazin-2-yl) acetonitrile. The title compound was synthesized following the procedure as described in Example S-10, Step 2 using tert-butyl (2S)-4-(7-(8-chloronaphthalen-1-yl)-2-((1-methyl-1H-pyrazol-4-yl)amino)-7,8-dihydro-5H-pyrano[4,3-d]pyrimidin-4-yl)-2-(cyanomethyl)-piperazine-1-carboxylate (0.36 g, 0.59 mmol, 1.0 eq). LCMS (m/z): 515.0 [M+H]⁺

Step 3: Synthesis of 2-((2S)-4-(7-(8-chloronaphthalen-1-yl)-2-((1-methyl-1H-pyrazol-4-yl)amino)-7,8-dihydro-5H-pyrano[4,3-d]pyrimidin-4-yl)-1-(2-fluoroacryloyl) piperazin-2-yl)-acetonitrile. The title compound was synthesized following the procedure as described in Example S-10, Step 3 using 2-((2S)-4-(7-(8-chloronaphthalen-1-yl)-2-((1-methyl-1H-pyrazol-4-yl)-amino)-7,8-dihydro-5H-pyrano[4,3-d]pyrimidin-4-yl)piperazin-2-yl)acetonitrile (0.18 g, 0.35 mmol, 1.0 eq), 2-fluoroacrylic acid (0.047 g, 0.50 mmol, 1.5 eq), T3P (0.33 mL, 1.05 mmol, 3.0 eq) and Et₃N (0.15 mL, 1.05 mmol, 3.0 eq). The crude compound was purified by prep TLC and chiral SFC to afford the title compound as Isomer A and Isomer B.

Isomer A: LCMS (m/z): 587.1 [M+H]⁺; ¹H NMR (400 MHz, CDCl₃): δ 7.97 (d, J=7.1 Hz, 1H), 7.83 (t, J=8.5 Hz, 2H), 7.63-7.52 (m, 4H), 7.38 (t, J=7.8 Hz, 1H), 6.50 (dd, J=11.0, 3.5 Hz, 1H), 5.50-5.28 (m, 2H), 4.95 (d, J=13.1 Hz, 1H), 4.80 (d, J=13.1 Hz, 2H), 4.25-4.23 (m, 1H), 3.87 (s, 3H), 3.73-3.60 (m, 3H), 3.39-3.35 (m, 4H), 2.98-2.86 (m, 2H), 2.81-2.79 (m, 1H).

Isomer B: LCMS (m/z): 587.2 [M+H]⁺; ¹H NMR (400 MHz, CDCl₃): δ 7.98 (d, J=7.1 Hz, 1H), 7.84 (t, J=8.9 Hz, 2H), 7.70-7.49 (m, 4H), 7.38 (t, J=7.8 Hz, 1H), 6.53 (dd, J=11.0, 3.3 Hz, 1H), 5.50-5.23 (m, 2H), 4.98 (d, J=13.5 Hz, 1H), 4.80 (d, J=13.5 Hz, 1H), 4.00 (d, J=14.2 Hz, 1H), 3.90-3.79 (m, 6H), 3.64-3.48 (m, 3H), 3.13-2.79 (m, 5H).

### Example S-27: Synthesis of 2-((2S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)-1-((E)-4-(dimethylamino)but-2-enoyl) piperazin-2-yl)acetonitrile (Compound 242, Isomer A and Isomer B)

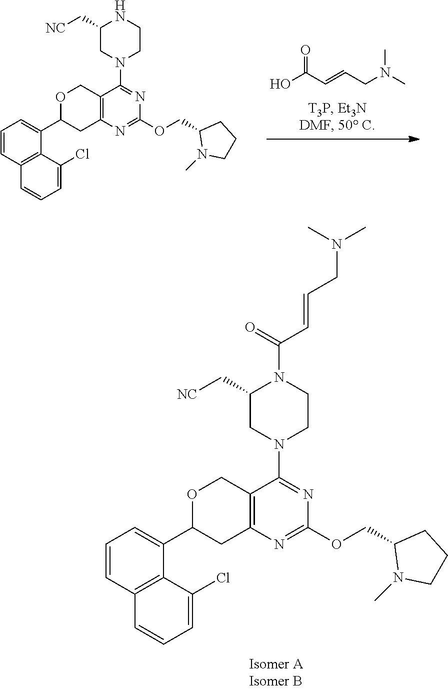

The title compound was synthesized following the procedure as described in Example S-10, Step 3 using 2-((2S)-4-(7-(8-chloronaphthalen-1-yl)-2-(((S)-1-methylpyrrolidin-2-yl)methoxy)-7,8-dihydro-5H-pyrano[4,3-d]pyrimidin-4-yl)piperazin-2-yl)acetonitrile (0.10 g, 0.18 mmol, 1.0 eq), (E)-4-(dimethylamino)but-2-enoic acid (0.029 g, 0.23 mmol, 1.2 eq), T3P (0.11 mL, 0.37 mmol, 2.0 eq) and Et₃N (0.08 mL, 0.56 mmol, 3.0 eq). The crude compound was purified by prep HPLC and chiral SFC to afford the title compound as Isomer A and Isomer B.

Isomer A: LCMS (m/z): 644.2 [M+H]⁺; ¹H NMR (400 MHz, CD₃OD): δ 8.01 (d, J=7.4 Hz, 1H), 7.92 (d, J=8.2 Hz, 2H), 7.65 (dd, J=7.5, 1.2 Hz, 1H), 7.58 (t, J=7.8 Hz, 1H), 7.43 (t, J=7.8 Hz, 1H), 6.83-6.69 (m, 2H), 6.49 (dd, J=10.7, 3.0 Hz, 1H), 5.11 (d, J=13.4 Hz, 1H), 4.57-4.32 (m, 5H), 4.11-4.08 (m, 1H), 3.92 (d, J=13.1 Hz, 1H), 3.50-3.46 (m, 2H), 3.28-3.17 (m, 6H), 3.13-2.98 (m, 4H), 2.77-2.58 (m, 5H), 2.31 (s, 3H), 2.21-2.07 (m, 1H), 1.89-1.72 (m, 3H).

Isomer B: LCMS (m/z): 644.2 [M+H]⁺; ¹H NMR (400 MHz, CD₃OD): δ 8.01 (d, J=7.5 Hz, 1H), 7.92 (d, J=8.2 Hz, 2H), 7.66 (d, J=7.4 Hz, 1H), 7.58 (t, J=7.8 Hz, 1H), 7.43 (t, J=7.8 Hz, 1H), 6.83-6.68 (m, 2H), 6.55-6.43 (m, 1H), 5.12 (d, J=14.1 Hz, 2H), 4.61-3.90 (m, 6H), 3.50-3.46 (m, 2H), 3.23-3.18 (m, 7H), 3.13-3.05 (m, 3H), 2.78-2.49 (m, 5H), 2.31 (s, 3H), 2.17-2.12 (m, 1H), 1.91-1.78 (m, 3H).

### Example S-28: Synthesis of 2-((2S)-4-(7-(8-chloronaphthalen-1-yl)-2-((1-(methylsulfonyl) piperidin-4-yl)amino)-7,8-dihydro-5H-pyrano[4,3-d]pyrimidin-4-yl)-1-(2-fluoroacryloyl) piperazin-2-yl)acetonitrile (Compound 231, Isomer A and Isomer B)

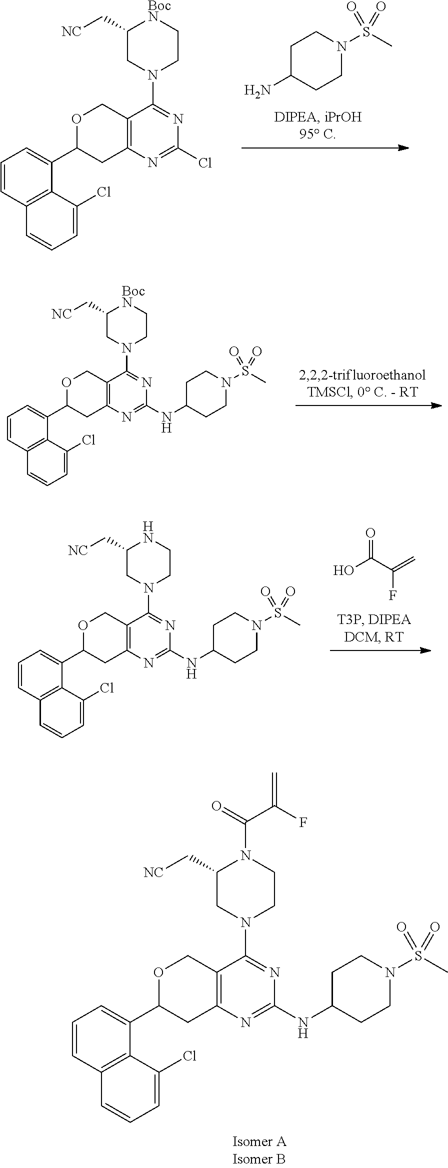

Step 1: Synthesis of tert-butyl (2S)-4-(7-(8-chloronaphthalen-1-yl)-2-((1-(methylsulfonyl) piperidin-4-yl)amino)-7,8-dihydro-5H-pyrano[4,3-d]pyrimidin-4-yl)-2-(cyanomethyl) piperazine-1-carboxylate. The title compound was synthesized following the procedure as described in Example S-10, Step 1 using 1-(methylsulfonyl)piperidin-4-amine (0.44 g, 2.50 mmol, 2.5 eq). LCMS (m/z): 696.0 [M+H]⁺

Step 2: Synthesis of 2-((2S)-4-(7-(8-chloronaphthalen-1-yl)-2-((1-(methylsulfonyl)piperidin-4-yl)amino)-7,8-dihydro-5H-pyrano[4,3-d]pyrimidin-4-yl)piperazin-2-yl)acetonitrile. The title compound was synthesized following the procedure as described in Example S-10, Step 2 using tert-butyl (2S)-4-(7-(8-chloronaphthalen-1-yl)-2-((1-(methylsulfonyl) piperidin-4-yl)amino)-7,8-dihydro-5H-pyrano[4,3-d]pyrimidin-4-yl)-2-(cyanomethyl) piperazine-1-carboxylate (0.15 g, 0.22 mmol, 1.0 eq). LCMS (m/z): 596.0 [M+H]⁺

Step 3: Synthesis of 2-((2S)-4-(7-(8-chloronaphthalen-1-yl)-2-((1-(methylsulfonyl) piperidin-4-yl)amino)-7,8-dihydro-5H-pyrano[4,3-d]pyrimidin-4-yl)-1-(2-fluoroacryloyl) piperazin-2-yl)acetonitrile. The title compound was synthesized following the procedure as described in Example S-10, Step 3 using 2-((2S)-4-(7-(8-chloronaphthalen-1-yl)-2-((1-(methylsulfonyl) piperidin-4-yl)amino)-7,8-dihydro-5H-pyrano[4,3-d]pyrimidin-4-yl)piperazin-2-yl)acetonitrile (0.10 g, 0.17 mmol, 1.0 eq), 2-fluoroacrylic acid (0.03 g, 0.34 mmol, 2.0 eq), T3P (0.15 mL, 0.50 mmol, 3.0 eq) and DIPEA (0.09 mL, 0.50 mmol, 3.0 eq). The crude compound was purified by chiral SFC to afford the title compound as Isomer A and Isomer B.

Isomer A: LCMS (m/z): 668.2 [M+H]⁺; ¹H NMR (400 MHz, CDCl₃): δ 7.95 (d, J=7.0 Hz, 1H), 7.80 (t, J=7.8 Hz, 2H), 7.59-7.52 (m, 2H), 7.38 (t, J=7.8 Hz, 1H), 6.44 (dd, J=11.0, 3.4 Hz, 1H), 5.47-5.22 (m, 2H), 4.91 (d, J=13.3 Hz, 1H), 4.76 (d, J=13.3 Hz, 1H), 4.22-4.01 (m, 3H), 3.65-3.62 (m, 3H), 3.48-3.44 (m, 1H), 3.27-2.98 (m, 6H), 2.79-2.72 (m, 6H), 2.17-2.05 (m, 2H), 1.67-1.58 (m, 3H).

Isomer B: LCMS (m/z): 668.2 [M+H]⁺; ¹H NMR (400 MHz, CDCl₃): δ 7.95 (d, J=7.1 Hz, 1H), 7.82 (t, J=8.2 Hz, 2H), 7.59-7.53 (m, 2H), 7.35 (t, J=7.8 Hz, 1H), 6.47 (dd, J=11.0, 3.3 Hz, 1H), 5.47-5.22 (m, 2H), 4.91 (d, J=13.4 Hz, 1H), 4.78 (d, J=13.4 Hz, 1H), 4.24-4.01 (m, 3H), 3.65-3.47 (m, 4H), 3.25-2.90 (m, 6H), 2.80-2.72 (m, 6H), 2.14-2.07 (m, 2H), 1.67-1.58 (m, 3H).

### Example S-29: Synthesis of 2,4-dichloro-7-(5-methyl-1H-indazol4-yl)-7,8-dihydro-5H-pyrano [4,3-d]pyrimidine

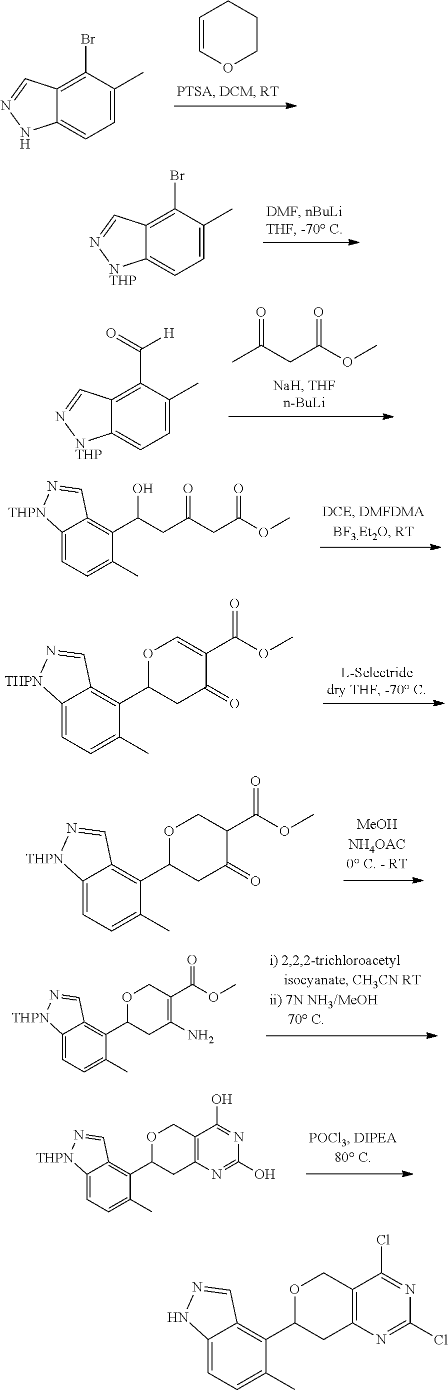

Step 1: Synthesis of 4-bromo-5-methyl-1-(tetrahydro-2H-pyran-2-yl)-1H-indazole. To a solution of 4-bromo-5-methyl-1H-indazole (50 g, 236.8 mmol, 1.0 eq) and 3,4-dihydro-2H-pyran (30 g, 356.6 mmol, 1.5 eq) in DCM (1000 mL) was added PTSA (3.90 g, 23.68 mmol, 0.1 eq) and stirred at RT overnight. The reaction mixture was concentrated under reduced pressure and to the crude residue was added EtOAc (300 mL) and extracted with brine (2×100 mL), dried over anhydrous Na₂SO₄, filtered and concentrated under reduced pressure to yield the title compound. LCMS (m/z): 295.0 [M+H]⁺

Step 2: Synthesis of 5-methyl-1-(tetrahydro-2H-pyran-2-yl)-1H-indazole-4-carbaldehyde. To a solution of 4-bromo-5-methyl-1-(tetrahydro-2H-pyran-2-yl)-1H-indazole (5.0 g, 16.93 mmol, 1.0 eq) in dry THF (60 mL) at −70° C. was added nBuLi (2.4 M in hexane; 8 mL, 20.10 mmol, 1.2 eq) and stirred for 30 min. Then DML (1.5 mL, 20.10 mmol, 1.2 eq) was added dropwise and stirred at −70° C. for 2 h. To the reaction mixture, EtOAc (150 mL) was added followed by ice cooled NH₄Cl (50 mL). The organic layer was washed with brine (2×100 mL), dried over anhydrous Na₂SO₄, filtered and concentrated under reduced pressure to yield the crude compound. The crude material was purified by silica gel chromatography to afford the title compound. LCMS (m/z): 245.1 [M+H]⁺

Step 3: Synthesis of methyl-5-hydroxy-5-(5-methyl-1-(tetrahydro-2H-pyran-2-yl)-1H-indazol-4-yl)-3-oxopentanoate. The title compound was synthesized using the procedure as described in Example S-9, Step 1 using 5-methyl-1-(tetrahydro-2H-pyran-2-yl)-1H-indazole-4-carbaldehyde (5.8 g, 0.05 mol, 1.0 eq). LCMS (m/z): 361.1 [M+H]⁺

Step 4: Synthesis of methyl-2-(5-methyl-1-(tetrahydro-2H-pyran-2-yl)-1H-indazol-4-yl)-4-oxo-3,4-dihydro-2H-pyran-5-carboxylate. The title compound was synthesized using the procedure as described in Example S-9, Step 2 using methyl-5-hydroxy-5-(5-methyl-1-(tetrahydro-2H-pyran-2-yl)-1H-indazol-4-yl)-3-oxopentanoate (13.0 g, 0.036 mol, 1.0 eq). LCMS (m/z): 371.0 [M+H]⁺

Step 5: Synthesis of methyl-6-(5-methyl-1-(tetrahydro-2H-pyran-2-yl)-1H-indazol-4-yl)-4-oxotetrahydro-2H-pyran-3-carboxylate. The title compound was synthesized using the procedure as described in Example S-9, Step 3 using methyl-2-(5-methyl-1-(tetrahydro-2H-pyran-2-yl)-1H-indazol-4-yl)-4-oxo-3,4-dihydro-2H-pyran-5-carboxylate (13.0 g, 0.035 mol, 1.0 eq). LCMS (m/z): 373.2 [M+H]⁺

Step 6: Synthesis of methyl-4-amino-6-(5-methyl-1-(tetrahydro-2H-pyran-2-yl)-1H-indazol-4-yl)-5,6-dihydro-2H-pyran-3-carboxylate. The title compound was synthesized using the procedure as described in Example S-9, Step 4 using methyl-6-(5-methyl-1-(tetrahydro-2H-pyran-2-yl)-1H-indazol-4-yl)-4-oxotetrahydro-2H-pyran-3-carboxylate (13.0 g, 0.035 mol, 1.0 eq). LCMS (m/z): 372.2 [M+H]⁺

Step 7: Synthesis of 7-(5-methyl-1-(tetrahydro-2H-pyran-2-yl)-1H-indazol-4-yl)-7,8-dihydro-5H-pyrano[4,3-d]pyrimidine-2,4-diol. The title compound was synthesized using the procedure as described in Example S-9, Step 5 using methyl-4-amino-6-(5-methyl-1-(tetrahydro-2H-pyran-2-yl)-1H-indazol-4-yl)-5,6-dihydro-2H-pyran-3-carboxylate (13.0 g, 0.035 mol, 1.0 eq). LCMS (m/z): 383.0 [M+H]⁺

Step 8: Synthesis of 2,4-dichloro-7-(5-methyl-1H-indazol-4-yl)-7,8-dihydro-5H-pyrano [4,3-d]pyrimidine. To a stirred solution of 7-(5-methyl-1-(tetrahydro-2H-pyran-2-yl)-1H-indazol-4-yl)-7,8-dihydro-5H-pyrano[4,3-d]pyrimidine-2,4-diol (5.0 g, 0.013 mol, 1.0 eq) in dry POCl₃ (70 mL) was added DIPEA (6.8 mL, 0.039 mol, 3.0 eq) and the reaction mixture was stirred at 80° C. for 18 h. The reaction mixture was cooled to RT and poured into ice cooled water dropwise and basified to pH ˜8-10 using saturated NaHCO₃ solution and extracted with DCM (3×10 mL). The combined organic layers were washed with brine, dried over anhydrous Na₂SO₄, filtered and concentrated under reduced pressure to yield the crude compound. The crude material was purified by silica gel chromatography to afford the title compound. LCMS (m/z): 335.2 [M+H]⁺

### Example S-30: Synthesis of 2-((2S)-1-(2-fluoroacryloyl)-4-(7-(5-methyl-1H-indazol4-yl)-2-(((S)-1-methylpyrrolidin-2-yl)methoxy)-7,8-dihydro-5H-pyrano[4,3-d]pyrimidin-4-yl)piperazin-2-yl) acetonitrile (Compound 52, Isomer A and Isomer B)

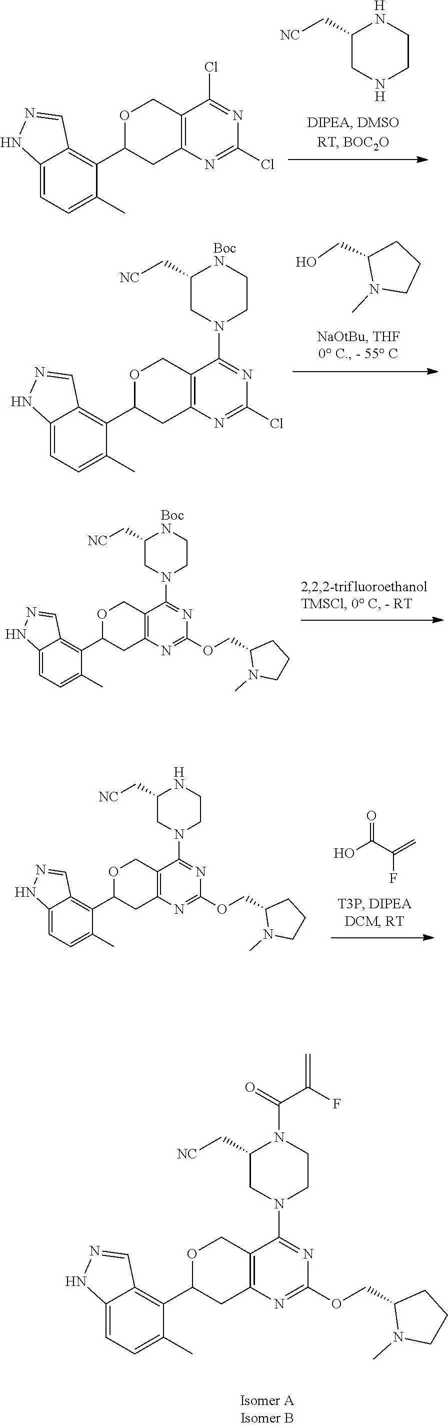

Step 1: Synthesis of tert-butyl (2S)-4-(2-chloro-7-(5-methyl-1H-indazol-4-yl)-7,8-dihydro-5H-pyrano[4,3-d]pyrimidin-4-yl)-2-(cyanomethyl)piperazine-1-carboxylate. A solution of 2,4-dichloro-7-(5-methyl-1H-indazol-4-yl)-7,8-dihydro-5H-pyrano[4,3-d]pyrimidine (0.50 g, 1.49 mmol, 1.0 eq), (S)-2-(piperazin-2-yl)acetonitrile (0.3 g, 2.39 mmol, 1.6 eq), DIPEA (1.3 mL, 7.45 mmol, 5.0 eq) in DMSO (10 mL) was stirred at RT for 4 h. Boc₂O (0.33 g, 1.49 mmol, 1.0 eq) was added to the reaction mixture and allowed to stir at RT for 14 h. The reaction mixture was quenched with water (200 mL) and filtered to produce the crude compound. The crude material was purified by silica gel chromatography to afford the title compound. LCMS (m/z): 524 [M+H]⁺

Step 2: Synthesis of tert-butyl (2S)-2-(cyanomethyl)-4-(7-(5-methyl-1H-indazol-4-yl)-2-(((S)-1-methylpyrrolidin-2-yl)methoxy)-7,8-dihydro-5H-pyrano[4,3-d]pyrimidin-4-yl)piperazine-1-carboxylate. The title compound was synthesized following the procedure as described in Example S-12, Step 2 using tert-butyl (2S)-4-(2-chloro-7-(5-methyl-1H-indazol-4-yl)-7,8-dihydro-5H-pyrano[4,3-d]pyrimidin-4-yl)-2-(cyanomethyl)piperazine-1-carboxylate (0.40 g, 0.76 mmol, 1.0 eq) and N-methyl-L-prolinol (0.13 g, 1.14 mmol, 1.5 eq). LCMS (m/z): 603.2 [M+H]⁺

Step 3: Synthesis of 2-((2S)-4-(7-(5-methyl-1H-indazol-4-yl)-2-(((S)-1-methyl pyrrolidin-2-yl)methoxy)-7,8-dihydro-5H-pyrano[4,3-d]pyrimidin-4-yl)piperazin-2-yl)acetonitrile. The title compound was synthesized following the procedure as described in Example S-10, Step 2 using tert-butyl (2S)-2-(cyanomethyl)-4-(7-(5-methyl-1H-indazol-4-yl)-2-(((S)-1-methyl pyrrolidin-2-yl)methoxy)-7,8-dihydro-5H-pyrano[4,3-d]pyrimidin-4-yl)piperazine-1-carboxylate (0.21 g, 0.35 mmol, 1.0 eq). LCMS (m/z): 503.1 [M+H]⁺

Step 4: Synthesis of 2-((2S)-1-(2-fluoroacryloyl)-4-(7-(5-methyl-1H-indazol-4-yl)-2-(((S)-1-methylpyrrolidin-2-yl)methoxy)-7,8-dihydro-5H-pyrano[4,3-d]pyrimidin-4-yl)piperazin-2-yl) acetonitrile. The title compound was synthesized following the procedure as described in Example S-10, Step 3 using 2-((2S)-4-(7-(5-methyl-1H-indazol-4-yl)-2-(((S)-1-methylpyrrolidin-2-yl)methoxy)-7,8-dihydro-5H-pyrano[4,3-d]pyrimidin-4-yl)piperazin-2-yl)acetonitrile (0.20 g, 0.39 mmol, 1.0 eq), 2-fluoroacrylic acid (0.057 g, 0.64 mmol, 1.6 eq), T3P (0.45 mL, 1.50 mmol, 3.8 eq) and DIPEA (0.26 mL, E50 mmol, 3.8 eq). The crude compound was purified by prep HPLC and chiral SFC to afford the title compound as Isomer A and Isomer B.

Isomer A: LCMS (m/z): 575.3 [M+H]⁺; ¹H NMR (400 MHz, CD₃OD): δ 8.12 (s, 1H), 7.41 (d, J=8.5 Hz, 1H), 7.25 (d, J=8.6 Hz, 1H), 5.44-5.29 (m, 3H), 5.04-4.82 (m, 6H), 4.62-4.55 (m, 2H), 3.95-3.71 (m, 3H), 3.48-3.31 (m, 2H), 3.24-3.03 (m, 9H), 2.47 (s, 3H), 2.39-2.01 (m, 4H).

Isomer B: LCMS (m/z): 575.2 [M+H]⁺; ¹H NMR (400 MHz, CD₃OD): δ 8.14 (s, 1H), 7.41 (d, J=8.3 Hz, 1H), 7.25 (d, J=8.6 Hz, 1H), 5.46-5.29 (m, 3H), 5.09-4.99 (m, 3H), 4.84-4.80 (dd, J=12.7, 3.1 Hz, 2H), 4.56 (dd, J=12.7, 7.2 Hz, 1H), 4.28 (d, J=13.6 Hz, 1H), 4.07-3.99 (m, 1H), 3.91-3.88 (m, 1H), 3.71-3.63 (m, 4H), 3.25-2.99 (m, 9H), 2.47 (s, 3H), 2.41-2.03 (m, 4H).

### Example S-31: Synthesis of 1-((R)-4-(7-(8-chloronaphthalen-1-yl)-2-(((S)-1-methylpyrrolidin-2-yl)methoxy)-5H-pyrano[2,3-d]pyrimidin-4-yl)-2-methylpiperazin-1-yl)-2-fluoroprop-2-en-1-one (Compound 154)

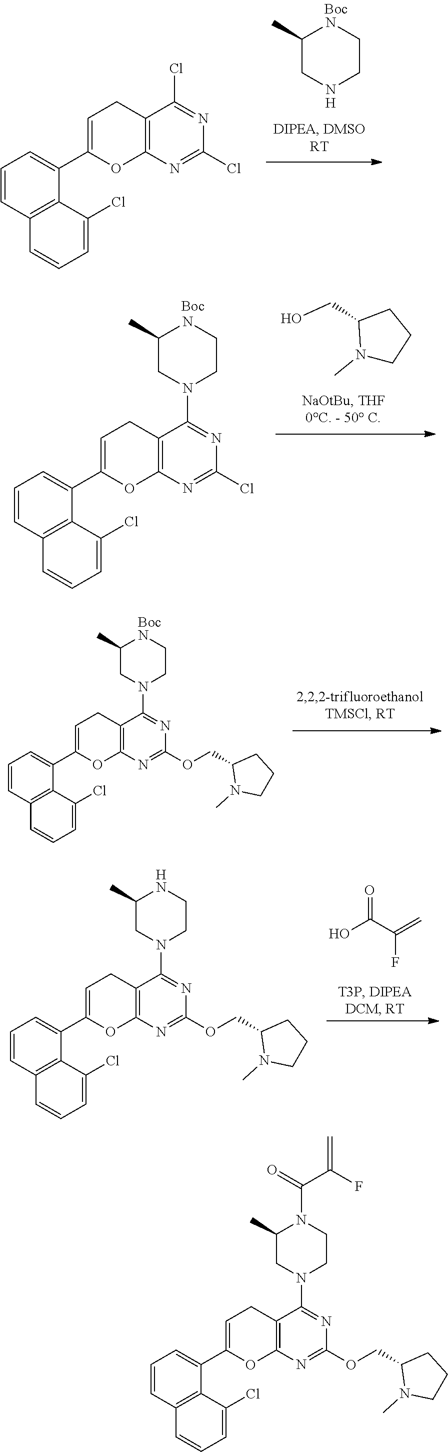

Step 1: Synthesis of tert-butyl (R)-4-(2-chloro-7-(8-chloronaphthalen-1-yl)-5H-pyrano[2,3-d]pyrimidin-4-yl)-2-methylpiperazine-1-carboxylate. A solution of 2,4-dichloro-7-(8-chloro naphthalen-1-yl)-5H-pyrano[2,3-d]pyrimidine (0.50 g, 1.38 mmol, 1.0 eq), tert-butyl (R)-2-methylpiperazine-1-carboxylate (0.27 g, 1.38 mmol, 1.0 eq) and DIPEA (0.48 mL, 2.76 mmol, 2.0 eq) in DMSO (20 mL) was stirred at RT for 14 h. The reaction mixture was quenched with water (50 mL) and filtered to produce the crude compound. The crude material was purified by silica gel chromatography to afford the title compound. LCMS (m/z): 527 [M+H]⁺

Step 2: Synthesis of tert-butyl (R)-4-(7-(8-chloronaphthalen-1-yl)-2-(((S)-1-methyl-pyrrolidin-2-yl)methoxy)-5H-pyrano[2,3-d]pyrimidin-4-yl)-2-methylpiperazine-1-carboxylate. The title compound was synthesized following the procedure as described in Example S-12, Step 2 using tert-butyl (R)-4-(2-chloro-7-(8-chloronaphthalen-1-yl)-5H-pyrano[2,3-d]pyrimidin-4-yl)-2-methylpiperazine-1-carboxylate (0.30 g, 0.57 mmol, 1.0 eq) and N-methyl-L-prolinol (0.10 g, 0.91 mmol, 1.6 eq). LCMS (m/z): 606.2 [M+H]⁺

Step 3: Synthesis of 7-(8-chloronaphthalen-1-yl)-4-((R)-3-methylpiperazin-1-yl)-2-(((S)-1-methylpyrrolidin-2-yl)methoxy)-5H-pyrano[2,3-d]pyrimidine. The title compound was synthesized following the procedure as described in Example S-10, Step 2 using tert-butyl (R)-4-(7-(8-chloronaphthalen-1-yl)-2-(((S)-1-methyl-pyrrolidin-2-yl)methoxy)-5H-pyrano[2,3-d]pyrimidin-4-yl)-2-methylpiperazine-1-carboxylate (0.10 g, 0.16 mmol, 1.0 eq). LCMS (m/z): 506.0 [M+H]⁺

Step 4: Synthesis of 1-((R)-4-(7-(8-chloronaphthalen-1-yl)-2-(((S)-1-methyl pyrrolidin-2-yl) methoxy)-5H-pyrano[2,3-d]pyrimidin-4-yl)-2-methylpiperazin-1-yl)-2-fluoroprop-2-en-1-one. The title compound was synthesized following the procedure as described in Example S-10, Step 3 using 7-(8-chloronaphthalen-1-yl)-4-((R)-3-methylpiperazin-1-yl)-2-(((S)-1-methylpyrrolidin-2-yl)methoxy)-5H-pyrano[2,3-d]pyrimidine (0.08 g, 0.16 mmol, 1.0 eq), 2-fluoroacrylic acid (0.057 g, 0.64 mmol, 4.0 eq), T3P (0.14 mL, 0.48 mmol, 3.0 eq) and DIPEA (0.11 mL, 0.64 mmol, 4.0 eq). The crude compound was purified by prep HPLC yielded the desired title compound.

LCMS (m/z): 578.0 [M+H]⁺; ¹H NMR (400 MHz, CDCl₃): δ 7.91 (dd, J=8.2, 1.2 Hz, 1H), 7.81-7.77 (m, 1H), 7.62-7.54 (m, 2H), 7.50-7.44 (m, 1H), 7.39 (t, J=7.8 Hz, 1H), 5.36-5.20 (m, 2H), 5.16 (dd, J=17.1, 3.5 Hz, 1H), 4.46-4.28 (m, 3H), 3.94-3.74 (m, 2H), 3.55-3.39 (m, 3H), 3.32-2.90 (m, 5H), 2.59 (s, 3H), 2.47 (br s, 1H), 2.09-1.81 (m, 4H), 1.43-1.41 (m, 3H).

### Example S-32: Synthesis of 1-(4-(7-(8-chloronaphthalen-1-yl)-2-(((S)-1-methylpyrrolidin-2-yl)methoxy)-6,7-dihydro-[1,4]dioxino[2,3-d]pyrimidin-4-yl)piperazin-1-yl)prop-2-en-1-one (Compound 135, Isomer A and Isomer B)

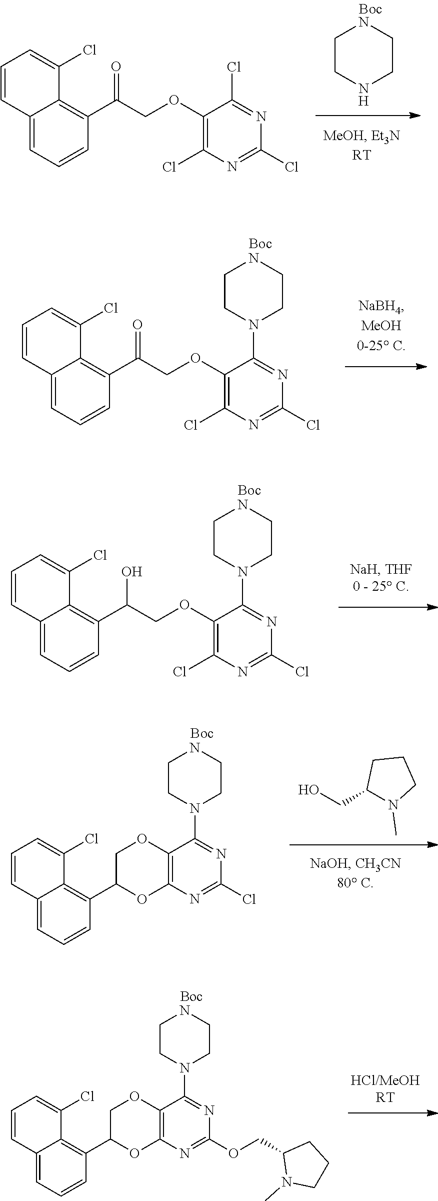

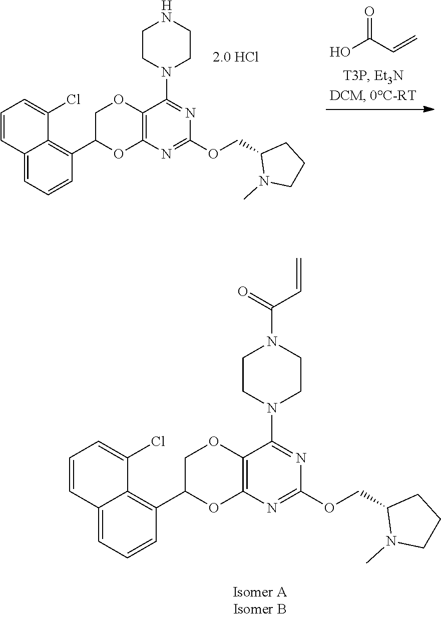

Step 1: Synthesis of tert-butyl 4-(2,6-dichloro-5-(2-(8-chloronaphthalen-1-yl)-2-oxoethoxy)pyrimidin-4-yl)piperazine-1-carboxylate. The title compound was synthesized following the procedure as described in Example S-8, Step 5 using tert-butyl piperazine-1-carboxylate (0.44 g, 2.36 mmol, 1.0 eq). LCMS (m/z): 551.1 [M+H]⁺

Step 2: Synthesis of tert-butyl 4-(2,6-dichloro-5-(2-(8-chloronaphthalen-1-yl)-2-hydroxy-ethoxy)pyrimidin-4-yl)piperazine-1-carboxylate. The title compound was synthesized following the procedure as described in Example S-8, Step 6 using tert-butyl 4-(2,6-dichloro-5-(2-(8-chloronaphthalen-1-yl)-2-oxoethoxy)pyrimidin-4-yl)piperazine-1-carboxylate (0.90 g, 1.63 mmol, 1.0 eq). LCMS (m/z): 553.3 [M+H]⁺

Step 3: Synthesis of tert-butyl 4-(2-chloro-7-(8-chloronaphthalen-1-yl)-6,7-dihydro-[1,4]dioxino[2,3-d]pyrimidin-4-yl)piperazine-1-carboxylate. The title compound was synthesized following the procedure as described in Example S-8, Step 7 using tert-butyl 4-(2,6-dichloro-5-(2-(8-chloronaphthalen-1-yl)-2-hydroxy-ethoxy)pyrimidin-4-yl)piperazine-1-carboxylate (1.20 g, 2.16 mmol, 1.0 eq). LCMS (m/z): 517.2 [M+H]⁺

Step 4: Synthesis of tert-butyl (2S)-4-(7-(8-chloronaphthalen-1-yl)-2-(((S)-1-methyl pyrrolidin-2-yl)methoxy)-6,7-dihydro-[1,4]dioxino[2,3-d]pyrimidin-4-yl)-2-(cyanomethyl) piperazine-1-carboxylate. The title compound was synthesized following the procedure as described in Example S-8, Step 8 using tert-butyl 4-(2-chloro-7-(8-chloronaphthalen-1-yl)-6,7-dihydro-[1,4]dioxino[2,3-d]pyrimidin-4-yl)piperazine-1-carboxylate (0.30 g, 0.58 mmol, 1.0 eq) and N-methyl-L-prolinol (0.33 g, 2.90 mmol, 5.0 eq). LCMS (m/z): 596.3 [M+H]⁺

Step 5: Synthesis of 2-((2S)-4-(7-(8-chloronaphthalen-1-yl)-2-(((S)-1-methylpyrrolidin-2-yl)methoxy)-6,7-dihydro-[1,4]dioxino[2,3-d]pyrimidin-4-yl)piperazin-2-yl)acetonitrile dihydrochloride. A solution of tert-butyl (2S)-4-(7-(8-chloronaphthalen-1-yl)-2-(((S)-1-methyl pyrrolidin-2-yl)methoxy)-6,7-dihydro-[1,4]dioxino[2,3-d]pyrimidin-4-yl)-2-(cyanomethyl) piperazine-1-carboxylate (0.14 g, 0.23 mmol, 1.0 eq) in HCl/MeOH (4N; 10 mL) was stirred at RT for 1 h. TLC indicated the completion of the reaction. The solvent was removed under reduced pressure and the crude material was washed with petroleum ether (2 mL) produced the title compound. LCMS (m/z): 496.3 [M+H]⁺

Step 6: Synthesis of 1-(4-(7-(8-chloronaphthalen-1-yl)-2-(((S)-1-methyl pyrrolidin-2-yl) methoxy)-6,7-dihydro-[1,4]dioxino[2,3-d]pyrimidin-4-yl)piperazin-1-yl)prop-2-en-1-one. To a stirred solution of 2-((2S)-4-(7-(8-chloronaphthalen-1-yl)-2-(((S)-1-methylpyrrolidin-2-yl)methoxy)-6,7-dihydro-[1,4]dioxino[2,3-d]pyrimidin-4-yl)piperazin-2-yl)acetonitrile dihydrochloride (0.056 g, 0.09 mmol, 1.0 eq) in DCM (5 mL) was added Et₃N (0.05 mL, 0.39 mmol, 4.0 eq) followed by the addition of acrylic acid (0.009 g, 0.12 mmol, 1.3 eq) and T3P (50% in EtOAc, 0.12 mL, 0.19 mmol, 2.0) dropwise at 0° C. The reaction mixture was stirred at RT for 2 h. The reaction mixture was quenched with aqueous saturated NaHCO₃ (10.0 mL) and extracted with CH₂Cl₂ (2×15 mL). The combined organic layers were washed with brine, dried over Na₂SO₄, filtered, and concentrated under reduced pressure to obtain crude material. The crude material was purified by prep HPLC and SLC to afford the title compound as Isomer A and Isomer B.

Isomer A: LCMS (m/z): 550.4 [M+H]⁺; ¹H NMR (400 MHz, CDCl₃): δ 7.87-7.77 (m, 3H), 7.59 (d, J=7.2 Hz, 1H), 7.51 (t, J=7.6 Hz, 1H), 7.34 (t, J=7.6 Hz, 1H), 6.99 (d, J=6.8 Hz, 1H), 6.57-6.50 (m, 1H), 6.29-6.25 (m, 1H), 5.69-5.66 (m, 1H), 4.86-4.83 (m, 2H), 4.54-4.47 (m, 1H), 3.88-3.63 (m, 12H), 2.95 (s, 3H), 2.27-2.25 (m, 2H), 2.09-2.06 (m, 2H).

Isomer B: LCMS (m/z): 550.4 [M+H]⁺; ¹H NMR (400 MHz, CD3OD): δ 7.92 (d, J=8.0 Hz, 1H), 7.88-7.85 (m, 1H), 7.79 (d, J=7.2 Hz, 1H), 7.64 (dd, J=7.6, 1.2 Hz, 1H), 7.52 (t, J=7.6 Hz, 1H), 7.39 (t, J=8.0 Hz, 1H), 7.01 (dd, J=8.0, 2.2 Hz, 1H), 6.73-6.66 (m, 1H), 6.15 (dd, J=16.8, 1.6 Hz, 1H), 5.69 (dd, J=10.8, 2.2 Hz, 1H), 4.85 (dd, J=11.8, 2.2 Hz, 1H), 4.57 (dd, J=12.4, 2.8 Hz, 2H), 3.83-3.56 (m, 12H), 2.96 (s, 3H), 2.30-2.24 (m, 1H), 2.15-2.04 (m, 1H), 2.01-1.86 (m, 2H).

### Example S-33: Synthesis of 1-((3S)-4-(7-(8-chloronaphthalen-1-yl)-2-(((S)-1-methylpyrrolidin-2-yl)methoxy)-6,7-dihydro-[1,4]dioxino[2,3-d]pyrimidin-4-yl)-3-methylpiperazin-1-yl)-2-fluoro prop-2-en-1-one (Compound 136, Isomer A and Isomer B)

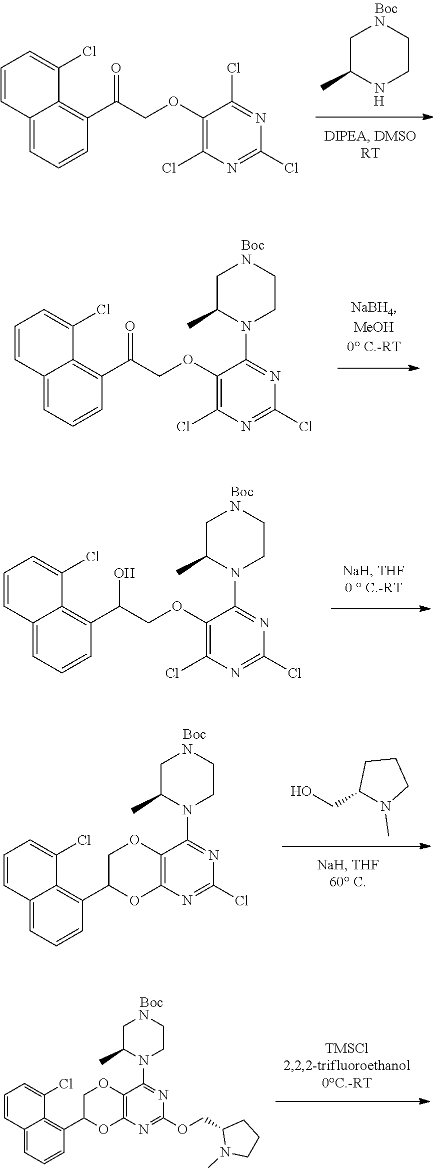

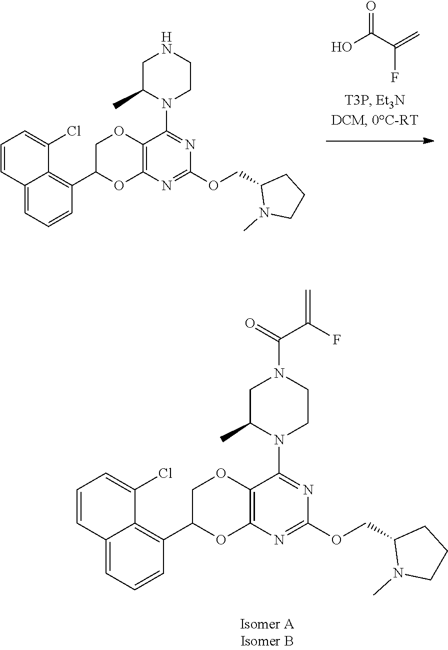

Step 1: Synthesis of tert-butyl (S)-4-(2,6-dichloro-5-(2-(8-chloronaphthalen-1-yl)-2-oxo ethoxy)pyrimidin-4-yl)-3-methylpiperazine-1-carboxylate. To a solution of 1-(8-chloro naphthalen-1-yl)-2-((2,4,6-trichloropyrimidin-5-yl)oxy)ethan-1-one (0.58 g, 1.44 mmol, 1.0 eq) in dry DMSO (6 mL) was charged with tert-butyl (S)-3-methylpiperazine-1-carboxylate (0.28 g, 1.44 mmol, 1.0 eq), DIPEA (0.75 mL, 4.32 mmol, 3.0 eq) and stirred at RT for 3 h. The reaction mixture was diluted with cold water (20 mL) and extracted with EtOAc (3×10 mL). The combined organic layers were dried over Na₂SO₄, filtered, and concentrated under reduced pressure to obtain crude material. The crude material was purified by silica gel chromatography to afford the title compound. LCMS (m/z): 565.0 [M+H]⁺

Step 2: Synthesis of tert-butyl (3S)-4-(2,6-dichloro-5-(2-(8-chloronaphthalen-1-yl)-2-hydroxyethoxy)pyrimidin-4-yl)-3-methylpiperazine-1-carboxylate. The title compound was synthesized following the procedure as described in Example S-8, Step 6 using tert-butyl (S)-4-(2,6-dichloro-5-(2-(8-chloronaphthalen-1-yl)-2-oxoethoxy)pyrimidin-4-yl)-3-methylpiperazine-1-carboxylate (0.28 g, 0.49 mmol, 1.0 eq). LCMS (m/z): 567.3 [M+H]⁺

Step 3: Synthesis of tert-butyl (3S)-4-(2-chloro-7-(8-chloronaphthalen-1-yl)-6,7-dihydro-[1,4]dioxino[2,3-d]pyrimidin-4-yl)-3-methylpiperazine-1-carboxylate. The title compound was synthesized following the procedure as described in Example S-8, Step 7 using tert-butyl (3S)-4-(2,6-dichloro-5-(2-(8-chloronaphthalen-1-yl)-2-hydroxyethoxy)pyrimidin-4-yl)-3-methylpiperazine-1-carboxylate (0.28 g, 0.49 mmol, 1.0 eq). LCMS (m/z): 531.0 [M+H]⁺

Step 4: Synthesis of tert-butyl (3S)-4-(7-(8-chloronaphthalen-1-yl)-2-(((S)-1-methyl pyrrolidin-2-yl)methoxy)-6,7-dihydro-[1,4]dioxino[2,3-d]pyrimidin-4-yl)-3-methyl piperazine-1-carboxylate. To a solution of N-methyl-L-prolinol (0.03 g, 0.25 mmol, 1.5 eq) in dry THF (5 mL) at 0° C. was added NaH (0.01 g, 0.51 mmol, 3.0 eq) and stirred at the same temperature for 3 h. Tert-butyl (3S)-4-(2-chloro-7-(8-chloronaphthalen-1-yl)-6,7-dihydro-[1,4]dioxino[2,3-d]pyrimidin-4-yl)-3-methylpiperazine-1-carboxylate (0.09 g, 0.17 mmol, 1.0 eq) in THF (0.50 mL) was added to the reaction mixture and stirred at 60° C. for 18 h. The reaction mixture was cooled to RT, quenched with ice cold water (10 mL), and extracted with EtOAc (2×10 mL). The combined organic layers were dried over Na₂SO₄, filtered, and concentrated under reduced pressure to obtain crude material. The crude material was purified by silica gel chromatography to afford the title compound. LCMS (m/z): 610.1[M+H]⁺

Step 5: Synthesis of 7-(8-chloronaphthalen-1-yl)-4-((S)-2-methylpiperazin-1-yl)-2-(((S)-1-methylpyrrolidin-2-yl)methoxy)-6,7-dihydro-[1,4]dioxino[2,3-d]pyrimidine. To a solution of tert-butyl (3S)-4-(7-(8-chloronaphthalen-1-yl)-2-(((S)-1-methyl pyrrolidin-2-yl)methoxy)-6,7-dihydro-[1,4]dioxino[2,3-d]pyrimidin-4-yl)-3-methyl piperazine-1-carboxylate (0.06 g, 0.09 mmol) in 2,2,2-trifluoroethanol (1 mL) at 0° C., was added chlorotrimethylsilane (0.09 mL) dropwise and stirred at RT for 2 h. The volatiles were removed under reduced pressure, diluted with water and saturated aqueous NaHCO₃ solution was added to adjust the pH to 8 and extracted with EtOAc (3×10 mL). The combined organic layers were dried over Na₂SO₄, filtered, and concentrated under reduced pressure to afford the title compound. LCMS (m/z): 510.1[M+H]⁺

Step 6: Synthesis of 1-((3S)-4-(7-(8-chloronaphthalen-1-yl)-2-(((S)-1-methylpyrrolidin-2-yl)methoxy)-6,7-dihydro-[1,4]dioxino[2,3-d]pyrimidin-4-yl)-3-methylpiperazin-1-yl)-2-fluoro prop-2-en-1-one. The title compound was synthesized following the procedure as described in Example S-32, Step 6 using 7-(8-chloronaphthalen-1-yl)-4-((S)-2-methylpiperazin-1-yl)-2-(((S)-1-methylpyrrolidin-2-yl)methoxy)-6,7-dihydro-[1,4]dioxino[2,3-d]pyrimidine (0.05 g, 0.09 mmol, 1.0 eq), Et₃N (0.04 mL, 0.26 mmol, 3.0 eq), 2-fluoroacrylic acid (0.01 g, 0.11 mmol, 1.2 eq) and T3P (50% in EtOAc; 0.10 mL, 0.18 mmol, 2.0 eq). The crude material was purified by silica gel chromatography and SFC to afford the title compound as Isomer A and Isomer B.

Isomer A: LCMS (m/z): 582.0 [M+H]⁺; ¹H NMR (400 MHz, CDCl₃): δ 7.95 (d, J=7.2 Hz, 1H), 7.90-7.87 (m, 1H), 7.85-7.83 (m, 1H), 7.65 (dd, J=1.2, 7.6 Hz, 1H), 7.58-7.54 (m, 1H), 7.42-7.38 (m, 1H), 7.05 (dd, J=2.0, 8.0 Hz, 1H), 5.37-5.25 (m, 1H), 5.16 (dd, J=3.6, 16.8 Hz, 1H), 5.07 (s, 1H), 4.86 (dd, J=2.4, 11.6 Hz, 1H), 4.33-4.29 (m, 3H), 4.16-4.12 (m, 1H), 3.91-3.73 (m, 2H), 3.38-3.35 (m, 1H), 3.09 (t, J=7.6 Hz, 1H) 2.64-2.63 (m, 1H), 2.57 (s, 3H), 2.28-2.26 (m, 1H), 2.06-2.01 (m, 2H), 1.84-1.70 (m, 4H), 1.25-1.23 (m, 3H).

Isomer B: LCMS (m/z): 582.0 [M+H]⁺; ¹H NMR (400 MHz, CDCl₃): δ 7.94 (d, J=7.6 Hz, 1H), 7.89-7.87 (m, 1H), 7.85-7.83 (m, 1H), 7.65 (dd, J=1.2, 7.2 Hz, 1H), 7.58-7.54 (m, 1H), 7.42-7.38 (m, 1H), 7.05 (dd, J=2.0, 8.0 Hz, 1H), 5.38-5.25 (m, 1H), 5.16 (dd, J=3.6, 17.2 Hz, 1H), 4.89-4.86 (m, 2H), 4.53-4.50 (m, 1H), 4.38 (s, 1H), 4.19-4.14 (m, 1H), 3.90-3.72 (m, 1H), 3.76-3.71 (m, 1H), 3.35-3.29 (m, 1H), 3.18-3.01 (m, 2H), 2.82-2.76 (m, 1H), 2.55 (s, 3H), 2.35-2.28 (m, 1H), 2.11-2.03 (m, 2H), 1.89-1.78 (m, 4H), 1.29-1.25 (m, 3H).

### Example S-34: Synthesis of 2-((2S)-4-(7-(8-chloronaphthalen-1-yl)-2-((2-methyl-1,2,3,4-tetrahydroisoquinolin-5-yl)oxy)-7,8-dihydro-5H-pyrano[4,3-d]pyrimidin-4-yl)-1-(2-fluoro acryloyl)piperazin-2-yl)acylonitrile (Compound 250, Isomer A and Isomer B)

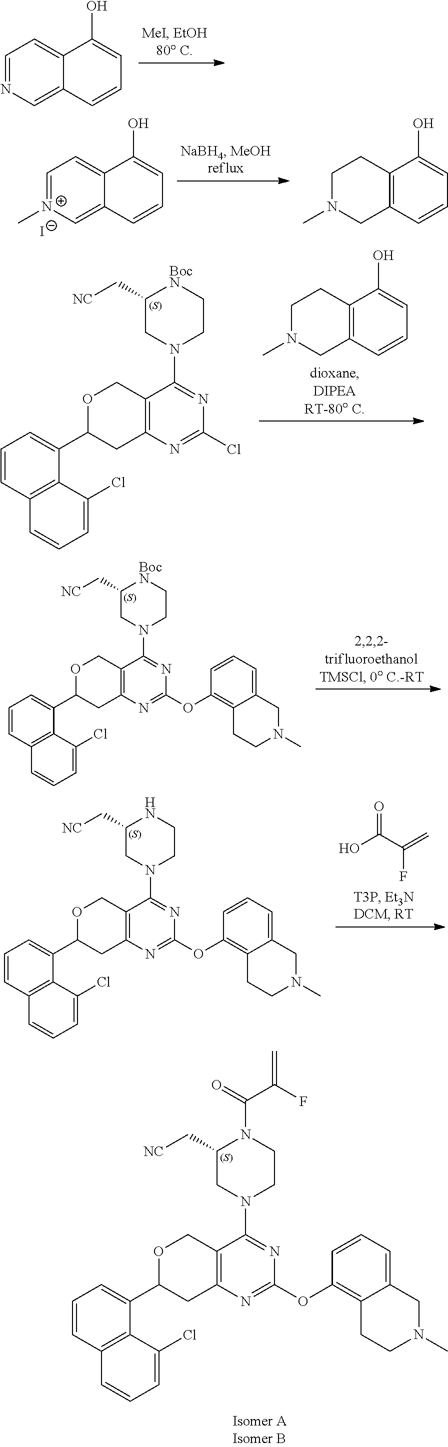

Step 1: Synthesis of 5-hydroxy-2-methylisoquinolin-2-ium iodide. The title compound was synthesized using the procedure as described in Example S-20, step 1 using isoquinolin-5-ol (3.0 g, 20.67 mmol, 1.0 eq) and CH₃I (2.6 mL, 41.33 mmol, 2.0 eq). LCMS (m/z): 160.2 [M]⁺

Step 2: Synthesis of 2-methyl-1,2,3,4-tetrahydroisoquinolin-5-ol. The title compound was synthesized using the procedure as described in Example S-20, step 2 using 5-hydroxy-2-methyl isoquinolin-2-ium iodide (2.0 g, 6.96 mmol, 1.0 eq) and NaBH₄ (1.18 g, 31.34 mmol, 4.5 eq). LCMS (m/z): 164.1 [M+H]⁺

Step 3: Synthesis of tert-butyl (2S)-4-(7-(8-chloronaphthalen-1-yl)-2-((2-methyl-1,2,3,4-tetrahydroisoquinolin-5-yl)oxy)-7,8-dihydro-5H-pyrano[4,3-d]pyrimidin-4-yl)-2-(cyano methyl)piperazine-1-carboxylate. To a solution of tert-butyl (2S)-4-(2-chloro-7-(8-chloro naphthalen-1-yl)-7,8-dihydro-5H-pyrano[4,3-d]pyrimidin-4-yl)-2-(cyanomethyl) piperazine-1-carboxylate (0.40 g, 0.72 mmol, 1.0 eq), 2-methyl-1,2,3,4-tetrahydroisoquinolin-5-ol (0.18 g, 1.08 mmol, 1.5 eq) in dioxane (8 mL) was added DIPEA (0.50 mL, 2.88 mmol, 4.0 eq) at RT and the reaction mixture was stirred at 80° C. for 2 h. The reaction mixture was diluted with H₂O (30 mL) and extracted with EtOAc (3×50 mL). The combined organic layers were washed with brine (30 mL), dried over Na₂SO₄, filtered, and concentrated under reduced pressure to afford the crude compound which was purified using silica gel chromatography to afford the title compound. LCMS (m/z): 681.0 [M+H]⁺

Step 4: Synthesis of 2-((2S)-4-(7-(8-chloronaphthalen-1-yl)-2-((2-methyl-1,2,3,4-tetra hydro isoquinolin-5-yl)oxy)-7,8-dihydro-5H-pyrano[4,3-d]pyrimidin-4-yl)piperazin-2-yl) acetonitrile. The title compound was synthesized using the procedure as described in Example S-10, step 2 using tert-butyl (2S)-4-(7-(8-chloronaphthalen-1-yl)-2-((2-methyl-1,2,3,4-tetra hydroisoquinolin-5-yl)oxy)-7,8-dihydro-5H-pyrano[4,3-d]pyrimidin-4-yl)-2-(cyano methyl) piperazine-1-carboxylate (0.09 g, 0.13 mmol, 1.0 eq). LCMS (m/z): 581.1 [M+H]⁺

Step 5: Synthesis of 2-((2S)-4-(7-(8-chloronaphthalen-1-yl)-2-((2-methyl-1,2,3,4-tetra hydroisoquinolin-5-yl)oxy)-7,8-dihydro-5H-pyrano[4,3-d]pyrimidin-4-yl)-1-(2-fluoro acryloyl)piperazin-2-yl)acetonitrile. The title compound was synthesized following the procedure as described in Example S-10, Step 3 using 2-((2S)-4-(7-(8-chloronaphthalen-1-yl)-2-((2-methyl-1,2,3,4-tetra hydro isoquinolin-5-yl)oxy)-7,8-dihydro-5H-pyrano[4,3-d]pyrimidin-4-yl)piperazin-2-yl) acetonitrile (0.08 g, 0.14 mmol, 1.0 eq), 2-fluoroacrylic acid (0.026 g, 0.29 mmol, 2.1 eq), T3P (50% in EtOAc; 0.26 mL, 0.44 mmol, 3.1 eq) and Et₃N (0.06 mL, 0.44 mmol, 3.1 eq). The crude material was purified by prep HPLC and SFC to produce the title compound as Isomer A and Isomer B.

Isomer A: LCMS (m/z): 653.2 [M+H]⁺; ¹H NMR (400 MHz, CDCl₃): δ 7.97 (d, J=7.2 Hz, 1H), 7.82 (dd, J=10.3, 8.8 Hz, 2H), 7.61-7.57 (m, 2H), 7.36 (t, J=7.6 Hz, 1H), 7.28-7.25 (m, 1H), 7.04 (d, J=8.0 Hz, 1H), 6.97 (d, J=8.0 Hz, 1H), 6.51 (dd, J=11.2, 3.2 Hz, 1H), 5.43 (s, 0.5H), 5.31 (s, 0.5H), 5.20 (dd, J=16.8, 3.6 Hz, 1H), 4.97 (d, J=13.8 Hz, 1H), 4.82 (d, J=13.8 Hz, 1H), 4.58 (br s, 1H), 4.00 (br s, 2H), 3.83-3.54 (m, 4H), 3.50-3.25 (m, 2H), 3.04-2.59 (m, 11H).

Isomer B: LCMS (m/z): 653.2 [M+H]⁺; ¹H NMR (400 MHz, CDCl₃): δ 7.97 (d, J=7.2 Hz, 1H), 7.83 (t, J=8.8 Hz, 2H), 7.63-7.51 (m, 2H), 7.40-7.28 (m, 2H), 7.11-6.98 (m, 2H), 6.49 (dd, J=11.2, 3.2 Hz, 1H), 5.41 (d, J=2.8 Hz, 0.5H), 5.30 (d, J=2.8 Hz, 0.5H), 5.19 (dd, J=16.8, 3.6 Hz, 1H), 4.93 (d, J=13.6 Hz, 1H), 4.75 (d, J=13.6 Hz, 1H), 4.65-4.50 (m, 1H), 4.39-3.76 (m, 4H), 3.70-3.53 (m, 3H), 3.35-2.99 (m, 6H), 2.97-2.69 (m, 6H).

### Example S-35: Synthesis of 2-((2S)-4-(7-(8-chloronaphthalen-1-yl)-2-((1,4-dimethylpyrrolidin-3-yl)oxy)-7,8-dihydro-5H-pyrano[4,3-d]pyrimidin-4-yl)-1-(2-fluoroacryloyl)piperazin-2-yl) acetonitrile (Compound 272, Isomer A, Isomer B, Isomer C and Isomer D)

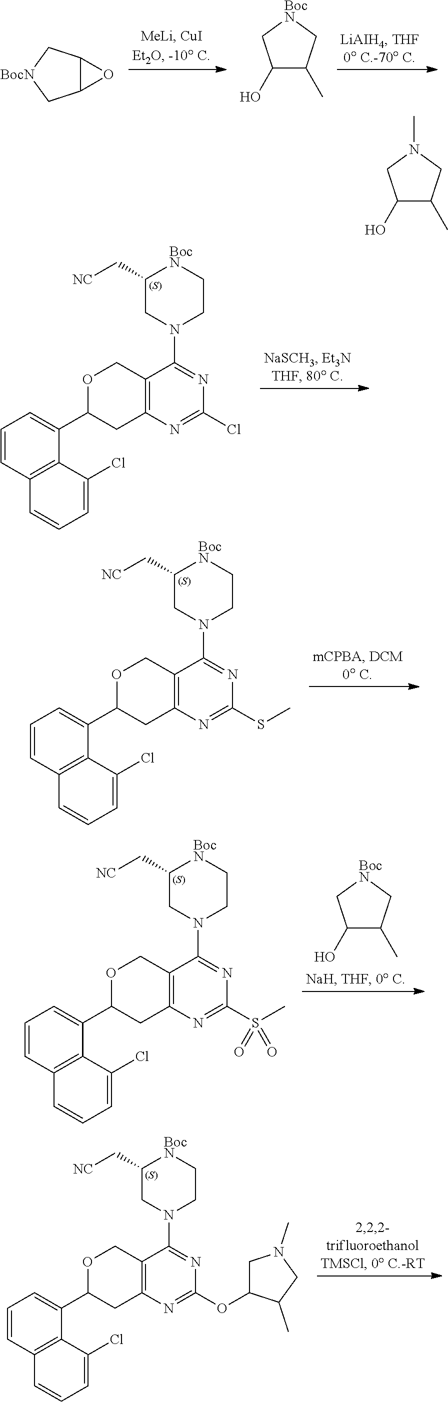

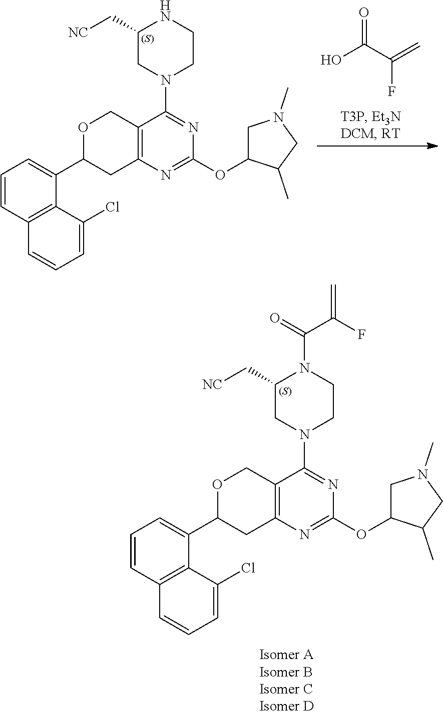

Step 1: Synthesis of tert-butyl 3-hydroxy-4-methylpyrrolidine-1-carboxylate. To a cooled suspension of CuI (4.70 g, 24.67 mmol, 2.3 eq) in dry Et₂O (40 mL) under nitrogen at −10° C., was added MeLi (1.6 M in Et₂O; 31.6 mL, 50.6 mmol, 4.7 eq) dropwise while maintaining internal temperature at −10° C. and the reaction mixture was stirred for 30 min. To the reaction mixture, tert-butyl 6-oxa-3-azabicyclo[3.1.0]hexane-3-carboxylate (2.0 g, 10.80, 1.0 eq) in dry Et₂O (25 mL) was added at −10° C. and stirred for 1 h. The reaction mixture was quenched with dropwise addition of water (25 mL) and DCM (25 mL). Filter the resulting mixture through a pad of Celite and wash thoroughly with DCM. Extract the aqueous layer with isopropanol/chloroform (30:70) and the combined organic layers were washed with brine, dried over Na₂SO₄, concentrated under reduced pressure to afford the title compound. LCMS (m/z): 202.1 [M+H]⁺

Step 2: Synthesis of 1,4-dimethylpyrrolidin-3-ol. To a solution of tert-butyl 3-hydroxy-4-methylpyrrolidine-1-carboxylate (0.50 g, 2.48 mmol, 1.0 eq) in dry THF (8 mL), was added LiAlH₄ (1M in THF; 8.7 mL, 8.68 mmol, 3.5 eq) dropwise at 0° C. and the reaction mixture was stirred at 70° C. for 6 h. The reaction mixture was quenched by sequential addition of water, 15% aqueous NaOH and water. The mixture was filtered through a Celite pad and the filtrate was concentrated under reduced pressure to give the title compound which was used in the next step without any further purification. LCMS (m/z): 116.1 [M+H]⁺

Step 3: Synthesis of tert-butyl (2S)-4-(7-(8-chloronaphthalen-1-yl)-2-(methylthio)-7,8-dihydro-5H-pyrano[4,3-d]pyrimidin-4-yl)-2-(cyanomethyl)piperazine-1-carboxylate. To a solution of tert-butyl (2S)-4-(2-chloro-7-(8-chloronaphthalen-1-yl)-7,8-dihydro-5H-pyrano[4,3-d]pyrimidin-4-yl)-2-(cyanomethyl)piperazine-1-carboxylate (5.54 g, 9.99 mmol, 1.0 eq) in THF (70 mL) was added NaSCH₃ (1.40 g, 19.98 mmol, 2.0 eq), Et₃N (4.2 mL, 29.97 mmol, 3.0 eq) under nitrogen and stirred at 80° C. for 12 h. The reaction mixture was quenched with water (10 mL) and extracted with EtOAc (3×50 mL). The combined organic layers were washed with brine (2×50 mL), dried over Na₂SO₄, concentrated under reduced pressure to afford the title compound which was used in the next step without any further purification. LCMS (m/z): 565.7 [M+H]⁺

Step 4: Synthesis of tert-butyl (2S)-4-(7-(8-chloronaphthalen-1-yl)-2-(methylsulfonyl)-7,8-dihydro-5H-pyrano[4,3-d]pyrimidin-4-yl)-2-(cyanomethyl)piperazine-1-carboxylate. To a solution of tert-butyl (2S)-4-(7-(8-chloronaphthalen-1-yl)-2-(methylthio)-7,8-dihydro-5H-pyrano[4,3-d]pyrimidin-4-yl)-2-(cyanomethyl)piperazine-1-carboxylate (5.66 g, 9.99 mmol, 1.0 eq) in DCM (100 mL) at 0° C. was added mCPBA (5.16 g, 29.99 mmol, 3.0 eq) and stirred at RT for 4 h. The reaction mixture was diluted with EtOAc (3×50 mL) and the combined organic layers were washed with brine (2×20 mL), dried over Na₂SO₄, concentrated under reduced pressure to give the crude compound which was purified by silica gel chromatography to afford the title compound. LCMS (m/z): 597.7 [M+H]⁺

Step 5: Synthesis of tert-butyl (2S)-4-(7-(8-chloronaphthalen-1-yl)-2-((1,4-dimethyl pyrrolidine-3-yl)oxy)-7,8-dihydro-5H-pyrano[4,3-d]pyrimidin-4-yl)-2-(cyanomethyl) piperazine-1-carboxylate. To a solution of 1,4-dimethylpyrrolidin-3-ol (0.30 g, 2.60 mmol, 3.9 eq) in THF at 0° C., was added NaH (0.21 g, 5.41 mmol, 8.1 eq) under nitrogen and stirred for 10 min. To the reaction mixture, tert-butyl (2S)-4-(7-(8-chloronaphthalen-1-yl)-2-(methylsulfonyl)-7,8-dihydro-5H-pyrano[4,3-d]pyrimidin-4-yl)-2-(cyanomethyl)piperazine-1-carboxylate (0.40 g, 0.67 mmol, 1.0 eq) in THF was added and stirred for 2 h. Water (20 mL) was added and extracted with EtOAc (3×10 mL). The combined organic layers were washed with brine (20 mL), dried over Na₂SO₄, concentrated under reduced pressure to give the crude compound which was purified by silica gel chromatography to afford the title compound. LCMS (m/z): 633.3 [M+H]⁺

Step 6: Synthesis of 2-((2S)-4-(7-(8-chloronaphthalen-1-yl)-2-((1,4-dimethylpyrrolidin-3-yl)oxy)-7,8-dihydro-5H-pyrano[4,3-d]pyrimidin-4-yl)piperazin-2-yl)acetonitrile. The title compound was synthesized using the procedure as described in Example S-10, step 2 using tert-butyl (2S)-4-(7-(8-chloronaphthalen-1-yl)-2-((1,4-dimethyl pyrrolidine-3-yl)oxy)-7,8-dihydro-5H-pyrano[4,3-d]pyrimidin-4-yl)-2-(cyanomethyl) piperazine-1-carboxylate (0.16 g, 0.25 mmol, 1.0 eq). LCMS (m/z): 533.2 [M+H]⁺

Step 7: Synthesis of 2-((2S)-4-(7-(8-chloronaphthalen-1-yl)-2-((1,4-dimethylpyrrolidin-3-yl)oxy)-7,8-dihydro-5H-pyrano[4,3-d]pyrimidin-4-yl)-1-(2-fluoroacryloyl)piperazin-2-yl) acetonitrile. The title compound was synthesized following the procedure as described in Example S-10, Step 3 using 2-((2S)-4-(7-(8-chloronaphthalen-1-yl)-2-((1,4-dimethylpyrrolidin-3-yl)oxy)-7,8-dihydro-5H-pyrano[4,3-d]pyrimidin-4-yl)piperazin-2-yl)acetonitrile (0.16 g, 0.30 mmol, 1.0 eq), 2-fluoroacrylic acid (0.066 g, 0.74 mmol, 2.5 eq), T3P (50% in EtOAc; 0.6 mL, 0.98 mmol, 3.3 eq) and Et₃N (0.14 mL, 0.99 mmol, 3.3 eq). The crude material was purified by prep HPLC and SFC to afford the title compound as Isomer A, Isomer B, Isomer C, and Isomer D.

Isomer A: LCMS (m/z): 605.3 [M+H]⁺; ¹H NMR (400 MHz, CDCl₃): δ 7.95 (d, J=6.8 Hz, 1H), 7.84 (t, J=9.2 Hz, 2H), 7.65-7.51 (m, 2H), 7.39 (t, J=7.8 Hz, 1H), 6.48 (dd, J=10.8, 3.2 Hz, 1H), 5.51-5.24 (m, 2H), 5.01 (d, J=13.9 Hz, 1H), 4.87 (d, J=13.9 Hz, 1H), 4.47-4.37 (m, 1H), 4.16-3.91 (m, 2H), 3.77 (t, J=12.6 Hz, 1H), 3.59 (t, J=13.8 Hz, 2H), 3.41-3.34 (m, 2H), 3.09-3.06 (m, 1H), 2.97 (s, 2H), 2.88-2.77 (m, 3H), 2.25 (br s, 6H), 1.41-1.25 (m, 3H).

Isomer B: LCMS (m/z): 605.1 [M+H]⁺; ¹H NMR (400 MHz, CDCl₃): δ 7.96 (d, J=7.2 Hz, 1H), 7.84 (t, J=8.1 Hz, 2H), 7.63-7.52 (m, 2H), 7.38 (t, J=7.8 Hz, 1H), 6.48 (dd, J=10.8, 3.2 Hz, 1H), 5.49 (s, 0.5H), 5.37 (s, 0.5H), 5.27 (dd, J=16.8, 3.7 Hz, 1H), 5.03-4.97 (m, 2H), 4.86 (d, J=13.7 Hz, 1H), 4.27-4.04 (m, 1H), 3.95-3.87 (m, 1H), 3.78 (d, J=10.6 Hz, 1H), 3.58 (dd, J=18.4, 2.8 Hz, 1H), 3.45-3.32 (m, 1H), 3.23-3.10 (m, 3H), 3.07 (dd, J=16.7, 8.3 Hz, 1H), 3.00-2.85 (m, 3H), 2.60 (br s, 4H), 1.27-1.21 (m, 6H).

Isomer C: LCMS (m/z): 605.2 [M+H]⁺; ¹H NMR (400 MHz, CDCl₃): δ 7.97 (d, J=7.2 Hz, 1H), 7.82 (t, J=8.8 Hz, 2H), 7.63-7.52 (m, 2H), 7.36 (t, J=7.8 Hz, 1H), 6.51 (dd, J=11.2, 3.6 Hz, 1H), 5.47 (s, 0.5H), 5.36 (s, 0.5H), 5.25 (dd, J=16.9, 3.4 Hz, 1H), 4.98 (d, J=13.8 Hz, 2H), 4.82 (d, J=13.8 Hz, 1H), 3.97 (d, J=13.6 Hz, 1H), 3.77 (d, J=10.0 Hz, 1H), 3.64-3.41 (m, 3H), 3.07-2.69 (m, 7H), 2.63-2.47 (m, 4H), 1.26-1.21 (m, 6H).

Isomer D: LCMS (m/z): 605.2 [M+H]⁺; ¹H NMR (400 MHz, CDCl₃): δ 7.96 (d, J=7.0 Hz, 1H), 7.84 (t, J=9.2 Hz, 2H), 7.62-7.52 (m, 2H), 7.38 (t, J=7.8 Hz, 1H), 6.51 (dd, J=10.8, 3.2 Hz, 1H), 5.51 (s, 0.5H), 5.39 (s, 0.5H), 5.26-5.20 (m, 2H), 5.02 (d, J=13.6 Hz, 1H), 4.85 (d, J=13.6 Hz, 1H), 4.50-4.45 (m, 1H), 4.10-3.90 (m, 2H), 3.80-3.75 (m, 1H), 3.70-3.50 (m, 3H), 3.36-3.03 (m, 3H), 2.96 (s, 2H), 2.83-2.75 (m, 4H), 1.85 (s, 3H), 1.25-1.23 (m, 3H).

### Example S-36: Synthesis of 2-((2S)-4-(7-(8-chloronaphthalen-1-yl)-2-((tetrahydro-1H-pyrrolizin-7a(5H)-yl)methoxy)-7,8-dihydro-5H-pyrano[4,3-d]pyrimidin-4-yl)-1-(2-fluoro acryloyl) piperazin-2-yl)acetonitrile (Compound 266, Isomer A and Isomer B)

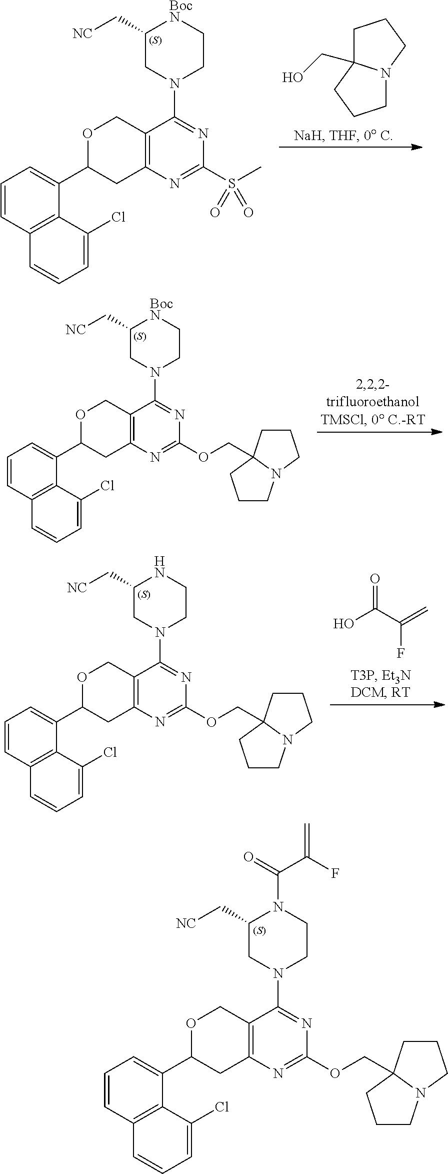

Step 1: Synthesis of tert-butyl (2S)-4-(7-(8-chloronaphthalen-1-yl)-2-((tetrahydro-1H-pyrrolizin-7a(5H)-yl)methoxy)-7,8-dihydro-5H-pyrano[4,3-d]pyrimidin-4-yl)-2-(cyano methyl)piperazine-1-carboxylate. The title compound was synthesized using the procedure as described in Example S-35, step 5 using tert-butyl (2S)-4-(7-(8-chloronaphthalen-1-yl)-2-(methylsulfonyl)-7,8-dihydro-5H-pyrano[4,3-d]pyrimidin-4-yl)-2-(cyanomethyl)piperazine-1-carboxylate (0.60 g, 1.00 mmol, 1.0 eq) and (tetrahydro-1H-pyrrolizin-7a(5H)-yl)methanol (0.28 g, 2.00 mmol, 2.0 eq). LCMS (m/z): 659.3 [M+H]⁺

Step 2: Synthesis of 2-((2S)-4-(7-(8-chloronaphthalen-1-yl)-2-((tetrahydro-1H-pyrrolizin-7 a (5H)-yl)methoxy)-7,8-dihydro-5H-pyrano[4,3-d]pyrimidin-4-yl)piperazin-2-yl) acetonitrile. The title compound was synthesized using the procedure as described in Example S-10, step 2 using tert-butyl (2S)-4-(7-(8-chloronaphthalen-1-yl)-2-((tetrahydro-1H-pyrrolizin-7 a (5H)-yl)methoxy)-7,8-dihydro-5H-pyrano[4,3-d]pyrimidin-4-yl)-2-(cyano methyl)piperazine-1-carboxylate (0.35 g, 0.53 mmol, 1.0 eq) and TMSCl (0.2 mL, 1.59 mmol, 3.0 eq). LCMS (m/z): 559.2 [M+H]⁺

Step 3: Synthesis of 2-((2S)-4-(7-(8-chloronaphthalen-1-yl)-2-((tetrahydro-1H-pyrrolizin-7a(5H)-yl)methoxy)-7,8-dihydro-5H-pyrano[4,3-d]pyrimidin-4-yl)-1-(2-fluoro acryloyl) piperazin-2-yl)acetonitrile. The title compound was synthesized following the procedure as described in Example S-10, Step 3 using 2-((2S)-4-(7-(8-chloronaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl) acetonitrile (0.15 g, 0.27 mmol, 1.0 eq), 2-fluoroacrylic acid (0.076 g, 0.85 mmol, 3.1 eq), T3P (50% in EtOAc; 0.6 mL, 0.98 mmol, 3.6 eq) and Et₃N (0.14 mL, 0.99 mmol, 3.7 eq). The crude material was purified by prep HPLC and SFC to afford the title compound as Isomer A and Isomer B.

Isomer A: LCMS (m/z): 631.0 [M+H]⁺; ¹H NMR (400 MHz, CDCl₃): δ 7.95 (d, J=7.2 Hz, 1H), 7.87-7.79 (m, 2H), 7.61-7.52 (m, 2H), 7.36 (t, J=7.6 Hz, 1H), 6.47 (dd, J=10.8, 3.2 Hz, 1H), 5.47 (s, 0.5H), 5.36 (s, 0.5H), 5.26 (dd, J=16.8, 3.6 Hz, 1H), 4.98-4.80 (m, 3H), 4.11 (br s, 4H), 3.77 (d, J=8.0 Hz, 1H), 3.60 (dd, J=18.2, 2.5 Hz, 1H), 3.50-3.00 (m, 6H), 2.90 (dd, J=18.2, 10.4 Hz, 2H), 2.67 (br s, 2H), 2.10-1.89 (m, 6H), 1.75-1.70 (m, 2H).

Isomer B: LCMS (m/z): 630.9 [M+H]⁺; ¹H NMR (400 MHz, CDCl₃): δ 7.96 (d, J=7.6 Hz, 1H), 7.86-7.79 (m, 2H), 7.62-7.53 (m, 2H), 7.36 (t, J=8.0 Hz, 1H), 6.50 (dd, J=10.8, 3.2 Hz, 1H), 5.46 (d, J=3.6 Hz, 0.5H), 5.34 (d, J=3.6 Hz, 0.5H), 5.23 (dd, J=16.9, 3.6 Hz, 1H), 5.02 (d, J=13.6 Hz, 1H), 4.82 (d, J=13.6 Hz, 2H), 4.66 (s, 2H), 4.24-3.71 (m, 7H), 3.60 (dd, J=18.4, 2.4 Hz, 1H), 3.30-3.15 (m, 1H), 3.05-2.83 (m, 5H), 2.45-2.35 (m, 2H), 2.30-2.20 (m, 2H), 2.16-2.07 (m, 2H), 2.05-1.95 (m, 2H).

### Example S-37: Synthesis of 2-((2S)-4-(7-(8-chloronaphthalen-1-yl)-2-(((R)-1-methylpyrrolidin-2-yl)methoxy)-7,8-dihydro-5H-pyrano[4,3-d]pyrimidin-4-yl)-1-(2-fluoroacryloyl)piperazin-2-yl) acetonitrile (Compound 267, Isomer A and Isomer B)

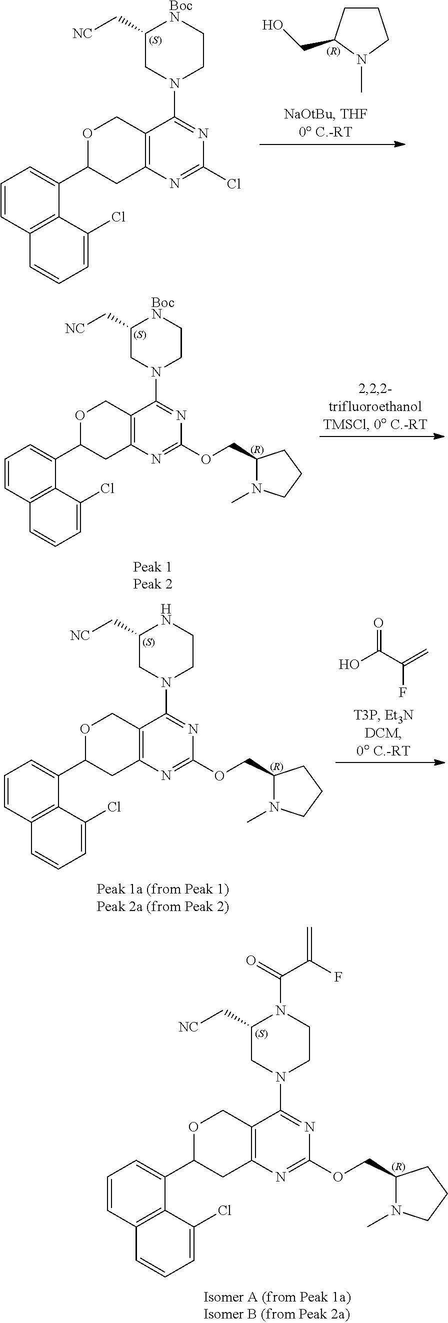

Step 1: Synthesis of tert-butyl (2S)-4-(7-(8-chloronaphthalen-1-yl)-2-(((R)-1-methyl pyrrolidin-2-yl)methoxy)-7,8-dihydro-5H-pyrano[4,3-d]pyrimidin-4-yl)-2-(cyanomethyl) piperazine-1-carboxylate (Peak 1 and Peak 2). To a solution of tert-butyl (2S)-4-(2-chloro-7-(8-chloronaphthalen-1-yl)-7,8-dihydro-5H-pyrano[4,3-d]pyrimidin-4-yl)-2-(cyanomethyl) piperazine-1-carboxylate (1.0 g, 1.80 mmol, 1.0 eq) and (R)-(1-methylpyrrolidin-2-yl)methanol (0.25 mL, 2.16 mmol, 1.2 eq) in dry THF (10 mL) at 0° C., was added NaOtBu (0.26 g, 2.70 mmol, 1.5 eq) and stirred at RT for 4 h. The reaction mixture was quenched with ice cooled water (50 mL) and extracted with EtOAc (2×20 mL). The combined organic layers were dried over Na₂SO₄, concentrated under reduced pressure to afford the crude material which was purified by silica gel chromatography to afford the title compound as a mixture of diastereomers. The diastereomeric mixture was separated by SFC to give the title compound as Peak 1 and Peak 2. Peak 1: LCMS (m/z): 633.1 [M+H]⁺; Peak 2: LCMS (m/z): 633.1 [M+H]⁺

Step 2: Synthesis of 2-((2S)-4-(7-(8-chloronaphthalen-1-yl)-2-(((R)-1-methylpyrrolidin-2-yl) methoxy)-7,8-dihydro-5H-pyrano[4,3-d]pyrimidin-4-yl)piperazin-2-yl)acetonitrile (Peak 1a and Peak 2a). The title compound Peak 1a was synthesized using the procedure as described in Example S-10, step 2 using tert-butyl (2S)-4-(7-(8-chloronaphthalen-1-yl)-2-(((R)-1-methyl pyrrolidin-2-yl)methoxy)-7,8-dihydro-5H-pyrano[4,3-d]pyrimidin-4-yl)-2-(cyanomethyl) piperazine-1-carboxylate (Peak 1; 0.28 g, 0.44 mmol, 1.0 eq). LCMS (m/z): 533.4 [M]⁺

The title compound Peak 2a was synthesized using the procedure as described in Example S-10, step 2 using tert-butyl (2S)-4-(7-(8-chloronaphthalen-1-yl)-2-(((R)-1-methyl pyrrolidin-2-yl) methoxy)-7,8-dihydro-5H-pyrano[4,3-d]pyrimidin-4-yl)-2-(cyanomethyl) piperazine-1-carboxylate (Peak 2; 0.28 g, 0.44 mmol, 1.0 eq). LCMS (m/z): 533.1 [M]⁺

Step 3: Synthesis of 2-((2S)-4-(7-(8-chloronaphthalen-1-yl)-2-(((R)-1-methylpyrrolidin-2-yl)methoxy)-7,8-dihydro-5H-pyrano[4,3-d]pyrimidin-4-yl)-1-(2-fluoroacryloyl)piperazin-2-yl) acetonitrile (Isomer A and Isomer B). The title compound (Isomer A) was synthesized following the procedure as described in Example S-10, Step 3 using 2-((2S)-4-(7-(8-chloronaphthalen-1-yl)-2-(((R)-1-methylpyrrolidin-2-yl) methoxy)-7,8-dihydro-5H-pyrano[4,3-d]pyrimidin-4-yl)piperazin-2-yl)acetonitrile (Peak 1a; 0.10 g, 0.18 mmol, 1.0 eq), 2-fluoro acrylic acid (0.025 g, 0.28 mmol, 1.5 eq), T3P (50% in EtOAc; 0.26 mL, 0.45 mmol, 2.5 eq) and Et₃N (0.08 mL, 0.54 mmol, 3.0 eq). The crude material was purified by silica gel chromatography to give the title compound as Isomer A.

Isomer A: LCMS (m/z): 605.4 [M+H]⁺; ¹H NMR (400 MHz, CDCl₃): δ 7.95 (d, J=7.2 Hz 1H), 7.84-7.80 (m, 2H), 7.61 (dd, J=7.2, 1.6 Hz, 1H), 7.56 (t, J=7.6 Hz, 1H), 7.36 (t, J=8.0 Hz, 1H), 6.47 (dd, J=10.8, 3.2 Hz, 1H), 5.48 (d, J=2.4 Hz, 0.5 H), 5.36 (d, J=2.4 Hz, 0.5H), 5.26 (dd, J=17.2, 3.6 Hz, 1H), 4.96 (d, J=13.6 Hz, 1H), 4.83 (d, J=13.6 Hz, 1H), 4.39-4.38 (m, 1H), 4.21-4.16 (m, 1H), 4.08-3.93 (m, 1H), 3.76 (d, J=9.6 Hz, 1H), 3.58 (dd, J=18.0, 2.4 Hz, 1H), 3.26-3.04 (m, 6H), 2.92-2.69 (m, 3H), 2.49 (s, 3H), 2.30 (d, J=7.6 Hz, 1H), 2.07-2.02 (m, 1H), 1.84-1.70 (m, 4H).

The title compound (Isomer B) was synthesized following the procedure as described in Example S-10, Step 3 using 2-((2S)-4-(7-(8-chloronaphthalen-1-yl)-2-(((R)-1-methylpyrrolidin-2-yl) methoxy)-7,8-dihydro-5H-pyrano[4,3-d]pyrimidin-4-yl)piperazin-2-yl)acetonitrile (Peak 2a; 0.10 g, 0.18 mmol, 1.0 eq), 2-fluoro acrylic acid (0.025 g, 0.28 mmol, 1.5 eq), T3P (50% in EtOAc; 0.27 mL, 0.45 mmol, 2.5 eq) and Et₃N (0.08 mL, 0.54 mmol, 3.0 eq). The crude material was purified by silica gel chromatography to give the title compound as Isomer B.

Isomer B: LCMS (m/z): 605.4 [M+H]⁺; δ 7.97 (d, J=7.2 Hz 1H), 7.84-7.80 (m, 2H), 7.60 (dd, J=7.2, 1.2 Hz, 1H), 7.56 (t, J=8.0 Hz, 1H), 7.36 (t, J=8.0 Hz, 1H), 6.51 (dd, J=11.2, 3.2 Hz, 1H), 5.48 (s, 0.5H), 5.36 (s, 0.5H), 5.25 (dd, J=16.8, 3.6 Hz, 1H), 4.98 (d, J=13.6 Hz, 1H), 4.83 (d, J=13.6 Hz, 1H), 4.38 (dd, J=10.4, 4.8 Hz, 1H), 4.16 (dd, J=10.4, 6.8 Hz, 1H), 3.98 (d, J=14.0 Hz, 1H), 3.76 (d, J=9.6 Hz, 1H), 3.58 (dd, J=18.4, 2.4 Hz, 1H), 3.51-3.48 (m, 2H), 3.11-3.07 (m, 2H), 2.92-2.65 (m, 5H), 2.49 (s, 3H), 2.28 (d, J=7.2 Hz, 1H), 2.07-2.02 (m, 1H), 1.84-1.70 (m, 4H).

### Example S-38: Synthesis of 1-(2,4-dichloro-7,8-dihydro-5H-pyrano[4,3-d]pyrimidin-7-yl)-N,N-bis(4-methoxybenzyl)isoquinolin-3-amine

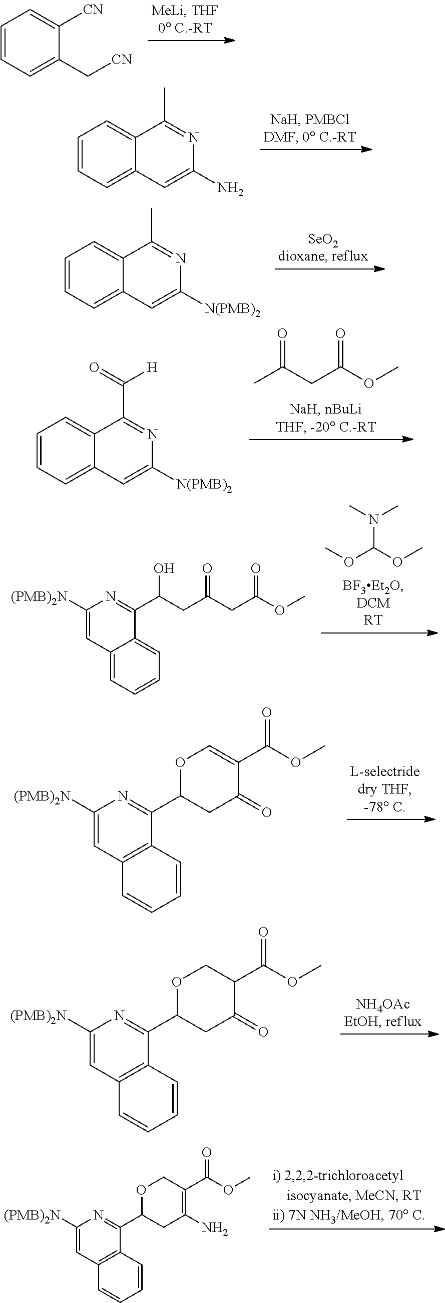

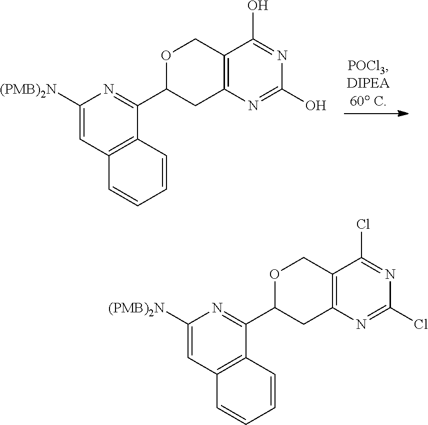

Step 1: Synthesis of 1-methylisoquinolin-3-amine. To a solution of 2-(cyanomethyl)benzonitrile (40.0 g, 281.37 mmol, 1.0 eq) in THF (800 mL) at 0° C., was added MeLi (1.6 M in Et₂O, 527.6 mL, 844.11 mmol, 3.0 eq) and stirred at RT for 2 h. The reaction mixture was quenched with aqueous ammonium chloride (500 mL) and extracted with DCM (3×300 mL). The combined organic layers were washed with brine (300 mL), dried over Na₂SO₄, filtered, and concentrated under reduced pressure to afford the crude material which was used in the next step without any further purification. LCMS (m/z): 159.0 [M+H]⁺

Step 2: Synthesis of N,N-bis(4-methoxybenzyl)-1-methylisoquinolin-3-amine. To a stirred solution of NaH (12.14 g, 316.05 mmol, 2.5 eq) in DMF (250 mL) at 0° C., was added 1-methyl isoquinolin-3-amine (20.0 g, 126.42 mmol, 1.0 eq) in DMF (150 mL) dropwise and the reaction mixture was stirred for 30 min. To the reaction mixture, p-methoxy benzylchloride (51.42 mL, 379.26 mmol, 3.0 eq) was added dropwise at 0° C. and the reaction mixture was stirred at RT for 16 h. The reaction mixture was quenched with ice-cold water (500 mL) and extracted with MTBE (3×300 mL). The combined organic layers were washed with brine (300 mL), dried over Na₂SO₄, filtered and concentrated under reduced pressure to afford the crude material which was purified by silica gel chromatography to give the title compound. LCMS (m/z): 399.1 [M+H]⁺

Step 3: Synthesis of 3-(bis(4-methoxybenzyl)amino)isoquinoline-1-carbaldehyde. To a stirred solution of N,N-bis(4-methoxybenzyl)-1-methylisoquinolin-3-amine (35.0 g, 87.82 mmol, 1.0 eq) in dioxane (350 mL) was added selenium dioxide (19.49 g, 175.65 mmol, 2.0 eq) portion wise at RT and heated to 100° C. for 8 h. The reaction mixture was cooled to RT, filtered through a Celite pad and washed with EtOAc (350 mL). The filtrate was concentrated under reduced pressure to afford the crude material which was purified by silica gel chromatography to give the title compound. LCMS (m/z): 413.1 [M+H]⁺

Step 4: Synthesis of methyl 5-(3-(bis(4-methoxybenzyl)amino)isoquinolin-1-yl)-5-hydroxy-3-oxopentanoate. To a stirred solution of NaH (3.97 g, 103.34 mmol, 1.2 eq) in THF (50 mL) at 0° C., was added methyl 3-oxobutanoate (10.0 g, 86.11 mmol, 1.0 eq) in THF (30 mL) dropwise and the reaction mixture was stirred at 0° C. for 1 h. The reaction mixture was cooled to −20° C. and n-BuLi (2.5 M in hexane, 41.3 mL, 103.34 mmol, 1.2 eq) was added dropwise and stirred at −20° C. for 1 h and then 3-(bis(4-methoxybenzyl)amino)isoquinoline-1-carbaldehyde (42.62 g, 103.34 mmol, 1.2 eq) in THF (80 mL) was added dropwise at the same temperature and stirred for 2 h. The reaction mixture was quenched with aqueous NH₄Cl solution (500 mL) and extracted with EtOAc (3×300 mL). The combined organic layers were washed with brine (300 mL), dried over Na₂SO₄, filtered, and concentrated under reduced pressure to give the crude material which was purified by silica gel chromatography to afford the title compound. LCMS (m/z): 529.1 [M+H]⁺

Step 5: Synthesis of methyl 2-(3-(bis(4-methoxybenzyl)amino)isoquinolin-1-yl)-4-oxo-3,4-dihydro-2H-pyran-5-carboxylate. The title compound was synthesized following the procedure as described in Example S-9, Step 2 using methyl 5-(3-(bis(4-methoxybenzyl)amino)isoquinolin-1-yl)-5-hydroxy-3-oxopentanoate (6.0 g, 11.35 mmol, 1.0 eq). LCMS (m/z): 539.0 [M+H]⁺

Step 6: Synthesis of methyl 6-(3-(bis(4-methoxybenzyl)amino)isoquinolin-1-yl)-4-oxotetra hydro-2H-pyran-3-carboxylate. The title compound was synthesized following the procedure as described in Example S-9, Step 3 using methyl 2-(3-(bis(4-methoxybenzyl)amino)isoquinolin-1-yl)-4-oxo-3,4-dihydro-2H-pyran-5-carboxylate (6.0 g, 11.13 mmol, 1.0 eq). LCMS (m/z): 541.1 [M+H]⁺

Step 7: Synthesis of methyl 4-amino-6-(3-(bis(4-methoxybenzyl)amino) isoquinolin-1-yl)-5,6-dihydro-2H-pyran-3-carboxylate. The title compound was synthesized following the procedure as described in Example S-9, Step 4 using methyl 6-(3-(bis(4-methoxybenzyl)amino)isoquinolin-1-yl)-4-oxotetra hydro-2H-pyran-3-carboxylate (4.0 g, 7.40 mmol, 1.0 eq) and NH₄OAc (1.71 g, 22.20 mmol, 3.0 eq) in ethanol (80 mL) under reflux. LCMS (m/z): 540.1 [M+H]⁺

Step 8: Synthesis of 7-(3-(bis(4-methoxybenzyl)amino)isoquinolin-1-yl)-7,8-dihydro-5H-pyrano[4,3-d]pyrimidine-2,4-diol. The title compound was synthesized following the procedure as described in Example S-9, Step 5 using methyl 4-amino-6-(3-(bis(4-methoxy benzyl)amino)isoquinolin-1-yl)-5,6-dihydro-2H-pyran-3-carboxylate (4.0 g, 7.41 mmol, 1.0 eq). LCMS (m/z): 551.0 [M+H]⁺

Step 9: Synthesis of 1-(2,4-dichloro-7,8-dihydro-5H-pyrano[4,3-d]pyrimidin-7-yl)-N,N-bis(4-methoxybenzyl)isoquinolin-3-amine. The title compound was synthesized following the procedure as described in Example S-1, Step 7 using 7-(3-(bis(4-methoxybenzyl) amino) isoquinolin-1-yl)-7,8-dihydro-5H-pyrano[4,3-d]pyrimidine-2,4-diol (2.0 g, 3.63 mmol, 1.0 eq). LCMS (m/z): 587.3 [M+H]⁺

### Example S-39: Synthesis of 2-((2S)-4-(7-(3-aminoisoquinolin-1-yl)-2-(((S)-1-methylpyrrolidin-2-yl)methoxy)-7,8-dihydro-5H-pyrano[4,3-d]pyrimidin-4-yl)-1-(2-fluoroacryloyl)piperazin-2-yl) acetonitrile (Compound 259, Isomer A and Isomer B)

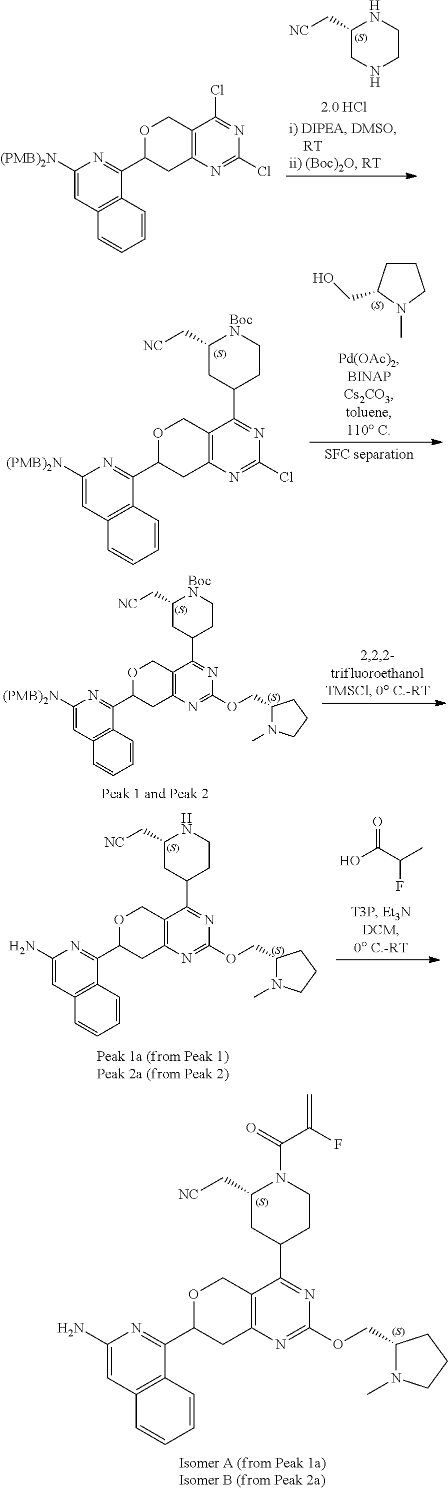

Step 1: Synthesis of tert-butyl (2S)-4-(7-(3-(bis(4-methoxybenzyl)amino)isoquinolin-1-yl)-2-chloro-7,8-dihydro-5H-pyrano[4,3-d]pyrimidin-4-yl)-2-(cyanomethyl)piperazine-1-carboxylate. The title compound was synthesized following the procedure as described in Example S-2, Step 1 using 1-(2,4-dichloro-7,8-dihydro-5H-pyrano[4,3-d]pyrimidin-7-yl)-N,N-bis(4-methoxybenzyl)isoquinolin-3-amine (0.80 g, 1.36 mmol, 1.0 eq), (S)-2-(piperazin-2-yl) acetonitrile dihydrochloride (0.27 g, 1.36 mmol, 1.0 eq), DIPEA (1.18 mL, 6.80 mmol, 5.0 eq) and Boc₂O (0.46 mL, 2.04 mmol, 1.5 eq). The crude material was purified by silica gel chromatography to give the title compound. LCMS (m/z): 776.2 [M+H]⁺

Step 2: Synthesis of tert-butyl (2S)-4-(7-(3-(bis(4-methoxybenzyl)amino)isoquinolin-1-yl)-2-(((S)-1-methylpyrrolidin-2-yl)methoxy)-7,8-dihydro-5H-pyrano[4,3-d]pyrimidin-4-yl)-2-(cyanomethyl)piperazine-1-carboxylate (Peak 1 and Peak 2). A solution of tert-butyl (2S)-4-(7-(3-(bis(4-methoxy benzyl)amino)isoquinolin-1-yl)-2-chloro-7,8-dihydro-5H-pyrano[4,3-d]pyrimidin-4-yl)-2-(cyanomethyl)piperazine-1-carboxylate (0.80 g, 1.03 mmol, 1.0 eq), N-methyl-L-prolinol (0.25 mL, 2.06 mmol, 2.0 eq) in dry toluene (20 mL) was degassed with argon at RT for 10 min followed by the addition of BINAP (0.13 g, 0.21 mmol, 0.20 eq), Pd(OAc)₂ (0.023 g, 0.10 mmol, 0.10 eq), Cs₂CO₃ (1.00 g, 3.09 mmol, 3.0 eq). The reaction mixture was degassed for 10 min and stirred at 110° C. for 5 h. The reaction mixture was cooled to RT, filtered through a Celite bed and washed with EtOAc (50 mL). The filtrate was concentrated under reduced pressure to give the crude material which was purified by silica gel chromatography to afford the title compound as a mixture of diastereomers. The diastereomeric mixtures was separated by SFC to give the title compound as Peak 1 and Peak 2. Peak 1: LCMS (m/z): 855.3 [M+H]⁺; Peak 2: LCMS (m/z): 855.3 [M+H]⁺

Step 3: Synthesis of 2-((2S)-4-(7-(3-aminoisoquinolin-1-yl)-2-(((S)-1-methylpyrrolidin-2-yl)methoxy)-7,8-dihydro-5H-pyrano[4,3-d]pyrimidin-4-yl)piperazin-2-yl)acetonitrile (Peak 1a and Peak 1b). The title compound Peak 1a was synthesized using the procedure as described in Example S-10, step 2 using tert-butyl (2S)-4-(7-(3-(bis(4-methoxybenzyl)amino)isoquinolin-1-yl)-2-(((S)-1-methylpyrrolidin-2-yl)methoxy)-7,8-dihydro-5H-pyrano[4,3-d]pyrimidin-4-yl)-2-(cyanomethyl)piperazine-1-carboxylate (Peak 1; 0.22 g, 0.25 mmol, 1.0 eq). LCMS (m/z): 515.2 [M+H]⁺

The title compound Peak 2a was synthesized using the procedure as described in Example S-10, step 2 using tert-butyl (2S)-4-(7-(3-(bis(4-methoxybenzyl)amino)isoquinolin-1-yl)-2-(((S)-1-methylpyrrolidin-2-yl)methoxy)-7,8-dihydro-5H-pyrano[4,3-d]pyrimidin-4-yl)-2-(cyanomethyl) piperazine-1-carboxylate (Peak 2; 0.20 g, 0.23 mmol, 1.0 eq). LCMS (m/z): 515.2 [M+H]⁺

Step 4: Synthesis of 2-((2S)-4-(7-(3-aminoisoquinolin-1-yl)-2-(((S)-1-methylpyrrolidin-2-yl)methoxy)-7,8-dihydro-5H-pyrano[4,3-d]pyrimidin-4-yl)-1-(2-fluoroacryloyl)piperazin-2-yl) acetonitrile (Isomer A and Isomer B). The title compound (Isomer A) was synthesized following the procedure as described in Example S-10, Step 3 using 2-((2S)-4-(7-(3-amino isoquinolin-1-yl)-2-(((S)-1-methylpyrrolidin-2-yl)methoxy)-7,8-dihydro-5H-pyrano[4,3-d]pyrimidin-4-yl)piperazin-2-yl)acetonitrile (Peak 1a; 0.13 g, 0.25 mmol, 1.0 eq), 2-fluoro acrylic acid (0.02 g, 0.22 mmol, 0.9 eq), T3P (50% in EtOAc; 0.22 mL, 0.37 mmol, 1.5 eq) and Et₃N (0.10 mL, 0.75 mmol, 3.0 eq). The crude material was purified by prep HPLC to give the title compound as Isomer A.

Isomer A: LCMS (m/z): 587.5 [M+H]⁺; δ 8.13 (d, J=8.0 Hz, 1H), 7.58 (d, J=8.0 Hz, 1H), 7.51-7.47 (m, 2H), 6.73 (s, 1H), 5.65 (dd, J=8.4, 4.8 Hz, 1H), 5.47-5.30 (m, 1H), 5.24 (dd, J=16.8, 3.6 Hz, 1H), 4.84 (d, J=13.6 Hz, 1H), 4.65 (d, J=13.6 Hz, 1H), 4.44-4.40 (m, 2H), 4.32-4.29 (m, 1H), 3.96-3.88 (m, 1H), 3.75-3.64 (m, 3H), 3.27-3.25 (m, 2H), 3.18-3.10 (m, 3H), 2.98-2.95 (m, 1H), 2.87-2.76 (m, 2H), 2.60-2.55 (m, 4H), 2.40-2.31 (m, 2H), 1.91-1.82 (m, 4H).

The title compound (Isomer B) was synthesized following the procedure as described in Example S-10, Step 3 using 2-((2S)-4-(7-(3-amino isoquinolin-1-yl)-2-(((S)-1-methylpyrrolidin-2-yl)methoxy)-7,8-dihydro-5H-pyrano[4,3-d]pyrimidin-4-yl)piperazin-2-yl)acetonitrile (Peak 2a; 0.14 g, 0.27 mmol, 1.0 eq), 2-fluoro acrylic acid (0.02 g, 0.24 mmol, 0.9 eq), T3P (50% in EtOAc; 0.24 mL, 0.40 mmol, 1.5 eq) and Et₃N (0.11 mL, 0.81 mmol, 3.0 eq). The crude material was purified by prep HPLC to give the title compound as Isomer B.

Isomer B: LCMS (m/z): 587.5 [M+H]⁺; ¹H NMR (400 MHz, CDCl₃): δ 8.09 (d, J=8.8 Hz, 1H), 7.58 (d, J=8.4 Hz, 1H), 7.49 (t, J=8.0 Hz, 1H), 7.31-7.28 (m, 1H), 6.74 (s, 1H), 5.61 (dd, J=10.8, 4.4 Hz, 1H), 5.55-5.35 (m, 1H), 5.24 (dd, J=17.2, 3.6 Hz, 1H), 4.98 (d, J=13.6 Hz, 1H), 4.79 (d, J=13.2 Hz, 1H), 4.46-4.30 (m, 3H), 4.19-4.16 (m, 1H), 3.96-3.83 (m, 2H), 3.73-3.64 (m, 1H), 3.50-3.49 (m, 1H), 3.19-3.08 (m, 3H), 2.76-2.71 (m, 3H), 2.50 (br s, 3H), 2.38-2.29 (m, 2H), 2.10-2.01 (m, 2H), 1.88-1.78 (m, 4H).

### Example S-40: Synthesis of 4-(2,4-dichloro-7,8-dihydro-5H-pyrano[4,3-d]pyrimidin-7-yl) benzo[d]thiazol-2-amine

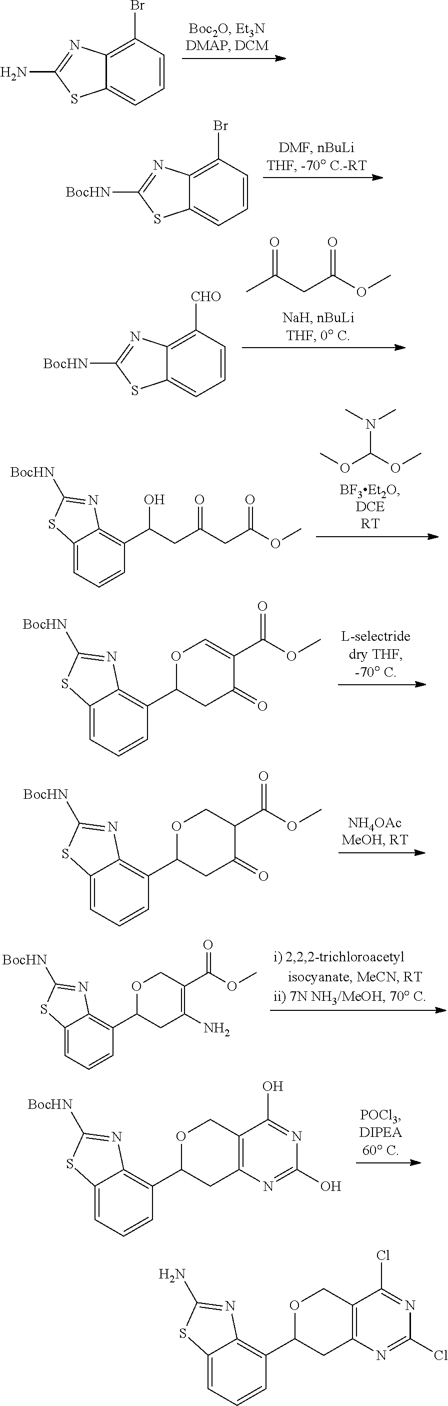

Step 1: Synthesis of tert-butyl (4-bromobenzo[d]thiazol-2-yl)carbamate. To a solution of 4-bromobenzo[d]thiazol-2-amine (80 g, 0.35 mol, 1.0 eq) in DCM (350 mL) was added Et₃N (146 mL, 1.05 mol, 3.0 eq), Boc₂O (87 mL, 0.38 mol, 1.1 eq), DMAP (42 g, 0.35 mol, 1.0 eq) and the reaction mixture was stirred at RT for 2 h. The reaction mixture was diluted with water (200 mL), extracted with DCM (3×200 mL). The combined organic layers were washed with brine (200 mL), dried over Na₂SO₄, filtered, and concentrated under reduced pressure to afford the crude material which was purified by silica gel chromatography to give the title compound. LCMS (m/z): 328.9 [M+H]⁺

Step 2: Synthesis of tert-butyl (4-formylbenzo[d]thiazol-2-yl)carbamate. To a solution of tert-butyl (4-bromobenzo[d]thiazol-2-yl)carbamate (20 g, 0.06 mol, 1.0 eq) in dry THL (80 mL) at −70° C. under nitrogen was added nBuLi (2.4 M in n-hexane; 75 mL, 0.18 mol, 3.0 eq) and stirred at the same temperature for 1 h. To the reaction mixture, DML (16.3 mL, 0.21 mol, 3.5 eq) was added and stirred at −70° C. for 2 h. The mixture was quenched with aqueous NH₄Cl (100 mL) and extracted with EtOAc (3×100 mL). The combined organic layers were washed with brine (100 mL), dried over Na₂SO₄, filtered, and concentrated under reduced pressure to afford the crude material which was purified by silica gel chromatography to give the title compound. LCMS (m/z): 279.1 [M+H]⁺

Step 3: Synthesis of methyl 5-(2-((tert-butoxycarbonyl)amino)benzo[d]thiazol-4-yl)-5-hydroxy-3-oxopentanoate. The title compound was synthesized following the procedure as described in Example S-9, Step 1 using tert-butyl (4-formylbenzo[d]thiazol-2-yl)carbamate (2.0 g, 7.18 mmol, 1.0 eq), methyl acetoacetate (1.67 g, 14.37 mmol, 2.0 eq), NaH (0.68 g, 17.23 mmol, 2.4 eq), nBuLi (2.4 M in n-hexane; 12.3 mL, 29.44 mmol, 4.1 eq). The crude material was purified by silica gel chromatography to afford the title compound. LCMS (m/z): 395.1 [M+H]⁺

Step 4: Synthesis of methyl 2-(2-((tert-butoxycarbonyl)amino)benzo[d]thiazol-4-yl)-4-oxo-3,4-dihydro-2H-pyran-5-carboxylate. The title compound was synthesized following the procedure as described in Example S-9, Step 2 using methyl 5-(2-((tert-butoxycarbonyl)amino)benzo[d]thiazol-4-yl)-5-hydroxy-3-oxopentanoate (1.0 g, 2.54 mmol, 1.0 eq) and DMEDMA (0.48 mL, 3.61 mmol, 1.4 eq). LCMS (m/z): 405.1 [M+H]⁺

Step 5: Synthesis of methyl 6-(2-((tert-butoxycarbonyl)amino)benzo[d]thiazol-4-yl)-4-oxotetrahydro-2H-pyran-3-carboxylate. The title compound was synthesized following the procedure as described in Example S-9, Step 3 using methyl 2-(2-((tert-butoxycarbonyl)amino)benzo[d]thiazol-4-yl)-4-oxo-3,4-dihydro-2H-pyran-5-carboxylate (1.0 g, 2.47 mmol, 1.0 eq) and L-selectride (1M in THE; 3.8 mL, 3.71 mmol, 1.5 eq). LCMS (m/z): 407.1 [M+H]⁺

Step 6: Synthesis of methyl 4-amino-6-(2-((tert-butoxycarbonyl)amino)benzo[d]thiazol-4-yl)-5,6-dihydro-2H-pyran-3-carboxylate. The title compound was synthesized following the procedure as described in Example S-9, Step 4 using methyl 6-(2-((tert-butoxycarbonyl)amino)benzo[d]thiazol-4-yl)-4-oxotetrahydro-2H-pyran-3-carboxylate (1.14 g, 2.80 mmol, 1.0 eq) and NH₄OAc (0.65 g, 8.41 mmol, 3.0 eq). LCMS (m/z): 406.0 [M+H]⁺

Step 7: Synthesis of tert-butyl (4-(2,4-dihydroxy-7,8-dihydro-5H-pyrano[4,3-d]pyrimidin-7-yl)benzo[d]thiazol-2-yl)carbamate. The title compound was synthesized following the procedure as described in Example S-9, Step 5 using methyl 4-amino-6-(2-((tert-butoxycarbonyl)amino)benzo[d]thiazol-4-yl)-5,6-dihydro-2H-pyran-3-carboxylate (0.31 g, 0.76 mmol, 1.0 eq), 2,2,2,-trichloroacetyl isocyanate (0.29 g, 1.52 mmol, 2.0 eq) and NH₃/MeOH (7N; 10 mL). LCMS (m/z): 417.1 [M+H]⁺

Step 8: Synthesis of 4-(2,4-dichloro-7,8-dihydro-5H-pyrano[4,3-d]pyrimidin-7-yl)benzo [d]thiazol-2-amine. To a solution of tert-butyl (4-(2,4-dihydroxy-7,8-dihydro-5H-pyrano[4,3-d]pyrimidin-7-yl)benzo[d]thiazol-2-yl)carbamate (0.50 g, 1.20 mmol, 1.0 eq) in POCl₃ (10 mL) at 0° C. under nitrogen, was added DIPEA (1 mL, 6.0 mmol, 5.0 eq) and stirred at 60° C. for 32 h. the solvent was evaporated under reduced pressure and the residue was diluted with ice water, basified to pH=8-10 using aqueous NaHCO₃ solution and extracted with DCM (3×30 mL). The combined organic layers were washed with brine (30 mL), dried over Na₂SO₄, filtered, and concentrated under reduced pressure to afford the title compound. LCMS (m/z): 353.7 [M+H]⁺

### Example S-41: Synthesis of 2-((2S)-4-(7-(2-aminobenzo[d]thiazol-4-yl)-2-(((S)-1-methyl pyrrolidin-2-yl)methoxy)-7,8-dihydro-5H-pyrano[4,3-d]pyrimidin-4-yl)-1-(2-fluoroacryloyl) piperazin-2-yl)acetonitrile (Compound 260, Isomer A and Isomer B)

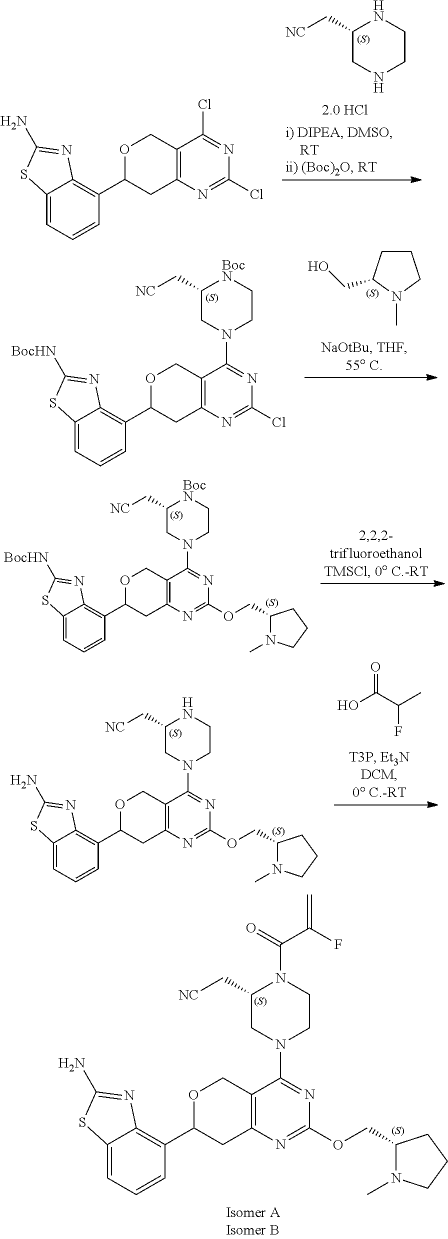

Step 1: Synthesis of tert-butyl (2S)-4-(7-(2-((tert-butoxycarbonyl)amino)benzo[d]thiazol-4-yl)-2-chloro-7,8-dihydro-5H-pyrano[4,3-d]pyrimidin-4-yl)-2-(cyanomethyl)piperazine-1-carboxylate. The title compound was synthesized following the procedure as described in Example S-2, Step 1 using 4-(2,4-dichloro-7,8-dihydro-5H-pyrano[4,3-d]pyrimidin-7-yl)benzo [d]thiazol-2-amine (0.17 g, 0.48 mmol, 1.0 eq), (S)-2-(piperazin-2-yl)acetonitrile dihydrochloride (0.09 g, 0.48 mmol, 1.0 eq), Boc₂O (0.22 mL, 0.96 mmol, 2.0 eq) and DIPEA (0.42 mL, 2.40 mmol, 5.0 eq). The crude material was purified by silica gel chromatography to afford the desired title compound. LCMS (m/z): 641.7 [M+H]⁺

Step 2: Synthesis of tert-butyl (2S)-4-(7-(2-((tert-butoxycarbonyl)amino)benzo[d]thiazol-4-yl)-2-(((S)-1-methylpyrrolidin-2-yl)methoxy)-7,8-dihydro-5H-pyrano[4,3-d]pyrimidin-4-yl)-2-(cyanomethyl)piperazine-1-carboxylate. The title compound was synthesized following the procedure as described in Example S-2, Step 2 using tert-butyl (2S)-4-(7-(2-((tert-butoxy carbonyl)amino)benzo[d]thiazol-4-yl)-2-chloro-7,8-dihydro-5H-pyrano[4,3-d]pyrimidin-4-yl)-2-(cyanomethyl)piperazine-1-carboxylate (0.10 g, 0.15 mmol, 1.0 eq), N-methyl-L-prolinol (0.027 g, 0.23 mmol, E5 eq), NaOtBu (0.022 g, 0.23 mmol, E5 eq). The crude material was purified by silica gel chromatography to afford the desired title compound. LCMS (m/z): 721.7 [M+H]⁺

Step 3: Synthesis of 2-((2S)-4-(7-(2-aminobenzo[d]thiazol-4-yl)-2-(((S)-1-methylpyrrolidin-2-yl)methoxy)-7,8-dihydro-5H-pyrano[4,3-d]pyrimidin-4-yl)piperazin-2-yl)acetonitrile. The title compound was synthesized using the procedure as described in Example S-10, step 2 using tert-butyl (2S)-4-(7-(2-((tert-butoxycarbonyl)amino)benzo[d]thiazol-4-yl)-2-(((S)-1-methyl pyrrolidin-2-yl)methoxy)-7,8-dihydro-5H-pyrano[4,3-d]pyrimidin-4-yl)-2-(cyanomethyl) piperazine-1-carboxylate (0.08 g, 0.11 mmol, TO eq). LCMS (m/z): 521.7 [M+H]⁺

Step 4: Synthesis of 2-((2S)-4-(7-(2-aminobenzo[d]thiazol-4-yl)-2-(((S)-1-methyl pyrrolidin-2-yl)methoxy)-7,8-dihydro-5H-pyrano[4,3-d]pyrimidin-4-yl)-1-(2-fluoroacryloyl) piperazin-2-yl)acetonitrile. The title compound was synthesized following the procedure as described in Example S-10, Step 3 using 2-((2S)-4-(7-(2-aminobenzo[d]thiazol-4-yl)-2-(((S)-1-methyl pyrrolidin-2-yl)methoxy)-7,8-dihydro-5H-pyrano[4,3-d]pyrimidin-4-yl)piperazin-2-yl) acetonitrile (0.045 g, 0.08 mmol, TO eq), 2-fluoro acrylic acid (0.015 g, 0.17 mmol, 2.0 eq), T3P (50% in EtOAc; 0.10 mL, 0.17 mmol, 2.0 eq) and Et₃N (0.04 mL, 0.26 mmol, 3.0 eq). The crude material was purified by prep HPLC and SFC to give the title compound as Isomer A and Isomer B.

Isomer A: LCMS (m/z): 593.7 [M+H]⁺; tH NMR (400 MHz, CDCl₃): δ 7.57 (d, J=8.0 Hz, 1H), 7.45 (d, J=7.2 Hz, 1H), 7.18 (t, J=7.6 Hz, 1H), 5.45 (dd, J=11.2, 3.6 Hz, 1H), 5.36 (br s, 1H), 5.26-5.22 (m, 3H), 4.92 (d, J=13.6 Hz, 1H), 4.79 (d, J=13.6 Hz, 1H), 4.39 (dd, J=10.4, 5.2 Hz, 1H), 4.15 (dd, J=10.4, 6.8 Hz, 1H), 3.94 (d, J=14.4 Hz, 1H), 3.75 (d, J=12.0 Hz, 1H), 3.49 (br s, 1H), 3.34 (dd, J=18.4, 3.2 Hz, 1H), 3.08-3.00 (m, 4H), 2.79-2.68 (m, 4H), 2.48 (s, 3H), 2.29-2.27 (m, 2H), 2.07-2.01 (m, 2H).

Isomer B: LCMS (m/z): 593.6 [M+H]⁺; tH NMR (400 MHz, CDCl₃): δ 7.57 (d, J=6.8 Hz, 1H), 7.42 (d, J=7.2 Hz, 1H), 7.18 (t, J=8.0 Hz, 1H), 5.45 (dd, J=10.8, 4.0 Hz, 1H), 5.35 (br s, 1H), 5.27-5.23 (m, 3H), 4.90 (d, J=13.6 Hz, 1H), 4.80 (d, J=13.6 Hz, 1H), 4.39-4.20 (m, 2H), 3.94-3.90 (m, 1H), 3.72 (d, J=10.9 Hz, 1H), 3.31 (dd, J=18.4, 4.0 Hz, 1H), 3.17-3.00 (m, 6H), 2.94-2.84 (m, 2H), 2.71 (br s, 1H), 2.50 (br s, 3H), 2.37-2.25 (m, 2H), 2.10-1.99 (m, 2H).

### Example S-42: Synthesis of 2,4-dichloro-7-(8-chloronaphthalen-1-yl)-8,8-dimethyl-7,8-dihydro-5H-pyrano[4,3-d]pyrimidine

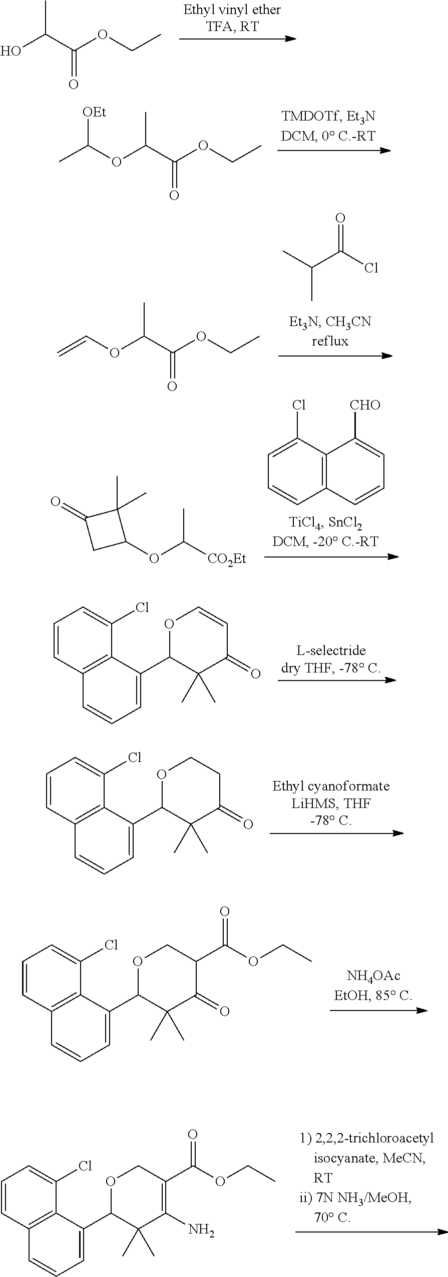

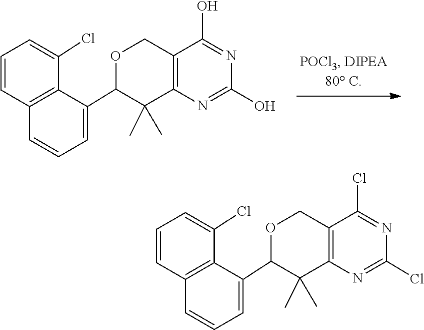 [SMILES: CC1(C)c2nc(O)nc(O)c2COC1c1cccc2cccc(Cl)c12 ; CC1(C)c2nc(Cl)nc(Cl)c2COC1c1cccc2cccc(Cl)c12]

Step 1: Synthesis of ethyl 2-(l-ethoxyethoxy)propanoate. To a solution of ethyl 2-hydroxy propanoate (50 g, 423.26 mmol, 1.0 eq) in ethyl vinyl ether (100 mL) was added TFA (1.61 mL, 21.16 mmol, 0.05 eq) and the reaction mixture was stirred at RT for 23 h. The reaction mixture was quenched with saturated NaHCO₃ solution (500 mL) and extracted with EtOAc (3×200 mL). The combined organic layers were washed with brine, dried over Na₂SO₄, filtered, and concentrated under reduced pressure to give the crude material which was purified by silica gel chromatography to afford the title compound.

Step 2: Synthesis of ethyl 2-(vinyloxy)propanoate. To a solution of ethyl 2-(l-ethoxyethoxy) propanoate (71.3 g, 374.78 mmol, 1.0 eq) in DCM (200 mL) at 0° C. was added Et₃N (73.1 mL, 524.70 mmol, 1.4 eq), TMSOTf (74.8 mL, 412.26 mmol, 1.1 eq) and stirred at RT for 30 min. The reaction mixture was quenched with saturated NaHCO₃ solution (500 mL) and extracted with DCM (3×200 mL). The combined organic layers were washed with brine, dried over Na₂SO₄, filtered, and concentrated under reduced pressure to give the crude material which was purified by silica gel chromatography to afford the title compound.

Step 3: Synthesis of ethyl 2-(2,2-dimethyl-3-oxocyclobutoxy)propanoate. To a solution of ethyl 2-(vinyloxy)propanoate (10 g, 69.36 mmol, 1.0 eq) in CH₃CN (20 mL) was added Et₃N (14.5 mL, 104.04 mmol, 1.5 eq) followed by dropwise addition of isobutyryl chloride (10.9 mL, 104.04 mmol, 1.5 eq) in CH₃CN (100 mL) over 1 h at reflux and the reaction mixture was stirred at reflux for 1 h. The reaction mixture was cooled to RT, MTBE (100 mL) was added, and the resulting mixture was filtered through a Celite pad. The filtrate was washed with water (100 mL) and brine (100 mL), dried over Na₂SO₄, filtered, and concentrated under reduced pressure to give the crude material which was purified by silica gel chromatography to afford the title compound.

Step 4: Synthesis of 2-(8-chloronaphthalen-1-yl)-3,3-dimethyl-2,3-dihydro-4H-pyran-4-one. To a suspension of TiCl₄ (1 N in DCM; 11.75 mL, 11.75 mmol, 1.6 eq) in DCM (15 mL) at −20° C., was added 8-chloro-1-naphthaldehyde (1.40 g, 7.34 mmol, 1.0 eq) in DCM (10 mL) followed by the addition of ethyl 2-(2,2-dimethyl-3-oxocyclobutoxy)propanoate (2.51 g, 11.75 mmol, 1.6 eq) in DCM (10 mL) and the reaction mixture was stirred at RT for 18 h. The reaction mixture was diluted with water (100 mL) and extracted with DCM (3×50 mL). The combined organic layers were washed with brine, dried over Na₂SO₄, filtered, and concentrated under reduced pressure to give the crude material which was purified by silica gel chromatography to afford the title compound. LCMS (m/z): 287.0 [M+H]⁺

Step 5: Synthesis of 2-(8-chloronaphthalen-1-yl)-3,3-dimethyltetrahydro-4H-pyran-4-one. To a solution of 2-(8-chloronaphthalen-1-yl)-3,3-dimethyl-2,3-dihydro-4H-pyran-4-one (1.20 g, 4.18 mmol, 1.0 eq) in dry THF (25 mL) at −78° C. was added L-selectride (1M in THF; 4.18 mL, 4.18 mmol, 1.0 eq) and stirred for 30 min. The reaction mixture was quenched with saturated NH₄Cl solution (50 mL) and extracted with EtOAc (3×50 mL). The combined organic layers were washed with brine, dried over Na₂SO₄, filtered, and concentrated under reduced pressure to give the crude material which was purified by silica gel chromatography to afford the title compound.

Step 6: Synthesis of ethyl 6-(8-chloronaphthalen-1-yl)-5,5-dimethyl-4-oxotetrahydro-2H-pyran-3-carboxylate. To a solution of 2-(8-chloronaphthalen-1-yl)-3,3-dimethyltetrahydro-4H-pyran-4-one (0.65 g, 2.25 mmol, 1.0 eq) in dry THF (15 mL) at −78° C., was added LiHMDS (1N in THF; 2.47 mL, 2.47 mmol, 1.1 eq) and stirred for 1 h. Ethyl cyanoformate (0.24 mL, 2.47 mmol, 1.1 eq) was added to the reaction mixture at −78° C. and stirred at the same temperature for 2 h. The reaction mixture was quenched with saturated NH₄Cl solution (20 mL) and extracted with EtOAc (3×20 mL). The combined organic layers were washed with brine, dried over Na₂SO₄, filtered, and concentrated under reduced pressure to give the crude material which was purified by silica gel chromatography to afford the title compound.

Step 7: Synthesis of ethyl 4-amino-6-(8-chloronaphthalen-1-yl)-5,5-dimethyl-5,6-dihydro-2H-pyran-3-carboxylate. The title compound was synthesized following the procedure as described in Example S-9, Step 4 using ethyl 6-(8-chloronaphthalen-1-yl)-5,5-dimethyl-4-oxotetrahydro-2H-pyran-3-carboxylate (0.38 g, 1.05 mmol, 1.0 eq) and NH₄OAc (0.24 g, 3.16 mmol, 3.0 eq) in ethanol (15 mL) under reflux. LCMS (m/z): 360.0 [M+H]⁺

Step 8: Synthesis of 7-(8-chloronaphthalen-1-yl)-8,8-dimethyl-7,8-dihydro-5H-pyrano[4,3-d]pyrimidine-2,4-diol. The title compound was synthesized following the procedure as described in Example S-9, Step 5 using ethyl 4-amino-6-(8-chloronaphthalen-1-yl)-5,5-dimethyl-5,6-dihydro-2H-pyran-3-carboxylate (0.40 g, 1.11 mmol, 1.0 eq), 2,2,2,-trichloroacetyl isocyanate (0.26 mL, 2.22 mmol, 2.0 eq). LCMS (m/z): 357.2 [M+H]⁺

Step 9: Synthesis of 2,4-dichloro-7-(8-chloronaphthalen-1-yl)-8,8-dimethyl-7,8-dihydro-5H-pyrano[4,3-d]pyrimidine. The title compound was synthesized following the procedure as described in Example S-1, Step 7 using 7-(8-chloronaphthalen-1-yl)-8,8-dimethyl-7,8-dihydro-5H-pyrano[4,3-d]pyrimidine-2,4-diol (0.30 g, 0.84 mmol, 1.0 eq). The crude material was purified by silica gel chromatography to afford the title compound.

### Example S-43: Synthesis of 2-((2S)-4-(7-(8-chloronaphthalen-1-yl)-8,8-dimethyl-2-(((S)-1-methylpyrrolidin-2-yl)methoxy)-7,8-dihydro-5H-pyrano[4,3-d]pyrimidin-4-yl)-1-(2-fluoro acryloyl)piperazin-2-yl)acetonitrile (Compound 258, Isomer A and Isomer B)

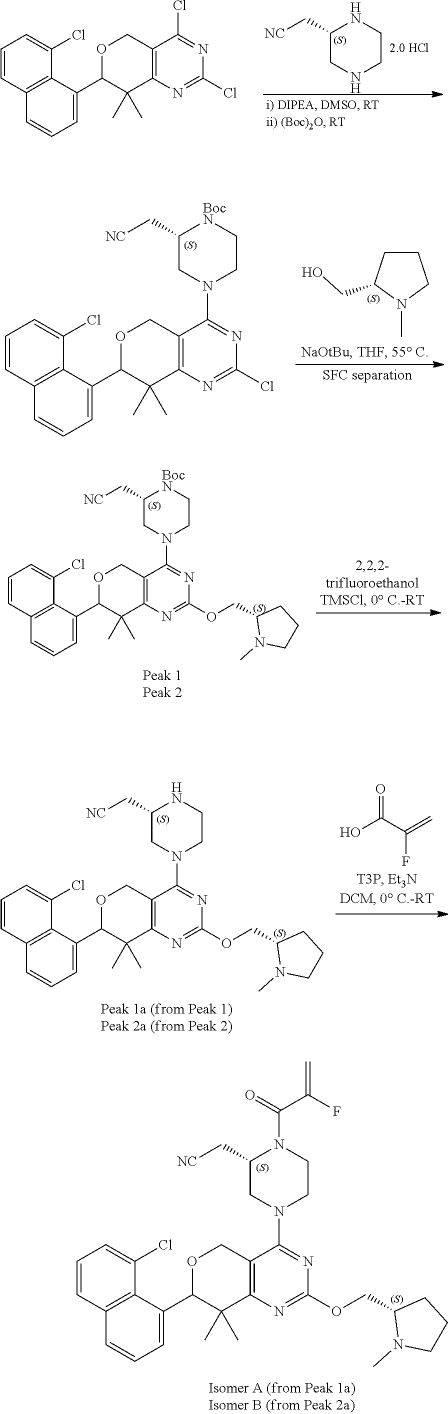

Step 1: Synthesis of tert-butyl (2S)-4-(2-chloro-7-(8-chloronaphthalen-1-yl)-8,8-dimethyl-7,8-dihydro-5H-pyrano[4,3-d]pyrimidin-4-yl)-2-(cyanomethyl)piperazine-1-carboxylate. The title compound was synthesized following the procedure as described in Example S-2, step 1 using 2,4-dichloro-7-(8-chloronaphthalen-1-yl)-8,8-dimethyl-7,8-dihydro-5H-pyrano[4,3-d]pyrimidine (0.17 g, 0.43 mmol, 1.0 eq), (S)-2-(piperazin-2-yl)acetonitrile dihydrochloride (0.09 g, 0.47 mmol, 1.1 eq), Boc₂O (0.15 mL, 0.64 mmol, 1.5 eq) and DIPEA (0.37 mL, 2.15 mmol, 5.0 eq). The crude material was purified by silica gel chromatography to afford the desired title compound. LCMS (m/z): 641.7 [M+H]⁺

Step 2: Synthesis of tert-butyl (2S)-4-(7-(8-chloronaphthalen-1-yl)-8,8-dimethyl-2-(((S)-1-methylpyrrolidin-2-yl)methoxy)-7,8-dihydro-5H-pyrano[4,3-d]pyrimidin-4-yl)-2-(cyanomethyl)piperazine-1-carboxylate (Peak 1 and Peak 2). The title compound was synthesized following the procedure as described in Example S-2, step 2 using tert-butyl (2S)-4-(2-chloro-7-(8-chloronaphthalen-1-yl)-8,8-dimethyl-7,8-dihydro-5H-pyrano[4,3-d]pyrimidin-4-yl)-2-(cyanomethyl)piperazine-1-carboxylate (0.18 g, 0.30 mmol, 1.0 eq), N-methyl-L-prolinol (0.06 mL, 0.46 mmol, 1.5 eq) and NaOtBu (0.04 g, 0.46 mmol, 1.5 eq). The crude material was purified by silica gel chromatography to afford the title compound as a mixture of diastereomers. The diastereomeric mixture was separated by SFC to give the title compound as Peak 1 and Peak 2.

Step 3: Synthesis of 2-((2S)-4-(7-(8-chloronaphthalen-1-yl)-8,8-dimethyl-2-(((S)-1-methyl pyrrolidin-2-yl)methoxy)-7,8-dihydro-5H-pyrano[4,3-d]pyrimidin-4-yl)piperazin-2-yl) acetonitrile (Peak 1a and Peak 2a). The title compound Peak 1a was synthesized using the procedure as described in Example S-10, step 2 using tert-butyl (2S)-4-(7-(8-chloronaphthalen-1-yl)-8,8-dimethyl-2-(((S)-1-methylpyrrolidin-2-yl)methoxy)-7,8-dihydro-5H-pyrano[4,3-d]pyrimidin-4-yl)-2-(cyanomethyl)piperazine-1-carboxylate (Peak 1; 0.043 g, 0.06 mmol, 1.0 eq). LCMS (m/z): 561.1 [M+H]⁺

The title compound Peak 2a was synthesized using the procedure as described in Example S-10, step 2 using tert-butyl (2S)-4-(7-(8-chloronaphthalen-1-yl)-8,8-dimethyl-2-(((S)-1-methyl pyrrolidin-2-yl)methoxy)-7,8-dihydro-5H-pyrano[4,3-d]pyrimidin-4-yl)-2-(cyanomethyl) piperazine-1-carboxylate (Peak 2; 0.04 g, 0.06 mmol, 1.0 eq). LCMS (m/z): 561.1 [M+H]⁺

Step 4: Synthesis of 2-((2S)-4-(7-(8-chloronaphthalen-1-yl)-8,8-dimethyl-2-(((S)-1-methyl pyrrolidin-2-yl)methoxy)-7,8-dihydro-5H-pyrano[4,3-d]pyrimidin-4-yl)-1-(2-fluoro acryloyl)piperazin-2-yl)acetonitrile (Isomer A and Isomer B). The title compound (Isomer A) was synthesized following the procedure as described in Example S-10, Step 3 using 2-((2S)-4-(7-(8-chloronaphthalen-1-yl)-8,8-dimethyl-2-(((S)-1-methyl pyrrolidin-2-yl)methoxy)-7,8-dihydro-5H-pyrano[4,3-d]pyrimidin-4-yl)piperazin-2-yl) acetonitrile (Peak 1a; 0.04 g, 0.07 mmol, 1.0 eq), 2-fluoro acrylic acid (0.01 g, 0.11 mmol, 1.5 eq), T3P (50% in EtOAc; 0.08 mL, 0.14 mmol, 2.0 eq) and Et₃N (0.03 mL, 0.21 mmol, 3.0 eq). The crude material was purified by prep HPLC to afford the title compound as Isomer A.

Isomer A: LCMS (m/z): 633.1 [M+H]⁺; ¹H NMR (400 MHz, CDCl₃): δ 7.89-7.85 (m, 2H), 7.82 (dd, J=8.0, 1.2 Hz, 1H), 7.52 (dd, J=7.2, 1.2 Hz, 1H), 7.47 (d, J=8.0 Hz, 1H), 7.31 (t, J=8.0 Hz, 1H), 5.29-5.11 (m, 3H), 4.79-4.69 (m, 4H), 4.65-4.61 (m, 2H), 4.45-4.41 (m, 1H), 4.21 (d, J=14.4 Hz, 1H), 3.81-3.78 (m, 2H), 3.61-3.55 (m, 1H), 3.15-2.96 (m, 6H), 2.28-2.15 (m, 2H), 2.11-1.92 (m, 4H), 1.02 (s, 3H), 0.78 (s, 3H).

The title compound (Isomer B) was synthesized following the procedure as described in Example S-10, Step 3 using 2-((2S)-4-(7-(8-chloronaphthalen-1-yl)-8,8-dimethyl-2-(((S)-1-methyl pyrrolidin-2-yl)methoxy)-7,8-dihydro-5H-pyrano[4,3-d]pyrimidin-4-yl)piperazin-2-yl) acetonitrile (Peak 2a; 0.04 g, 0.07 mmol, 1.0 eq), 2-fluoro acrylic acid (0.009 g, 0.10 mmol, 1.5 eq), T3P (50% in EtOAc; 0.08 mL, 0.14 mmol, 2.0 eq) and Et₃N (0.03 mL, 0.20 mmol, 3.0 eq). The crude material was purified by prep HPLC to afford the title compound as Isomer B.

Isomer B: LCMS (m/z): 633.1 [M+H]⁺; ¹H NMR (400 MHz, CDCl₃): δ 7.90-7.81 (m, 3H), 7.51 (dd, J=7.6, 1.2 Hz, 1H), 7.48 (d, J=8.0 Hz, 1H), 7.31 (t, J=8.0 Hz, 1H), 5.29-5.12 (m, 3H), 4.85-4.68 (m, 4H), 4.63 (dd, J=12.8, 3.2 Hz, 1H), 4.43 (dd, J=12.8, 6.8 Hz, 1H), 4.01-3.98 (m, 1H), 3.86-3.83 (m, 1H), 3.80-3.71 (m, 1H), 3.61-3.41 (m, 1H), 3.39-3.16 (m, 1H), 3.04-2.86 (m, 6H), 2.30-2.05 (m, 3H), 2.00-1.91 (m, 3H), 1.00 (s, 3H), 0.77 (s, 3H).

### Example S-44: Synthesis of 2-((2S)-1-(but-2-ynoyl)-4-(7-(8-chloronaphthalen-1-yl)-2-((tetra hydro-1H-pyrrolizin-7a(5H)-yl)methoxy)-7,8-dihydro-5H-pyrano[4,3-d]pyrimidin-4-yl) piperazin-2-yl)acetonitrile (Compound 279, Isomer A and Isomer B)

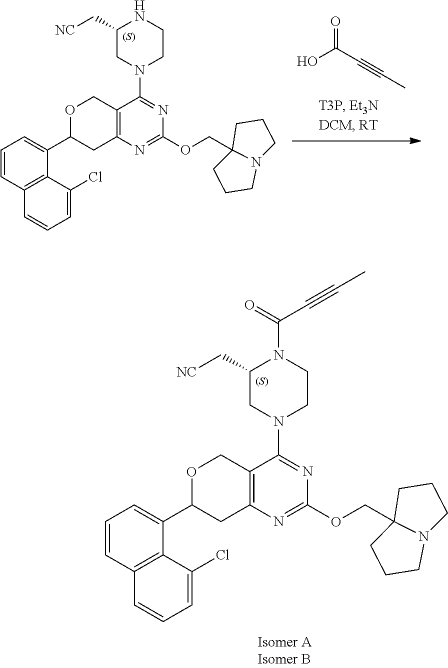

Step 1: Synthesis of 2-((2S)-1-(but-2-ynoyl)-4-(7-(8-chloronaphthalen-1-yl)-2-((tetra hydro-1H-pyrrolizin-7a(5H)-yl)methoxy)-7,8-dihydro-5H-pyrano[4,3-d]pyrimidin-4-yl) piperazin-2-yl)acetonitrile. The title compound was synthesized following the procedure as described in Example S-10, Step 3 using 2-((2S)-4-(7-(8-chloronaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl) acetonitrile (0.29 g, 0.52 mmol, 1.0 eq), but-2-ynoic acid (0.13 g, 1.56 mmol, 3.0 eq), T3P (50% in EtOAc; 0.62 mL, 1.04 mmol, 2.0 eq) and Et₃N (0.22 mL, 1.56 mmol, 3.0 eq). The crude material was purified by prep HPLC and SFC to afford the title compound as Isomer A and Isomer B.

Isomer A: LCMS (m/z): 625.2 [M+H]⁺; ¹H NMR (400 MHz, CDCl₃): δ 7.99-7.89 (m, 1H), 7.81 (t, J=8.8 Hz, 2H), 7.59-7.57 (m, 1H), 7.54 (t, J=7.8 Hz, 1H), 7.35 (t, J=7.2 Hz, 1H), 6.45 (dd, J=10.8, 3.2 Hz, 1H), 5.04-4.91 (m, 2H), 4.84-4.77 (m, 1H), 4.61-4.33 (m, 1H), 4.32-3.83 (m, 3H), 3.83-3.67 (m, 1H), 3.59 (dd, J=18.1, 3.2 Hz, 1H), 3.49-3.00 (m, 5H), 2.94-2.80 (m, 2H), 2.78-2.55 (m, 3H), 2.20-2.08 (m, 2H), 2.07-2.05 (m, 3H), 1.98-1.82 (m, 4H), 1.77-1.62 (m, 2H).

Isomer B: LCMS (m/z): 625.2 [M+H]⁺; ¹H NMR (400 MHz, CDCl₃): δ 7.95 (dd, J=7.6, 0.8 Hz, 1H), 7.81 (dd, J=10.1, 8.5 Hz, 2H), 7.58-7.56 (m, 1H), 7.57-7.52 (m, 1H), 7.35 (td, J=8.0, 1.6 Hz, 1H), 6.49 (dd, J=10.8, 3.2 Hz, 1H), 5.08-4.86 (m, 2H), 4.82-4.78 (m, 1H), 4.60-4.32 (m, 1H), 4.31-4.08 (m, 2H), 3.95 (dd, J=13.9, 2.1 Hz, 1H), 3.71-3.59 (m, 3H), 3.47-3.30 (m, 2H), 3.21-3.06 (m, 1H), 2.98-2.79 (m, 3H), 2.78-2.63 (m, 3H), 2.25-2.15 (m, 2H), 2.09-2.04 (m, 3H), 2.10-1.92 (m, 4H), 1.82-1.73 (m, 2H).

### Example S-45: Synthesis of 2-((2S)-1-((E)-but-2-enoyl)-4-(7-(8-chloronaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile (Compound 278, Isomer A and Isomer B)

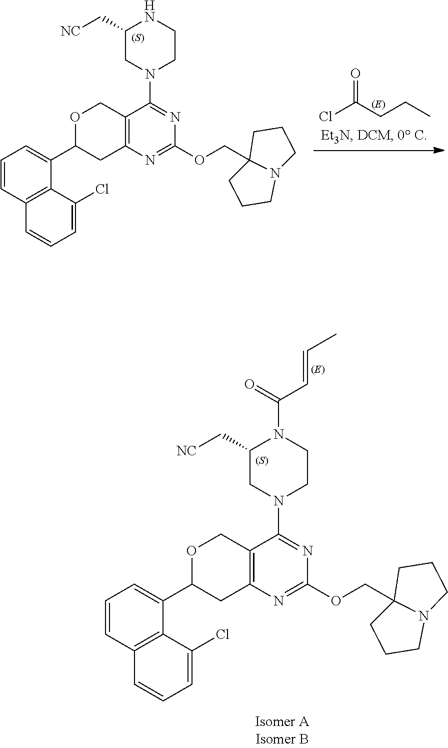

Step 1: Synthesis of 2-((2S)-1-((E)-but-2-enoyl)-4-(7-(8-chloronaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile. To a solution of 2-((2S)-4-(7-(8-chloronaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl) acetonitrile (0.16 g, 0.28 mmol, 1.0 eq) and Et₃N (0.12 mL, 0.84 mmol, 3.0 eq) in DCM (8 mL) at 0° C., was added (E)-but-2-enoyl chloride (0.03 mL, 0.28 mmol, 1.0 eq) and stirred at the same temperature for 3 min. The reaction mixture was quenched with saturated NaHCO₃ solution (10 mL) and extracted with DCM (2×20 mL). The combined organic layers were washed with brine, dried over Na₂SO₄, filtered, and concentrated under reduced pressure to give the crude material which was purified by prep HPLC and SEC to afford the title compound as Isomer A and Isomer B.

Isomer A: LCMS (m/z): 627.2 [M+H]⁺; ¹H NMR (400 MHz, CDCl₃): δ 7.94 (d, J=7.6 Hz, 1H), 7.81 (t, J=8.4 Hz, 2H), 7.62-7.49 (m, 2H), 7.35 (t, J=7.8 Hz, 1H), 7.01-6.92 (m, 0.5H), 6.45 (dd, J=10.9, 3.1 Hz, 1H), 5.99-5.89 (m, 0.5H), 5.33-5.21 (m, 1H), 5.09-4.90 (m, 2H), 4.86-4.75 (m, 1H), 4.50-4.10 (m, 2H), 3.97-3.65 (m, 3H), 3.59 (dd, J=18.1, 2.6 Hz, 1H), 3.42-3.28 (m, 2H), 3.25-3.09 (m, 4H), 2.88-2.53 (m, 4H), 2.19-1.92 (m, 7H), 1.76-1.60 (m, 4H).

Isomer B: LCMS (m/z): 627.2 [M+H]⁺; ¹H NMR (400 MHz, CDCl₃): δ 7.95 (d, J=7.2 Hz, 1H), 7.81 (t, J=9.2 Hz, 2H), 7.62-7.50 (m, 2H), 7.35 (t, J=7.8 Hz, 1H), 6.98-6.92 (m, 0.5H), 6.49 (dd, J=11.2, 3.2 Hz, 1H), 6.00-5.89 (m, 0.5H), 5.28-5.14 (m, 1H), 5.11-4.82 (m, 2H), 4.81 (d, J=13.6 Hz, 1H), 4.50-4.20 (m, 2H), 4.05-3.90 (m, 2H), 3.79-3.51 (m, 4H), 3.48-3.27 (m, 2H), 3.22-3.03 (m, 2H), 2.93-2.66 (m, 4H), 2.21-1.95 (m, 9H), 1.87-1.80 (m, 2H).

### Example S-46: Synthesis of 2-((2S)-1-(2-chloroacryloyl)-4-(7-(8-chloronaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile (Compound 277, Isomer A and Isomer B)

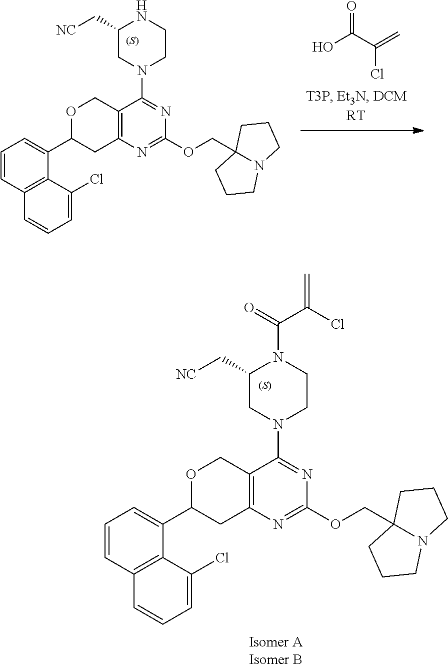

Step 1: Synthesis of 2-((2S)-1-(2-chloroacryloyl)-4-(7-(8-chloronaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl)acetonitrile. The title compound was synthesized following the procedure as described in Example S-10, Step 3 using 2-((2S)-4-(7-(8-chloronaphthalen-1-yl)-2-((tetrahydro-1H-pyrrolizin-7a(5H)-yl)methoxy)-7,8-dihydro-5H-pyrano[4,3-d]pyrimidin-4-yl)piperazin-2-yl) acetonitrile (0.12 g, 0.21 mmol, 1.0 eq), 2-chloroacrylic acid (0.046 g, 0.43 mmol, 2.0 eq), T3P (50% in EtOAc; 0.26 mL, 0.43 mmol, 2.0 eq) and Et₃N (0.06 mL, 0.43 mmol, 2.0 eq). The crude material was purified by prep HPLC and SFC to afford the title compound as Isomer A and Isomer B.

Isomer A: LCMS (m/z): 647.1 [M+H]⁺; ¹H NMR (400 MHz, CDCl₃): δ 7.96 (d, J=7.1 Hz, 1H), 7.82-7.79 (m, 2H), 7.59-7.52 (m, 2H), 7.35 (t, J=8.0 Hz, 1H), 6.48 (dd, J=10.8, 3.2 Hz, 1H), 5.78 (br s, 1H), 5.72 (d, J=1.9 Hz, 1H), 4.98 (d, J=14.0 Hz, 1H), 4.80 (d, J=13.2 Hz, 1H), 4.50 (br s, 2H), 4.14-4.01 (m, 2H), 3.91-3.78 (m, 2H), 3.69-3.52 (m, 5H), 3.09-2.82 (m, 6H), 2.32-2.20 (m, 2H), 2.15-2.05 (m, 2H), 2.03-1.95 (m, 2H), 1.89-1.83 (m, 2H).

Isomer B: LCMS (m/z): 647.1 [M+H]⁺; ¹H NMR (400 MHz, CDCl₃): δ 7.92 (d, J=7.2 Hz, 1H), 7.83 (dd, J=13.6, 8.0 Hz, 2H), 7.61-7.54 (m, 2H), 7.37 (t, J=7.6 Hz, 1H), 6.54-6.50 (m, 1H), 5.73-5.70 (m, 2H), 5.01-4.65 (m, 3H), 4.49 (d, J=10.4 Hz, 1H), 3.92-3.70 (m, 4H), 3.42-3.40 (m, 2H), 3.36-3.34 (m, 2H), 2.96-2.94 (m, 7H), 2.52-2.50 (m, 2H), 2.37-2.28 (m, 2H), 2.10-2.08 (m, 2H), 1.96-1.93 (m, 2H).

It is understood that compounds disclosed herein are synthesized using the general synthetic schemes 1 to 8 or using the experimental details as described above. The steps involved in the synthetic routes are clearly familiar to those skilled in the art, wherein the substituents described in the formulae disclosed herein can be varied with a choice of appropriate starting materials and reagents utilized in the steps presented.

### BIOLOGICAL EXAMPLES

### Example B1. Nucleotide Exchange Assay

Method I. A biochemical assay to assess activity of test compounds in inhibiting SOS1-mediated exchange of GDP for GTP on the KRAS G12C protein is performed at Reaction Biology Corporation (Malvern, Pa., USA). In this assay, exchange of BODIPY-GDP to GTP results in the decrease in BODIPY-GDP fluorescence. Briefly, recombinant KRAS G12C protein (aa 2-169, prepared by Reaction Biology) is labelled with 5× excess of BODIPY-GDP. Excess BODIPY-GDP is separated from loaded protein using a gravity desalting column. Compounds are incubated with 0.15 μM KRAS G12C/BODIPY-GDP for 30 minutes. A mixture of GTP (25 μM) and SOS1 (70 nM; aa 564-1049, prepared by Reaction Biology) is added to the reaction to initiate the nucleotide exchange reaction. Reaction progress is monitored in real time (Ex/Em=(483/530)) in CLARIOstar (BMG LabTech). After signal normalization, the data are fitted using the “one phase exponential decay” model in GraphPad Prism and rate constants obtained. Rate constants are converted to % activity values which are fit to a 4-parameter sigmoidal dose-response curve in Prism to derive IC₅₀ values for inhibition of exchange activity by test compounds.

Method II. Alternatively or additionally, an assay using Mant-GDP instead of BODIPY-GDP is performed at Reaction Biology Corporation (Malvern, Pa., USA). In this assay, exchange of Mant-GDP to GTP results in the decrease in Mant-GDP fluorescence. Briefly, recombinant KRAS G12C protein (aa 2-169, prepared by Reaction Biology) is labelled with 10× excess of Mant-GDP. Excess Mant-GDP is separated from loaded protein using a desalting column (AKTA system). Compounds are incubated with 0.5 μM KRAS G12C/Mant GDP for 30 minutes. GTP (25 μM) is added to the reaction mixture and the mixture further incubated for 5 minutes. 100 nM SOS1 (aa 564-1049, prepared by Reaction Biology) is added to the reaction to initiate nucleotide exchange reaction. Reaction progress is monitored in real time (Ex/Em=(355/460)) in CLARIOstar (BMG LabTech). After signal normalization, the data are fitted using the “one phase exponential decay” model in GraphPad Prism and rate constants obtained. Rate constants are converted to % activity values which are fit to a 4-parameter sigmoidal dose-response curve in Prism to derive IC₅₀ values for inhibition of exchange activity by test compounds.

Method III. Inhibition of SOS1-mediated exchange of GDP for GTP on the KRAS G12C protein was measured in a biochemical assay that monitors association of KRAS G12C protein with the Ras-binding domain of Raf (Raf-RBD), where the association is coupled to exchange of GDP to GTP on the KRAS G12C protein. The assay was performed at WuXi AppTec (Shanghai, China). Compounds were serially diluted in 3-fold dilutions to 11 concentrations and spotted into the wells of an assay plate in a total volume of 150 nL DMSO per well. A 2× enzyme mix was prepared by diluting GDP-bound, His-tagged KRAS G12C to 25 nM in assay buffer. 10 μL/well of 2× enzyme mix was added to all wells, and the plate was sealed and incubated overnight (18 hr) at 23° C. 4×SOS1/GDP and 4×SOS1/GTP were prepared in assay buffer. 5 μL/well of 4×SOS1/GDP mix was added to negative control wells, 5 μL/well of 4×SOS1/GTP mix was added to the remaining wells, and the plate was further incubated at 23° C. for 1 hr. Finally, FLAG-tagged Raf-RBD is added to the reaction mixture along with a pair of FRET donor- and acceptor-conjugated antibodies specific for the FLAG-tagged Raf-RBD and His-tagged KRAS G12C proteins. A 4×Raf-RBD/Tb-anti-FLAG/D2-anti-His mix was prepared in assay buffer, and 5 μL/well of the mix was added to all wells, followed by incubation at room temperature for 2 hr. The TR-FRET signals, which increase with association of KRAS G12C and Raf-RBD, were measured on a Perkin Elmer Envision 2104 plate reader, and data were analyzed by non-linear regression to obtain IC50 values for inhibition by compounds. Table 2 includes results of the assay of Method III. ‘+++’ indicates IC₅₀ values≤300 nM, ‘++’ indicates IC₅₀ values between 300 nM-3 μM, and ‘+’ indicates IC₅₀ values≥3 μM.

TABLE 2
Nucleotide exchange assay results

| Compound No. | IC₅₀ | Method |
|---|---|---|
| 38 (Isomer A) | ++ | III |
| 38 (Isomer B) | +++ | III |
| 40 (Isomer A) | +++ | III |
| 40 (Isomer B) | + | III |
| 42 (Isomer A) | + | III |
| 42 (Isomer B) | + | III |
| 43 (Isomer A) | + | III |
| 43 (Isomer B) | +++ | III |
| 44 (Isomer A) | + | III |
| 44 (Isomer B) | ++ | III |
| 52 (Isomer A) | ++ | III |
| 52 (Isomer B) | +++ | III |
| 64 (Isomer A) | ++ | III |
| 64 (Isomer B) | ++ | III |
| 135 (Isomer A) | + | III |
| 135 (Isomer B) | + | III |
| 136 (Isomer A) | + | III |
| 136 (Isomer B) | + | III |
| 154 | + | III |
| 174 | + | III |
| 178 | +++ | III |
| 181 | +++ | III |
| 228 (Isomer A) | +++ | III |
| 228 (Isomer B) | + | III |
| 229 (Isomer A) | +++ | III |
| 229 (Isomer B) | + | III |
| 230 (Isomer A) | + | III |
| 230 (Isomer B) | ++ | III |
| 231 (Isomer A) | + | III |
| 231 (Isomer B) | + | III |
| 232 (Isomer A) | ++ | III |
| 232 (Isomer B) | + | III |
| 233 (Isomer A) | + | III |
| 233 (Isomer B) | + | III |
| 234 (Isomer A) | + | III |
| 234 (Isomer B) | ++ | III |
| 235 (Isomer A) | + | III |
| 235 (Isomer B) | + | III |
| 237 (Isomer A) | + | III |
| 237 (Isomer B) | + | III |
| 238 (Isomer A) | + | III |
| 238 (Isomer B) | + | III |
| 239 (Isomer A) | + | III |
| 239 (Isomer B) | +++ | III |
| 240 (Isomer A) | + | III |
| 240 (Isomer B) | + | III |
| 241 (Isomer A) | +++ | III |
| 241 (Isomer B) | ++ | III |
| 242 (Isomer A) | + | III |
| 242 (Isomer B) | +++ | III |
| 243 (Isomer A) | + | III |
| 243 (Isomer B) | +++ | III |
| 249 (Isomer A) | ++ | III |
| 249 (Isomer B) | + | III |
| 250 (Isomer A) | ++ | III |
| 250 (Isomer B) | ++ | III |
| 258 (Isomer A) | + | III |
| 258 (Isomer B) | +++ | III |
| 259 (Isomer A) | ++ | III |
| 259 (Isomer B) | +++ | III |
| 260 (Isomer A) | +++ | III |
| 260 (Isomer B) | +++ | III |
| 266 (Isomer A) | ++ | III |
| 266 (Isomer B) | +++ | III |
| 267 (Isomer A) | ++ | III |
| 267 (Isomer B) | +++ | III |
| 272 (Isomer A) | + | III |
| 272 (Isomer B) | + | III |
| 272 (Isomer C) | +++ | III |
| 272 (Isomer D) | ++ | III |

### Example B2. Determination of Potency of Compounds in Cell Proliferation Assays

MIA PaCa-2 (ATCC CRL-1420), a pancreatic carcinoma cell line having the KRAS G12C mutation, is seeded in medium (DMEM with 10% FBS and 2.5% horse serum) at a cell count of 1700 cells per 100 μL per well in a 96 well edge plate (167425; ThermoFisher). Cells are incubated at 37° C. for 24 hr in 5% CO₂ environment (culture conditions) in a Nuaire incubator (humidified). Serially diluted test compounds (100 μL) within the desired testing concentration ranges are added to the culture plate and the cells are further incubated in culture conditions for 72 hr. The experiment is terminated at the designated incubation time by replacing the medium with 100 μL of 1 mM of resazurin (R7017; Sigma) prepared in culture medium, and the plates are further incubated in culture conditions for 4-6 hr. Fluorescence is recorded using a multimodal plate reader (Biotek Synergy Neo) at an excitation wavelength of 535 nm and emission wavelength of 590 nm to obtain relative fluorescence units. Data are analyzed as follows: the background fluorescence (blank containing only medium) value is subtracted from each reading and normalized with the vehicle control (DMSO treated cells) to obtain percent survival/proliferation. Percent survival is subtracted from 100 to get the percent inhibition of proliferation which is used to calculate IC₅₀ values. Potency of compounds in A549 (ATCC CCL-185), a non-small cell lung cancer cell line harboring the KRAS G12S mutation, is determined in an analogous manner, except that the cells are seeded in medium (DMEM with 10% FBS) at a cell count of 2000 cells per 100 μL per well.

Potency of compounds in various other cancer cell lines (such as NCI-H358 and AsPC-1) is determined in an analogous manner.

### Example B3. Determination of Potency of Compounds in Cell Proliferation Assay Panel

Potency of compounds was determined in cell proliferation assays in a panel of cancer cell lines. Cells were seeded in 384-well plates (Corning, Cat. #3765) at specified densities in 40 μL of culture medium and incubated overnight at 37° C. in a 5% CO₂ incubator (culture conditions). Medium and seeding density for each cell line is shown in Table 3. With a robotic dispenser, serially diluted test compounds were added in a volume of 20 nL DMSO to wells in the culture plate to achieve the final desired concentrations of test compounds, and the cells were further incubated in culture conditions for 72 hr. The assay was terminated by addition of 25 μL of Cell Titer-Glo reagent (Promega, Madison, Wis.) to each well. Contents were mixed, the plate was incubated for 10 min at room temperature and luminescence was measured. The IC₅₀ value of each test compound was calculated with XLFit curve fitting software. Results are shown in Table 4. ‘+++’ indicates IC₅₀ values≤300 nM, ‘++’ indicates IC₅₀ values between 300 nM-3 μM, and ‘+’ indicates IC₅₀ values≥3 μM.

TABLE 3
Cell proliferation assay panel cell lines

| | | | | Seeding Density |
|---|---|---|---|---|
| Cell Line | Vendor | Cat# | Culture medium | (cells/well) |
| MIA PaCa-2 | ATCC | CRL-1420 | DMEM + 10% FBS | 500 |
| NCI-H358 | ATCC | CRL-5807 | RPMI1640 + 10% FBS | 700 |
| A549 | ATCC | CCL-185 | F-12K + 10% FBS | 500 |
| NCI-H2122 | ATCC | CRL-5985 | RPMI1640 + 10% FBS | 500 |
| SW 1573 | ATCC | CRL-2170 | Leibovitz's L-15 + | 1000 |
| | | | 10% FBS | |
| KYSE-410 | ECACC | 94072023 | RPMI1640 + 10% FBS | 500 |
| NCI-H1373 | ATCC | CRL-5866 | RPMI1640 + 10% FBS | 500 |
| NCI-H2030 | ATCC | CRL-5914 | RPMI1640 + 10% FBS | 500 |

TABLE 4
Cell proliferation assay panel results

| Compound No. | Cell line | IC₅₀ |
|---|---|---|
| 43 (Isomer B) | A549 | + |
| | KYSE-410 | + |
| | MIA PaCa-2 | +++ |
| | NCI-H2122 | ++ |
| | NCI-H358 | +++ |
| | NCI-H1373 | ++ |
| | NCI-H2030 | ++ |
| | SW1573 | + |
| 52 (Isomer B) | MIA PaCa-2 | ++ |
| 229 (Isomer A) | A549 | + |
| | KYSE-410 | + |
| | MIA PaCa-2 | ++ |
| | NCI-H2122 | + |
| | NCI-H358 | +++ |
| | NCI-H1373 | + |
| | NCI-H2030 | ++ |
| | SW1573 | + |
| 228 (Isomer A) | MIA PaCa-2 | ++ |
| 239 (Isomer B) | MIA PaCa-2 | +++ |
| 241 (Isomer A) | MIA PaCa-2 | ++ |
| 243 (Isomer B) | MIA PaCa-2 | +++ |
| 258 (Isomer B) | MIA PaCa-2 | +++ |
| 259 (Isomer A) | MIA PaCa-2 | + |
| 259 (Isomer B) | MIA PaCa-2 | +++ |
| 260 (Isomer A) | MIA PaCa-2 | ++ |
| 266 (Isomer A) | MIA PaCa-2 | ++ |
| 266 (Isomer B) | MIA PaCa-2 | +++ |
| 267 (Isomer A) | MIA PaCa-2 | + |
| 267 (Isomer B) | MIA PaCa-2 | +++ |

### Example B4. Determination of Potency of Compounds in 3D Cell Proliferation Assays

NCI-H358 cells (ATCC® CRL-5807™) were plated in a 384-well Black/Clear Round Bottom Ultra-Low Attachment Microplate (Corning) in medium (RPMI-1640+10% FBS) at a density of 1000 cells in 50 μL per well, then incubated overnight at 37° C. and 5% CO₂. Cells were treated with test compounds at 9 concentrations within a specified concentration range (e.g., 0.5 nM-10 μM) for generation of dose response curves. Test compounds were prepared by 1:3 serial dilution in DMSO from a specified starting concentration (e.g., 20 μM, 2 μM or 0.2 μM) and then added to wells of the assay plate in a total volume of 150 nL DMSO. The cells were further incubated at 37° C. and 5% CO₂ for 7 days. Cell viability was assessed by adding 40 μL of Cell Titer-Glo® 3D reagent (Cat. No.: G9683, Promega) to each well and reading luminescence after mixing and incubation of the samples. The IC₅₀ value of each test compound was calculated with XLFit curve fitting software. Results are shown in Table 5. ‘+++’ indicates IC₅₀ values≤100 nM, ‘++’ indicates IC₅₀ values between 100 nM-2 μM, and ‘+’ indicates IC₅₀ values≥2 μM.

TABLE 5
3D Cell proliferation assay results

| Compound No. | IC₅₀ |
|---|---|
| 38 (Isomer B) | +++ |
| 40 (Isomer A) | ++ |
| 40 (Isomer B) | + |
| 43 (Isomer A) | + |
| 43 (Isomer B) | +++ |
| 52 (Isomer B) | ++ |
| 178 | ++ |
| 181 | +++ |
| 228 (Isomer A) | +++ |
| 228 (Isomer B) | + |
| 229 (Isomer A) | +++ |
| 239 (Isomer B) | +++ |
| 241 (Isomer A) | +++ |
| 243 (Isomer B) | +++ |
| 258 (Isomer B) | +++ |
| 260 (Isomer A) | ++ |
| 266 (Isomer B) | +++ |

### Example B5. Immunoblotting Detection of KRAS, Phospho-ERK and Related Proteins

MIA PaCa-2, NCI-H358 or other cancer cells are seeded in 10-cm dishes and incubated overnight in a 5% CO₂ incubator at 37° C. Cells are then treated with test compound or vehicle which is added to the culture dish for a specified duration. Following treatment, they are washed twice with PBS and lysed in lysis buffer (25 mM Tris-HCl, pH 7.2, 150 mM NaCl, 5 mM MgCl₂, 5% glycerol, 1% NP40) supplemented with phosphatase and protease inhibitors. Proteins from the lysates are separated by standard SDS-PAGE methods. Following separation, proteins are blotted onto nitrocellulose membranes which are then blocked and incubated with primary antibodies specific for target proteins such as RAS, KRAS, phospho-ERK, ERK, phospho-AKT, AKT, phospho-S6 or others. After the primary antibody incubation, membranes are incubated with HRP-linked secondary antibody, and proteins are detected by a chemiluminescence detection method.

### Example B6. Phospho-ERK Signaling Analysis

Method I. Phosphorylation of ERK is assessed by an AlphaScreen® assay (e.g., AlphaScreen® SureFire® pERK1/2 (Thr202/Tyr204) assay, Perkin Elmer). In brief, MIA PaCa-2, NCI-H358 or other cancer cells are seeded in 96-well plates and incubated overnight in a 5% CO₂ incubator at 37° C. The culture medium is then removed and replaced with serum-free medium containing serially diluted compound or DMSO. After further incubation at 37° C., medium is removed and cells are lysed by addition of IX Lysis Buffer and agitation on a plate shaker. A portion of the lysate is transferred to an assay plate. After Reaction Mix containing Reaction buffer, Activation buffer and AlphaScreen beads is added to the lysate, the plate is briefly agitated on a plate shaker and then incubated at room temperature for a desired duration. The plate is read on an AlphaScreen-compatible fluorescence plate reader (520-620 nm emission) and dose response curves are analyzed using 4-parameter non-linear regression.

Method II. Phosphorylation of ERK was assessed by a homogeneous TR-FRET assay (Advanced phospho-ERK (Thr202/Tyr204) cellular kit; Cat. #64AERPEH, Cisbio). In brief, NCI-H358 cells were seeded in a 384-well culture plate at a density of 6000 cells in 40 μL medium/well and grown overnight at 37° C. and 5% CO₂. Each test compound was dosed in duplicate in a 10-dose 3-fold titration starting at 10 μM or 2 μM. The culture plate was then incubated for 4 hrs in the incubator. At the end of the compound treatment, the medium was removed, 35 μL of lysis buffer was added to each well, and the plate was agitated on a plate shaker for 40 mins at 4° C. An antibody cocktail of phospho-ERK1/2 Eu Cryptate antibody and Phospho-ERK1/2 d2 antibody was prepared by separately diluting the antibodies 20-fold with detection buffer and then mixing them together. 3.4 μL of antibody cocktail was dispensed to each well of an empty 384-well white ProxiPlus assay plate (Perkin Elmer), and the plate briefly centrifuged. 13.6 μL of cell lysate was then added to the antibody-containing wells, and the plate was briefly centrifuged and then incubated 2 hrs at room temperature. The plate was read on a fluorescence plate reader (Envision2104, Perkin Elmer), with an excitation wavelength of 337 nm and emission wavelengths of 615 nm and 665 nm. The IC₅₀ value of each test compound was calculated with XLFit curve fitting software. Results are shown in Table 6. ‘+++’ indicates IC₅₀ values≤300 nM, ‘++’ indicates IC₅₀ values between 300 nM-3 μM, and ‘+’ indicates IC₅₀ values≥3 μM.

TABLE 6
pERK assay results (Method II)

| Compound No. | IC₅₀ |
|---|---|
| 43 (Isomer B) | +++ |
| 52 (Isomer B) | ++ |
| 241 (Isomer A) | +++ |
| 243 (Isomer B) | +++ |

### Example B7. Biochemical Modification Assay

Formation of covalent adducts of KRAS G12C protein with test compound is assessed by a biochemical modification assay, such as described previously (Patricelli MP el al. Cancer discovery. 2016 Mar. 1; 6(3):316-29). In brief, GDP-bound recombinant KRAS G12C protein is incubated with test compound in modification buffer (20 mM HEPES pH 7.5, 150 mM NaCl, 1 mM MgCl₂, and 1 mM DTT) for a specified duration at room temperature, and the reaction is subsequently quenched with acid. LC-MS (electrospray interface) analysis is performed with a Q-Exactive (Thermo Scientific) or similar mass spectrometer to assess modification of intact KRAS G12C protein.

### Example B8. Electrophoretic Mobility Shift Assay

Formation in cancer cells of covalent adducts of endogenous KRAS G12C protein with test compound is assessed by an electrophoretic mobility shift assay, such as described previously (Hallin J et al. Cancer discovery. 2020 Jan. 1; 10(1):54-71). In brief, MIA PaCa-2, NCI-H358 or other cancer cells are seeded in culture dishes or plates and incubated overnight in a 5% CO₂ incubator at 37° C. Cells are then treated with compound at a specified concentration or range of concentrations (e.g., ranging from 0.5 nM-30 μM) for a desired length of time (e.g., single or multiple time points from 1-72 hr). Following treatment, they are washed twice with PBS and lysed in lysis buffer. Proteins from the lysates are separated by gel electrophoresis and blotted onto nitrocellulose membranes which are then blocked and incubated with primary antibodies specific for KRAS protein. After the primary antibody incubation, membranes are incubated with HRP-linked secondary antibody, and proteins are detected by a chemiluminescence detection method. An upward electrophoretic mobility shift of the KRAS G12C protein band indicates covalent modification of KRAS G12C by test compound.

### Example B9. hERG Inhibition Assay

Compounds were tested for the potential to inhibit the hERG potassium channel. It is known that inhibition of the hERG channel may cause QT prolongation and increase the risk of cardiac arrhythmias. Therefore, to avoid cardiotoxicity, a compound with relatively low or no inhibition of the channel is desirable. Manual whole-cell patch-clamp recordings were made in Chinese hamster ovary (CHO) cells stably expressing hERG channels (B'Sys, Switzerland). Standard intracellular and bath solutions for hERG current detection were used. Cells were exposed to test compounds at 6 concentrations prepared by 1:3 serial dilution of compounds from a top starting concentration of 30 μM. Currents were elicited by stepping the voltage from a holding potential of −80 mV to −50 mV (20 ms) and then stepping to +40 mV (4 s). A subsequent step to −50 mV (4 s) was used to elicit a tail current which was used for data analysis. Current recordings were made in triplicate using three different cells. Data normalized to the activity in vehicle (0.1 or 0.3% DMSO) were fit with 4-parameter non-linear regression, and IC₅₀ values were determined from the dose-response curves, using GraphPad Prism software. Results are shown in Table 7. Comparator compounds X (AMG 510) and Y (MRTX849) had IC₅₀ values of >30 and 0.71 μM, respectively.

TABLE 7
hERG assay results

| Compound No. | IC₅₀ |
|---|---|
| 43 (Isomer B) | 3.61 |
| X (AMG 510) | >30 |
| Y (MRTX 849) | 0.71 |

### Example B10. Evaluation of Test Compound in NCI-H358 Mouse Xenograft Model

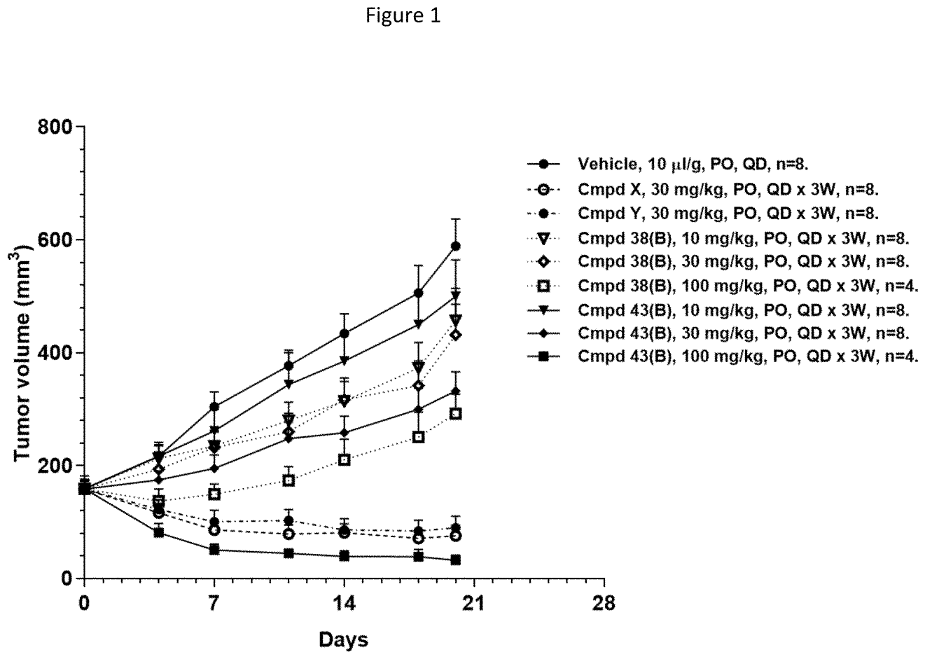

To examine the in vivo antitumor activity of test compound, tumor growth experiments were performed in an NCI-H358 mouse xenograft model. NCI-H358 cells were injected subcutaneously (5×10⁶ cells in 0.1 mL of PBS) into the right flanks of 6-8-week-old female BALB/c nude mice and allowed to grow until the average tumor size reached 150-200 mm³. The mice were then stratified into treatment groups of 4-8 animals and treatment initiated on Day 0. Test compound or vehicle (1% Tween 80, 2% HPMC, and 97% water) was administered orally, once daily, for 21 days. Tumor size was measured twice weekly in two dimensions using a caliper, and the volume was expressed in mm³ using the formula: V=(a×b²/2) where a and b are the long and short diameters of the tumor, respectively. Tumor Growth Inhibition (TGI) was calculated for each group using the formula: TGI %=(1−Ti/Vi)×100, where Ti is the mean tumor volume of the treatment group on the measurement day, and Vi is the mean tumor volume of the control group on the measurement day. Animal body weight was monitored regularly as an indicator of toxicity. Administration of compound 38 (isomer B) once daily at dose levels of 10, 30 and 100 mg/kg resulted in TGI values of 22.4%, 26.7% and 50.4%, respectively, on Day 20 after treatment start. One of the mice that received compound 38 (isomer B) at 100 mg/kg exhibited weight loss between 10-15% and had treatment suspended on Days 3 and 4. Administration of compound 43 (isomer B) once daily at dose levels of 10, 30 and 100 mg/kg resulted in TGI values of 15.1%, 43.6% and 94.6%, respectively, on Day 20 after treatment start. None of the mice that received compound 43 (isomer B) exhibited weight loss greater than 10%, indicating that it was well-tolerated. Administration of a comparator compound, compound X (AMG510), once daily at a dose level of 30 mg/kg resulted in a TGI value of 87.1%. Administration of a comparator compound, compound Y (MRTX849), once daily at a dose level of 30 mg/kg resulted in a TGI value of 84.7%. Two of eight mice that received compound X (AMG510) at 30 mg/kg exhibited weight loss between 10-15% and had treatment suspended intermittently. Tumor growth curves for the different treatment groups are shown in FIG. 1. Data points represent the group mean, and error bars represent the standard error of the mean (SEM).

### Example B11 Evaluation of Test Compound in MIA PaCa-2 Mouse Xenograft Model

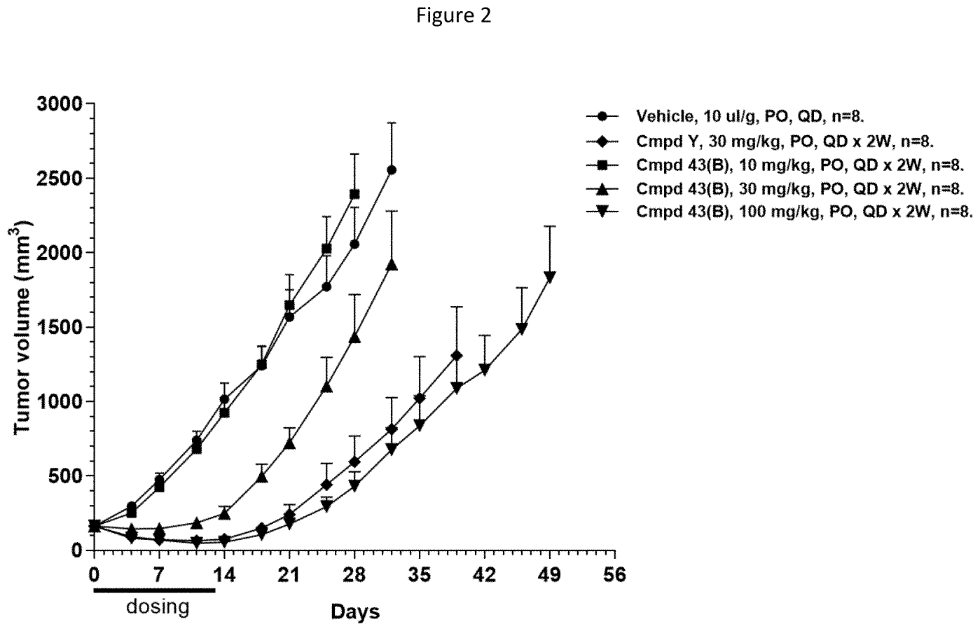

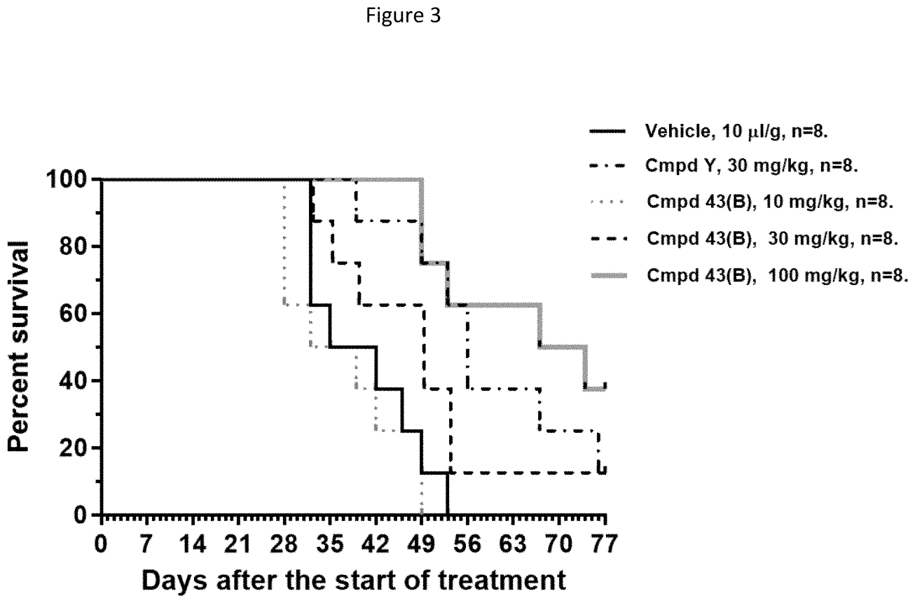

To examine the in vivo antitumor activity of test compound, tumor growth experiments were performed in a MIA PaCa-2 mouse xenograft model. MIA PaCa-2 cells were injected subcutaneously (10×10⁶ cells in 0.2 mL of PBS with Matrigel (1:1)) into the right flanks of 6-8-week-old female BALB/c nude mice and allowed to grow until the average tumor size reached 150-200 mm³. The mice were then stratified into treatment groups of 8 animals and treatment initiated on Day 0. Test compound or vehicle (1% Tween 80, 2% HPMC, and 97% water) was administered orally, once daily, for 14 days with an observational phase to monitor tumor growth post-treatment. Tumor size was measured twice weekly in two dimensions using a caliper, and the volume was expressed in mm³ using the formula: V=(a×b²/2) where a and b are the long and short diameters of the tumor, respectively. Tumor Growth Inhibition (TGI) was calculated for each group using the formula: TGI %=(1−Ti/Vi)×100, where Ti is the mean tumor volume of the treatment group on the measurement day, and Vi is the mean tumor volume of the control group on the measurement day. Animal body weight was monitored regularly as an indicator of toxicity. Administration of compound 43 (isomer B) once daily at dose levels of 10, 30, and 100 mg/kg resulted in TGI values of 9.0%, 75.5% and 94.6%, respectively, on Day 14 after treatment start. Administration of a comparator compound, compound Y (MRTX849), once daily at a dose level of 30 mg/kg resulted in a TGI value of 92.5%. None of the mice that received compound 43 (isomer B) or compound Y (MRTX849) exhibited weight loss greater than 10%, indicating that the compounds were well-tolerated. Tumor growth curves for the different treatment groups are shown in FIG. 2. Data points represent the group mean, and error bars represent the standard error of the mean (SEM). Survival curves were plotted using the Kaplan-Meier method and are shown in FIG. 3.

### Example B12. Single Crystal Studies of Compound 43 (Isomer A)

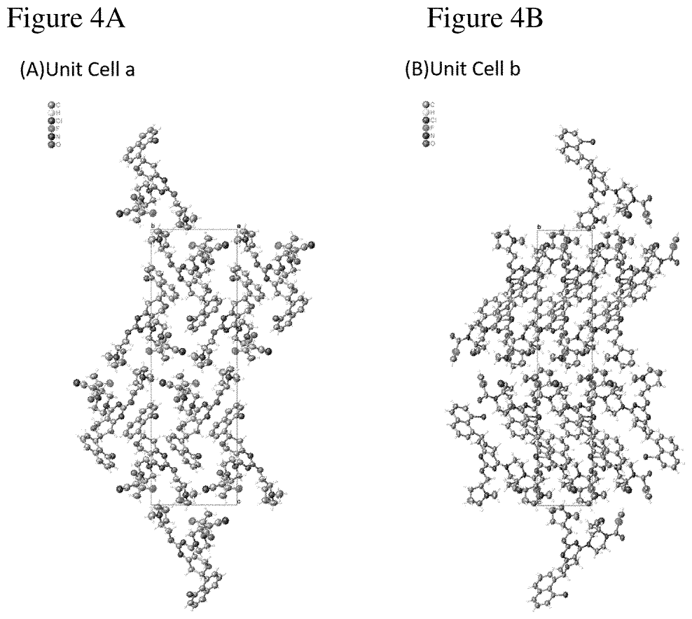

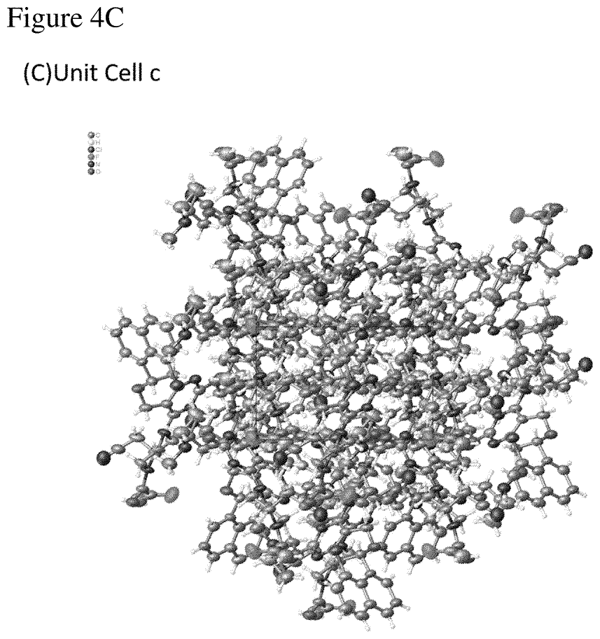

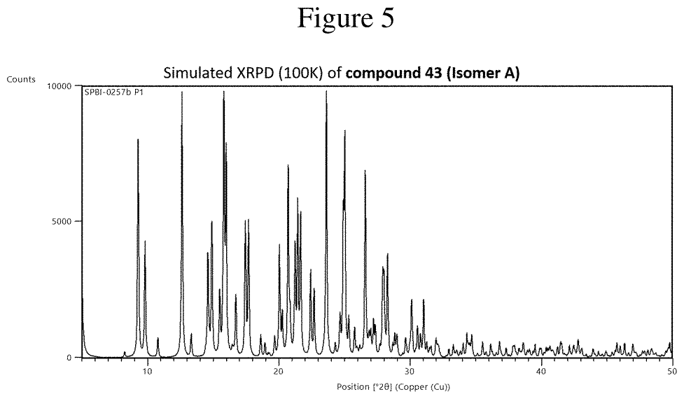

Single crystal studies of compound 43 (Isomer A) produced an orthorhombic P2₁2₁2₁ unit cell with the parameters shown below in Table 8. The unit cells and simulated XRPD (100K) of compound 43 (Isomer A) are shown in FIGS. 4 and 5 respectively.

TABLE 8
Unit cell parameters for Single Crystal of compound 43 (Isomer A)

| a | 7.145(2) Å |
|---|---|
| b | 11.219(5) Å |
| c | 36.062(12) Å |
| V | 2890.7(18) Å³ |
| Rᵢₙₜ | 16.43% |
| α | 90° |
| β | 90° |
| γ | 90° |
| Z | 4 |
| Z′ | 1 |
| R₁ (I > σ(I)) | 9.77% |
| S | 0.949 |
| wR₂ (all data) | 29.62% |
| ρ | 1.390 g · cm⁻³ |
| Flack parameter | −0.07 (6) |

All publications, including patents, patent applications, and scientific articles, mentioned in this specification are herein incorporated by reference in their entirety for all purposes to the same extent as if each individual publication, including patent, patent application, or scientific article, were specifically and individually indicated to be incorporated by reference.

1. A compound of formula (I): 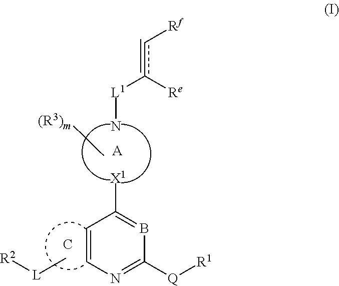
or a pharmaceutically acceptable salt thereof, wherein:
A is a 4-12 membered saturated or partially saturated monocyclic, bridged or spiro ring;
B is N or CR^{a}; [symbol]
is 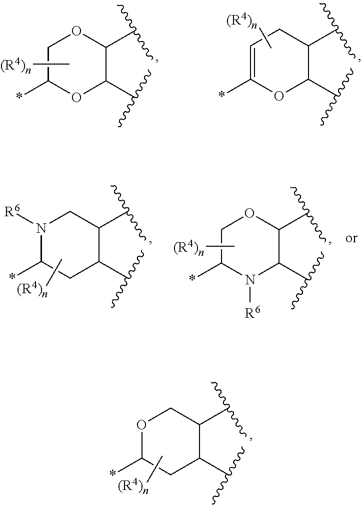
wherein * denotes the point of attachment to L;
X¹ is C, CH, or N;
Q is —O—, —S—, —NR⁵—, C₁-C₃ alkylene, or a bond;
m is 0, 1, 2, or 3;
n is 0, 1, 2, or 3;
R¹ is 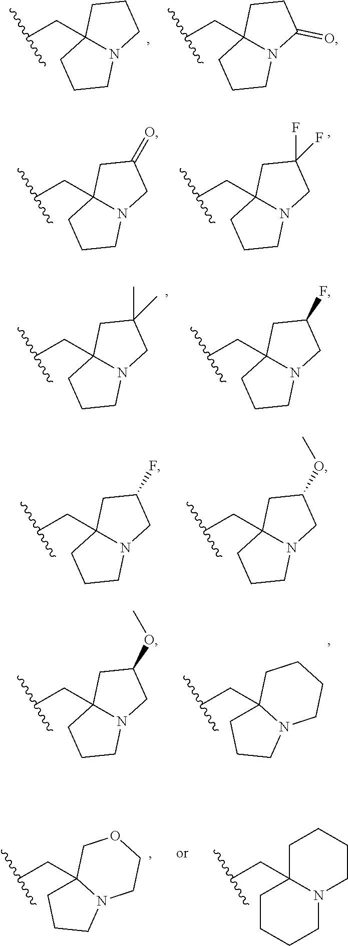
R² is C₃-C₁₂ cycloalkyl, 3-12 membered heterocyclyl, 5-12 membered heteroaryl, or C₆-C₁₂ aryl, each of which is optionally substituted with one or more R^{2a};
L is a bond, —C(O)—, C₁-C₃ alkylene, —S(O)—, —S(O)₂—, or —NR⁵—;
L¹ is —C(O)— or —S(O)₂—;
R^{2a} and R³ are each independently oxo, C₃-C₈ cycloalkyl, 3-12 membered heterocyclyl, halogen, hydroxyl, C₁-C₆ alkoxy, C₁-C₆ haloalkyl, C₁-C₆ haloalkoxy, C₁-C₆ alkyl, C₂-C₆ alkynyl, C₁-C₆ heteroalkyl, —CN, —OR^{g}, —C(O)OR^{g}, —C(O)N(R^{g}R^{h}), or —N(R^{g}R^{h}), each of which is optionally substituted with one or more substituents selected from —CN, halogen, —ORⁱ, —N(RⁱR^{j}) and 5-12 membered heteroaryl;
R⁴ is hydrogen, oxo, —C(O)R^{g}, hydroxyl, —(C₁-C₆ alkylene)OR^{g}, —CN, halogen, C₁-C₆ alkyl, —(C₁-C₆ alkylene) C₆-C₁₂ aryl, C₁-C₆ haloalkyl, C₁-C₆ heteroalkyl, C₃-C₈ cycloalkyl, 3-12 membered heterocyclyl, —(C₁-C₆ alkylene) 3-12 membered heterocyclyl, C₁-C₆ alkoxy, —(C₁-C₆, alkylene)C(O)N(R^{g}R^{h}), —(C₁-C₆, alkylene)N(R^{g}R^{h}), —S(O)₂R^{g}, —C(O)OR^{g}, —C(O)N(R^{g}R^{h}), or —N(R^{g}R^{h}), each of which is optionally substituted with one or more substituents selected from —ORⁱ, C₃-C₁₂ cycloalkyl, and —CN;
each R⁵ is independently hydrogen, C₁-C₃ alkyl, or C₃-C₆ cycloalkyl;
each R⁶ is independently H, C₁-C₆ haloalkyl, C₁-C₆ alkyl, C₃-C₈ cycloalkyl, C₁-C₆ haloalkoxy, C₁-C₆ alkoxy, —C(O)R^{c}, —S(O)₂R^{b}, —C(O)OR^{c}, —C(O)N(R^{c}R^{d}), C₆-C₁₂ aryl, 3-12 membered heterocyclyl, or 5-12 membered heteroaryl, each of which is optionally substituted with one or more R³;
each R^{a} is independently hydrogen, —CN, C₁-C₆ alkoxy, C₁-C₆ haloalkoxy, —C(O)R^{c}, —S(O)₂R^{b}, —C(O)OR^{c}, —C(O)N(R^{c}R^{d}), —N(R^{c}R^{d}), C₆-C₁₂ aryl, 3-12 membered heterocyclyl, 5-12 membered heteroaryl, C₁-C₆ alkoxy, halogen, —N(R^{c})C(O)N(R^{c}R^{d}), or —N(R^{c})C(O)R^{d}, each of which is optionally substituted with one or more R³;
each R^{b} is independently hydrogen, —CN, C₁-C₆ alkyl, C₁-C₆ haloalkyl, C₃-C₈ cycloalkyl, —C(O)R^{c}, —C(O)OR^{c}, —C(O)N(R^{c}R^{d}), —N(R^{c}R^{d}), C₆-C₁₂ aryl, 3-12 membered heterocyclyl, 5-12 membered heteroaryl, C₁-C₆ alkoxy, halogen, —N(R^{c})C(O)N(R^{c}R^{d}), or —N(R^{c})C(O)R^{d}, each of which is optionally substituted with one or more R³;
R^{c} and R^{d} are each independently hydrogen, C₁-C₆ alkyl, C₆-C₁₂ aryl, 3-12 membered heterocyclyl, 5-12 membered heteroaryl, C₁-C₆ haloalkyl, or C₃-C₈ cycloalkyl, each of which is optionally substituted with one or more R³,
or R^{c} and R^{d} are taken together with the atom to which they attach to form a 3-12 membered heterocyclyl or 5-12 membered heteroaryl, [symbol]
is a double bond or a triple bond, provided that
when [symbol] is a double bond, then R^{e} and R^{f} are each independently H, halogen, —CN, —C(O)OR^{g}, C₁-C₆ haloalkyl, C₁-C₆ alkyl, C₁-C₆ heteroalkyl, —C(O)N(R^{g}R^{h}), C₆-C₁₂ aryl, 5-12 membered heteroaryl, 3-12 membered heterocyclyl, —(C₁-C₆ alkylene)OR^{g}, or —(C₁-C₆ alkylene)N(R^{g}R^{h}), or
R^{e} and R^{f} are taken together with the atoms to which they attach to form a C₃-C₁₂ cycloalkyl, 3-12 membered heterocyclyl, or 5-12 membered heteroaryl, and
when [symbol] is a triple bond, then R^{e} is absent and R^{f} is H, halogen, —CN, —C(O)OR^{g}, C₁-C₆ haloalkyl, C₁-C₆ alkyl, C₁-C₆ heteroalkyl, —C(O)N(R^{g}R^{h}), C₆-C₁₂ aryl, 5-12 membered heteroaryl, 3-12 membered heterocyclyl, —(C₁-C₆ alkylene)OR^{g}, or —(C₁-C₆ alkylene)N(R^{g}R^{h}); and
R^{g}, R^{h}, Rⁱ, and R^{j} are each independently H, C₁-C₆ alkyl, C₆-C₁₂ aryl, 3-12 membered heterocyclyl, 5-12 membered heteroaryl, C₁-C₆ haloalkyl, C₃-C₈ cycloalkyl, or —NH₂, or
R^{g} and R^{h} or Rⁱ and R^{j} are taken together with the atom to which they attach to form a 3-12 membered heterocyclyl or 5-12 membered heteroaryl.

2. The compound of claim 1, or a pharmaceutically acceptable salt thereof, wherein [symbol] is

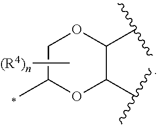

3. The compound of claim 1, or a pharmaceutically acceptable salt thereof, wherein [symbol] is

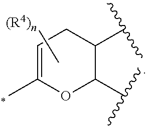

4. The compound of claim 1, or a pharmaceutically acceptable salt thereof, wherein [symbol] is

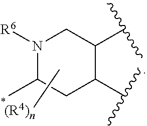

5. The compound of claim 1, or a pharmaceutically acceptable salt thereof, wherein [symbol] is

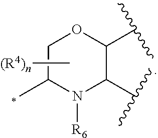

6. The compound of claim 1, or a pharmaceutically acceptable salt thereof, wherein [symbol] is

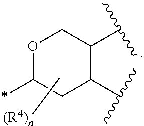

7. The compound of claim 1, or a pharmaceutically acceptable salt thereof, wherein R⁶ is hydrogen or C₁-C₆ alkyl.

8. The compound of claim 1, or a pharmaceutically acceptable salt thereof, wherein B is N.

9. The compound of claim 1, or a pharmaceutically acceptable salt thereof, wherein B is CR^{a}.

10. The compound of claim 1, or a pharmaceutically acceptable salt thereof, wherein Q is —O—.

11. The compound of claim 1, or a pharmaceutically acceptable salt thereof, wherein Q is —S—.

12-15. (canceled)

16. The compound of claim 1, or a pharmaceutically acceptable salt thereof, wherein X¹ is N.

17. The compound of claim 1, or a pharmaceutically acceptable salt thereof, wherein A and R³ together are 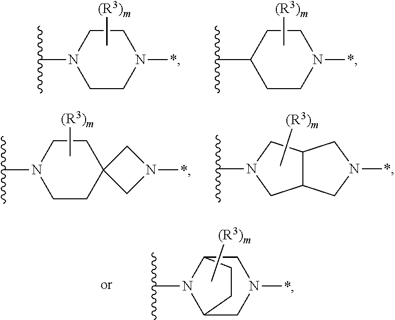
wherein * denotes the point of attachment to L¹.

18. The compound of claim 1, or a pharmaceutically acceptable salt thereof, wherein A and R³ together are 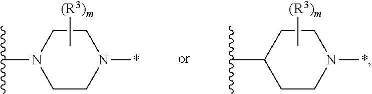
wherein * denotes the point of attachment to L¹.

19. The compound of claim 1, or a pharmaceutically acceptable salt thereof, wherein A and R³ together are 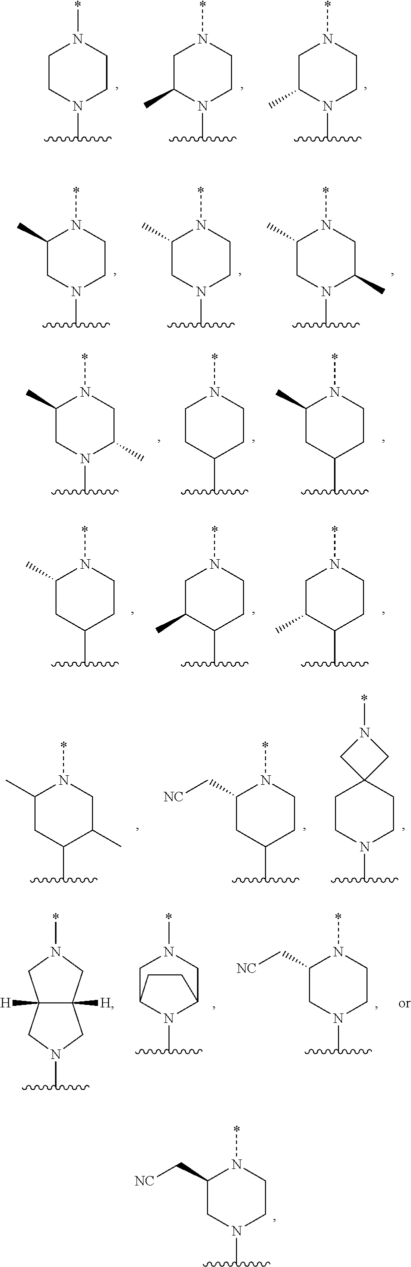
wherein * denotes the point of attachment to L¹.

20. The compound of claim 1, or a pharmaceutically acceptable salt thereof, wherein L¹ is —C(O)—.

21. (canceled)

22. The compound of claim 1, or a pharmaceutically acceptable salt thereof, wherein L is a bond.

23-25. (canceled)

26. The compound of claim 1, or a pharmaceutically acceptable salt thereof, wherein [symbol] is a double bond.

27. (canceled)

28. The compound of claim 1, or a pharmaceutically acceptable salt thereof, wherein R^{e} is H, halogen, —CN, or C₁-C₆ alkyl.

29. The compound of claim 28, or a pharmaceutically acceptable salt thereof, wherein R^{f} is H, C₁-C₆ alkyl, or —(C₁-C₆ alkylene)N(R^{g}R^{h}).

30. The compound of claim 1, or a pharmaceutically acceptable salt thereof, wherein R² is 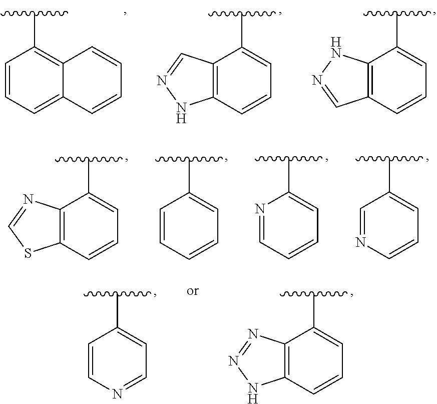
each of which is optionally substituted with one or more R^{2a}.

31. (canceled)

32. The compound of claim 1, or a pharmaceutically acceptable salt thereof, wherein R²

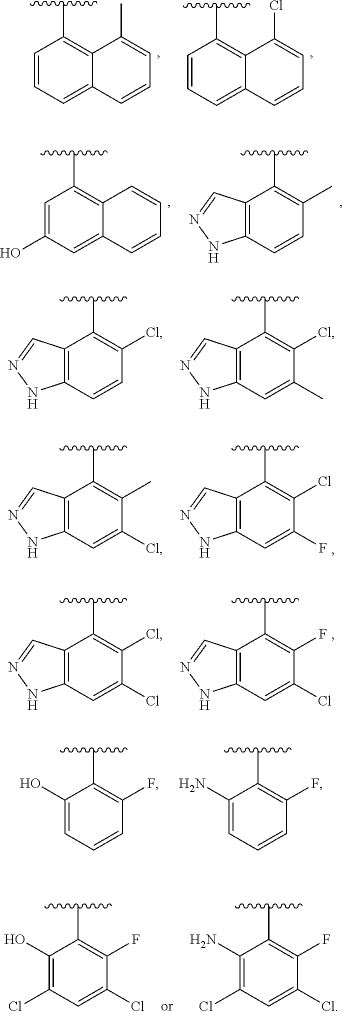

33. The compound of claim 1, or a pharmaceutically acceptable salt thereof, wherein

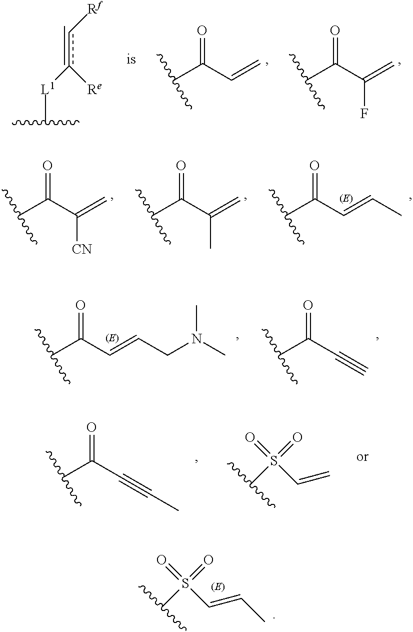

34-36. (canceled)

37. A pharmaceutical composition comprising the compound of claim 1 or a pharmaceutically acceptable salt thereof, and a pharmaceutically acceptable excipient.

38. (canceled)

39. A method of treating a KRAS-mediated disease in a subject in need thereof, comprising administering a therapeutically effective amount of the compound of claim 1, or a pharmaceutically acceptable salt thereof, wherein the KRAS-mediated disease is cancer.

40-51. (canceled)

52. The compound of claim 1, or a pharmaceutically acceptable salt thereof, wherein A and R³ together are 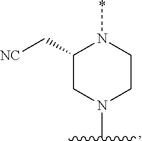
wherein * denotes the point of attachment to L¹.

53. The compound of claim 1, or a pharmaceutically acceptable salt thereof, wherein A and R³ together are 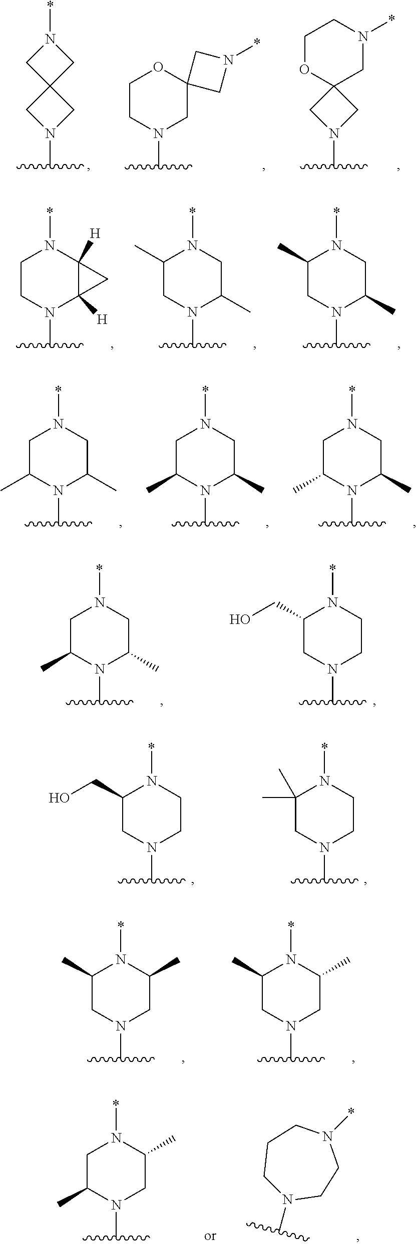
wherein * denotes the point of attachment to L¹.

54. The compound of claim 1, or a pharmaceutically acceptable salt thereof, wherein R² is

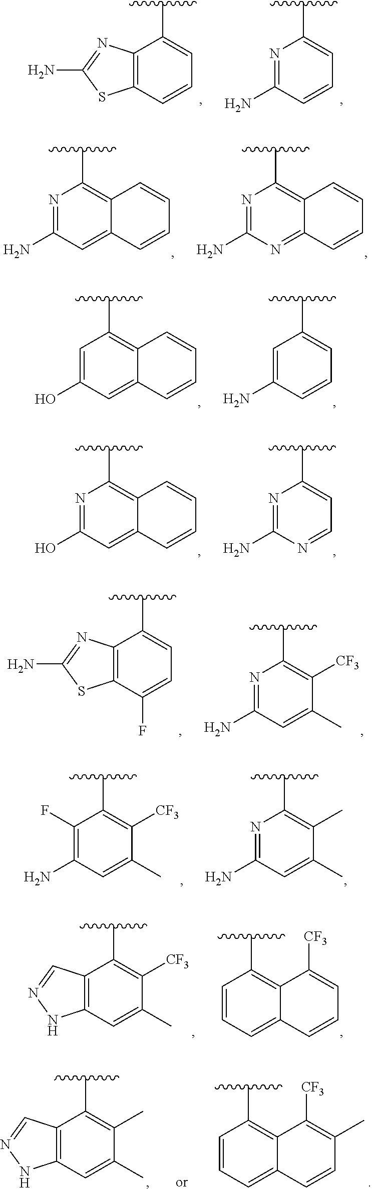

55. The compound of claim 1, or a pharmaceutically acceptable salt thereof, wherein R² is 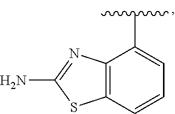 [SMILES: *c1cccc2sc(N)nc12]
optionally substituted by one or more R^{2a}.

56. The compound of claim 1, or a pharmaceutically acceptable salt thereof, wherein R² is 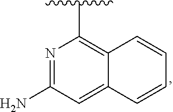 [SMILES: *c1nc(N)cc2ccccc12]
optionally substituted by one or more R^{2a}.

57. A compound of formula (VI-g′), 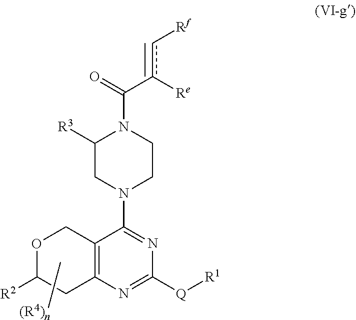
or a pharmaceutically acceptable salt thereof, wherein, R¹ is 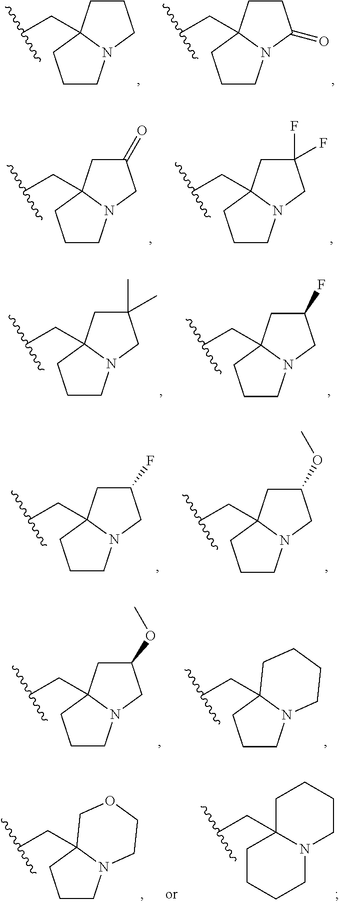
R² is C₃-C₁₂ cycloalkyl, 3-12 membered heterocyclyl, 5-12 membered heteroaryl, or C₆-C₁₂ aryl, each of which is optionally substituted with one or more R^{2a};
R^{2a} and R³ are each independently oxo, C₃-C₈ cycloalkyl, 3-12 membered heterocyclyl, halogen; hydroxyl, C₁-C₆ alkoxy, C₁-C₆ haloalkyl, C₁-C₆ haloalkoxy, C₁-C₆ alkyl, C₂-C₆ alkynyl, C₁-C₆ heteroalkyl, —CN, —OR^{g}, —C(O)OR^{g}, —C(O)N(R^{g}R^{h}), or —N(R^{g}R^{h}), each of which is optionally substituted with one or more substituents selected from —CN, halogen, —ORⁱ, —N(RⁱR^{j}) and 5-12 membered heteroaryl;
R⁴ is hydrogen, oxo, —C(O)R^{g}, hydroxyl, —(C₁-C₆ alkylene)OR^{g}, —CN, halogen, C₁-C₆ alkyl, —(C₁-C₆ alkylene) C₆-C₁₂ aryl, C₁-C₆ haloalkyl, C₁-C₆ heteroalkyl, C₃-C₈ cycloalkyl, 3-12 membered heterocyclyl, —(C₁-C₆ alkylene) 3-12 membered heterocyclyl, C₁-C₆ alkoxy, —(C₁-C₆ alkylene)C(O)N(R^{g}R^{h}), —(C₁-C₆ alkylene)N(R^{g}R^{h}), —S(O)₂R^{g}, —C(O)OR^{g}, —C(O)N(R^{g}R^{h}), or —N(R^{g}R^{h}), each of which is optionally substituted with one or more substituents selected from —ORⁱ, C₃-C₁₂ cycloalkyl, and —CN; [symbol]
is a double bond or a triple bond, provided that
when [symbol] is a double bond, then R^{e} and R^{f} are each independently H, halogen, —CN, —C(O)OR^{g}, C₁-C₆ haloalkyl, C₁-C₆ alkyl, C₁-C₆ heteroalkyl, —C(O)N(R^{g}R^{h}), C₆-C₁₂ aryl, 5-12 membered heteroaryl, 3-12 membered heterocyclyl, —(C₁-C₆ alkylene)OR^{g}, or —(C₁-C₆ alkylene)N(R^{g}R^{h}), or
R^{e} and R^{f} are taken together with the atoms to which they attach to form a C₃-C₁₂ cycloalkyl, 3-12 membered heterocyclyl, or 5-12 membered heteroaryl, and
when [symbol] is a triple bond, then R^{e} is absent and R^{f} is H, halogen, —CN, —C(O)OR^{g}, C₁-C₆ haloalkyl, C₁-C₆ alkyl, C₁-C₆ heteroalkyl, —C(O)N(R^{g}R^{h}), C₆-C₁₂ aryl, 5-12 membered heteroaryl, 3-12 membered heterocyclyl, —(C₁-C₆ alkylene)OR^{g}, or —(C₁-C₆ alkylene)N(R^{g}R^{h});
R^{g}, R^{h}, Rⁱ, and R^{j} are each independently H, C₁-C₆ alkyl, C₆-C₁₂ aryl, 3-12 membered heterocyclyl, 5-12 membered heteroaryl, C₁-C₆ haloalkyl, C₃-C₈ cycloalkyl, or —NH₂, or R^{g} and R^{h} or Rⁱ and R^{j} are taken together with the atom to which they attach to form a 3-12 membered heterocyclyl or 5-12 membered heteroaryl;
Q is —O—, —S—, —NR⁵—, C₁-C₃ alkylene, or a bond;
R⁵ is hydrogen, C₁-C₃ alkyl, or C₃-C₆ cycloalkyl; and
n is 0, 1, 2, or 3.

58. The compound of claim 57, or a pharmaceutically acceptable salt thereof, wherein R³ is —CH₂CN.

59. The compound of claim 57, or a pharmaceutically acceptable salt thereof, wherein Q is —O—.

60. The compound of claim 57, or a pharmaceutically acceptable salt thereof, wherein

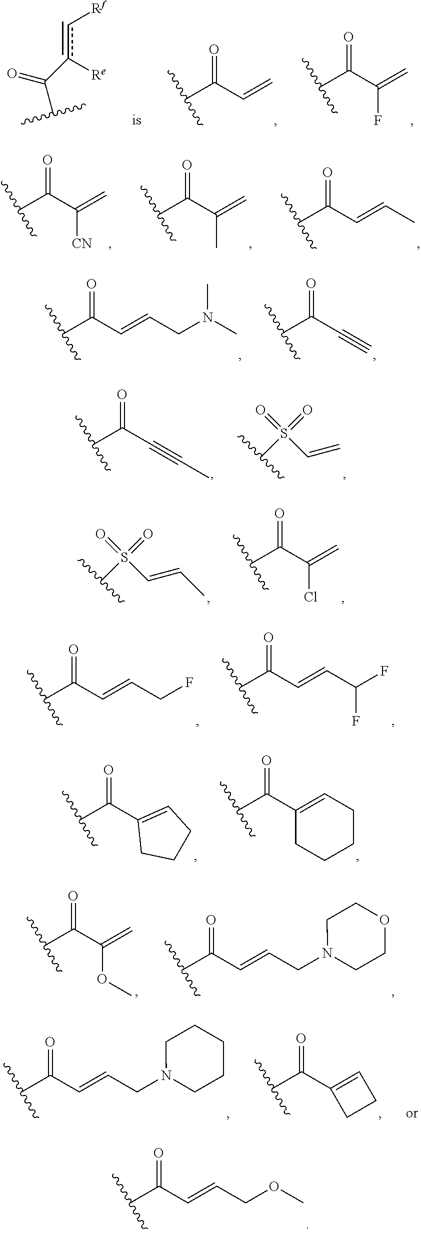

61. The compound of claim 60, or a pharmaceutically acceptable salt thereof, wherein

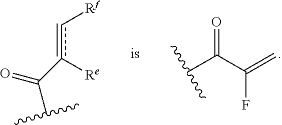

62. The compound of claim 60, or a pharmaceutically acceptable salt thereof, wherein

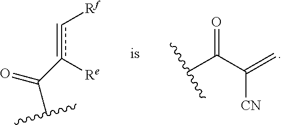

63. The compound of claim 60, or a pharmaceutically acceptable salt thereof, wherein R¹ is

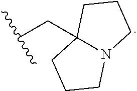 [SMILES: *CC12CCCN1CCC2]

64. The compound of claim 63, or a pharmaceutically acceptable salt thereof, wherein Q is —O—.

65. The compound of claim 60, or a pharmaceutically acceptable salt thereof, wherein R¹ is

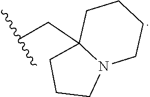 [SMILES: *CC12CCCCN1CCC2]

66. The compound of claim 65, or a pharmaceutically acceptable salt thereof, wherein Q is —O—.

67. The compound of claim 57, or a pharmaceutically acceptable salt thereof, wherein R² is

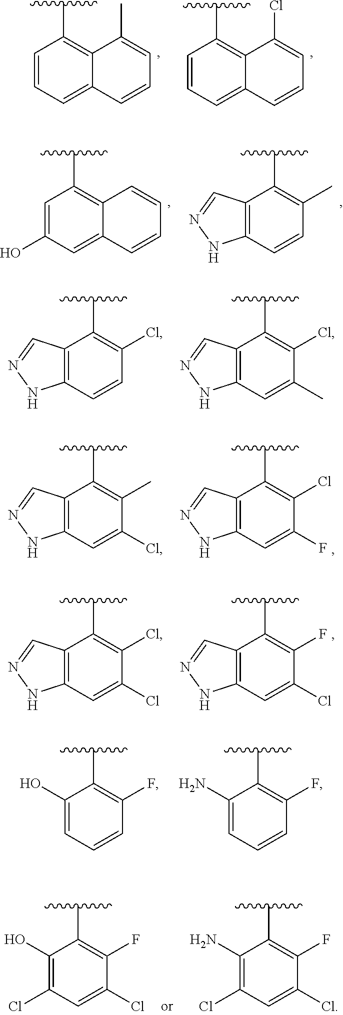

68. The compound of claim 67, or a pharmaceutically acceptable salt thereof, wherein R² is

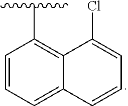 [SMILES: *c1cccc2cccc(Cl)c12]

69. The compound of claim 68, or a pharmaceutically acceptable salt thereof, wherein

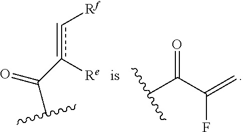

70. The compound of claim 1, or a pharmaceutically acceptable salt thereof, wherein the compound is

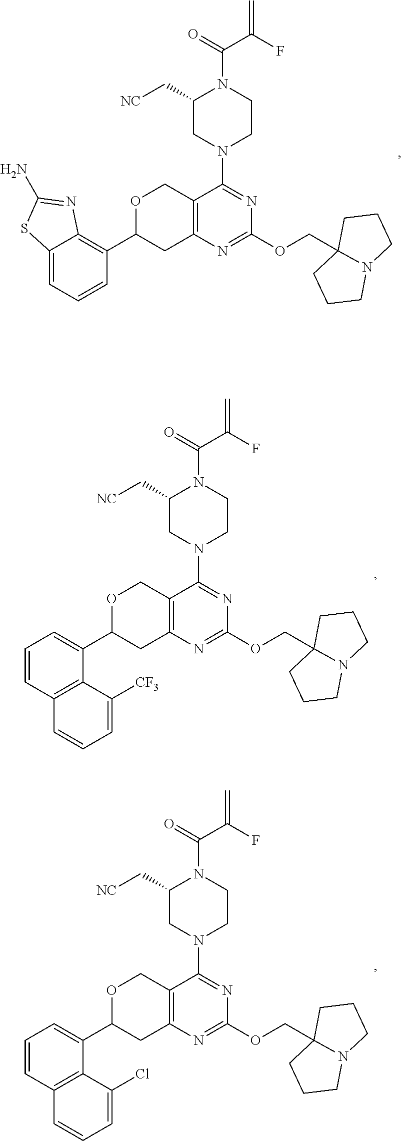 [SMILES: C=C(F)C(=O)N1CCN(c2nc(OCC34CCCN3CCC4)nc3c2COC(c2cccc4sc(N)nc24)C3)C[C@@H]1CC#N ; C=C(F)C(=O)N1CCN(c2nc(OCC34CCCN3CCC4)nc3c2COC(c2cccc4cccc(C(F)(F)F)c24)C3)C[C@@H]1CC#N ; C=C(F)C(=O)N1CCN(c2nc(OCC34CCCN3CCC4)nc3c2COC(c2cccc4cccc(Cl)c24)C3)C[C@@H]1CC#N]

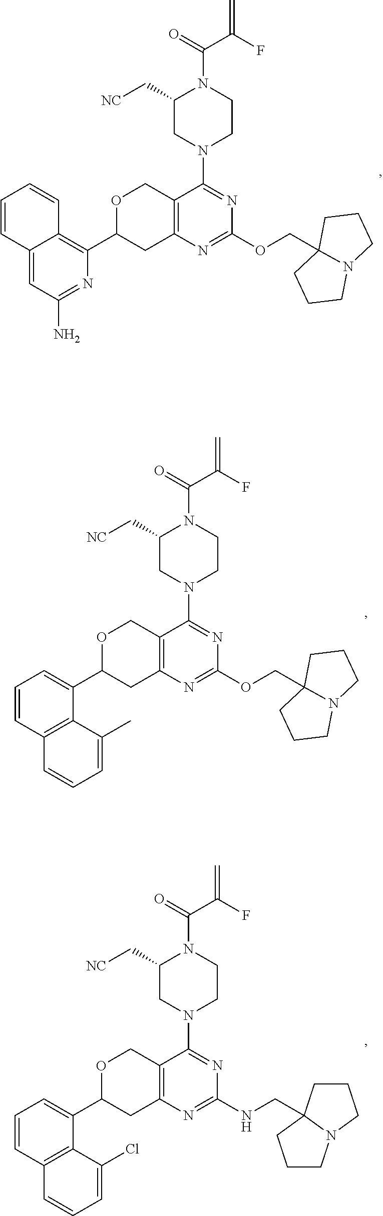 [SMILES: C=C(F)C(=O)N1CCN(c2nc(OCC34CCCN3CCC4)nc3c2COC(c2nc(N)cc4ccccc24)C3)C[C@@H]1CC#N ; C=C(F)C(=O)N1CCN(c2nc(OCC34CCCN3CCC4)nc3c2COC(c2cccc4cccc(C)c24)C3)C[C@@H]1CC#N ; C=C(F)C(=O)N1CCN(c2nc(NCC34CCCN3CCC4)nc3c2COC(c2cccc4cccc(Cl)c24)C3)C[C@@H]1CC#N]

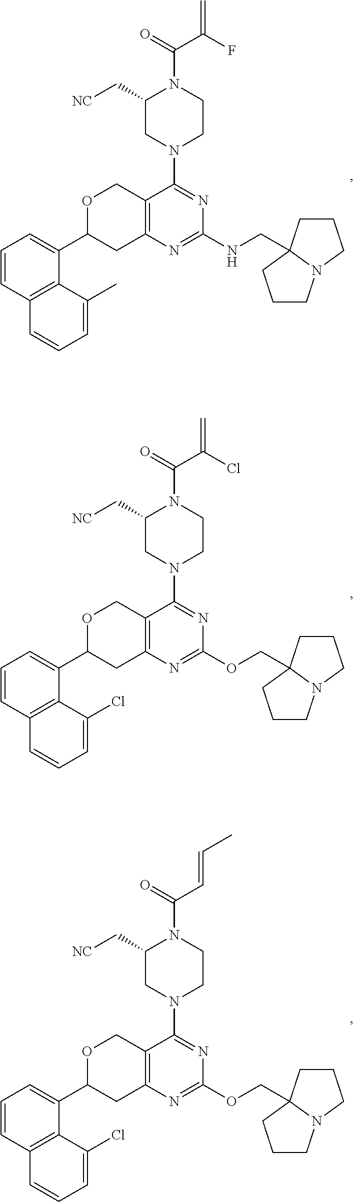 [SMILES: C=C(F)C(=O)N1CCN(c2nc(NCC34CCCN3CCC4)nc3c2COC(c2cccc4cccc(C)c24)C3)C[C@@H]1CC#N ; C=C(Cl)C(=O)N1CCN(c2nc(OCC34CCCN3CCC4)nc3c2COC(c2cccc4cccc(Cl)c24)C3)C[C@@H]1CC#N ; C/C=C/C(=O)N1CCN(c2nc(OCC34CCCN3CCC4)nc3c2COC(c2cccc4cccc(Cl)c24)C3)C[C@@H]1CC#N]

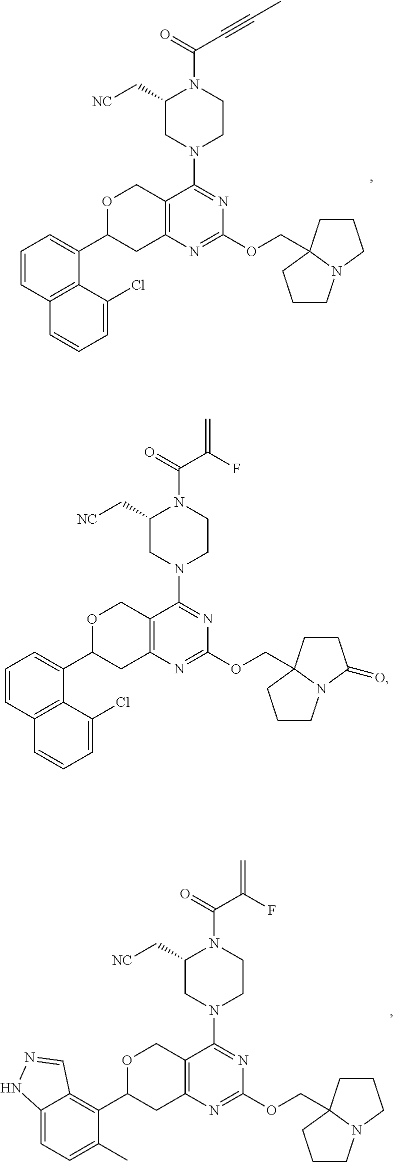 [SMILES: CC#CC(=O)N1CCN(c2nc(OCC34CCCN3CCC4)nc3c2COC(c2cccc4cccc(Cl)c24)C3)C[C@@H]1CC#N ; C=C(F)C(=O)N1CCN(c2nc(OCC34CCCN3C(=O)CC4)nc3c2COC(c2cccc4cccc(Cl)c24)C3)C[C@@H]1CC#N ; C=C(F)C(=O)N1CCN(c2nc(OCC34CCCN3CCC4)nc3c2COC(c2c(C)ccc4[nH]ncc24)C3)C[C@@H]1CC#N]

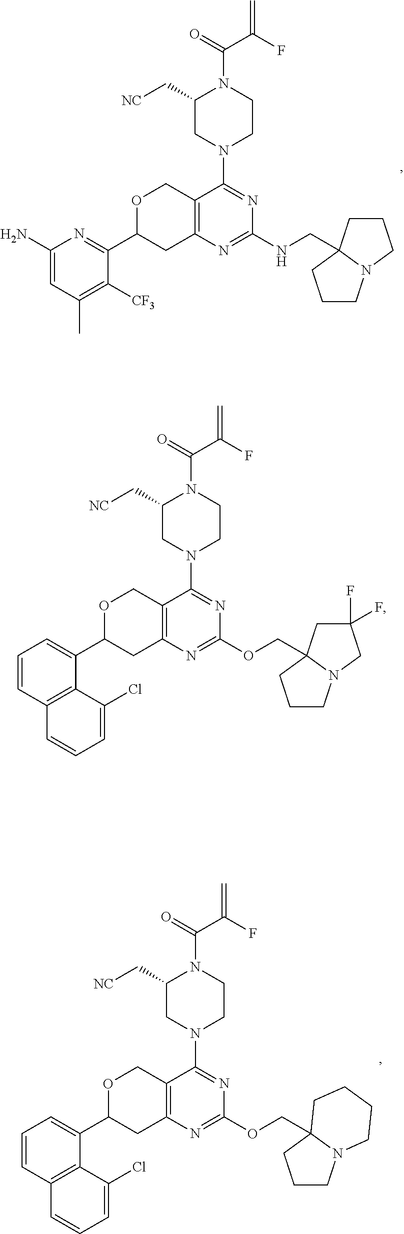 [SMILES: C=C(F)C(=O)N1CCN(c2nc(NCC34CCCN3CCC4)nc3c2COC(c2nc(N)cc(C)c2C(F)(F)F)C3)C[C@@H]1CC#N ; C=C(F)C(=O)N1CCN(c2nc(OCC34CCCN3CC(F)(F)C4)nc3c2COC(c2cccc4cccc(Cl)c24)C3)C[C@@H]1CC#N ; C=C(F)C(=O)N1CCN(c2nc(OCC34CCCCN3CCC4)nc3c2COC(c2cccc4cccc(Cl)c24)C3)C[C@@H]1CC#N]

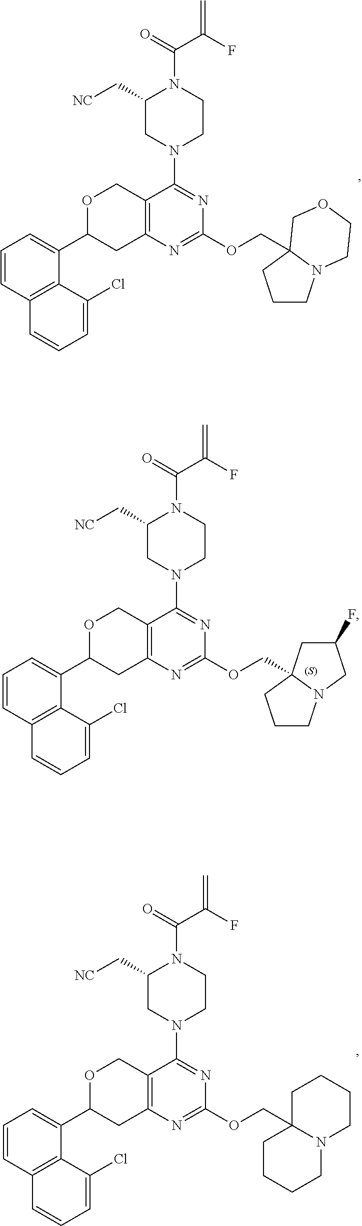

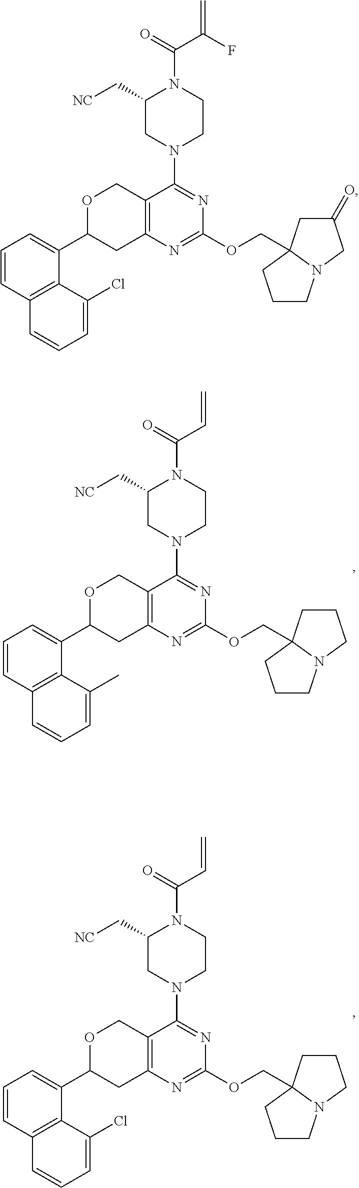 [SMILES: C=C(F)C(=O)N1CCN(c2nc(OCC34CCCN3CC(=O)C4)nc3c2COC(c2cccc4cccc(Cl)c24)C3)C[C@@H]1CC#N ; C=CC(=O)N1CCN(c2nc(OCC34CCCN3CCC4)nc3c2COC(c2cccc4cccc(C)c24)C3)C[C@@H]1CC#N ; C=CC(=O)N1CCN(c2nc(OCC34CCCN3CCC4)nc3c2COC(c2cccc4cccc(Cl)c24)C3)C[C@@H]1CC#N]

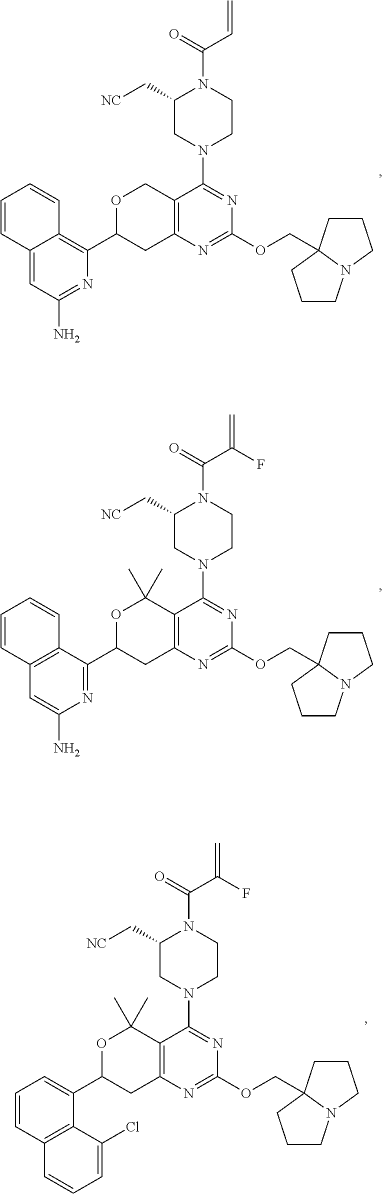 [SMILES: C=CC(=O)N1CCN(c2nc(OCC34CCCN3CCC4)nc3c2COC(c2nc(N)cc4ccccc24)C3)C[C@@H]1CC#N ; C=C(F)C(=O)N1CCN(c2nc(OCC34CCCN3CCC4)nc3c2C(C)(C)OC(c2nc(N)cc4ccccc24)C3)C[C@@H]1CC#N ; C=C(F)C(=O)N1CCN(c2nc(OCC34CCCN3CCC4)nc3c2C(C)(C)OC(c2cccc4cccc(Cl)c24)C3)C[C@@H]1CC#N]

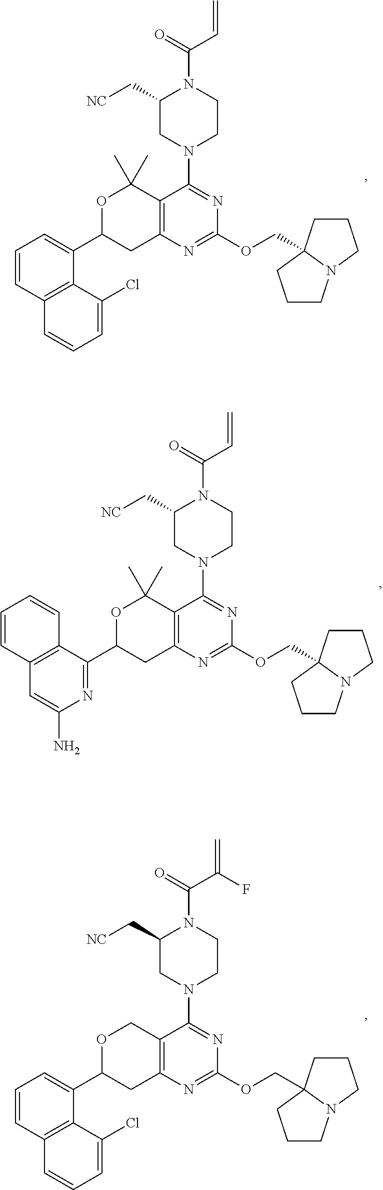

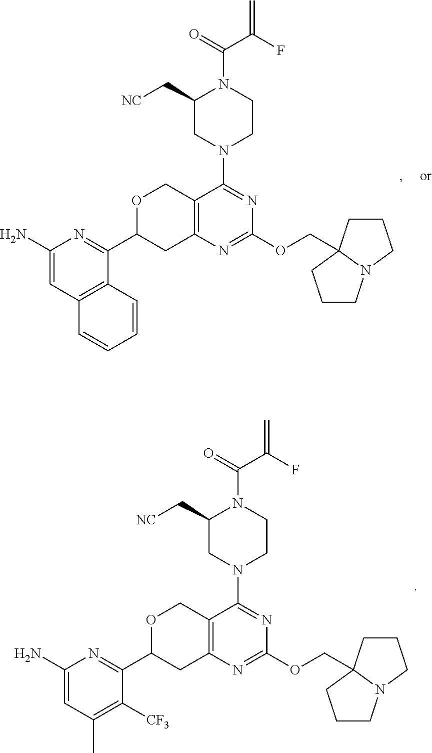 [SMILES: C=C(F)C(=O)N1CCN(c2nc(OCC34CCCN3CCC4)nc3c2COC(c2nc(N)cc4ccccc24)C3)C[C@H]1CC#N ; C=C(F)C(=O)N1CCN(c2nc(OCC34CCCN3CCC4)nc3c2COC(c2nc(N)cc(C)c2C(F)(F)F)C3)C[C@H]1CC#N]

71. The compound of claim 70, or a pharmaceutically acceptable salt thereof, wherein the compound is

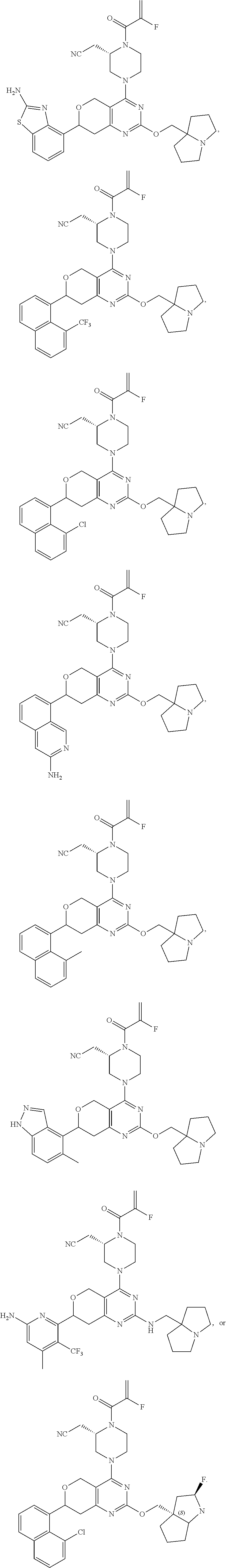

72. The compound of claim 70, or a pharmaceutically acceptable salt thereof, wherein the compound is

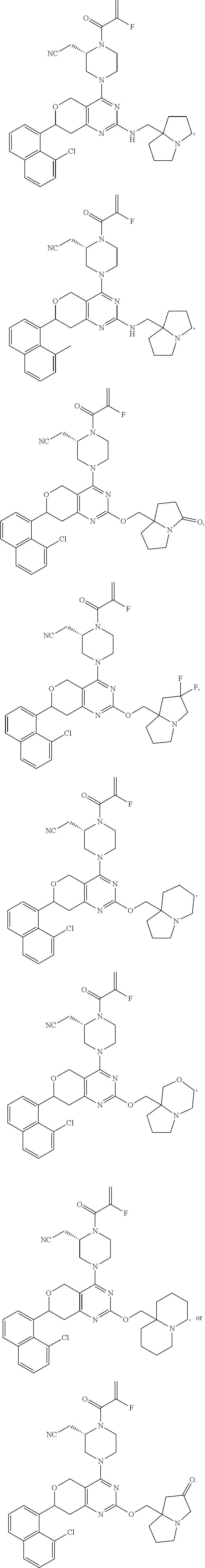

73. The compound of claim 70, or a pharmaceutically acceptable salt thereof, wherein the compound is

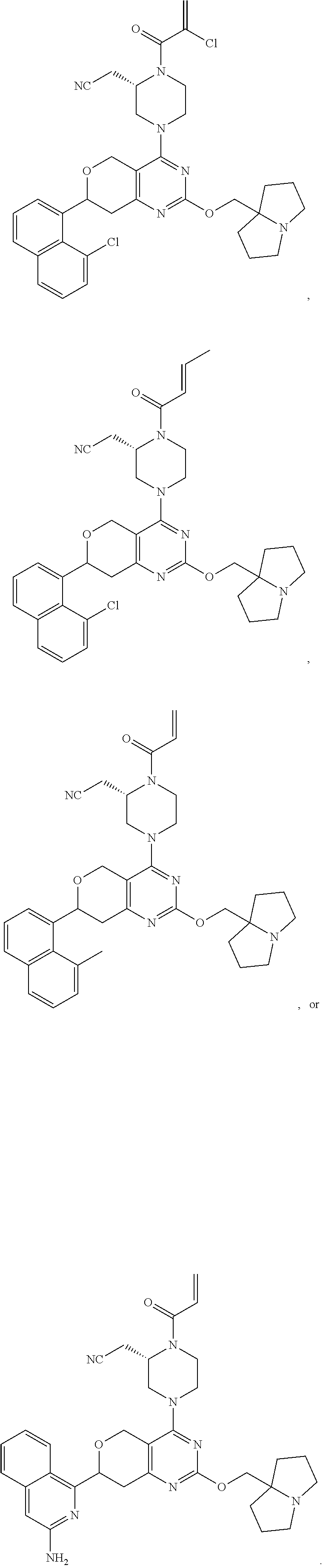 [SMILES: C=C(Cl)C(=O)N1CCN(c2nc(OCC34CCCN3CCC4)nc3c2COC(c2cccc4cccc(Cl)c24)C3)C[C@@H]1CC#N ; C/C=C/C(=O)N1CCN(c2nc(OCC34CCCN3CCC4)nc3c2COC(c2cccc4cccc(Cl)c24)C3)C[C@@H]1CC#N ; C=CC(=O)N1CCN(c2nc(OCC34CCCN3CCC4)nc3c2COC(c2cccc4cccc(C)c24)C3)C[C@@H]1CC#N ; C=CC(=O)N1CCN(c2nc(OCC34CCCN3CCC4)nc3c2COC(c2nc(N)cc4ccccc24)C3)C[C@@H]1CC#N]

74. The compound of claim 70, or a pharmaceutically acceptable salt thereof, wherein the compound is

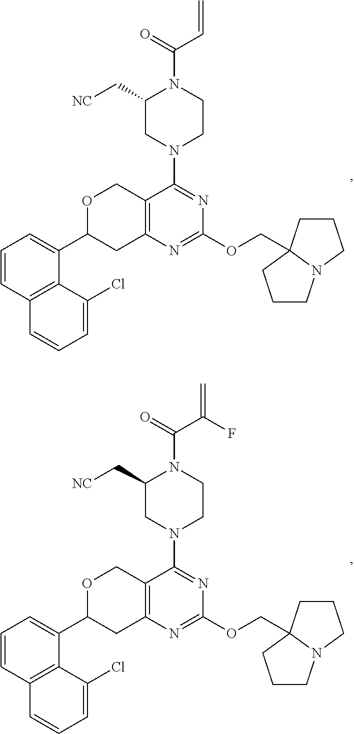 [SMILES: C=CC(=O)N1CCN(c2nc(OCC34CCCN3CCC4)nc3c2COC(c2cccc4cccc(Cl)c24)C3)C[C@@H]1CC#N ; C=C(F)C(=O)N1CCN(c2nc(OCC34CCCN3CCC4)nc3c2COC(c2cccc4cccc(Cl)c24)C3)C[C@H]1CC#N]

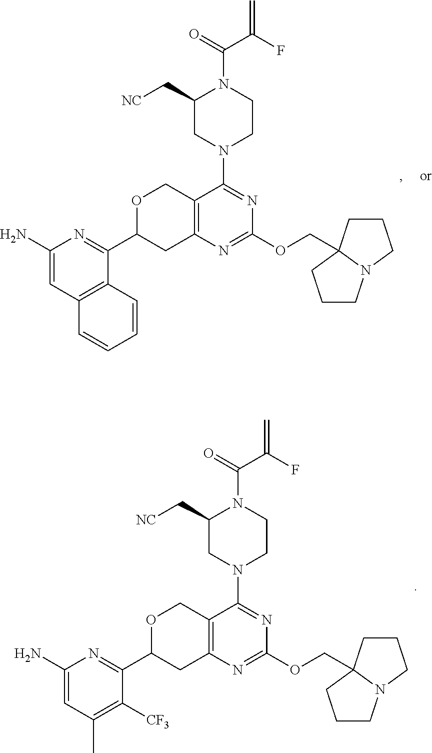 [SMILES: C=C(F)C(=O)N1CCN(c2nc(OCC34CCCN3CCC4)nc3c2COC(c2nc(N)cc4ccccc24)C3)C[C@H]1CC#N ; C=C(F)C(=O)N1CCN(c2nc(OCC34CCCN3CCC4)nc3c2COC(c2nc(N)cc(C)c2C(F)(F)F)C3)C[C@H]1CC#N]

75. The compound of claim 70, or a pharmaceutically acceptable salt thereof, wherein the compound is

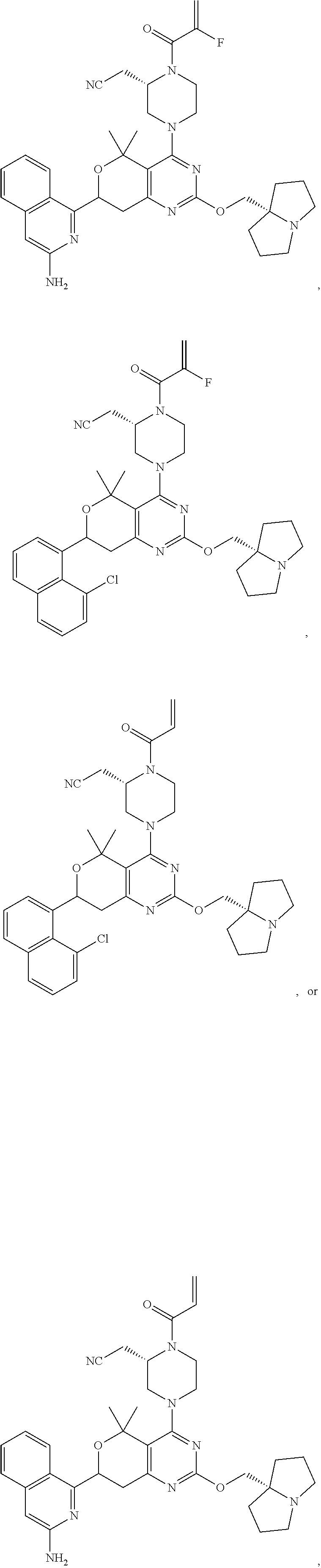

76. The compound of claim 70, or a pharmaceutically acceptable salt thereof, wherein the compound is

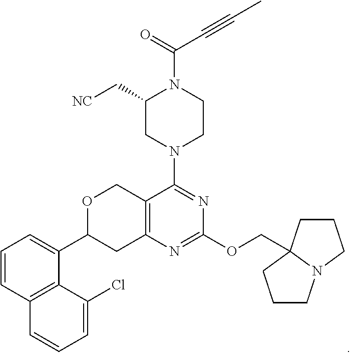 [SMILES: CC#CC(=O)N1CCN(c2nc(OCC34CCCN3CCC4)nc3c2COC(c2cccc4cccc(Cl)c24)C3)C[C@@H]1CC#N]

77. The compound of claim 76, wherein the compound is

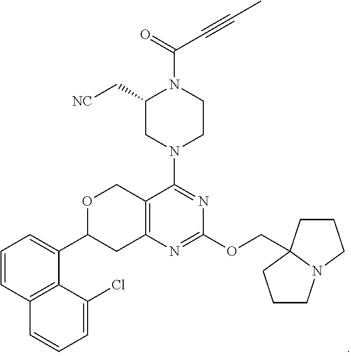 [SMILES: CC#CC(=O)N1CCN(c2nc(OCC34CCCN3CCC4)nc3c2COC(c2cccc4cccc(Cl)c24)C3)C[C@@H]1CC#N]

78. The compound of claim 70, or a pharmaceutically acceptable salt thereof, wherein the compound is

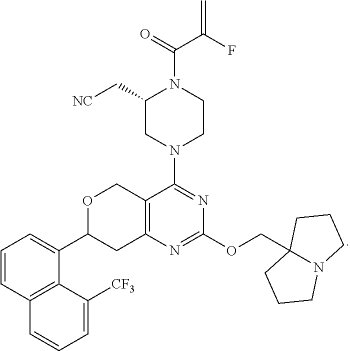 [SMILES: C=C(F)C(=O)N1CCN(c2nc(OCC34CCCN3CCC4)nc3c2COC(c2cccc4cccc(C(F)(F)F)c24)C3)C[C@@H]1CC#N]

79. The compound of claim 78, wherein the compound is

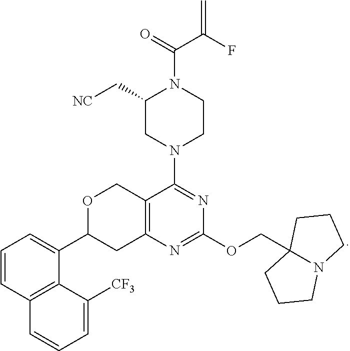 [SMILES: C=C(F)C(=O)N1CCN(c2nc(OCC34CCCN3CCC4)nc3c2COC(c2cccc4cccc(C(F)(F)F)c24)C3)C[C@@H]1CC#N]

80. The compound of claim 70, or a pharmaceutically acceptable salt thereof, wherein the compound is

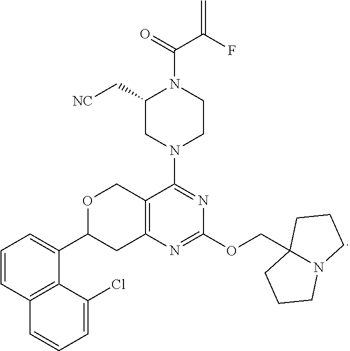 [SMILES: C=C(F)C(=O)N1CCN(c2nc(OCC34CCCN3CCC4)nc3c2COC(c2cccc4cccc(Cl)c24)C3)C[C@@H]1CC#N]

81. The compound of claim 80, wherein the compound is

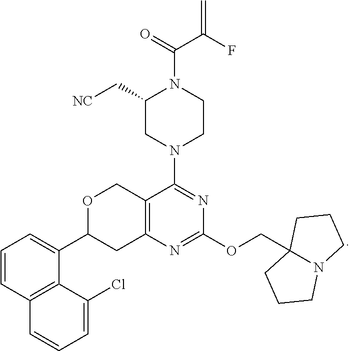 [SMILES: C=C(F)C(=O)N1CCN(c2nc(OCC34CCCN3CCC4)nc3c2COC(c2cccc4cccc(Cl)c24)C3)C[C@@H]1CC#N]

82. The compound of claim 70, or a pharmaceutically acceptable salt thereof, wherein the compound is

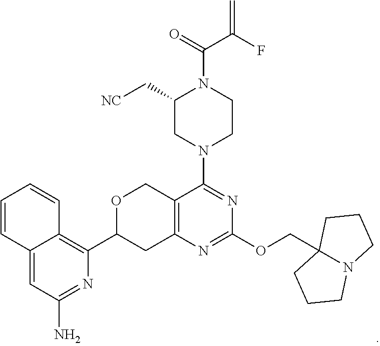 [SMILES: C=C(F)C(=O)N1CCN(c2nc(OCC34CCCN3CCC4)nc3c2COC(c2nc(N)cc4ccccc24)C3)C[C@@H]1CC#N]

83. The compound of claim 82, wherein the compound is

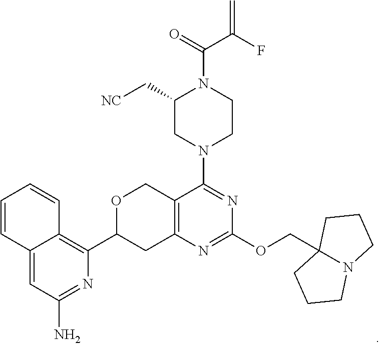 [SMILES: C=C(F)C(=O)N1CCN(c2nc(OCC34CCCN3CCC4)nc3c2COC(c2nc(N)cc4ccccc24)C3)C[C@@H]1CC#N]

84. The compound of claim 70, or a pharmaceutically acceptable salt thereof, wherein the compound is

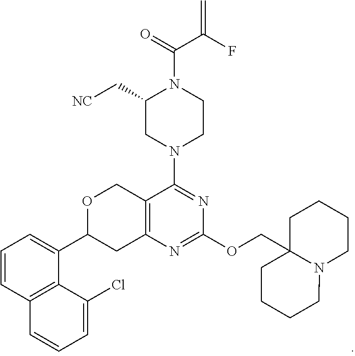 [SMILES: C=C(F)C(=O)N1CCN(c2nc(OCC34CCCCN3CCCC4)nc3c2COC(c2cccc4cccc(Cl)c24)C3)C[C@@H]1CC#N]

85. The compound of claim 84, wherein the compound is

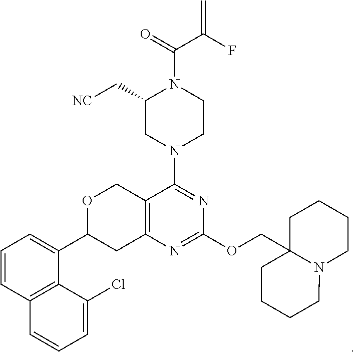 [SMILES: C=C(F)C(=O)N1CCN(c2nc(OCC34CCCCN3CCCC4)nc3c2COC(c2cccc4cccc(Cl)c24)C3)C[C@@H]1CC#N]

86. The compound of claim 70, or a pharmaceutically acceptable salt thereof, wherein the compound is

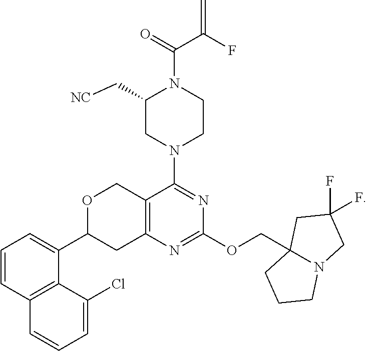 [SMILES: C=C(F)C(=O)N1CCN(c2nc(OCC34CCCN3CC(F)(F)C4)nc3c2COC(c2cccc4cccc(Cl)c24)C3)C[C@@H]1CC#N]

87. The compound of claim 86, wherein the compound is

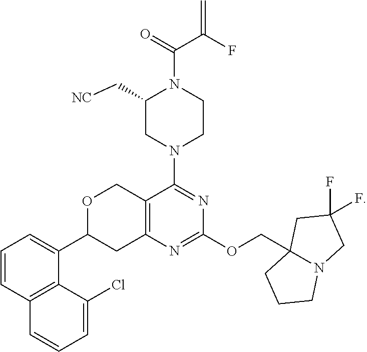 [SMILES: C=C(F)C(=O)N1CCN(c2nc(OCC34CCCN3CC(F)(F)C4)nc3c2COC(c2cccc4cccc(Cl)c24)C3)C[C@@H]1CC#N]

88. The compound of claim 70, or a pharmaceutically acceptable salt thereof, wherein the compound is

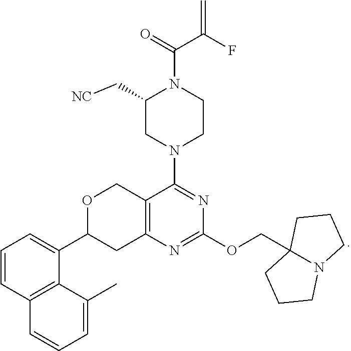 [SMILES: C=C(F)C(=O)N1CCN(c2nc(OCC34CCCN3CCC4)nc3c2COC(c2cccc4cccc(C)c24)C3)C[C@@H]1CC#N]

89. The compound of claim 88, wherein the compound is

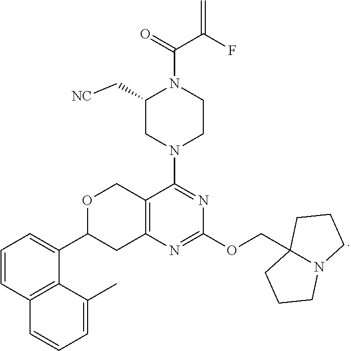 [SMILES: C=C(F)C(=O)N1CCN(c2nc(OCC34CCCN3CCC4)nc3c2COC(c2cccc4cccc(C)c24)C3)C[C@@H]1CC#N]

90. The compound of claim 70, or a pharmaceutically acceptable salt thereof, wherein the compound is

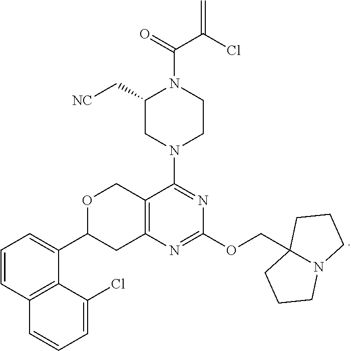 [SMILES: C=C(Cl)C(=O)N1CCN(c2nc(OCC34CCCN3CCC4)nc3c2COC(c2cccc4cccc(Cl)c24)C3)C[C@@H]1CC#N]

91. The compound of claim 90, wherein the compound is

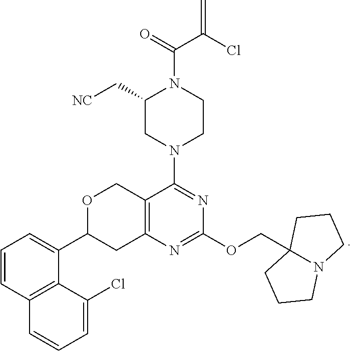 [SMILES: C=C(Cl)C(=O)N1CCN(c2nc(OCC34CCCN3CCC4)nc3c2COC(c2cccc4cccc(Cl)c24)C3)C[C@@H]1CC#N]

92. The compound of claim 70, or a pharmaceutically acceptable salt thereof, wherein the compound is

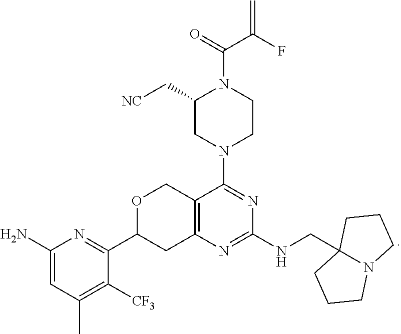 [SMILES: C=C(F)C(=O)N1CCN(c2nc(NCC34CCCN3CCC4)nc3c2COC(c2nc(N)cc(C)c2C(F)(F)F)C3)C[C@@H]1CC#N]

93. The compound of claim 92, wherein the compound is

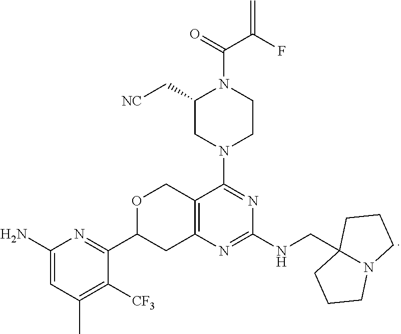 [SMILES: C=C(F)C(=O)N1CCN(c2nc(NCC34CCCN3CCC4)nc3c2COC(c2nc(N)cc(C)c2C(F)(F)F)C3)C[C@@H]1CC#N]

94. The compound of claim 70, or a pharmaceutically acceptable salt thereof, wherein the compound is

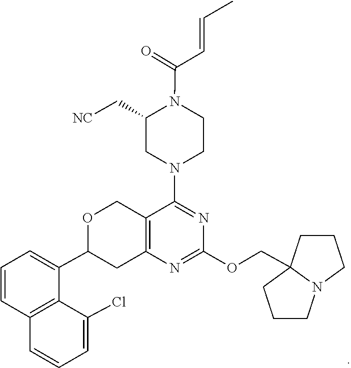 [SMILES: C/C=C/C(=O)N1CCN(c2nc(OCC34CCCN3CCC4)nc3c2COC(c2cccc4cccc(Cl)c24)C3)C[C@@H]1CC#N]

95. The compound of claim 94, wherein the compound is

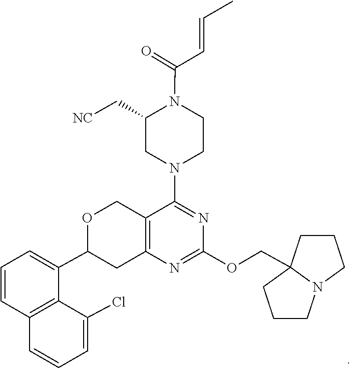 [SMILES: C/C=C/C(=O)N1CCN(c2nc(OCC34CCCN3CCC4)nc3c2COC(c2cccc4cccc(Cl)c24)C3)C[C@@H]1CC#N]

96. The compound of claim 70, or a pharmaceutically acceptable salt thereof, wherein the compound is

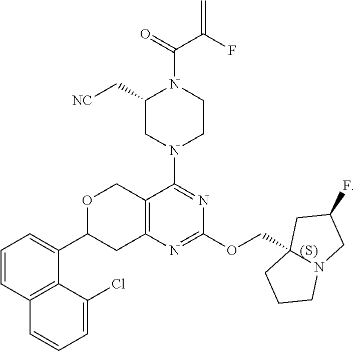

97. The compound of claim 96, wherein the compound is

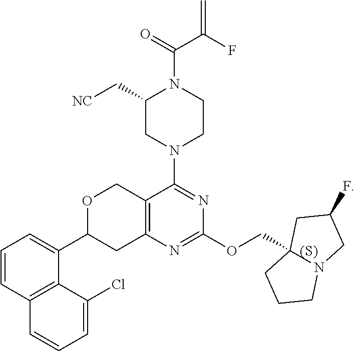

98. The compound of claim 70, or a pharmaceutically acceptable salt thereof, wherein the compound is

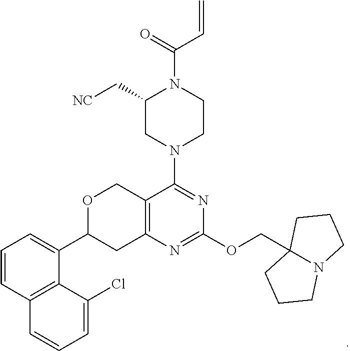 [SMILES: C=CC(=O)N1CCN(c2nc(OCC34CCCN3CCC4)nc3c2COC(c2cccc4cccc(Cl)c24)C3)C[C@@H]1CC#N]

99. The compound of claim 98, wherein the compound is

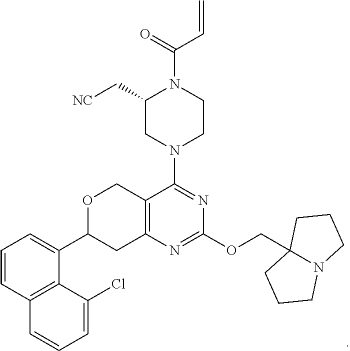 [SMILES: C=CC(=O)N1CCN(c2nc(OCC34CCCN3CCC4)nc3c2COC(c2cccc4cccc(Cl)c24)C3)C[C@@H]1CC#N]

100. The compound of claim 70, or a pharmaceutically acceptable salt thereof, wherein the compound is

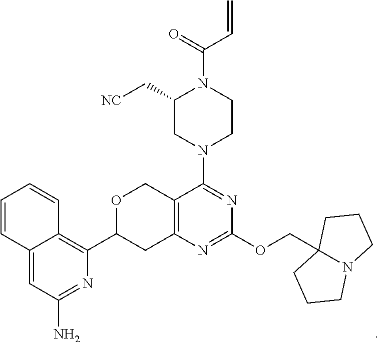 [SMILES: C=CC(=O)N1CCN(c2nc(OCC34CCCN3CCC4)nc3c2COC(c2nc(N)cc4ccccc24)C3)C[C@@H]1CC#N]

101. The compound of claim 100, wherein the compound is 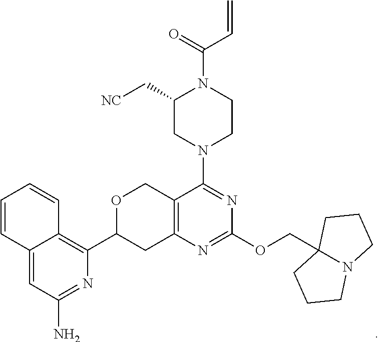 [SMILES: C=CC(=O)N1CCN(c2nc(OCC34CCCN3CCC4)nc3c2COC(c2nc(N)cc4ccccc24)C3)C[C@@H]1CC#N]
